(19) United States
(12) Patent Application Publication
(10) Pub. No.: US 2003/0203843 A1
(43) Pub. Date: Oct. 30, 2003

(54) Proteins and nucleic acids encoding same

(76) Inventors: Carol E. A. Pena, New Haven, CT (US); Xiaojia (Sasha) Guo, Branford, CT (US); Richard A. Shimkets, Guilford, CT (US); Muralidhara Padigaru, Branford, CT (US); Ramesh Kekuda, Danbury, CT (US); Kimberly A. Spytek, New Haven, CT (US); Fuad Mehraban, Trumbull, CT (US); James Newman Topper, Los Altos, CA (US); Uriel M. Malyankar, Branford, CT (US); Scott Michael Wasserman, San Francisco, CA (US); Shlomit R. Edinger, New Haven, CT (US); Glennda Smithson, Guilford, CT (US); Erik Gunther, Branford, CT (US); Laszlo Komuves, San Francisco, CA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/120,801

(22) Filed: Apr. 11, 2002

Related U.S. Application Data
(60) Provisional application No. 60/285,609, filed on Apr. 20, 2001.
(60) Provisional application No. 60/285,748, filed on Apr. 23, 2001.
(60) Provisional application No. 60/286,068, filed on Apr. 24, 2001.
(60) Provisional application No. 60/286,292, filed on Apr. 25, 2001.
(60) Provisional application No. 60/288,334, filed on May 3, 2001.
(60) Provisional application No. 60/291,241, filed on May 16, 2001.
(60) Provisional application No. 60/322,284, filed on Sep. 14, 2001.

(51) Int. Cl. A61K 38/17; C07K 14/435; C12P 21/02; C12N 5/06; C07H 21/04

(52) U.S. Cl. 514/12; 530/350; 536/23.5; 435/69.1; 435/325; 435/320.1

(57) ABSTRACT

Disclosed are polypeptides and nucleic acids encoding same. Also disclosed are vectors, host cells, antibodies and recombinant methods for producing the polypeptides and polynucleotides, as well as methods for using same.

### RELATED APPLICATIONS

This application claims priority from Provisional Applications U.S. S. No. 60/285,609 filed Apr. 20, 2001, U.S. S. No. 60/285,748, filed Apr. 23, 2001, U.S. S. No. 60/286,068, filed Apr. 24, 2001, U.S. S. No. 60/286,292, filed Apr. 25, 2001, U.S. S. No. 60/288,334, filed May 3, 2001, U.S. S. No. 60/322,284, filed Sep. 14, 2001, U.S. S. No. 60/291,241, filed May 16, 2001, each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, and methods for using the same.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

Heart disease is the primary cause of death in most western societies. Death from heart disease is often induced by platelet-dependent ischemic syndromes which are initiated by atherosclerosis and arteriosclerosis and include, but are not limited to, acute myocardial infarction, chronic unstable angina, transient ischemic attacks and strokes, peripheral vascular disease, arterial thrombosis, preeclampsia, embolism, restenosis and/or thrombosis following angioplasty, carotid endarterectomy, anastomosis of vascular grafts, and chronic cardiovascular devices (e.g., in-dwelling catheters or shunts “extracorporeal circulating devices”). These syndromes represent a variety of stenotic and occlusive vascular disorders thought to be initiated by platelet activation either on vessel walls or within the lumen by blood-borne mediators but are manifested by platelet aggregates which form thrombi that restrict blood flow.

For example, Thrombospondin-1-like proteins associate with the extracellular matrix and inhibits angiogenesis in vivo. In vitro, Thrombospondin-like proteins block capillary-like tube formation and endothelial cell proliferation. The antiangiogenic activity is mediated by a region that contains 3 type 1 (properdin or thrombospondin) repeats.

In addition, Selectin-like proteins such as P-selectin, also called GMP-140, CD62, or selectin P, is a 140-kD adhesion molecule, expressed at the surface of activated cells, that mediates the interaction of activated endothelial cells or platelets with leukocytes. In endothelial cells, the protein is localized to the membranes of Weibel-Palade bodies, the intracellular storage granules for von Willebrand factor.

Many disease states are characterized by uncontrolled cell proliferation. These diseases involve a variety of cell types and include disorders such as cancer, psoriasis, pulmonary fibrosis, glomerulonephritis, atherosclerosis and restenosis following angioplasty. Vital cellular functions such as cell proliferation and signal transduction are regulated in part by the balance between the activities of protein-tyrosine kinases (PTK) and protein-tyrosine phosphatases (PTPase). Oncogenesis can result from an imbalance.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10a, NOV10b, NOV11, NOV12, NOV13, NOV14, NOV15, and NOV16 nucleic acids and polypeptides. These nucleic acids and polypeptides, as well as variants, derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31, and 33. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32, and 34. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31, and 33.

Also included in the invention is an oligonucleotide, e.g., an oligonucleotide which includes at least 6 contiguous nucleotides of a NOVX nucleic acid (e.g. SEQ ID NOS:1, 3, 5, 7, 9, 11, 13, 15, 17, 19, 21, 23, 25, 27, 29, 31, and 33) or a complement of said oligonucleotide.

Also included in the invention are substantially purified NOVX polypeptides (SEQ ID NOS:2, 4, 6, 8, 10, 12, 14, 16, 18, 20, 22, 24, 26, 28, 30, 32, and 34). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., those described for the individual NOVX nucleotides and polypeptides herein, and/or other pathologies and disorders of the like.

The therapeutic can be, e.g., a NOVX nucleic acid, a NOVX polypeptide, or a NOVX-specific antibody, or biologically-active derivatives or fragments thereof. For example, the compositions of the present invention will have efficacy for treatment of patients suffering from the diseases and disorders disclosed below and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | Internal | SEQ ID | SEQ ID | |
|---|---|---|---|---|
| NOVX | Identification | NO (nt) | NO (aa) | Homology |
| 1 | CG93221-01 | 1 | 2 | Paladin |
| 2 | CG93210-01 | 3 | 4 | Plasma membrane |
| | | | | ring finger protein |
| 3 | CG93275-01 | 5 | 6 | Thrombospondin-1 |
| | | | | domain containing |
| | | | | protein |
| 4 | CG93187-01 | 7 | 8 | Protocadherin alpha |
| | | | | C2 short form |
| 5 | COR CG95083-01 | 9 | 10 | Nuclear protein |
| 6 | COR CG94989-01 | 11 | 12 | Secretory protein |
| 7 | COR CG94978-01 | 13 | 14 | Transmission- |
| | | | | blocking target |
| | | | | antigen S230 |
| | | | | precursor |
| 8 | COR CG94713-01 | 15 | 16 | Nuclear protein |
| 9 | COR CG94702-01 | 17 | 18 | Hemicentin |
| | | | | precursor |
| 10a | COR CG94661-01 | 19 | 20 | Selectin |
| 10b | COR CG94661-02 | 21 | 22 | Selectin |
| 11 | COR CG94325-01 | 23 | 24 | Nuclear protein |
| 12 | COR CG94282-01 | 25 | 26 | Plasma membrane |
| | | | | protein |
| 13 | COR CG94399-01 | 27 | 28 | BHLH Factor MATH6 |
| 14 | COR CG94366-01 | 29 | 30 | Putative protein- |
| | | | | tyrosine |
| | | | | phosphatase |
| 15 | CG95387-02 | 31 | 32 | LRR protein |
| 16 | CG95419-02 | 33 | 34 | RhoGEF |

NOVX nucleic acids and their encoded polypeptides are useful in a variety of applications and contexts. The various NOVX nucleic acids and polypeptides according to the inventions are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the sixteen genes, based on the tissues in which they are most highly expressed. Uses include developing products for the diagnosis or treatment of a variety of diseases and disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., cell growth, cell metabolism, cell differentiation, cell proliferation, and/or cell signaling.

In one embodiment of the present invention, NOVX or a fragment or derivative thereof may be administered to a subject to treat or prevent a disorder associated with decreased expression or activity of NOVX. Examples of such disorders include, but are not limited to, cancers such as adenocarcinoina, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; neurological disorders such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, akathesia, amnesia, catatonia, diabetic neuropathy, tardive dyskinesia, dystonias, paranoid psychoses, postherpetic neuralgia, and Tourette's disorder; and disorders of vesicular transport such as cystic fibrosis, glucose-galactose malabsorption syndrome, hypercholesteroleinia, diabetes mellitus, diabetes insipidus, hyper- and hypoglycemia, Grave's disease, goiter, Cushing's disease, Addison's disease, gastrointestinal disorders including ulcerative colitis, gastric and duodenal ulcers, other conditions associated with abnormal vesicle trafficking including acquired immunodeficiency syndrome (AIDS), allergic reactions, autoimmune hemolytic anemia, proliferative glomerulonephritis, inflammatory bowel disease, multiple sclerosis, inyasthenia gravis, rheumatoid arthritis, osteoarthritis, scleroderma, Chediak-Higashi syndrome, Sjogren's syndrome, systemic lupus erythiematosus, toxic shock syndrome, traumatic tissue damage, and viral, bacterial, fungal, helminthic, and protozoal infections, as well as additional indications listed for the individual NOVX clones.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon. Additional utilities for the NOVX nucleic acids and polypeptides according to the invention are disclosed herein.

NOV1

A NOV1 polypeptide has been identified as a Paladin-like protein (also referred to as CG93221-01). The disclosed novel NOV1 nucleic acid (SEQ ID NO:1) of 2600 nucleotides is shown in Table 1A. The novel NOV1 nucleic acid sequences maps to the chromosome 10.

An ORF begins with an ATG initiation codon at nucleotides 15-17 and ends with a TAG codon at nucleotides 2583-2585. A putative untranslated region and/or downstream from the termination codon is underlined in Table 1A, and the start and stop codons are in bold letters.

TABLE 1A
NOV1 Nucleotide Sequence

(SEQ ID NO:1)

GCTCCTGCCAGACTATGGGTACAACGGCCAGCACAGCCCAGCAGACGGTCTCGGCAGGCACCCCATT
TGAGCGCCTACAGGGCAGTGGCACGATGGACAGTCGGCACTCCGTCAGCATCCACTCCTTCCAGAGC
ACTAGCTTGCATAACAGCAAGGCCAAGTCCATCATCCCCAACAAGGTGGCCCCTGTTGTGATCACGT
ACAACTGCAAGGAGGAGTTCCAGATCCATGATGAGCTGCTCAAGGCTCATTACACGTTGGGCCGGCT
CTCGGACAACACCCCTGAGCACTACCTGGTGCAAGGCCGCTACTTCCTGGTGCGGGATGTCACTGAG
AAGATGCATGTGCTGCGCACCGTGGGAAGCTGTGGGGCCCCCAACTTCCGGCAGGTGCAGGGTGGGC
TCACTCTGTTCGGCATGGGACACCCCAGCCTCTTAGGGTTCAGGCGGGTCCTCCAGAAACTCCAGAA
CCACGCACATAGGGAGTGTGTCATCTTCTGTGTGCGGGACGAACCTGTGCTTTTCCTGCGTGCAGAT
GAGGACTTTGTGTCCTACACACCTCGAGACAAGCAGAACCTTCATGAGAACCTCCAGGGCCTTGGAC
CCGGGGTCCGGCTGGAGAGCCTGGAGCTGGCCATCCGGAAAGAGATCCACGACTTTGCCCACCTGAG
CGAGAACACATACCATCTGTACCATAACACCGAGGACCTGTGGCGGGAGCCCCATGCTGTGGCCATC
CATGGTGAGGACGACTTGCATGTGACGGAGGAGGTGTACAAGCCGCCCCTCTTCCTGCAGCCCACCT
ACAGGTACCACCGCCTCCCCCTGCCCGAGCAACGCACTCCCCTCGAGGCCCAGTTGCACGCCTTTGT
CAGTGTTCTCCGCGAGACCCCCAGCCTGCTGCACCTCCGTGATGCCCACGCGCCTCCCCCAGCCCTC
GTCTTCAGCTGCCAGATCCGCGTGCGCAGCACCAACCTGGCCATGGTCCTGGGCACCCTCATCCTCC
TTCACCGCAGTGGGACCACCTCCCAGCCAGAGGCTGCCCCCACGCAGGCCAACCCCCTGCCTATCGA
GCAGTTCCAGGTGATCCACAGCTTTCTCCGCATGGTGCCCCACGGAAGGACGATGGTGGAAGAGGTG
GACAGAGCCATCACTGCCTGTGCCGAGTTGCATGACCTGAAACAAGTGGTCTTGGAAAACCAGAAGA
AGTTAGAAGGTATCCGACCGGAGAGCCCAGCCCAGGCAAGCGGCAGCCGACACAGCGTCTGGCAGAG
GGCGCTGTGGAGCCTGGAGCGATACTTCTACCTGATCCTGTTTAACTACTACCTTCATGAGQAGTAC
CCGCTGGCCTTTGCCCTCAGTTTCAGCCCCTGGCTGTGTGCCCACCCTGAGCTGTACCGCCTGCCCC
TCACGCTGACCTCAGCAGCCCCTGTGGCTCCCAGGCACCTCATCGCCAGGGGCTCCCTACGGGAGGA
CGATCTGGTCTCCCCGGACGCGCTCAGCACTGTCAGAGAGATGGATGTGGCCAACTTCCGGCGGGTC
CCCCGCATGCCCATCTACGGCACGGCCCAGCCCAGCGCCAAGGCCCTGCGGAGCATCCTGGCCTACC
TGACGGACGCCAAGAGCAGGCTCCGGAAGGTTGTCTGGGTGAGCCTTCGGGACGAGGCCCTGTTGGA
CTGTGACGGGCACACCTACACCCTGCGGTGGCCTGGGCCCCCTGTGGCTCCTGACCAGCTGGACACC
CTGGAGGCCCAGCTGAAGGCCCATCTAAGCGAGCCTCCCCCAGGCAAGGAGGGCCCCCTGACCTACA
GGTTCCACACCTGCCTTACCATGCAGGAGGTCTTCAGCCACCACCGCAGCGCCTGTCCTGGCCTCAC
CTACCACCCCATCCCCATGCCGGACTTCTGTGCCCCCCGAGAGGAGGACTTTGACCAGCTGCTGGAG
GCCCTGCGGGCCGCCCTCTCCAACGACCCAGGCACTGCCTTCGTGTTCAGCTGCCTCAGCGGCCACG
GCCGTACCACAACTGCGATGCTGGTGCCTGTCCTGGCCTTCTGGCACATCCAAGCCTTCCCCCAGGT
GGGTGAGGAGGAGCTCGTGAGTCTGCCTGATGCCAAGTTCACTAAGGGTGAATTTCAGGTAGTAATG
AAGGTGGTGCAGCTGCTACCCGATGGGCACCGTGTGAAGAAGGAGGTGCACGCACCGCTGGACACTG
TCAGCCAGACCATGACGCCCATCCACTACCACCTGCGGGAGATCATCATCTGCACCTACCCCCAGGC
GAAGGCAGCGAAAGAGGCGCAGGAAATGCOGACGCTCCACCTGCGGAGCCTGCAGTACTTCCACCGC
TATGTCTGCCTGATTCTCTTCAACGCGTACCTCCACCTGGAGAAGGCCGACTCCTGGCAGAGGCCCT
TCAGCACCTGGATGCAGGAGCTGGCATCGAAGGCTCGCATCTACGAGATCCTTAACGAGCTGGGCTT
CCCCGAGCTGGAGAGCCGGCAGGACCAGCCCTTCTCCAGGCTGCCCTACCGGTGGCAGGAGCAGAGC
TCCAGCCTCGAGCCCTCTGCCCCCGACCACTTGCTCTAGGGGGCCTTACTCCCT

Variant sequences of NOV1 are included in Example 3, Table 18. A variant sequence can include a single nucleotide polymorphism (SNP). A SNP can, in some instances, be referred to as a “cSNP” to denote that the nucleotide sequence containing the SNP originates as a cDNA.

The NOV1 protein (SEQ ID NO:2) encoded by SEQ ID NO:1 is 856 amino acid residues in length and is presented using the one-letter amino acid code in Table 1B. Psort analysis predicts the NOV1 protein of the invention to be localized in the cytoplasm with a certainty of 0.4500.

TABLE 1B
Encoded NOV1 protein sequence

(SEQ ID NO:2)

MGTTASTAQQTVSAGTPFEGLQGSGTMDSRHSVSIHSFQSTSLHNSKAKSIIPNKVAPVVITYNC
KEEFQIHDELLKAHYTLGRLSDNTPEHYLVQGRYFLVRDVTEKMDVLGTVGSCGAPNFRQVQGGL
TVFGMGQPSLLGFRRVLQKLQKDGHRECVIFCVREEPVLFLRADEDFVSYTPRDKQNLHENLQGL
GPGVRVESLELAIRKEIHDFAQLSENTYHVYHNTEDLWGEPHAVAIHGEDDLHVTEEVYKRPLFL
QPTYRYHRLPLPEQGSPLEAQLDAFVSVLRETPSLLQLRDAHGPPPALVFSCQMGVGRTNLGMVL
GTLILLHRSGTTSQPEAAPTQAKPLPMEQFQVIQSFLRMVPQGRRMVEEVDRAITACAELHDLKE
VVLENQKKLEGIRPESPAQGSGSRHSVWQRALWSLERYFYLILFNYYLHEQYPLAFALSFSRWLC
AHPELYRLPVTLSSAGPVAPRDLIARGSLREDDLVSPDALSTVREMDVANFRRVPRMPIYGTAQP
SAKALGSILAYLTDAKRRLRKVVWVSLREEAVLECEGHTYSLRWPGPPVAPDQLETLEAQLKAHL
SEPPPGKEGPLTYRFQTCLTMQEVFSQHRRACPGLTYHRIPMPDFCAPREEDFDQLLEALRAALS
KDPGTGFVFSCLSGQCRTTTAMVVAVLAFWHIQGFPEVGEEELVSVPDAKFTKGEFQVVMKVVQL
LPDGHRVKKEVDAALDTVSETMTPMHYHLREIIICTYRQAKAAKEAQEMRRLQLRSLQYLERYVC
LILFNAYLHLEKADSWQRPFSTWMQEVASKAGIYEILNELGFPELESGEDQPFSRLRYRWQEQSC
SLEPSAPEDLL

In all BLAST alignments described herein, the “E-value” or “Expect” value is a numeric indication of the probability that the aligned sequences could have achieved their similarity to the BLAST query sequence by chance alone, within the database that was searched. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used to create a significance threshold for reporting results. The default value used for blasting is typically set to 0.0001, with the filter to remove low complexity sequence turned off. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits The filter substitutes any low-complexity sequence that it finds with the letter “N” in nucleotide sequence (e.g., “NNNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, (1996).

A search against the Patp database, a proprietary database that contains sequences published in patents and patent publications, yielded several homologous proteins shown in Table 1C.

TABLE 1C
Patp results for NOV1

| | | | Smallest |
|---|---|---|---|
| | | | Sum |
| Sequences producing High-scoring | Reading | High | Prob |
| Segment Pairs: | Frame | Score | P (N) |
| >patp:AAB41108 Human ORFX ORF872 | +1 | 4187 | 0.0 |
| polypeptide | | | |
| >patp:AAB35276 Murine dual specificity | +1 | 120 | 5.2e−06 |
| phosphatase DSP-11 | | | |
| >patp:AAB73211 Murine phosphatase | +1 | 120 | 5.2e−06 |
| AA023073_m | | | |
| >patp:AAB73231 Human phosphatase | +1 | 115 | 1.8e−05 |
| BAA91172_h | | | |
| >patp:AAG67455 Amino acid sequence of | +1 | 115 | 1.8e−05 |
| a human polypeptide | | | |

In a BLAST search of public sequence databases, it was found, for example, that the nucleic acid sequence of this invention has 2063 of 2508 bases (82%) identical to a gb:GENBANK-ID:MMPAL|acc:X99384.1 mRNA from Mus musculus (Paladin gene). The full amino acid sequence of the protein of the invention was found to have 695 of 859 amino acid residues (80%) identical to, and 754 of 859 amino acid residues (87%) similar to, the 859 amino acid residue ptnr:SPTREMBL-ACC:P70261 protein from Mus musculus (PALADIN GENE). NOV1 also has homology to the proteins shown in the BLASTP data in Table 1D.

TABLE 1D
BLAST results for NOV1

| Gene Index/ | | | | | |
|---|---|---|---|---|---|
| Identifier | Protein/Organism | Length (aa) | Identity (%) | Positives (%) | Expect |
| gi\|6331287\|dbj\| | KIAA1274 protein | 752 | 752/752 | 752/752 | 0.0 |
| BAA86588.1\| | [Homo sapiens] | | (100%) | (100%) | |
| (AB033100) | | | | | |
| gi\|14738662\|ref\|XP_{—} | KIAA protein | 748 | 747/748 | 747/748 | 0.0 |
| 046314.1\| | (similar to mouse | | (99%) | (99%) | |
| (XM_046314) | paladin) [Homo | | | | |
| | sapiens] | | | | |
| gi\|7305365\|ref\|NP_{—} | paladin [Mus | 859 | 673/841 | 730/841 | 0.0 |
| 038781.1\| | musculus] | | (80%) | (86%) | |
| (NM_013753) | | | | | |
| gi\|15228672\|ref\|NP_{—} | putative protein | 1232 | 207/821 | 340/821 | 7e−45 |
| 191760.1\| | [Arabidopsis | | (25%) | (41%) | |
| (NM_116066) | thaliana] | | | | |
| gi\|12836455\|dbj\| | data source: SPTR, | 144 | 24/60 | 33/60 | 2e−04 |
| BAB23663.1\|(AK004912) | source | | (40%) | (55%) | |
| | key: Q9NX48, | | | | |
| | evidence: ISS˜homo | | | | |
| | log to CDNA | | | | |
| | FLJ20442 FIS, | | | | |
| | CLONE | | | | |
| | KAT04828˜putative | | | | |
| | [Mus musculus] | | | | |

A multiple sequence alignment is given in Table 1E, with the NOV1 protein being shown on line 1 in Table 1E in a ClustalW analysis, and comparing the NOV1 protein with the related protein sequences shown in Table 1D. This BLASTP data is displayed graphically in the ClustalW in Table 1E.

[image]

[image]

[image]

[image]

The NOV1 Clustal W alignment shown in Table 1E was modified to begin at amino residue 1050. The data in Table 1E includes all of the regions overlapping with the NOV1 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 1F lists the domain description from DOMAIN analysis results against NOV1.

TABLE 1F
Domain Analysis of NOV1

| | Region of | | |
|---|---|---|---|
| Model | Homology | Score (bits) | E value |
| #PD396342 | 19-118 | 431 | 4e−43 |
| PALADIN GENE | | | |
| #PD148197 | 119-399 | 1175 | e−129 |
| PALADIN GENE | | | |
| #PD222597 DOMAIN | 119-231 | 119 | 6e−07 |
| OF UNKNOWN | | | |
| #PD306865 | 354-454 | 132 | 2e−08 |
| PALADIN GENE | | | |
| #PD024454 | 356-445 | 84 | 0.007 |
| PLASMID ORFS | | | |
| #PD325716 | 400-604 | 800 | 6e−86 |
| PALADIN GENE | | | |
| #PD326847 | 458-594 | 97 | 2e−04 |
| CG18442 | | | |
| #PD222597 DOMAIN | 505-602 | 113 | 3e−06 |
| OF UNKNOWN | | | |
| #PD277963 | 595-648 | 97 | 2e−04 |
| HYDROLASE | | | |
| KAT04828 FIS CDNA | | | |
| #PD148197 | 605-678 | 340 | 1e−32 |
| PALADIN GENE | | | |
| #PD306865 | 680-856 | 765 | 7e−82 |
| PALADIN GENE | | | |
| #PD325716 | 751-820 | 86 | 0.004 |
| PALADIN GENE | | | |

Consistent with other known members of the Paladin-like family of proteins, NOV1 has, for example, multiple Paladin gene signature sequences and homology to other members of the Paladin-like Protein Family. NOV1 nucleic acids, and the encoded polypeptides, according to the invention are useful in a variety of applications and contexts. For example, NOV1 nucleic acids and polypeptides can be used to identify proteins that are members of the Paladin like family of proteins. The NOV1 nucleic acids and polypeptides can also be used to screen for molecules, which inhibit or enhance NOV1 activity or function. Specifically, the nucleic acids and polypeptides according to the invention may be used as targets for the identification of small molecules that modulate or inhibit, e.g., cellular activation, cellular metabolism and signal transduction. These molecules can be used to treat, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, diabetes, Von Hippel-Lindau (VHL) syndrome, pancreatitis, obesity, hyperthyroidism and hypothyroidism, hypercalceimia, ulcers, cirrhosis, transplantation, inflammatory bowel disease, diverticular disease, hemophilia, hypercoagulation, idiopathic thrombocytopenic purpura, autoimmume disease, allergies, immunodeficiencies, transplantation, graft vesus host, hemophilia, hypercoagulation, idiopathic thrombocytopenic purpura, autoimmume disease, allergies, immunodeficiencies, transplantation, graft versus host disease (GVHD), lymphedema, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancer, trauma, regeneration (in vitro and in vivo), viral/bacterial/parasitic infections, as well as other diseases, disorders and conditions.

In addition, various NOV1 nucleic acids and polypeptides according to the invention are useful, inter alia, as novel members of the protein families according to the presence of domains and sequence relatedness to previously described proteins. For example, the NOV1 nucleic acids and their encoded polypeptides include structural motifs that are characteristic of proteins belonging to the Paladin-like protein family.

Paladin proteins are a family of protein-tyrosine phosphatases. The protein phosphatases can be divided into 2 large families: the serine/threonine phosphatases, which are metalloproteins, and the protein-tyrosine phosphatases, which proceed via a thiol-phosphate enzyme intermediate. The protein-tyrosine phosphatase family includes the VHI 1-like dual-specificity phosphatases. These phosphatases dephosphorylate phosphotyrosine- as well as phosphoserine- and phosphothreonine-containing substrates. Members of the dual-specificity phosphatase protein family inactivate mitogen-activated protein (MAP) kinase through dephosphorylation of critical threonine and tyrosine residues. Members of the MAP kinase family play a pivotal role in cellular signal transduction. Using a subtractive screen of mouse gastrulation, Pearce et al. (1996) identified a novel mouse gene, paladin, with similarity to the dual specificity protein phosphatase family.

The NOV1 nucleic acids and polypeptides, antibodies and related compounds according to the invention will be useful in therapeutic and diagnostic applications in the mediation of cardiac and endocrine physiology. As such, the NOV1 nucleic acids and polypeptides, antibodies and related compounds according to the invention may be used to treat muscle and nervous system disorders, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scieroderma, obesity, transplantation, adrenoleukodystrophy, congenital adrenal hyperplasia, diabetes, Von Hippel-Lindau (VHL) syndrome, pancreatitis, obesity, hyperthyroidism and hypothyroidism, hypercalceimia, ulcers, cirrhosis, transplantation, inflammatory bowel disease, diverticular disease, hemophilia, hypercoagulation, idiopathic thrombocytopenic purpura, autoimmume disease, allergies, immunodeficiencies, transplantation, graft vesus host, hemophilia, hypercoagulation, idiopathic thrombocytopenic purpura, autoimmume disease, allergies, immunodeficiencies, transplantation, graft versus host disease (GVHD), lymphedema, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancer, trauma, regeneration (in vitro and in vivo), viral/bacterial/parasitic infections, as well as other diseases, disorders and conditions.

The NOV1 nucleic acids and polypeptides are useful for detecting specific cell types. For example, expression analysis has demonstrated that a NOV1 nucleic acid is expressed in brown adipose, heart, aorta, vein, umbilical vein, adrenal gland/suprarenal gland, pancreas, thyroid, salivary glands, parotid salivary glands, stomach, liver, gall bladder, small intestine, colon, bone marrow, lymphoid tissue, spleen, lymph node, tonsils, thymus, cartilage, muscle, brain, thalamus, hypothalamus, pituitary gland, amygdala, substantia nigra, hippocampus, spinal chord, cervix, mammary gland/breast, ovary, placenta, uterus, vulva, prostate, testis, lung, lung pleura, kidney, retina, dermis.

Additional utilities for NOV1 nucleic acids and polypeptides according to the invention are disclosed herein.

NOV2

A NOV2 polypeptide has been identified as a Plasma Membrane Ring Finger-like protein (also referred to as CG93210-01). The disclosed novel NOV2 nucleic acid (SEQ ID NO:3) of 1205 nucleotides is shown in Table 2A. The novel NOV2 nucleic acid sequences maps to the chromosome 22.

An ORF begins with an ATG initiation codon at nucleotides 17-19 and ends with a ATT codon at nucleotides 1149-1151. A putative untranslated region and/or downstream from the termination codon is underlined in Table 2A, and the start and stop codons are in bold letters.

TABLE 2A
NOV2 Nucleotide Sequence

(SEQ ID NO:3)

CTCCCCCGGTCCGGCCATGGGCCCCCCCGCTCCCCCCGCGCTGAGATCGCCGCCGCCGCCTCCGCCG
CCGCCTCCGTCTCCGCTGCTGCTGCTGCTGCCCCTGCTGCCGCTGTGGCTGGGCCTGGCCGGGCCCC
GCGCCGCGGCGGACCGCAGCCACCCGGCGGCCGGGGCGCGGCGGGCCGGAGCCCCCGCCGTGCGGGT
GGACCTGAGACTCCCGCGCCAGGACGCTCTGGTCCTGGAGGGCGTCAGGATCGGCTCCGAAGCCGAC
CCGGCCCCCCTGCTGGGCGGTCGTCTGCTGCTGATGGACATCGTGGATGCCGAGCAGGAGGCACCAG
TGGAAGGCTGGATTGCAGTCGCATACGTGGGCAAGGAGCAGGCGGCCCAGTTCCACCAGGAGAATAA
GGGCAGTGGCCCGCAGGCCTATCCCAACGCCCTGGTCCAGCAGATCCGGCGCGCCCTCTTCCTGGGT
GCCTCTGCCCTGCTTCTTCTCATCCTGAACCACAACGTGGTCCGACAGCTGGACATATCCCAGCTTC
TGCTCAGGCCAGTGATCGTCCTCCATTATTCCTCCAATGTCACCAAGCTGTTGGATGCATTGCTGCA
GAGGACCCAGCCCACGGCTGACATCACCAGCGGAGAGTCCCTGTCTGCCAATATCGAGTCGAAGTTG
ACCTTGTGGACCACCTGTGGCCTCTCCAAGGATGGCTATGGAGGATGGCACGACTTGGTCTGCCTTG
GAGGCAGTCCTGCCCAGGAGCACAAACCCCTGCAGCAGCTGTGGAACCCCATCCTGCTCGTGGCCAT
GCTCCTGTGCACAGGCCTCGTGGTCCAGGCCCAGCGGCAGGCGTCGCCGCAGAGCCAGCGGGAGCTC
GGAGGCCACGTGGACCTGTTTAAGCGCCGCGTGCTGCGGAGACTGGCATCCCTCAAGACACGGCGCT
GCCGGCTGAGCAGGGCAGCGCAGGGCCTCCCAGATCCGCGTGCTGAGACCTGTGCGGTGTGCCTGGA
CTACTTCTGCAACAAACAGTGGCTCCGGGTGCTGCCCTGTAAGCACGAGTTTCACCGAGACTGTGTG
GACCCCTGGCTGATGCTCCACCAGACCTGCCCACTGTGCAAATTCAACGTCCTGCGTGAGCACCGCT
ACTCCGATGATTAGCTGCCCAGCTGGACTCTGCACATCGCGATGGACCCCTCCTCCCTGCACCCCC

The NOV2 protein (SEQ ID NO:4) encoded by SEQ ID NO:3 is 378 amino acid residues in length and is presented using the one-letter amino acid code in Table 2B. Psort analysis predicts the NOV2 protein of the invention to be localized at the plasma membrane with a certainty of 0.6400.

TABLE 2B
Encoded NOV2 protein sequence

(SEQ ID NO:4)

MGPAARPALRSPPPPPPPPPSPLLLLLPLLPLWLGLACPGAAADGSEPAAGAGRGGARAVRVDVR
LPRQDALVLEGVRIGSEADPAPLLGGRLLLMDIVDAEQEAPVEGWIAVAYVGKEQAAQFHQENKG
SGPQAYPKALVQQMRRALFLGASALLLLILNHNVVRELDISQLLLRPVIVLHYSSNVTKLLDALL
QRTQATAEITSGESLSANIEWKLTLWTTCGLSKDCYGGWQDLVCLGGSRAQEQKPLQQLWNAILL
VAMLLCTGLVVQAQRQASRQSQRELGGQVDLFKRRVVRRLASLKTRRCRLSRAAQGLPDPGAETC
AVCLDYFCNKQWLRVLPCKHEFHRDCVDPWLMLQQTCPLCKFNVLGEHRYSDD

A search against the Patp database, a proprietary database that contains sequences published in patents and patent publications, yielded several homologous proteins shown in Table 2C.

TABLE 2C
Patp results for NOV2

| | | | Smallest |
|---|---|---|---|
| | | | Sum |
| Sequences producing High-scoring | Reading | High | Prob |
| Segment Pairs: | Frame | Score | P (N) |
| >patp:AAB42695 Human ORFX ORF2459 | +1 | 1715 | 3.0e−176 |
| polypeptide | | | |
| >patp:AAM79288 Human protein SEQ | +1 | 612 | 2.3e−59 |
| ID NO 1950 | | | |
| >patp:AAM80272 Human protein SEQ | +1 | 534 | 4.2e−51 |
| ID NO 3918 | | | |
| >patp:AAU28202 Novel human | +1 | 201 | 5.1e−13 |
| secretory protein | | | |
| >patp:ABB50251 Human transcription | +1 | 148 | 5.5e−13 |
| factor TRFX-102 | | | |

In a BLAST search of public sequence databases, it was found, for example, that the nucleic acid sequence of this invention has 287 of 489 bases (58%) identical to a gb:GENBANK-ID:SS1132828|acc:AJ132828.1 mRNA from Spermatozopsis similis (mRNA for p210 protein, partial). The full amino acid sequence of the protein of the invention was found to have 341 of 379 amino acid residues (89%) identical to, and 355 of 379 amino acid residues (93%) similar to, the 379 amino acid residue ptnr:SPTREMBL-ACC:Q9DCW1 protein from Mus musculus (0610009J22RIK PROTEIN).

NOV2 also has homology to the proteins shown in the BLASTP data in Table 2D.

TABLE 2D
BLAST results for NOV2

| Gene Index/ | | | | | |
|---|---|---|---|---|---|
| Identifier | Protein/Organism | Length (aa) | Identity (%) | Positives (%) | Expect |
| gi\|12832380\|dbj\| | data source: SPTR, | 379 | 340/380 | 354/380 | e−173 |
| BAB22082.1\|(AK002414) | source | | (89%) | (92%) | |
| | key: Q9Y6U7, | | | | |
| | evidence: ISS˜homo | | | | |
| | log to | | | | |
| | WUGSC: H_DJ130H16.6 | | | | |
| | PROTEIN (FRAGMENT)˜ | | | | |
| | putative | | | | |
| | [Mus musculus] | | | | |
| gi\|5441942\|gb\| | supported by | 347 | 336/336 | 336/336 | e−148 |
| AAD43187.1\|AC004997_5 | mouse EST | | (100%) | (100%) | |
| (AC004997) | AA538043 | | | | |
| | (NID: g2284036) | | | | |
| | [Homo sapiens] | | | | |
| gi\|17485136\|ref\|XP_{—} | similar to data | 272 | 271/283 | 272/283 | e−146 |
| 066294.1\| | source: SPTR, | | (95%) | (95%) | |
| (XM_066294) | source key: Q9Y6U7 | | | | |
| | evidence: ISS˜homo | | | | |
| | log to | | | | |
| | WUGSC: H_DJ130H16.6 | | | | |
| | PROTEIN (FRAGMENT)˜ | | | | |
| | putative | | | | |
| | [Homo sapiens] | | | | |
| gi\|17861674\|gb\|AAL3 | GH20973p | 461 | 26/57 | 42/57 | 9e−13 |
| 9314.1\|(AY069169) | [Drosophila | | (45%) | (73%) | |
| | melanogaster] | | | | |
| gi\|18485962\|ref\|XP_{—} | similar to | 461 | 26/57 | 42/57 | 1e−12 |
| 080778.1\| | goliath (H. | | (45%) | (73%) | |
| (XM_080778) | sapiens) | | | | |
| | [Drosophila | | | | |
| | melanogaster] | | | | |

A multiple sequence alignment is given in Table 2E, with the NOV2 protein being shown on line 1 in Table 2E in a ClustalW analysis, and comparing the NOV2 protein with the related protein sequences shown in Table 2D. This BLASTP data is displayed graphically in the ClustalW in Table 2E.

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 2F lists the domain description from DOMAIN analysis results against NOV2.

TABLE 2F
Domain Analysis of NOV2

| | Region of | | |
|---|---|---|---|
| Model | Homology | Score (bits) | E value |
| Ring Finger | 325-365 | 49.3 | 4.0e−07 |
| zf-C3HC4 | 325-365 | 34.7 | 2.2e−09 |
| PHD | 324-368 | −10.4 | 1.1 |

Consistent with other known members of the Membrane Ring Finger-like family of proteins, NOV2 has, for example, a Ring Finger signature sequence and homology to other members of the Plasma Membrane Ring Finger-like Protein Family. NOV2 nucleic acids, and the encoded polypeptides, according to the invention are useful in a variety of applications and contexts. For example, NOV2 nucleic acids and polypeptides can be used to identify proteins that are members of the Plasma Membrane Ring Finger-like Protein Family. The NOV2 nucleic acids and polypeptides can also be used to screen for molecules, which inhibit or enhance NOV1 activity or function. Specifically, the nucleic acids and polypeptides according to the invention may be used as targets for the identification of small molecules that modulate or inhibit, e.g., cellular activation, cellular metabolism and signal transduction. These molecules can be used to treat, e.g., anemia, ataxia-telangiectasia, autoimmume disease, immunodeficiencies, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, cancer, trauma, regeneration (in vitro and in vivo), viral/bacterial/parasitic infections, as well as other diseases, disorders and conditions.

In addition, various NOV2 nucleic acids and polypeptides according to the invention are useful, inter alia, as novel members of the protein families according to the presence of domains and sequence relatedness to previously described proteins. For example, the NOV2 nucleic acids and their encoded polypeptides include structural motifs that are characteristic of proteins belonging to the Plasma Membrane Ring Finger-like Protein Family.

The NOV2 nucleic acids and polypeptides, antibodies and related compounds according to the invention will be useful in therapeutic and diagnostic applications in the mediation of immune and renal physiology. As such, the NOV2 nucleic acids and polypeptides, antibodies and related compounds according to the invention may be used to treat muscle and nervous system disorders, e.g., anemia, ataxia-telangiectasia, autoimmume disease, immunodeficiencies, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, cancer, trauma, regeneration (in vitro and in vivo), viral/bacterial/parasitic infections, as well as other diseases, disorders and conditions.

The NOV2 nucleic acids and polypeptides are useful for detecting specific cell types. For example, expression analysis has demonstrated that a NOV2 nucleic acid is expressed in peripheral blood, and a pool of various mammalian tissues. Expression information was derived from the tissue sources of the sequences that were included in the derivation of the sequence of CuraGen Acc. No. CG93210-01. The sequence is predicted to be expressed in the following tissues because of the expression pattern of (GENBANK-ID: gb:GENBANK-ID:SSI132828|acc:AJ132828.1) a closely related Spermatozopsis similis mRNA for p210 protein, partial hoinolog in species Spermatozopsis similis: kidney.

Additional utilities for NOV2 nucleic acids and polypeptides according to the invention are disclosed herein.

NOV3

A NOV3 polypeptide has been identified as a Thrombospondin type 1 (tsp_{—}1) domain containing protein (also referred to as CG93275-01). The disclosed novel NOV3 nucleic acid (SEQ ID NO:5) of 799 nucleotides is shown in Table 3A. The novel NOV3 nucleic acid sequences maps to the chromosome 16.

An ORF begins with an ATG initiation codon at nucleotides 51-53 and ends with a TGA codon at nucleotides 744-746. A putative untranslated region and/or downstream from the termination codon is underlined in Table 3A, and the start and stop codons are in bold letters.

TABLE 3A
NOV3 Nucleotide Sequence

(SEQ ID NO:5)

GAATATATTTAGTGTGTTGTTTTTTTTTTTAATGTGGCTACTGAAACCTAATGGGAATGCAAATACA
ACTTTTTTGTCTTCTCAAGTGTTCCAAGACCTCTGGACGAGGGGTGAGCAACCGTGAACTCCTCTGC
AAGGGCTCTGCCGCAGAAACCCTCCCCGAGAGCCAGTGTACCAGTCTCCCCAGACCTGAGCTGCACG
AGGGCTGTGTGCTTCGACGATGCCCCAAGAACACCCGGCTACAGTGGGTCGCTTCTTCGTGGAGCGA
GTGTTCTGCAACCTGTGGTTTGGGTGTGAGGAAGAGGCACATGAAGTGCAGCGAGAAGGGCTTCCAG
GCAAAGCTGATAACTTTCCCAGACCGAAGATGCCGTAATATTAACAAACCAAATCTGGACTTGGAAG
AGACCTGCAACCGACGGGCTTCCCCAGCCCATCCAGTGTACAACATGGTAGCTGGATGGTATTCATT
GCCGTGCCAGCAGTCCACAGTCACCTGTGGGGGAGGCGTCCAGACCCGGTCAGTCCACTGTGTTCAG
CAAGGCCGGCCTTCCTCAAGTTGTCTGCTCCAATCAGAACCTCCGGTGCTACGAGCCTGTAATACAA
ACTTCTGTCCAGCTCCTGAAAAGAGAGAGGATCCATCCTGCGTAGATTTCTTCAACTGGTGTCACCT
AGTTCCTCACCATGGTCTCTGCAACCACAAGTTTTACGGAAAACAATGCTGCAAGTCATGCACAAGG
AAGATCTGATCTTGGTGTCCTCCCCAGCCTTAGGGCCAGCGCCTTACCTTTCAACCTCTACA

The NOV3 protein (SEQ ID NO:6) encoded by SEQ ID NO:5 is 231 amino acid residues in length and is presented using the one-letter amino acid code in Table 3B. Psort analysis predicts the NOV3 protein of the invention to be localized in the cytoplasm with a certainty of 0.4500.

TABLE 3B
Encoded NOV3 protein seqnence

(SEQ ID NO:6)

MGMQIELFCLLKCSKTCCRGVRKRELLCKGSAAETLPESQCTSLPRPELQEGCVLGRCPKNSRLQ
WVASSWSECSATCGLGVRKREMKCSEKGFQGKLITFPERRCRNIKKPNLDLEETCNRRACPAHPV
YNMVAGWYSLPWQQCTVTCGGGVQTRSVHCVQQGRPSSSCLLHQKPPVLRACNTNFCPAPEKRED
PSCVDFFNWCHLVPQHGVCNHKFYGKQCCKSCTRKI

A search against the Patp database, a proprietary database that contains sequences published in patents and patent publications, yielded several homologous proteins shown in Table 3C.

TABLE 3C
Patp results for NOV3

| | | | Smallest |
|---|---|---|---|
| | | | Sum |
| Sequences producing High-scoring | Reading | High | Prob |
| Segment Pairs: | Frame | Score | P (N) |
| >patp:AAE09696 Human gene 7 encoding | +1 | 1248 | 9.2e−127 |
| protein HE8CY61 | | | |
| >patp:AAE09699 Human gene 10 encoding | +1 | 1245 | 1.9e−126 |
| protein HUVHR16 | | | |
| >patp:AAU72893 Human metalloprotease | +1 | 1204 | 4.2e−122 |
| partial sequence #5 | | | |
| >patp:AAU72891 Human metalloprotease | +1 | 693 | 3.5e−70 |
| partial sequence #3 | | | |
| >patp:AAB21253 Human metalloproteinase | +1 | 327 | 5.5e−28 |
| KIAA0605 | | | |

In a BLAST search of public sequence databases, it was found, for example, that the nucleic acid sequence of this invention has 392 of 396 bases (98%) identical to an EST AA057409 mRNA from human). The full amino acid sequence of the protein of the invention was found to have 74 of 216 amino acid residues (34%) identical to, and 107 of 216 amino acid residues (49%) similar to, the 237 amino acid residue ptnr:SPTREMBL-ACC:Q9HBS6 protein from Homo sapiens (HYPOTHETICAL 25.7 KDA PROTEIN).

NOV3 also has homology to the proteins shown in the BLASTP data in Table 3D.

TABLE 3D
BLAST results for NOV3

| Gene Index/ | | | | | |
|---|---|---|---|---|---|
| Identifier | Protein/Organism | Length (aa) | Identity (%) | Positives (%) | Expect |
| gi\|18598706\|ref\|XP_{—} | hypothetical | 1123 | 181/183 | 183/183 | e−100 |
| 091253.1\|(XM_091253) | protein XP_091253 | | (98%) | (99%) | |
| | [Homo sapiens] | | | | |
| gi\|19171150\|emb\| | ADAMTS18 protein | 1081 | 61/62 | 62/62 | 4e−27 |
| CAC83612.1\|(AJ311903) | [Homo sapiens] | | (98%) | (99%) | |
| gi\|7662202\|ref\|NP_{—} | KIAA0605 gene | 951 | 79/216 | 99/216 | 9e−23 |
| 055509.1\|(NM_014694) | product | | (36%) | (45%) | |
| | [Homo sapiens] | | | | |
| gi\|18561227\|ref\|XP_{—} | hypothetical | 1365 | 51/112 | 74/112 | 4e−21 |
| 094442.1\|(XM_094442) | protein XP_094442 | | (45%) | (65%) | |
| | [Homo sapiens] | | | | |
| gi\|17432918\|sp\| | HUMAN ADAMTS-10 | 223 | 74/223 | 104/223 | 5e−20 |
| Q9H324\|AT10_{—} | precursor (A | | (33% | (46%) | |
| | disintegrin and | | | | |
| | metalloproteinase | | | | |
| | with | | | | |
| | thrombospondin | | | | |
| | motifs 10) (ADAM- | | | | |
| | TS 10) (ADAM- | | | | |
| | TS10) (Fragment) | | | | |

A multiple sequence alignment is given in Table 3E, with the NOV3 protein being shown on line 1 in Table 3E in a ClustalW analysis, and comparing the NOV3 protein with the related protein sequences shown in Table 3D. This BLASTP data is displayed graphically in the ClustalW in Table 3E.

[image]

[image]

The NOV3 Clustal W alignment shown in Table 3E was modified to begin at amino residue 1321. The data in Table 3E includes all of the regions overlapping with the NOV3 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 3F lists the domain description from DOMAIN analysis results against NOV3.

TABLE 3F
Domain Analysis of NOV3

| | Region of | | |
|---|---|---|---|
| Model | Homology | Score (bits) | E value |
| tsp_1 | 12-58 | −6.8 | 4.1 |
| tsp_1 | 66-125 | 14.6 | 0.015 |
| tsp_1 | 141-187 | 19.3 | 0.0041 |

Consistent with other known members of the Thrombospondin type 1 (tsp_{—}1) family of proteins, NOV3 has, for example, three tsp_{—}1 domain signature sequences and homology to other members of the tsp_{—}1 Domain-containing Protein Family. NOV3 nucleic acids, and the encoded polypeptides, according to the invention are useful in a variety of applications and contexts. For example, NOV3 nucleic acids and polypeptides can be used to identify proteins that are members of the tsp_{—}1 Domain-containing Protein Family. The NOV3 nucleic acids and polypeptides can also be used to screen for molecules, which inhibit or enhance NOV3 activity or function. Specifically, the nucleic acids and polypeptides according to the invention may be used as targets for the identification of small molecules that modulate or inhibit, e.g., cellular activation, cellular metabolism, and signal transduction. These molecules can be used to treat, e.g., Von Hippel-Lindau (VHL) syndrome, diabetes, tuberous sclerosis, fertility, hypogonadism, as well as other diseases, disorders and conditions.

In addition, various NOV3 nucleic acids and polypeptides according to the invention are useful, inter alia, as novel members of the protein families according to the presence of domains and sequence relatedness to previously described proteins. For example, the NOV3 nucleic acids and their encoded polypeptides include structural motifs that are characteristic of proteins belonging to the tsp_{—}1 Domain-containing Protein Family.

Thrombospondin type 1 domain (TSP1, IPR000884) is a repeat found in the thrombospondin protein where it is repeated 3 times. Likewise, the tsp_{—}1 domain is repeated three times in the NOV3 polypeptide. Now a number of proteins involved in the complement pathway (properdin, C6, C7, C8A, C8B, C9) (Patthy, L., J. Mol. Biol. 202: 689-696 (1988)) as well as extracellular matrix protein like mindin, F-spondin (Okainoto, et al., Development 126: 3637-3648 (1999)), SCO-spondin and even the circumsporozoite surface protein 2 and TRAP proteins of Plasmodium (Wengelnik, et al., EMBO J. 18: 5195-5204 (1999); Rogers, et al., Mol. Biochem. Parasitol. 53: 45-51 (1992)) contain one or more instance of this repeat. It has been involved in cell-cell interraction, inhibition of angiogenesis (Krutzsch, et al., Circulation 100: 1423-1431 (1999)), apoptosis [Krutzsch, et al., Cancer Res. 57: 1735-1742 (1997)).

The NOV3 nucleic acids and polypeptides, antibodies and related compounds according to the invention will be useful in therapeutic and diagnostic applications in the mediation of urogenital physiology. As such, the NOV3 nucleic acids and polypeptides, antibodies and related compounds according to the invention may be used to treat reproductive and metabolic disorders, e.g., Von Hippcl-Lindau (VHL) syndrome, diabetes, tuberous sclerosis, fertility, hypogonadism, as well as other diseases, disorders and conditions.

The NOV3 nucleic acids and polypeptides arc useful for detecting specific cell types. For example, expression analysis has demonstrated that a NOV3 nucleic acid is expressed in eye and testis.

Additional utilities for NOV3 nucleic acids and polypeptides according to the invention are disclosed herein.

NOV4

A NOV4 polypeptide has been identified as a Protocadherin Alpha C2 Short Form-like protein (also referred to as CG93187-01). The disclosed novel NOV4 nucleic acid (SEQ ID NO:7) of 600 nucleotides is shown in Table 4A. The novel NOV4 nucleic acid sequences maps to the chromosome 11.

An ORF begins with an ATG initiation codon at nucleotides 41-43 and ends with a TAG codon at nucleotides 2546-2548. A putative untranslated region and/or downstream from the termination codon is underlined in Table 4A, and the start and stop codons are in bold letters.

TABLE 4A
NOV4 Nucleotide Sequence

(SEQ ID NO:7)

CACCATAAAACCTCAGAAAATAGACTTTTCCTCTGCCTCTATGCAGGGGCAGGCCAGATCTGGGGAA
GGGATGGGACAGCCTGGCATGAAGAGCCCCAGGCCCCACCTCCTGCTACCATTGCTGCTGCTGCTGC
TGCTGCTGCTGTCTTCCCCTCGCCGTGCACGCGTGCGCCTCCCAGAGGACCAGCCGCCTGGGCCCCC
GGCTGGCACGCTCCTAGCCCGCGACCCGCATCTGGGCGAGGCTGCACGCGTGTCCTATCGGCTGGCA
TCTGGCGGGGACGGCCACTTCCGGCTGCACTCAAGCACTGGAGCGCTGTCCGTGGTGCGGCCGTTGC
ACCGCGAACAACGAGCTGAGCACGTACTGACAGTGGTGGCCTCAGACCGAGCTCCCCGCCCGCGCTC
GGCCACGCAGGTCCTGACCGTCAGTGTCGCTCACGTCAACGACGAGGCGCCTACTTTCCAGCAGCAG
GAGTACAGCGTCCTCTTGCGTGAGAACAACCCTCCTGGCACATCTCTGCTCACCCTGCGAGCAACCG
ACCCCGACGTGGGGGCCAACGGGCAAGTGACTTATGGAGGCGTCTCTAGCGAAAGCTTTTCTCTGCA
TCCTGACACTGGTGTTCTCACGACTCTTCCGGCCCTGGATCGAGAGGAACACGAGGAGATCAACCTG
ACAGTGTATGCCCACGACAGCCGCTCACCTCCTCACTTAACGCATGTCACTGTTCGAGTGGCTGTGG
AGGATGAGAATGACCATGCACCAACCTTTGGGAGTGCCCATCTCTCTCTGGAGGTGCCTGAGGGCCA
GGACCCCCAGACCCTTACCATGCTTCGGGCCTCTGATCCAGATGTGGCAGCCAATGGGCAGTTGCAG
TACCGCATCCTAGATCGGGACCCATCAGGAGCCTTTGTCCTAGACCTTGCTTCTGGAGACTTTCGCA
CCATGCGCCCACTAGACAGAGAAGTGGAGCCACCTTTCCAGCTGAGGATAGAGCCCCGGGATGGAGG
CCACCCAGCTCTCACTGCCACGCTCCTTTTGACACTGACACTGCTGGATGCCAATGACCATGCTCCA
GCCTTTCCTGTGCCTGCCTACTCGGTGGAGGTGCCGGAGGATGTGCCTCCAGGCACCCTGCTGCTGC
AGCTACAGGCTCATGACCCTGATGCTGGAGCTAATGGCCATCTGACCTACTACCTGGGCGCCGGTAC
AGCAGGAGCCTTCCTGCTGGAGCCCAGCTCTGGAGAACTGGTGTTGCTTGAACCTCTACACTTTGAA
AGCCTGACACAGTACAATCTAACAGTCGCTGCAGCTGACCGTGGGCAGCCACCCCAAAGCTCAGTCG
TGCCAGTCACTGTCACTGTACTAGATGTCAATGACAACCCACCTGTCTTTACCCGAGCATCCTACCG
TGTGACAGTACCTCAGCACACACCTGTTGGAGCTCAGCTGCTGCATGTAGAGGCCTCTGACGCTGAC
CCTCCCCTCATGCCCTCCTCAGGCGACCCATCAGGGCTCTTTGAGCTGGATGAGAGCTCAGGCACCT
TGCCACTGGCCCATGCCCTCGACTCTGAGACCCAGGCTCGACATCACCTTGTAGTACAGGCTGCTGA
CCCTGCTGGTGCACACTTTGCTTTGGCACCAGTGACAATTGAGGTCCACGATGTGAATGATCATGCC
CCAGCCTTCCCACTGAACTTACTCAGCACCAGCGTGGCCGAGAATCAGCCTCCACGCACTCTCCTGA
CCACTCTGCATGCAATCGACGGGGATGCTGGCGCTTTTGGGAGGCTCCGTTACAGCCTGTTCGAGGC
TGGGCCAGGACCTGAGGGCCGTGAGGCATTTGCACTGAACAGCTCAACAGGGGAGTTGCGTCCGCGA
GTGCCCTTTGACTATGAGCACACACAAAGCTTCCGGCTGCTGGTGGCTGCTGCTGATGCTGGGAATC
TCTCAGCCTCTGTCACTGTGTCGGTGCTAGTGACTGGAGAGGATGAGTATCACCCTGTATTTCTGGC
ACCAGCTTTCCACTTCCAAGTGCCCGAAGGTGCCCGGCGTGGCCACAGCTTCGGTCACGTCCAGCCC
ACAGATGAGGATGGGGGTGCCCATGCCCTGGTTCTGTATTCCCTTGCCACCTCTTCCCCCTATTTTG
GTATTAACCACACTACAGGAGCCCTGTACCTGCGGGTGGACAGTCGGGCACCAGGCAGCGGAACAGC
CACCTCTGGGGGTCGGGCCCGGACCCGGCGGGAAGCACCACGGGAGCTGGGGCTCCACCTGGACTCT
TACCAGAGTCACTCCAAGTCCTGTCTCAGGCAGAATACTCACATCTATTCCAAGCACCTTCCCTGGG
ATCTCAGGCGCATACTGAGAACCAGTGGGACAGGGTTGAGAGAGAGAGCCAACCGAGAATCTCAAAT
GAACCAAACTGAGAAAGATGCCCCTCAGTGCGGCTACAGACCGACACCCCACCATGGCCCAACAGAA
AAACCAACACCCCCTCCCCAAAGGAATCAAACCAATCGGGAAAAGGAAGGAGGCGTTCGCCGTGCCT
AGGATAT

The NOV4 protein (SEQ ID NO:8) encoded by SEQ ID NO:7 is 835 amino acid residues in length and is presented using the one-letter amino acid code in Table 4B. Psort analysis predicts the NOV4 protein of the invention to be localized at the plasma membrane with a certainty of 0.7900.

TABLE 4B
Encoded NOV4 protein sequence

(SEQ ID NO:8)

MEGQARSGEGMCQPCMKSPRPHLLLPLLLLLLLLLSSPRRARVRLPEDQPPGPAAGTLLARDPHL
GEAARVSYRLASCGDGHFRLHSSTGALSVVRPLDREQRAEHVLTVVASDRAPRPRSATQVLTVSV
ADVNDEAPTFQQQEYSVLLRENNPPGTSLLTLRATDPDVGANCQVTYGGVSSESFSLDPDTGVLT
TLRALDREEQEEINLTVYAQDRGSPPQLTHVTVRVAVEDENDHAPTFGSAHLSLEVPEGQDPQTL
TMLRASDPDVGANGQLQYRILDCDPSGAFVLDLASGEFGTMRPLDREVEPAFQLRIEARDGGQPA
LSATLLLTVTVLDANDHAPAFPVPAYSVEVPEDVPAGTLLLQLQAHDPDAGANGHVTYYLGACTA
GAFLLEPSSGELVLLEPLDFESLTQYNLTVAAADRGQPPQSSVVPVTVTVLDVNDNPPVFTRASY
RVTVPEDTPVGAELLHVEASDADPALMASSGDPSCLFELDESSGTLRLAHALDCETQARHQLVVQ
AADPAGAHFALAPVTIEVQDVNDHGPAFPLNLLSTSVAENQPPCTLVTTLHAIDGDAGAFGRLRY
SLLEAGPGPEGREAFALNSSTGELRARVPFDYEHTESFRLLVGAADAGNLSASVTVSVLVTGEDE
YDPVFLAPAFHFQVPEGARRGHSLGHVQATDEDGGADGLVLYSLATSSPYFGINQTTGALYLRVD
SRAPGSGTATSGGGGRTRREAPRELGLHLDSYQSHSKSCLRQNTQIYSKHLPWDLRRILRTSGTG
LRERANRESQMNQTEKDAPQWCYRPTPHHGATEKPRPPPQRNQTNREKEGGVGRA

A search against the Patp database, a proprietary database that contains sequences published in patents and patent publications, yielded several homologous proteins shown in Table 4C.

TABLE 4C
Patp results for NOV4

| | | | Smallest |
|---|---|---|---|
| | | | Sum |
| Sequences producing High-scoring | Reading | High | Prob |
| Segment Pairs: | Frame | Score | P (N) |
| >patp:AAU07054 Human Flamingo protein | +1 | 968 | 1.8e−98 |
| >patp:AAU07053 Human Flamingo | +1 | 968 | 2.0e−98 |
| polypeptide | | | |
| >patp:ABG21921 Novel human diagnostic | +1 | 642 | 3.1e−64 |
| protein #21912 | | | |
| >patp:ABG21921 Novel human diagnostic | +1 | 642 | 3.1e−64 |
| protein #21912 | | | |

In a BLAST search of public sequence databases, it was found, for example, that the nucleic acid sequence of this invention has 273 of 415 bases (65%) identical to a gb:GENBANK-ID:AF061573|acc:AF061573.2 mRNA from Homo sapiens (protocadherin (PCDH8) mRNA, complete cds). The full amino acid sequence of the protein of the invention was found to have 273 of 415 amino acid residues (65%) identical to, and 273 of 415 amino acid residues (65%) similar to, the 4076 amino acid residue gb:GENBANK-ID:AF061573|acc:AF061573.2 protein from Homo sapiens (protocadherin (PCDH8) mRNA, complete cds).

NOV4 also has homology to the proteins shown in the BLASTP data in Table 4D.

TABLE 4D
BLAST results for NOV4

| Gene Index/ | | | | | |
|---|---|---|---|---|---|
| Identifier | Protein/Organism | Length (aa) | Identity (%) | Positives (%) | Expect |
| gi\|17461472\|ref\|XP_{—} | similar to | 1415 | 459/682 | 503/682 | 0.0 |
| 052786.2\|(XM_052786) | protocadherin 16 | | (67%) | (73%) | |
| | [Homo sapiens] | | | | |
| gi\|16933557\|ref\|NP_{—} | protocadherin 16 | | 443/676 | 490/676 | 0.0 |
| 003728.1\|(NM_003737) | precursor; | | (65%) | (71%) | |
| | fibroblast | | | | |
| | cadherin FIB1; | | | | |
| | cadherin 19; | | | | |
| | fibroblast | | | | |
| | cadherin 1; | | | | |
| | dachsous | | | | |
| | homologue | | | | |
| | [Homo sapiens] | | | | |
| gi\|6753408\|ref\|NP_{—} | cadherin EGF LAG | 3034 | 48/693 | 358/693 | 1e−98 |
| 034016.1\|(NM_009886) | seven-pass G-type | | (35%) | (50%) | |
| | receptor | | | | |
| | [Mus musculus] | | | | |
| gi\|13325064\|ref\|NP_{—} | cadherin EGF LAG | 2923 | 246/679 | 345/679 | 2e−98 |
| 001399.1\|(NM_001408) | seven-pass G-type | | (36%) | (50%) | |
| | receptor 2; | | | | |
| | EGF-like-domain, | | | | |
| | multiple 2; | | | | |
| | epidermal growth | | | | |
| | factor-like 2; | | | | |
| | multiple | | | | |
| | epidermal growth factor- | | | | |
| | like | | | | |
| | domains 3; | | | | |
| | cadherin, EGF LAG | | | | |
| | seven-pass G-type | | | | |
| | receptor 2, | | | | |
| | flamingo | | | | |
| | (Drosophila)homolog | | | | |
| gi\|10727655\|gb\| | stan gene product | 3606 | 241/700 | 361/700 | 3e−98 |
| AAF58763.2\|(AE003828) | [Drosophila | | (34% | (51%) | |
| | melanogaster] | | | | |

A multiple sequence alignment is given in Table 4E, with the NOV4 protein being shown on line 1 in Table 4E in a ClustalW analysis, and comparing the NOV4 protein with the related protein sequences shown in Table 4D. This BLASTP data is displayed graphically in the ClustalW in Table 4E.

[image]

[image]

[image]

[image]

[image]

The NOV4 Clustal W alignment shown in Table 4E was modified to begin at amino residue 1201 and end at amino acid residue 2760. The data in Table 1E includes all of the regions overlapping with the NOV4 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 4F lists the domain description from DOMAIN analysis results against NOV4.

TABLE 4F
Domain Analysis of NOV4

| | Region of | | |
|---|---|---|---|
| Model | Homology | Score (bits) | E value |
| cadherin | 41-131 | 97.7 | 2.4e−25 |
| T2SP_N | 16-223 | −117.2 | 1.3 |
| cadherin | 145-233 | 104.1 | 2.7e−27 |
| cadherin | 247-337 | 78.1 | 1.8e−19 |
| cadherin | 351-441 | 112.9 | 6e−30 |
| cadherin | 455-539 | 64.7 | 2e−15 |
| cadherin | 553-646 | 77.5 | 2.8e−19 |
| cadherin | 660-745 | 15.4 | 0.036 |

Consistent with other known members of the Protocadherin Alpha C2 Short Form Protein-like family of proteins, NOV4 has, for example, seven Cadherin domain signature sequences and homology to other members of the Protocadherin Alpha C2 Short Form Protein-like Protein Family. NOV4 nucleic acids, and the encoded polypeptides, according to the invention are useful in a variety of applications and contexts. For example, NOV4 nucleic acids and polypeptides can be used to identify proteins that are members of the Protocadherin Alpha C2 Short Form Protein-like Protein Family. The NOV4 nucleic acids and polypeptides can also be used to screen for molecules, which inhibit or enhance NOV4 activity or function. Specifically, the nucleic acids and polypeptides according to the invention may be used as targets for the identification of small molecules that modulate or inhibit, e.g., cellular activation, cellular metabolism, and signal transduction. These molecules can be used to treat, e.g., Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderma, allergy, ARDS, fertility, endometriosis, hypogonadism, hemophilia, hypercoagulation, idiopathic thrombocytopenic purpura, autoimmune disease, allergies, immunodeficiencies, transplantation, graft versus host disease (GVHD), lymphaedema, as well as other diseases, disorders and conditions.

In addition, various NOV4 nucleic acids and polypeptides according to the invention are useful, inter alia, as novel members of the protein families according to the presence of domains and sequence relatedness to previously described proteins. For example, the NOV4 nucleic acids and their encoded polypeptides include structural motifs that are characteristic of proteins belonging to the Protocadherin Alpha C2 Short Form Protein-like Protein Family.

Cadherins (Takeichi, Annu. Rev. Biochem. 59: 237-252 (1990); Takeichi Trends Genet. 3: 213-217 (1987)), first discovered in mouse teratocarcinoma cells (Liaw, EMBO J. 9: 2701-2708 (1990)), are a family of animal glycoproteins responsible for calcium-dependent cell-cell adhesion. Cadherins preferentially interact with themselves in a homophilic manner in connecting cells; thus acting as both receptor and ligand. There are a number of different isoforms distributed in a tissue-specific manner in a wide variety of organisms. Cells containing different cadherins tend to segregate in vitro, while those that contain the same cadherins tend to preferentially aggregate together. This observation is linked to the finding that cadherin expression causes morphological changes involving the positional segregation of cells into layers, suggesting they may play an important role in the sorting of different cell types during morphogenesis, histogenesis and regeneration. They may also be involved in the regulation of tight and gap junctions, and in the control of intercellular spacing. Cadherins are evolutionary related to the desmogleins which are component of intercellular desmosome junctions involved in the interaction of plaque proteins.

Structurally, cadherins comprise a number of domains: these include a signal sequence; a propeptide of around 130 residues; an extracellular domain of around 600 residues; a single transmembrane domain; and a well-conserved C-terminal cytoplasmic domain of about 150 residues. The extracellular domain can be subdivided into 5 parts, 4 of which are repeats of about 110 residues, and the fifth contains 4 conserved cysteines. The calcium-binding region of cadherins is thought to be located in the extracellular domain. This indicates that the sequence of the invention has properties similar to those of other proteins known to contain this/these domain(s) and similar to the properties of these domains.

Maniatis et al. has identified 52 novel human cadherin-like genes organized into three closely linked clusters (Wu and Maniatis, Cell 97(6):779-90 (1999).) Comparison of the genomic DNA sequences with those of representative cDNAs reveals a striking genomic organization similar to that of immunoglobulin and T cell receptor gene clusters. The N-terminal extracellular and transmembrane domains of each cadherin protein are encoded by a distinct and unusually large exon. These exons are organized in a tandem array. By contrast, the C-terminal cytoplasmic domain of each protein is identical and is encoded by three small exons located downstream from the cluster of N-terminal exons. This unusual organization has interesting implications regarding the molecular code required to establish complex networks of neuronal connections in the brain and the mechanisms of cell-specific cadherin-like gene expression.

The NOV4 nucleic acids and polypeptides, antibodies and related compounds according to the invention will be useful in therapeutic and diagnostic applications in the mediation of urogenital, nerve, and endocrine physiology. As such, the NOV4 nucleic acids and polypeptides, antibodies and related compounds according to the invention may be used to treat reproductive and nervous system disorders, e.g., Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderima, allergy, ARDS, fertility, endometriosis, hypogonadism, hemophilia, hypercoagulation, idiopathic thrombocytopenic purpura, autoimmune disease, allergies, immunodeficiencies, transplantation, graft versus host disease (GVHD), lymphaedema, as well as other diseases, disorders and conditions.

The NOV4 nucleic acids and polypeptides are useful for detecting specific cell types. For example, expression analysis has demonstrated that a NOV4 nucleic acid is expressed in Heart, Aorta, Umbilical Vein, Thyroid, Colon, Peripheral Blood, Spleen, Lymph node, Bone, Cartilage, Brain, Left cerebellum, Right Cerebellum, Parietal Lobe, Temporal Lobe, Cerebral Medulla/Cerebral white matter, Hippocampus, Cervix, Mammary gland/Breast, Ovary, Placenta, Uterus, Testis, Lung, and Retina.

Additional utilities for NOV4 nucleic acids and polypeptides according to the invention are disclosed herein.

NOV5

A NOV5 polypeptide has been identified as a Nuclear protein-like protein (also referred to as CG95083-01). The disclosed novel NOV5 nucleic acid (SEQ ID NO:9) of 2322 nucleotides is shown in Table 5A.

An ORF begins with an ATG initiation codon at nucleotides 70-72 and ends with a TAA codon at nucleotides 2320-2322. A putative untranslated region and/or downstream from the termination codon is underlined in Table 5A, and the start and stop codons are in bold letters.

TABLE 5A
NOV5 Nucleotide Sequence

(SEQ ID NO:9)

GTGCAAGGACAGTCCAGAGCCCTTGTCATCCCACAGGAACTGCTATCTTCAGAGAAAGCATACGTCG
AGATGCTCCAGCACTTAAATCTGTTCCTGCCAGACCAGGCTATCAGCAGGAGAGGCCAGCCCTCCAA
AGCCCCACGGGAAATCTGCCAAGGACGACTTCTGCTCAGTCCTATCAACCTGTGCGTAACAGACCTT
TTGGTGTTTCACCATTTCCATGGACCTGTCATGAGGGCCTTGGATGACATGGACCATGAAGGCACAG
ACACATTGGCCCGCGAGGACCTCAGGCAGGGCCTGAGTGAACTCCCAGCCATCCACGACCTTCATCA
AGGCATCCTGGAGCAGCTGGAGGAAACGCTGTCAAATTGCCAGAGCCAGCAGAAGGTAGCTGACGTC
TTCCTTGCCCGGGAGCAGGGGTTTGATCACCACCCCACTCACATCCTGCAGTTCGACAGGTACCTAG
GTCTGCTCAGTGAGAATTGCCTCCACTCTCCCCGGCTGGCAGCTGCTGTCCGTGAATTTCAGCACAG
TGTACAAGGAGGCAGCCAGACTCCGAAGCATCGGCTGCTGCGGGTGGTTCAACGCCTCTTCCAGTAC
CAAGTGCTCCTCACAGACTATTTAAACAACCTTTGTCCGCACTCCGCCCAGTACCACAACACACAGG
GTGCACTGAGCCTCATCTCCAAAGTCACAGACCGTGCCAACGACAGCATGGAGCAACGGGAAAACCT
GCAGAAGCTCGTCCACATTGAGCACAGCGTCCCGGGCCAAGGGGATCTCCTCCAGCCAGGAAAGCAG
TTTCTGAAGGAAGGGACGCTGATGAAAGTAACAGGGAAAAACAGACGGCCCCGCCACCTATTTCTGA
TGAACGATGTGCTCCTGTACACCTATCCCCAGAACGATGGGAAGTACCGGCTGAACAACACATTGGC
TGTGGCCAACATGAAGGCTCTTTACCATGGGCAAGCGGAAGGAGCAAACACCTTTCTCAGCATGGAG
GTTTGTTCCCTTTTGGAACCAAACGCTCCACCGAGGAGCCTGTTAGAAAAACGCATGGGACACGTGG
TCACTGGCAGGTACTTCTCCAACATCACAGTGCACCTGCGGTTGCCCGGGCTGCGCCCTGAGCATGA
CGCTCTGCAGCCTTCCCAGCCGTGGCTCAGCCGCCCTGTGATCGAGAAAGTGCCCTACGCTCTAAAG
ATTGAGACTTCCGACTCCTGCCTGATGCTGTCTGCGAGGCTGCACGTCAGGAAGTCCAAGGTCAAGG
CACTGACTCATTCGGTGTCTGCAGCCCTCGGAGTTAGCGGAATATCATTATTCCAGTGTAAGAACAA
ACAGACCCAAGCACAGCTAATGGACCAGTGGTCTGCTCGTAAACCTAGTCTCGCAGGTCATCTCTTC
TTTCCTGGTGGTTCTGGGCAGTGTGAGAGGTGCACGCTCAAGGGGCATCTGAGTGAGAACCTCATCC
ATGCCGAGATGGAGGCCCATGCCCGCAGCTCCTGTGCACAGACGGACGAGTGGTATGCCTCTCTGAG
CAGAGCCCTCCCTGAGGACTACAAGGCCCAGGCGCTCGCTGCATTCCACCATAGCGTGGAGATACGA
GAGAGGCTGGGCGTTACCCTTCGGGAGAGCCCCCCCACCCTGGTGCCTGTCACACACGTCATGATGT
GCATGAACTGCGCCTGCGACTTCTCCCTCACCCTGCGGCGTCATCACTGTCACGCCTGTGGCAAGCA
GATCGTGTGCCGGAACTGTTCGCGGAACAAGTACCCGCTGAAGTACCTCAAGGACAGGATGGCCAAG
GTCTGCGACGGCTCCTTCGGGGAGCTGAAGAAGCGGCGCAGGGCTGTCCCGGGCCTGATGAGACTTA
CAGAGCGGCCTGTGAGCATGAGCTTCCCGCTGTCTTCACCCCGCTTCTCGGGCAGTGCCTTTTCATC
CGTCTTCCAGAGCATTAACCCCTCGACCTTCAAGAAGCAGAAGAAAGTCCCTTCAGCCCTGACAGAG
GTAGCTGCCTCTGGAGAGGGCTCTGCCATCAGTGCCTATCTCAGCCGGTGTAAGAGGGGCAAGCGGC
ACTGGAAGAAGCTCTGGTTTGTCATCAAAGGCAAAGTTCTCTACACCTACATGGCCAGTGAGGACAA
AGTGGCCTTGGAGAGTATGCCTCTGCTAGGCTTCACCATTGCTCCAGAAAAGGAACAGGGCAGCAGT
GAAGTAGGACCTATTTTTCACCTTTACCACAAGAAAACCCTATTTTATAGCTTCAAAGCAGAAGATA
CCAATTCATCGATCGAGGCCATGGAAGATGCGAGTGTGTTATAG

Variant sequences of NOV5 are included in Example 3, Table 19. A variant sequence can include a single nucleotide polymorphism (SNP). A SNP can, in some instances, be referred to as a “cSNP” to denote that the nucleotide sequence containing the SNP originates as a cDNA.

The NOV5 protein (SEQ ID NO:10) encoded by SEQ ID NO:9 is 750 amino acid residues in length and is presented using the one-letter amino acid code in Table 5B. Psort analysis predicts the NOV5 protein of the invention to be localized in the nucleus with a certainty of 0.3000.

TABLE 5B
Encoded NOV5 protein sequence

(SEQ ID NO:10)

MLQHLNLFLAEQAISRRGQGSKAPGEICQGGLVLSPINLWVTDLLVFQDFHGAVMRALDDMDHEG
RDTLAREELRQGLSELPAIHDLHQGTLEELEERLSNWESQQKVADVFLAREQGFDHHATHTLQFD
RYLGLLSENCLHSPRLAAAVREFEQSVQGGSQTAKHRLLRVVQRLPQYQVLLTDYLNNLCPDSAE
YDNTQGALSLISKVTDRANDSMEQGENLQKLVHIEHSVRGQCDLLQPGREFLKEGTLMKVTCKNR
RPRHLFLMNDVLLYTYPQKDGKYRLKNTLAVANMKALYHGEGEGGSTFLSMEVCSLLEPKAPPRS
LLEKGMGDVVTGRYLSNMTVHLGLPCLGPEHDALQPSQRWVSRPVMEKVPYALKIETSESCLMLS
ARLQVRKSKVKALTDSVSAALGVRGISLFQCKKKQTQCQLMDQWSARKPSLAGDLFFAGGSCQCE
RCRLKGHLSENLIHAEMEAHARSSCAERDEWYGCLSRALPEDYKAQALAAFHHSVEIRERLGVSL
GERPPTLVPVTHVMMCMNCGCDFSLTLRRHHCHACGKQIvCRNCSRNKYPLKYLKDRMAKVCDGC
FGELKKRGRAVPGLMRVTERPVSMSFPLSSPRFSGSAFSSVFQSINPSTFKKQKKVPSALTEVAA
SGEGSATSGYLSRCKRGKRHWKKLWFVIKGKVLYTYMASEDKVALESMPLLGFTIAPEKEECSSE
VAAPIFHLYHKKTLEYSFKAEDTNSWIEANEDASVL

A search against the Patp database, a proprietary database that contains sequences published in patents and patent publications, yielded several homologous proteins shown in Table 5C.

TABLE 5C
Patp results for NOV5

| | | | Smallest |
|---|---|---|---|
| | | | Sum |
| Sequences producing High-scoring | Reading | High | Prob |
| Segment Pairs: | Frame | Score | P (N) |
| >patp:AAB93568 Human protein sequence | +1 | 577 | 1.7e−95 |
| SEQ ID NO: 12972 | | | |
| >patp:AAY51248 Rat actin-binding | +1 | 312 | 1.9e−41 |
| protein frabin | | | |
| >patp:AAU21630 Novel human neoplastic | +1 | 256 | 1.6e−38 |
| disease polypeptide | | | |
| >patp:AAU27818 Human full-length | +1 | 300 | 2.6e−29 |
| polypeptide #143 | | | |
| >patp:ABG00573 Novel human diagnostic | +1 | 261 | 1.8e−26 |
| protein #564 | | | |

In a BLAST search of public sequence databases, it was found, for example, that the nucleic acid sequence of this invention has 443 of 754 bases (58%) identical to a gb:GENBANK-ID:AB037783|acc:AB037783.1 mRNA from Homo sapiens (mRNA for KIAA1362 protein, partial cds). The full amino acid sequence of the protein of the invention was found to have 114 of 263 amino acid residues (43%) identical to, and 173 of 263 amino acid residues (65%) similar to, the 699 amino acid residue ptnr:SPTREMBL-ACC:Q9P215 protein from Homo sapiens (KIAA1362 PROTEIN).

NOV5 also has homology to the proteins shown in the BLASTP data in Table 5D.

TABLE 5D
BLAST results for NOV5

| Gene Index/ | | | | | |
|---|---|---|---|---|---|
| Identifier | Protein/Organism | Length (aa) | Identity (%) | Positives (%) | Expect |
| gi\|8922921\|ref\|NP_{—} | hypothetical | 432 | 135/284 | 169/284 | 5e−57 |
| 060821.1\|(NM_018351) | protein FLJ11183 | | (47%) | (58%) | |
| | [Homo sapiens] | | | | |
| gi\|16716345\|ref\|NP_{—} | ethanol decreased | 431 | 131/284 | 171/284 | 2e−55 |
| 444302.1\|(NM_053072) | 4 [Mus musculus] | | (46%) | (60%) | |
| gi\|7243105\|dbj\| | KIAA1362 protein | 699 | 111/251 | 166/251 | 2e−54 |
| BAA92600.1\|(AB037783) | [Homo sapiens] | | (44%) | (65%) | |
| gi\|13648298\|ref\|XP_{—} | hypothetical | 204 | 115/222 | 141/222 | 1e−49 |
| 012133.2\|(XM_012133) | protein FLJ11183 | | (51%) | (62%) | |
| | [Homo sapiens] | | | | |
| gi\|15426438\|gb\| | Similar to | 376 | 103/221 | 129/221 | 4e−40 |
| AAH13319.1\|AAH13319 | hypothetical | | (46%) | (57%) | |
| (BC013319) | protein FLJ11183 | | | | |
| | [Homo sapiens] | | | | |

A multiple sequence alignment is given in Table 5E, with the NOV5 protein being shown on line 1 in Table 5E in a ClustalW analysis, and comparing the NOV5 protein with the related protein sequences shown in Table 5D. This BLASTP data is displayed graphically in the ClustalW in Table 5E.

[image]

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 5F lists the domain description from DOMAIN analysis results against NOV5.

TABLE 5F
Domain Analysis of NOV5

| | Region of | | |
|---|---|---|---|
| Model | Homology | Score (bits) | E value |
| RhoGEF | 33-215 | −1.9 | 1.2e−05 |
| FYVE Ring Finger | 525-591 | 55.6 | 6.4e−14 |
| Plekstrin (PH) | 657-748 | 49.3 | 8.0e−7 |

Consistent with other known members of the Nuclear Protein-like family of proteins, NOV5 has, for example, an RhoGEF signature sequence and a FYVE Zinc Finger signature sequence, aw well as homology to other members of the Nuclear Protein-like Protein Family. NOV5 nucleic acids, and the encoded polypeptides, according to the invention are useful in a variety of applications and contexts. For example, NOV5 nucleic acids and polypeptides can be used to identify proteins that are members of the Nuclear Protein-like Protein Family. The NOV5 nucleic acids and polypeptides can also be used to screen for molecules, which inhibit or enhance NOV5 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection as well as other diseases, disorders and conditions.

In addition, various NOV5 nucleic acids and polypeptides according to the invention are useful, inter alia, as novel members of the protein families according to the presence of domains and sequence relatedness to previously described proteins. For example, the NOV5 nucleic acids and their encoded polypeptides include structural motifs that are characteristic of proteins belonging to the Nuclear Protein-like Protein Family.

The NOV5 nucleic acids and polypeptides, antibodies and related compounds according to the invention will be useful in therapeutic and diagnostic applications in the mediation of cardiac and nerve physiology. As such, the NOV5 nucleic acids and polypeptides, antibodies and related compounds according to the invention may be used to treat cardiovascular and nervous system disorders, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection as well as other diseases, disorders and conditions.

The NOV5 nucleic acids and polypeptides are useful for detecting specific cell types. For example, expression analysis has demonstrated that a NOV5 nucleic acid is expressed in Brown adipose, Vein, Umbilical Vein, Adrenal Gland/Suprarenal gland, Gall Bladder, Small Intestine, Colon, Lymphoid tissue, Spleen, Lymph node, Thymus, Brain, Temporal Lobe, Basal Ganglia/Cerebral nuclei, Substantia Nigra, Spinal Chord, Cervix, Ovary, Uterus, Testis, Lung, Lung Pleura, Larynx, Urinary Bladder, Kidney.

Additional utilities for NOV5 nucleic acids and polypeptides according to the invention are disclosed herein.

NOV6

A NOV6 polypeptide has been identified as a Secretory Protein-like protein (also referred to as CG94989-01). The disclosed novel NOV6 nucleic acid (SEQ ID NO:11) of 2372 nucleotides is shown in Table 6A. The novel NOV6 nucleic acid sequences maps to the chromosome 17.

An ORF begins with an ATG initiation codon at nucleotides 99-101 and ends with a TAA codon at nucleotides 1710-1712. A putative untranslated region and/or downstream from the termination codon is underlined in Table 6A, and the start and stop codons are in bold letters.

TABLE 6A
NOV6 Nucleotide Sequence

(SEQ ID NO:11)

CCGGCAAGGATGACGCCTCCGGAGGCCCTGGCCTCACTCCCACCTGGGCGCTAGGAGCCATCCCGGG
GCTCCAGCCAGGAGCCCTGCTGCCCAGGGGCATGGCCAAACCTTTCTTCCGACTCCAGAAGTTTCTC
CGCCGAACACAGTTCCTGCTGTTCTTCCTCACGGCTGCCTACCTGATGACCGGCAGCCTGCTGCTGC
TGCAGCGGGTCCGCGTGGCTCTCCCACAGGGCCCCCGGGCACCCGGCCCCCTGCAGACCTTGCCAGT
GGCCGCCGTGGCGCTGGGCGTGGGCTTGCTGGACAGCAGAGCCCTGCACGACCCTCGAGTCAGCCCA
GAGCTGCTGCTGGGTGTGGACATGCTGCAGAGCCCCCTGACCCGGCCCCGGCCCGGCCCCCGCTGGC
TCCGGAGCCGCAACTCGGAGCTGCGTCAGTTGCGTCGCCGCTGGTTCCACCACTTCATGAGTNGACT
CCCAGGCACCGCCCGCCCTGGGCCCCGAGGCTGCCAGGCCCGCCATCCACAGCCGAGGTCCTATGTC
TACGCCGGCTTGGAGGCCGGGGCGGAGTGTTACTGCGGGAACCGGCTGCCAGCGGTGAGCGTGGGGC
TGGAAGAGTGTAACCATGAGTGCAAAGGCGAGAAGGGCTCTGTGTGCGGGGCTGTGGACCGGCTCTC
CGTGTACCGTGTGGACGAGCTGCAGCCGGGCTCCAGGAAGCGGCGGACCGCCACCTACCGCGGATGC
TTCCGACTGCCAGAGAACATCACACATGCCTTCCCCAGCTCCCTGATACAGGCCAATGTGACCGTGG
GGACTTGCTCGGGCTTTTGTTCCCAGAAAGAGTTCCCCTTGGCCATTCTCAGGGGCTGGGAATGCTA
CTGTGCTTACCCTACCCCCCGGTTCAACCTGCGGGATGCCATGGACAGCTCAGTATGTGGCCAGGAC
CCTGAGGCACAGAGGCTGGCAGAATACTGTGAGGTCTACCAGACACCTGTGCAAGACACTCGTTGTA
CAGACAGGAGGTTCCTGCCTAACAAATCCAAAGTGTTTGTGGCTTTGTCAAGCTTCCCAGGAGCCGG
GAACACGTGGGCACGGCACCTCATTGAGCATGCCACTGGCTTCTATACAGGGAGCTACTACTTTGAT
GGAACCCTCTACAACAAAGGGTTCAAGGGCGAAAAGGACCACTGGCGGAGCCGACGCACCATCTGTG
TCAAAACCCACGAGAGTGGCAGGAGGGAGATTGAGATGTTTGATTCAGCCATCCTGCTAATCCGGAA
CCCATACAGGTCCCTGGTGGCAGAATTCAACAGAAAATGTGCCGGGCACCTGGGATATGCAGCTGAC
CGCAACTGGAAGAGCAAAGAGTGGCCGGACTTTGTCAACAGCTACGCCTCGTGGTGGTCCTCGCACG
TCCTGGACTGGCTCAAGTACGGGAAGCGGCTGCTGGTGGTGCACTACGAGGAGCTGCGGCGCAGCCT
GGTGCCCACGTTACGGGAGATGGTGGCCTTCCTCAACGTGTCTGTGAGCGAGGAGCGGCTGCTCTGC
GTGGAGAACAACAAGGAGGGCAGCTTCCGGCGGCGCGGCCGGCGCTCCCACGACCCTGAGCCCTTCA
CCCCGGAGATGAAAGACTTGATCAATGGCTACATCCGGACGGTGGACCAAGCCCTGCGTGACCACAA
CTGGACGGGGCTGCCCAGGGAGTATGTGCCCAGATGATAGGCCTGGCCCACGCCGCCGCCCCCGCTG
AGTGACGCAATCGCACCACGGGGCTGCGCTCCCCACTCTGATGCTCAGGCCCGTGGCCTCACTGGGA
CGAACGGTGGGTGGGGGGCTCACCCTGGTGCTGCCTCCCGCACAAGGAGACCTGGACACAACAGACA
CACATCACAAGGCGAACACAAATGGACACACATACCTGGCCACGAACCCACACCTCCTCAGACACTC
AGACACCACTCCAGGCTCATAGCCCCGTCTTGATGCAGAGAAGCCACCCACGTGGGGTGTGCCAGGC
ACCCCCAGCTACAAATGCAGCCACGCACAGACGTAACACACAGGTGCCAGGCCGTGTGCTCCTGGAG
GCTGGCTGGCTGTCTCTCTCACACAGATACACGTGCGCTCCCTGGGATCCGGGAGGCCCTGGGCTTC
CTGTGTGTAGCCCTGGCATAGACTTGCTCGTCAGGGTGTTTGACTCTGGGATGCTGGGCCGGGCAGA
CATTTATGCTCTGAGCAGCAAGGACCATTGGGATGGAGGTGGGCACAAAGACTGCTGCTTCCAGGGT
GTGCGGCCCTGGCCGTGTGTCTGACATCCCATAAATGTGTGTGTGGTGTGACTACGGGCACCACAAA
CTCCGCAAAAAAAAAAAAAAAAAAAAA

The NOV6 protein (SEQ ID NO:12) encoded by SEQ ID NO:11 is 537 amino acid residues in length and is presented using the one-letter amino acid code in Table 6B. Psort analysis predicts the NOV6 protein of the invention to be localized outside the cell with a certainty of 0.6997.

TABLE 6B
Encoded NOV6 protein sequencehz,1/41

(SEQ ID NO:12)

MAKPFFRLQKFLRRTQFLLFFLTAAYLMTGSLLLLQRVRVALPQGPRAPGPLQTLPVAAVALGVG
LLDSRALHDPRVSPELLLGVDMLQSPLTRPRPGPRWLRSRNSELRQLRRRWFHHFMSXLPGTARP
GPRGCQARHPQPRSYVYAGLEAGAECYCGNRLPAVSVGLEECNHECKGEKGSVCGAVDRLSVYRV
DELQPGSRKRRTATYRGCFRLPENITHAFPSSLIQANVTVGTCSGFCSQKEFPLAILRGWECYCA
YPTPRFNLRDAMDSSVCGQDPEAQRLAEYCEVYQTPVQDTRCTDRRFLPNKSKVFVALSSFPGAG
NTWARHLIEHATGFYTGSYYFDGTLYNKGFKGEKDHWRSRRTICVKTHESGRREIEMFDSAILLI
RNPYRSLVAEFNRKCAGHLGYAADRNWKSKEWPDFVNSYASWWSSHVLDWLKYGKRLLVVHYEEL
RRSLVPTLREMVAFLNVSVSEERLLCVENNKEGSFRRRGRRSHDPEPFTPEMKDLINGYIRTVDQ
ALRDHNWTGLPREYVPR

A search against the Patp database, a proprietary database that contains sequences published in patents and patent publications, yielded several homologous proteins shown in Table 6C.

TABLE 6C
Patp results for NOV6

| | | | Smallest |
|---|---|---|---|
| | | | Sum |
| Sequences producing High- | Reading | High | Prob |
| scoring Segment Pairs: | Frame | Score | P (N) |
| >patp:ABB15485 Human nervous system | +1 | 92 | 0.0036 |
| related polypeptide | | | |
| >patp:AAU50001 Propionibacterium acnes | +1 | 82 | 0.042 |
| immunogenic protein | | | |
| >patp:AAU50001 Propionibacterium acnes | +1 | 82 | 0.042 |
| immunogenic protein | | | |
| >patp:AAU18674 Renal and cardiovascular- | +1 | 79 | 0.085 |
| associated protein | | | |
| >patp:AAB95341 Human protein sequence | +1 | 99 | 0.17 |
| SEQ ID NO: 17621 | | | |

In a BLAST search of public sequence databases, it was found, for example, that the nucleic acid sequence of this invention has 2188 of 2189 bases (99%) identical to a gb:GENBANK-ID:AK000243|acc:AK000243.1 mRNA from Homo sapiens (cDNA FLJ20236 fis, clone COLF5810, highly similar to AB011095 Homo sapiens mRNA for KIAA0523 protein). The full amino acid sequence of the protein of the invention was found to have 395 of 395 amino acid residues (100%) identical to, and 395 of 395 amino acid residues (100%) similar to, the 468 amino acid residue ptnr:SPTREMBL-ACC:060276 protein from Homo sapiens (KIAA0523 PROTEIN)(FIG. 3B).

NOV6 also has homology to the proteins shown in the BLASTP data in Table 6D.

TABLE 6D
BLAST results for NOV6

| Gene Index/ | | | | | |
|---|---|---|---|---|---|
| Identifier | Protein/Organism | Length (aa) | Identity (%) | Positives (%) | Expect |
| gi\|14602977\|gb\| | Similar to | 575 | 523/575 | 524/575 | 0.0 |
| AAH09975.1\|AAH09975 | KIAA0789 gene | | (90%) | (90%) | |
| (BC009975) | product | | | | |
| | [Homo sapiens] | | | | |
| gi\|3043570\|dbj\| | KIAA0523 protein | 468 | 417/468 | 417/468 | 0.0 |
| BAA25449.1\|(AB011095) | [Homo sapiens] | | (89%) | (89%) | |
| gi\|18489296\|ref\|XP_{—} | CG9164 | 317 | 76/206 | 15/206 | 3e−28 |
| 082751.1\|(XM_082751) | [Drosophila | | (36%) | (54%) | |
| | melanogaster] | | | | |
| gi\|16944644\|emb\| | hypothetical | 2117 | 43/131 | 62/131 | 5e−08 |
| CAD11404.1\|(AL513445) | protein | | (32%) | (46%) | |
| | [Neurospora | | | | |
| | crassa] | | | | |
| gi\|11359357\|pir\|\| | beta-1,3 | 1032 | 40/128 | 55/128 | 2e−05 |
| T43257 | exoglucanase (EC | | (31%) | (42%) | |
| | 3.2.1.-) | | | | |
| | precursor - | | | | |
| | fungus | | | | |
| | (Trichoderma | | | | |
| | harzianum) | | | | |

A multiple sequence alignment is given in Table 6E, with the NOV6 protein being shown on line 1 in Table 6E in a ClustalW analysis, and comparing the NOV6 protein with the related protein sequences shown in Table 6D. This BLASTP data is displayed graphically in the ClustalW in Table 6E.

[image]

[image]

[image]

[image]

The NOV6 Clustal W alignment shown in Table 6E was modified to begin at amino residue 841 and end at amino acid residue 1860. The data in Table 6E includes all of the regions overlapping with the NOV6 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 6F lists the domain description from DOMAIN analysis results against NOV6.

TABLE 6F
Domain Analysis of NOV6

| | Region of | | |
|---|---|---|---|
| Model | Homology | Score (bits) | E value |
| Disintegrin | 151-159 | 5.8 | 0.73 |
| WSC domain | 120-186 | 36.8 | 5e−07 |
| Peptidase family | 346-354 | −0.2 | 8.4 |
| M1 | | | |
| Sulfotransferase | 288-518 | −143.3 | 0.14 |
| proteins | | | |

Consistent with other known members of the Secretory Protein-like family of proteins, NOV6 has, for example, has homology to other members of the Secretory Protein-like Protein Family. NOV6 nucleic acids, and the encoded polypeptides, according to the invention are useful in a variety of applications and contexts. For example, NOV6 nucleic acids and polypeptides can be used to identify proteins that are members of the Secretory Protein-like Protein Family. The NOV6 nucleic acids and polypeptides can also be used to screen for molecules, which inhibit or enhance NOV6 activity or function. Specifically, the nucleic acids and polypeptides according to the invention may be used as targets for the identification of small molecules that modulate or inhibit, e.g., cellular activation and signal transduction. These molecules can be used to treat, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus as well as other diseases, disorders and conditions.

In addition, various NOV6 nucleic acids and polypeptides according to the invention are useful, inter alia, as novel members of the protein families according to the presence of domains and sequence relatedness to previously described proteins. For example, the NOV6 nucleic acids and their encoded polypeptides include structural motifs that are characteristic of proteins belonging to the Secretory Protein-like Protein Family.

The NOV6 nucleic acids and polypeptides, antibodies and related compounds according to the invention will be useful in therapeutic and diagnostic applications in the mediation of cardiac physiology. As such, the NOV6 nucleic acids and polypeptides, antibodies and related compounds according to the invention may be used to treat cardiac and vascular system disorders, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus as well as other diseases, disorders and conditions

The NOV6 nucleic acids and polypeptides are useful for detecting specific cell types. For example, expression analysis has demonstrated that a NOV6 nucleic acid is expressed in Aorta.

Additional utilities for NOV6 nucleic acids and polypeptides according to the invention are disclosed herein.

NOV7

A NOV7 polypeptide has been identified as a Transmission Blocking Target Antigen S230 Precursor-like protein (also referred to as CG94978-01). The disclosed novel NOV7 nucleic acid (SEQ ID NO:13) of 1629 nucleotides is shown in Table 7A. The novel NOV7 nucleic acid sequences maps to the chromosome 1.

An ORF begins with an ATG initiation codon at nucleotides 1-3 and ends with a TGA codon at nucleotides 1627-1629. A putative untranslated region and/or downstream from the termination codon is underlined in Table 7A, and the start and stop codons are in bold letters.

TABLE 7A
NOV7 Nucleotide Sequence

(SEQ ID NO:13)

ATGGCGGTGCCCGGCCAGGCGGAGGAGGAGGCGACAGTTTACCTGGTAGTGAGCGGTATCCCCTCCG
TCTTGCGCTCGGCCCATTTACGGAGCTATTTTAGCCAGTTCCGAGAAGAGCGCGGCGGTGGCTTCCT
CTGTTTCCACTACCGGCATCGGCCTGAGCGGGCCCCTCCGCAGGCCGCTCCTAACTCTGCCCTAATT
CCTACCGACCCAGCCGCTGAGGGCCAGCTTCTCTCTCAGACTTCGGCCACCGATGTCCGGCCTCTCT
CCACTCGAGACTCTACTCCAATCCAGACCCGCACCTGCTGCTGCGTCATCTCGGTAAGGGGGTTGGC
TCAAGCTCAGAGGCTTATTCGCATGTACTCGGGCCGCCGGTGGCTGGATTCTCACGGGACTTGGCTA
CCGGGTCGCTGTCTCATCCGCAGACTTCGGCTACCTACGGAGGCATCAGGTCTGGGCTCCTTTCCCT
TCAAGACCCGGAAGGAACTGCAGAGTTGGAAGGCAGAGAATGAAGCCTTCACCCTGGCTGACCTGAA
GCAACTGCCGGAGCTGAACCCACCAGTGCTGATGCCCAGAGGGAATGTGGGGACTCCCCTGCGGGTC
TTTTTGGAGTTGATCCGGGCCTGCCGCCTACCCCCTCGGATCATCACCCAGCTGCAGCTCCAGTTCC
CCAAGACAGGTTCCTCCCGGCGCTACGGCAATGTGCCTTTTGAGTATGAGGACTCAGAGACTGTGGA
GCAGGAAGAGCTTGTGTATACAGCAGAGGGTGAAGAAATACCCCAAGGAACCTACCTGGCAGATATA
CCAGCCAGCCCCTGTGGAGAGCCTGAGGAAGAAGTGGGGAAGGAAGAGGAAGAAGAGTCTCACTCAG
ATGAGCTGTTCGGGTGTGCTGTGGTCATCCTCCCTGCGCACCTACAGCCGCAGACCGCCGGTGGGGG
GCGGGGGATGCCGGGCTGCCGCATCAGCGCCTGCGGCCCGGGGGCCCAGGAGGGGACGGCAGAGCAG
AGGTCGCCGCCGCCGCCCTGGGATCCCATGCCGTCCTCTCAGCCCCCGCCCCCAACTCCGACCTTGA
CTCCTACCCCGACCCCGGGTCAGTCCCCGCCGCTGCCGGACGCAGCTGGGGCTTCAGCAGGCGCGGC
CGAGGACCAGGAGCTGCAGCGCTGGCGCCAGGGCGCTAGCGGGATCGCGGGGCTCGCCGGCCCCGGA
GGGGGCTCTGGCGCGGCTGCGGGGGCGGGGGGCCGCGCGCTGGAGCTGGCCGAAGCACGGCGGCGGC
TGCTGGAGGTGGAGGGCCGCCGGCGCCTGGTGTCGGAGCTGGAGAGCCGCGTGCTGCAGCTGCACCG
CGTTTTCTTGGCGGCCGAGCTGCGCCTGGCGCACCGCGCGGAGAGCCTGAGCCGCCTGAGCGGCGGC
GTGGCGCAGGCCGAGCTCTACCTGGCGGCTCACGGGTCGCGCCTCAAGAAGGGCCCGCGCCGCGGCC
GCCGCGGCCGACCCCCCGCGCTGCTGGCCTCGGCGCTGGGCCTGGGCGGCTGCGTGCCCTGGGGTGC
CGGGCGACTGCGGCGCGGCCACGGCCCCGAGCCCGACTCGCCCTTCCGCCGCAGCCCGCCCCGCGGC
CCCGCCTCCCCGCAGCGCTGA

The NOV7 protein (SEQ ID NO:14) encoded by SEQ ID NO:13 is 542 amino acid residues in length and is presented using the one-letter amino acid code in Table 7B. Psort analysis predicts the NOV7 protein of the invention to be localized in the cytoplasm with a certainty of 0.4500.

TABLE 7B
Encoded NOV7 protein sequence

(SEQ ID NO:14)

MAVPGEAEEEATVYLVVSGIPSVLRSAHLRSYFSQFREERGGGFLCFHYRHRPERAPPQAAPNSA
LIPTDPAAEGQLLSQTSATDVRPLSTRDSTPIQTRTCCCVISVRGLAQAQRLIRMYSGRRWLDSH
GTWLPGRCLIRRLRLPTEASGLGSFPFKTRKELQSWKAENEAFTLADLKQLPELNPPVLMPRGNV
GTPLRVFLELIRACRLPPRIITQLQLQFPKTGSSRRYGNVPFEYEDSETVEQEELVYTAEGEEIP
QGTYLADIPASPCGEPEEEVGKEEEEESHSDELFGCAVVILPAHLQPQTAGGGRGMPGCRISACG
PGAQEGTAEQRSPPPPWDPMPSSQPPPPTPTLTPTPTPGQSPPLPDAAGASAGAAEDQELQRWRQ
GASGIAGLAGPGGGSGAAAGAGGRALELAEARRRLLEVEGRRRLVSELESRVLQLHRVFLAAELR
LAHRAESLSRLSGGVAQAELYLAAHGSRLKKGPRRGRRGRPPALLASALGLGGCVPWGAGRLRRG
HGPEPDSPFRRSPPRGPASPQR

A search against the Patp database, a proprietary database that contains sequences published in patents and patent publications, yielded several homologous proteins shown in Table 7C.

TABLE 7C
Patp results for NOV7

| | | | Smallest |
|---|---|---|---|
| | | | Sum |
| Sequences producing High-scoring | Reading | High | Prob |
| Segment Pairs: | Frame | Score | P (N) |
| >patp:AAU33166 Novel human secreted | +1 | 1533 | 5.8e−157 |
| protein #3657 | | | |
| >patp:AAE04880 Human protease protein- | +1 | 1533 | 5.8e−157 |
| 7 (PRTS-7) | | | |
| >patp:AAB94023 Human protein sequence | +1 | 1519 | 1.8e−155 |
| SEQ ID NO: 14157 | | | |
| >patp:AAU33124 Novel human secreted | +1 | 390 | 7.7e−36 |
| protein #3615 | | | |
| >patp:AAG02700 Human secreted protein, | +1 | 268 | 3.5e−22 |
| SEQ ID NO: 6781 | | | |

In a BLAST search of public sequence databases, it was found, for example, that the nucleic acid sequence of this invention has 874 of 876 bases (99%) identical to a gb:GENBANK-ID:AK022517|acc:AK022517.1 mRNA from Homo sapiens (cDNA FLJ12455 fis, clone NT2RM1000563, weakly similar to TRANSMISSION-BLOCKING TARGET ANTIGEN S230 PRECURSOR). The full amino acid sequence of the protein of the invention was found to have 290 of 292 amino acid residues (99%) identical to, and 290 of 292 amino acid residues (99%) similar to, the 525 amino acid residue ptnr:SPTREMBL-ACC:Q9H9Z3 protein from Homo sapiens (cDNA FLJ12455 FIS, CLONE NT2RM1000563, WEAKLY SIMILAR TO TRANSMISSION-BLOCKING TARGET ANTIGEN S230 PRECURSOR).

NOV7 also has homology to the proteins shown in the BLASTP data in Table 7D.

TABLE 7D
BLAST results for NOV7

| Gene Index/ | | | | | |
|---|---|---|---|---|---|
| Identifier | Protein/Organism | Length (aa) | Identity (%) | Positives (%) | Expect |
| gi\|18545154\|ref\|XP_{—} | hypothetical | 525 | 274/274 | 274/274 | e−147 |
| 084046.1\|(XM_084046) | protein FLJ12455 | | (100%) | (100%) | |
| | [Homo sapiens] | | | | |
| gi\|11545793\|ref\|NP_{—} | hypothetical | 525 | 272/274 | 272/274 | e−145 |
| 071361.1\|(NM_022078) | protein FLJ12455 | | (99%) | (99%) | |
| | [Homo sapiens] | | | | |
| gi\|18545156\|ref\|XP_{—} | similar to | 107 | 83/84 | 84/84 | 6e−37 |
| 086159.1\|(XM_086159) | hypothetical | | (98%) | (99%) | |
| | protein FLJ12455 | | | | |
| | [Homo sapiens] | | | | |
| gi\|18545158\|ref\|XP_{—} | hypothetical | 141 | 135/137 | 136/137 | 2e−33 |
| 097448.1\|(XM_097448) | protein XP_097448 | | (98%) | (98%) | |
| | [Homo sapiens] | | | | |
| gi\|17562286\|ref\|NP_{—} | K07B1.7b.p | 487 | 76/237 | 119/237 | 3e−30 |
| 505420.1\|(NM_073019) | [Caenorhabditis | | (32%) | (50%) | |
| | elegans] | | | | |

A multiple sequence alignment is given in Table 7E, with the NOV7 protein being shown on line 1 in Table 7E in a ClustalW analysis, and comparing the NOV7 protein with the select related protein sequences shown in Table 7D. This BLASTP data is displayed graphically in the ClustalW in Table 7E.

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 7F lists the domain description from DOMAIN analysis results against NOV7.

TABLE 7F
Domain Analysis of NOV7

| | Region of | | |
|---|---|---|---|
| Model | Homology | Score (bits) | E value |
| #PD343750 | 14-54 | 222 | 4e−19 |
| BLOCKING | | | |
| NT2RM1000563 | | | |
| TRANSMISSION- | | | |
| FIS | | | |
| #PD229850 | 55-90 | 179 | 4e−14 |
| BLOCKING | | | |
| NT2RM1000563 | | | |
| TRANSMISSION- | | | |
| FIS | | | |
| #PD138963 | 91-258 | 872 | 2e−94 |
| BLOCKING | | | |
| NT2RM1000563 | | | |
| TRANSMISSION- | | | |
| FIS | | | |

Consistent with other known members of the Transmission Blocking Target Antigen S230 Precursor-like family of proteins, NOV7 has, for example, three Blocking NT2RM1000563 Transmission-FIS Antigen Weakly Precursor Peptidase A2 signature sequences and homology to other members of the Transmission Blocking Target Antigen S230 Precursor-like Protein Family. NOV7 nucleic acids, and the encoded polypeptides, according to the invention are useful in a variety of applications and contexts. For example, NOV7 nucleic acids and polypeptides can be used to identify proteins that are members of the Transmission Blocking Target Antigen S230 Precursor-like Protein Family. The NOV7 nucleic acids and polypeptides can also be used to screen for molecules, which inhibit or enhance NOV7 activity or function. Specifically, the nucleic acids and polypeptides according to the invention may be used as targets for the identification of small molecules that modulate or inhibit, e.g., cellular activation and signal transduction. These molecules can be used to treat, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection

In addition, various NOV7 nucleic acids and polypeptides according to the invention are useful, inter alia, as novel members of the protein families according to the presence of domains and sequence relatedness to previously described proteins. For example, the NOV7 nucleic acids and their encoded polypeptides include structural motifs that are characteristic of proteins belonging to the Transmission Blocking Target Antigen S230 Precursor-like Protein Family.

The NOV7 nucleic acids and polypeptides, antibodies and related compounds according to the invention will be useful in therapeutic and diagnostic applications in the mediation of cardiac and nerve physiology. As such, the NOV7 nucleic acids and polypeptides, antibodies and related compounds according to the invention may be used to treat cardiovascular and nervous system disorders, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection

The NOV7 nucleic acids and polypeptides are useful for detecting specific cell types. For example, expression analysis has demonstrated that a NOV7 nucleic acid is expressed in Adipose, Heart, Aorta, Coronary Artery, Umbilical Vein, Pancreas, Liver, Gall Bladder, Colon, Bone Marrow, Thymus, Bone, Cartilage, Synovium/Synovial membrane, Skeletal Muscle, Brain, Left cerebellum, Right Cerebellum, Thalamus, Hypothalamus, Pituitary Gland, Frontal Lobe, Parietal Lobe, Cerebral Medulla/Cerebral white matter, Basal Ganglia/Cerebral nuclei, Substantia Nigra, Hippocampus, Cervix, Mammary gland/Breast, Uterus, Oviduct/Uterine Tube/Fallopian tube, Prostate, Testis, Lung, Bronchus, Larynx, Kidney, Retina, Skin, Epidermis.

Additional utilities for NOV7 nucleic acids and polypeptides according to the invention are disclosed herein.

NOV8

A NOV8 polypeptide has been identified as a Nuclear Protein-like protein (also referred to as CG94713-01). The disclosed novel NOV8 nucleic acid (SEQ ID NO:15) of 3807 nucleotides is shown in Table 8A. The novel NOV8 nucleic acid sequences maps to the chromosome 1.

An ORF begins with an ATG initiation codon at nucleotides 16-18 and ends with a TGA codon at nucleotides 3793-3795. A putative untranslated region and/or downstream from the termination codon is underlined in Table 8A, and the start and stop codons are in bold letters.

TABLE 8A
NOV8 Nucleotide Sequence

(SEQ ID NO:15)

ATGATAAAATAGAAGATGAATTGCAAACCTTCTTTACCAGTGATAAAGATGGAAATTACACATGCAT
ACAACCCGAAATCACCACCTACACAAAACTCTTCAGCCAGCAGTGTGAACTGGAATTCTGCCAACCC
AGATGACATGGTGGTTGATTATGAAACTGACCCTGCTGTAGTTACTGGTGAAAATATTTCTTTAAGC
CTTCAGGGTGTTGAAGTATTTGGTCATGAAAAGTCTTCTAGTGATTTCATTAGTAAGCAGGTGTTAG
ATATGCATAAAGATTCTATTTGTCAGTGTCCTGCACTTGTAGGTACTGAGAAGCCCAAATATCTGCA
ACACAGTTGTCATTCCCTAGAAGCAGTTGAGGGCCAGAGTGTTGAGCCATCTTTGCCTTTTGTGTGG
AAGCCTAATGACAATTTGAACTGTGCAGGCTACTGTGATGCCTTGGAGCTGAACCAAACATTTGACA
TGACAGTGGATAAAGTTAACTGCACCTTTATATCACATCATGCCATCGGAAAGAGTCAGTCCTTCCA
TACTGCTGGAAGCCTGCCACCAACTGGTAGGAGAAGTGGAAGTACATCTTCTTTATCCTATTCCACT
TGGACATCTTCCCATTCTGATAAGACGCATGCAAGAGAAACTACTTATGATAGAGAAAGCTTTGAAA
ACCCTCAAGTCACACCATCAGAAGCCCAAGACATGACTTACACAGCATTTTCTGATGTGGTGATGCA
AAGTGAGGTTTTTGTTTCAGATATTGGAAATCAGTGTGCATGTTCTTCAGGAAAGGTCACCAGTGAG
TACACAGATGGATCACAACAAAGACTAGTTGGAGAAAAAGAGACACAAGCACTAACACCAGTTTCTG
ATGGCATGGAAGTCCCCAATGATTCTGCATTACAAGAGTTCTTTTGTTTATCCCATGATGAATCCAA
TAGCGAACCACATTCACAGAGCTCATACAGGCACAAGGAAATGGGCCAAAATCTGAGAGAGACAGTG
TCCTATTGTCTTATTGATGATGAATGCCCTTTAATGGTGCCAGCTTTTGATAAGAGCGAAGCTCAAG
TGCTGAACCCAGAGCATAAAGTCACTGAGACTGAAGACACACAAATGGTCTCCAAAGGAAAGGATTT
GGGAACCCAAAATCATACCTCAGAATTGATTCTAAGTAGCCCGCCAGGACAAAAGGTGGGCTCGTCA
TTTGGACTGACTTGGGATGCAAATGATATGGTCATTAGCACAGACAAAACGATGTGCATGTCAACAC
CAGTCCTAGAACCCACAAAAGTAACCTTTTCTGTTTCACCGATTGAAGCGACGGAGAAATGTAAGAA
AGTGGAGAAGGGTAATCGAGGGCTTAAAAACATACCAGACTCGAAGGAGGCACCTGTGAACCTGTGT
AAACCCAGTTTAGGAAAATCAACAATCAAAACGAATACCCCAATAGGCTGCAAAGTTAGAAAAACTG
AAATTATAAGTTACCCAAGACCAAACTTCAAGAATGTCAAAGCAAAAGTTATGTCTAGAGCAGTGTT
GCAGCCCAAAGATGCTGCTTTATCAAAGGTCACGCCCAGACCTCAGCAGACCAGTGCCTCATCACCC
TCATCAGTGAATTCAAGACAACAAACAGTCTTGAGCAGAACACCGAGATCTGACTTGAATGCAGACA
AAAAAGCAGAAATTCTAATTAACAAGACACATAAGCAGCAGTTTAATAAACTCATTACTAGCCAGGC
TGTGCATGTTACAACTCATTCTAAAAATGCTTCACACAGGGTTCCAAGAACAACATCTGCCGTGAAA
TCGAATCAGGAAGATGTTGACAAAGCCAGTTCTTCTAACTCAGCATGCGAGACCGGGTCCGTTTCTG
CGTTGTTTCAGAAGATCAAAGGCATACTCCCTGTTAAAATGGAAAGTGCAGAATGTTTGGAAATGAC
CTATGTTCCCAACATTGATAGGATTAGCCCTGAAAAGAAGGGTGAAAAAGAAAATGGGACATCTATG
GAAAAACAAGAGCTGAAACAAGAGATTATGAATGAGACTTTTGAATATGGTTCTCTGTTTTTGGGCT
CTGCTTCAAAAACAACGACCACCTCAGGTAGGAATATATCCAAGCCTGACTCCTGCGGTTTGAGGCA
AATAGCTGCTCCAAAAGCCAAAGTGGGGCCCCCTGTTTCCTGTTTGAGGCGGAACAGTGACAATAGA
AATCCCAGTGCTGATCGAGCCGTATCTCCTCAGAGGATCAGCCGTGTGTCCAGTTCTGGAAAGCCTA
CATCCTTGAAAACTGCACAGTCGTCATGGGTGAATTTGCCTAGACCACTTCCTAAATCCAAAGCATC
TTTGAAAAGTCCTGCGCTGCGGAGGACAGGAAGCACCCCCTCAATAGCCAGCACCCACAGTGAGCTG
AGCACTTACAGCAACAATTCTGGTAATGCCGCTGTCATCAAATATGAGGAGAAACCTCCAAAACCAG
CATTTCAGAATGGTTCCTCAGGATCCTTTTATTTGAAGCCTTTGGTATCCAGGGCTCATGTTCACTT
GATGAAAACTCCTCCAAAAGGTCCTTCGAGAAAAAATTTATTTACAGCTCTTAATGCAGTTGAAAAG
AGCAGGCAAAAGAATCCTCGAAGCTTATGTATCCAGCCACAGACAGCTCCCGATGCGCTCCCCCCTG
AGAAAACACTTGAATTGACGCAATATAAAACAAAATGTGAAAACCAAAGTGGATTTATCCTGCAGCT
CAAGCAGCTTCTTGCCTGTGGTAATACCAAGTTTGAGGCATTGACAGTTGTGATTCAGCACCTGCTG
TCTGAGCGGGAGGAAGCACTGAAACAACACAAAACCCTATCTCAAGAACTTGTTAACCTCCGGGGAG
AGCTAGTCACTGCTTCAACCACCTGTGAGAAATTAGAAAAAGCCAGGAATGAGTTACAAACAGTGTA
TGAAGCATTCGTCCAGCAGCACCAGGCTGAAAAAACAGAACGAGAGAATCGGCTTAAAGAGTTTTAC
ACCAGGGAGTATGAAAAGCTTCGGGACACTTACATTGAAGAAGCAGAGAAGTACAAAATGCAATTGC
AAGAGCAGTTTGACAACTTAAATGCTGCGCATGAAACCTCTAAGTTGGAAATTGAAGCTAGCCACTC
AGAGAAACTTGAATTGCTAAAGAAGGCCTATGAAGCCTCCCTTTCAGAAATTAAGAAAGGCCATGAA
ATAGAAAAGAAATCGCTTGAAGATTTACTTTCTGAGAAGCAGGAATCGCTAGAGAAGCAAATCAATG
ATCTGAAGAGTGAAAATGATGCTTTAAATGAAAAATTGAAATCAGAAGAACAAAAAAGAAGAGCAAG
AGAAAAAGCAAATTTGAAAAATCCTCAGATCATGTATCTAGAACAGGAGTTAGAAAGCCTGAAAGCT
GTGTTAGAGATCAAGAATGAGAAACTGCATCAACAGGACATCAAGTTAATGAAAATGGAGAAACTGG
TGGACAACAACACAGCATTGGTTGACAAATTGAAGCGTTTCCAGCAGGAGAATGAAGAATTGAAAGC
TCGGATGGACAAGCACATGGCAATCTCAAGGCAGCTTTCCACGGAGCAGGCTGTTCTGCAAGAGTCG
CTGGAGAAGGAGTCGAAAGTCAACAAGCGACTCTCTATGGAAAACGAGGAGCTTCTGTGGAAACTGC
ACAATGGGGACCTGTGTAGCCCCAAGAGATCCCCCACATCCTCCGCCATCCCTTTGCAGTCACCAAG
GAATTCGGGCTCCTTCCCTAGCCCCAGCATTTCACCCAGATGACACCTCCCCAAA

Variant sequences of NOV8 are included in Example 3, Table 20. A variant sequence can include a single nucleotide polymorphism (SNP). A SNP can, in some instances, be referred to as a “cSNP” to denote that the nucleotide sequence containing the SNP originates as a cDNA.

The NOV8 protein (SEQ ID NO:16) encoded by SEQ ID NO:15 is 1259 amino acid residues in length and is presented using the one-letter amino acid code in Table 8B. Psort analysis predicts the NOV8 protein of the invention to be localized in the nucleus with a certainty of 0.7600.

TABLE 8B
Encoded NOV8 protein sequence

(SEQ ID NO:16)

MNCKPSLPVIKMEITHAYNPKSPPTQNSSASSVNWNSANPDDMVVDYETDPAVVTGENISLSLQG
VEVFGHEKSSSDFISKQVLDMHKDSICQCPALVGTEKPKYLQHSCHSLEAVEGQSVEPSLPFVWK
PNDNLNCAGYCDALELNQTFDMTVDKVNCTFISHHAIGKSQSFHTAGSLPPTGRRSGSTSSLSYS
TWTSSHSDKTHARETTYDRESFENPQVTPSEAQDMTYTAFSDVVMQSEVFVSDIGNQCACSSGKV
TSEYTDGSQQRLVGEKETQALTPVSDGMEVPNDSALQEFFCLSHDESNSEPHSQSSYRHKEMGQN
LRETVSYCLIDDECPLMVPAFDKSEAQVLNPEHKVTETEDTQMVSKGKDLGTQNHTSELILSSPP
GQKVGSSFGLTWDANDMVISTDKTMCMSTPVLEPTKVTFSVSPIEATEKCKKVEKGNRGLKNIPD
SKEAPVNLCKPSLGKSTIKTNTPIGCKVRKTEIISYPRPNFKNVKAKVMSRAVLQPKDAALSKVT
PRPQQTSASSPSSVNSRQQTVLSRTPRSDLNADKKAEILINKTHKQQFNKLITSQAVHVTTHSKN
ASHRVPRTTSAVKSNQEDVDKASSSNSACETGSVSALFQKIKGILPVKMESAECLEMTYVPNIDR
ISPEKKGEKENGTSMEKQELKQEIMNETFEYGSLFLGSASKTTTTSGRNISKPDSCGLRQIAAPK
AKVGPPVSCLRRNSDNRNPSADRAVSPQRIRRVSSSGKPTSLKTAQSSWVNLPRPLPKSKASLKS
PALRRTGSTPSIASTHSELSTYSNNSGNAAVIKYEEKPPKPAFQNGSSGSFYLKPLVSRAHVHLM
KTPPKGPSRKNLFTALNAVEKSRQKNPRSLCIQPQTAPDALPPEKTLELTQYKTKCENQSGFILQ
LKQLLACGNTKFEALTVVIQHLLSEREEALKQHKTLSQELVNLRGELVTASTTCEKLEKARNELQ
TVYEAFVQQHQAEKTERENRLKEFYTREYEKLRDTYIEEAEKYKMQLQEQFDNLNAAHETSKLEI
EASHSEKLELLKKAYEASLSEIKKGHEIEKKSLEDLLSEKQESLEKQINDLKSENDALNEKLKSE
EQKRRAREKANLKNPQIMYLEQELESLKAVLEIKNEKLHQQDIKLMKMEKLVDNNTALVDKLKRF
QQENEELKARMDKHMAISRQLSTEQAVLQESLEKESKVNKRLSMENEELLWKLHNGDLCSPKRSP
TSSAIPLQSPRNSGSFPSPSISPR

A search against the Patp database, a proprietary database that contains sequences published in patents and patent publications, yielded several homologous proteins shown in Table 8C.

TABLE 8C
Patp results for NOV8

| | | | Smallest |
|---|---|---|---|
| | | | Sum |
| Sequences producing High-scoring | Reading | High | Prob |
| Segment Pairs: | Frame | Score | P (N) |
| >patp:AAG63542 Amino acid sequence of | +1 | 6389 | 0.0 |
| a human ATIP isoform | | | |
| >patp:AAG63529 Amino acid sequence of | +1 | 6233 | 0.0 |
| a human ATIP isoform | | | |
| >patp:AAG63541 Amino acid sequence of | +1 | 3928 | 0.0 |
| a human ATIP isoform | | | |
| >patp:AAG63537 Amino acid sequence of | +1 | 3279 | 0.0 |
| a ATIP isoform | | | |
| >patp:AAG63530 Amino acid sequence of | +1 | 2954 | 1.5e−307 |
| a human ATIP isoform | | | |

In a BLAST search of public sequence databases, it was found, for example, that the nucleic acid sequence of this invention has 3751 of 3751 bases (100%) identical to a gb:GENBANK-ID:AB033114|acc:AB033114.1 mRNA from Homo sapiens (mRNA for KIAA1288 protein, partial cds). The full amino acid sequence of the protein of the invention was found to have 1245 of 1245 amino acid residues (100%) identical to, and 1245 of 1245 amino acid residues (100%) similar to, the 1245 amino acid residue ptnr:SPTREMBL-ACC:Q9ULD2 protein from Homo sapiens (KIAA1288 PROTEIN).

NOV8 also has homology to the proteins shown in the BLASTP data in Table 8D.

TABLE 8D
BLAST results for NOV8

| Gene Index/ | | | | | |
|---|---|---|---|---|---|
| Identifier | Protein/Organism | Length (aa) | Identity (%) | Positives (%) | Expect |
| gi\|6331407\|dbj\| | KIAA1288 protein | 1245 | 1245/1245 | 1245/1245 | 0.0 |
| BAA86602.1\|(AB033114) | [Homo sapiens] | | (100%) | (100%) | |
| gi\|17865632\|ref\|NP_{—} | AT2 receptor- | 436 | 404/436 | 409/436 | 0 .0 |
| 065800.1\|(NM_020749) | interacting | | (92%) | (93%) | |
| | protein 1 | | | | |
| | [Homo sapiens] | | | | |
| gi\|10436722\|dbj\| | unnamed protein | 240 | 239/240 | 239/240 | e−107 |
| BAB14894.1\|(AK024357) | product | | (99%) | (99%) | |
| | [Homo sapiens] | | | | |
| gi\|3882269\|dbj\| | KIAA0774 protein | 1163 | 135/366 | 224/366 | 9e−49 |
| BAA34494.1\|(AB018317) | [Homo sapiens] | | (36%) | (60%) | |
| gi\|17475630\|ref\|XP_{—} | KIAA0774 protein | 901 | 135/366 | 224/366 | 3e−48 |
| 029364.3\|(XM_029364) | [Homo sapiens] | | (36%) | (60%) | |

A multiple sequence alignment is given in Table 8E, with the NOV8 protein being shown on line 1 in Table 8E in a ClustalW analysis, and comparing the NOV8 protein with the related protein sequences shown in Table 8D. This BLASTP data is displayed graphically in the ClustalW in Table 8E.

[image]

[image]

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 8F lists the domain description from DOMAIN analysis results against NOV8.

TABLE 8F
Domain Analysis of NOV8

| | Region of | | |
|---|---|---|---|
| Model | Homology | Score (bits) | E value |
| RNA polymerase | 1008-1094 | −12.8 | 5.7 |
| omega subunit | | | |
| Intermediate | 967-1193 | 48.9 | 2.0e−06 |
| filament | | | |

Consistent with other known members of the Nuclear Protein-like family of proteins, NOV8 has, for example, an RNA polymerase omega subunit signature sequence and homology to other members of the Nuclear Protein-like Protein Family. NOV8 nucleic acids, and the encoded polypeptides, according to the invention are useful in a variety of applications and contexts. For example, NOV8 nucleic acids and polypeptides can be used to identify proteins that are members of the Nuclear Protein-like Protein Family. The NOV8 nucleic acids and polypeptides can also be used to screen for molecules, which inhibit or enhance NOV8 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Hyperparathyroidism, Hypoparathyroidism as well as other diseases, disorders and conditions.

In addition, various NOV8 nucleic acids and polypeptides according to the invention are useful, inter alia, as novel members of the protein families according to the presence of domains and sequence relatedness to previously described proteins. For example, the NOV8 nucleic acids and their encoded polypeptides include structural motifs that are characteristic of proteins belonging to the Nuclear Protein-like Protein Family.

The NOV8 nucleic acids and polypeptides, antibodies and related compounds according to the invention will be useful in therapeutic and diagnostic applications in the mediation of cardiac or endocrine physiology. As such, the NOV8 nucleic acids and polypeptides, antibodies and related compounds according to the invention may be used to treat infection, cardiovascular system, immune system, and nervous system disorders, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Hyperparathyroidism, Hypoparathyroidism as well as other diseases, disorders and conditions.

The NOV8 nucleic acids and polypeptides are useful for detecting specific cell types. For example, expression analysis has demonstrated that a NOV8 nucleic acid is expressed in Heart, Aorta, Coronary Artery, Vein, Umbilical Vein, Adrenal Gland/Suprarenal gland, Pancreas, Islets of Langerhans, Parathyroid Gland, Thyroid, Pineal Gland, Tongue, Salivary Glands, Stomach, Liver, Small Intestine, Colon, Ascending Colon, Lymphoid tissue, Spleen, Brain, Thalamus, Hypothalamus, Temporal Lobe, Amygdala, Cerebral Medulla/Cerebral white matter, Basal Ganglia/Cerebral nuclei, Substantia Nigra, Hippocampus, Spinal Chord, Cervix, Mammary gland/Breast, Ovary, Placenta, Uterus, Prostate, Testis, Lung, Nasoepithelium, Larynx, Urinary Bladder, Kidney, Kidney Cortex, Retina, Skin, Foreskin, Epidermis, Dermis.

Additional utilities for NOV8 nucleic acids and polypeptides according to the invention are disclosed herein.

NOV9

A NOV9 polypeptide has been identified as a Hemicentin precursor-like protein (also referred to as CG94702-01). The disclosed novel NOV9 nucleic acid (SEQ ID NO:17) of 11796 nucleotides is shown in Table 9A. The novel NOV9 nucleic acid sequences maps to the chromosome 9.

An ORF begins with an ATG initiation codon at nucleotides 1-3 and ends with a TAA codon at nucleotides 11794-11796. A putative untranslated region and/or downstream from the termination codon is underlined in Table 9A, and the start and stop codons are in bold letters.

TABLE 9A
NOV9 Nucleotide Sequence

(SEQ ID NO:17)

ATGTCTGCCTTATTTGCAGCTGTGTACCAGATGCTAAAACCACGCCTGGTCCATAACAGCCCACATC
CGGTGACCTATCAAATTGAGGCAAGTTTAAAGCCAGAGCAGCCTGGTGTCACGCTGGTGTCCATCCC
AGTCTTCCTGGCACCTTCCTGGCACAAAGCCTCAGAGCTGATCCCGACCCAGTCCTTCCGAGCACAG
GGGGCAGGCAAGCAGCTCCTCGGCTCTCCTTGCCCCCAAGTGCCCCCCAGCATCCGGGAGGACGGGC
GCAAGGCCAACGTGTCGGGTATGGCCGGGCAGTCCCTGACGCTGGAGTGTGACGCGAACGGCTTTCC
AGTCCCTGAGATCGTGTGGCTGAAGGACGCGCAGCTGATTCCTAAGGTGGGCGGCCACCGCCTCCTG
GACGAGGGCCAGTCCCTCCACTTCCCCAGGATCCAGGAGGGTGATTCTGGGCTCTACTCCTGCCGGG
CAGAGAACCAGGCTGGCACCGCCCAGAGGGACTTCCATCTCCTTGTGCTCACCCCTCCTTCCGTGCT
TGGAGCCGGGGCCGCTCAGGAGGTGCTAGGATTGGCCGGTGCAGACGTGGAGCTGCAGTGTTGGACC
TCAGGGGTCCCCACGCCCCAGGTGGAGTGGACCAAGGACAGGCAGCCTGTCCTTCCGGCAGGCCCTC
ACCTGCAGGTCCAGGAGGATGGCCAGGTTCTCAGGATCACCGGCAGTCACGTGGGGGATGAGGGACG
ATACCAGTGCGTGGCCTTCAGCCCAGCTGGTCAGCAGGCCAGGGACTTCCAGCTCCGAGTTCATGCG
CCCCCCACTATCTGGGGCTCCAACGAGACAGGCGAGGTGGCCGTCATGGAGGACCACCTAGTGCAGC
TCCTGTGTGACGGTCGAGGAGTGCCCACCCCAAACATCACCTGGTTCAAGGACGGGGCCCTGCTCCC
CACCAGCACCAAGGTGGTCTACACTAGGGGCGGTCGGCAGTTGCAGCTGGGGAGGGCCCAGAGCTCC
GATGCCGCCGTCTACACCTGCAAGGCCAGCAATGCTGTGGGGGCCGCAGAGAAGGCCACCAGGCTGG
ATGTTTATGTCCCACCTACCATCGAGGGCGCCGGTGGAAGACCATACGTGGTGAAGGCTGTGGCTGG
GAGGCCTGTGGCGCTGGAGTGCGTGGCCAGAGGCCACCCGTCCCCCACCCTCTCCTGGCACCACGAG
GGGCTGCCCGTGGCAGAGAGCAACGAGTCGCGGCTGGAGACAGACGGGAGTGTGCTGAGGCTGGAGA
GCCCGGGGGAGGCATCCAGTGGCCTGTACAGCTGTGTGGCCAGCAGTCCTGCCGGGGAAGCCGTCCT
GCAGTACTCCGTGGAGGTTCAGGTGCCCCCACAGCTCCTGGTGGCTGAAGGCTTGGGACAGGTGACC
ACCATCGTGGGACAGCCCCTGGAACTTCCCTGCCAGGCCTCAGGCTCCCCAGTACCCACTATCCAGT
GGCTGCAGAATGGCCGCCCAGCCGAGGAGCTGGCTGGGGTGCAGGTGGCCTCGCAGGGGACCACACT
GCACATTGACCATGTGGAGCTGGACCACTCAGGCCTCTTCGCCTGCCAGGCCACCAATGAGGCGGGC
ACTGCCGGGGCCGAGGTGGAGGTGTCTGTGCATGAGTTCCCATCGGTCAGTATCATTGGGGGTGAGA
ACATCACAGCTCCTTTCCTGCAGCCTGTGACCCTCCAGTGCATAGGGGATGGGGTGCCCACCCCAAG
CCTCCGTTGGTGGAAGGATGGTGTAGCCCTGGCAGCCTTTGGGGGGAACCTACAGATTGAGAAGGTG
GACCTGAGGGACGAGGGCATCTACACTTGTGCTGCTACCAACCTGGCTGGGGAGAGCAAGAGGGAAG
TGGCGCTGAAAGTTTTGGTGCCCCCCAACATCGAGCCAGGCCCAGTCAACAAGGCAGTGCTGGAAAA
TGCCTCAGTGACCTTGGAGTGTCTGGCTTCGGGCGTGCCCCCTCCTGATGTCTCCTGGTTCAAGGGC
CACCAACCTGTCTCTTCATGGATGGGAGTGACAGTATCAGTGGATGGGAGAGTTCTCCGCATTGAGC
AAGCCCAGCTTTCTGATGCTGGGAGCTACCGCTGTGTGGCATCCAATGTGGCAGGTAGCACAGAGCT
GCGGTATGGCCTACGGGTCAATGTGCCCCCTCGAATCACACTGCCACCCAGCCTGCCAGGCCCTGTG
TTGGTCAACACCCCTGTCCGGCTGACCTGCAATGCCACCGGTGCCCCCAGCCCCACACTGATGTGGC
TGAAGGATGGAAACCCTGTGTCCCCTGCAGGGACCCCTGGCCTGCAGGTCTTCCCTGGGGGCCGGGT
CCTCACCTTGGCTAGTGCCCGGGCCTCCGACTCTGGGAGGTACTCCTGCGTGGCTGTGAGCGCGGTG
GGCGAGGACCGCCAGGATGTTGTCCTGCAACTCCACATGCCCCCGAGTATCCTTGGAGAAGAGCTGA
ATGTGTCCGTTGTGGCCAATGAGTCAGTGGCCCTGGAGTGCCAGAGCCACGCCATGCCCCCTCCTGT
GCTGAGCTGGTGGAAGGACGGGCGGCCCCTGGAACCACGGCCTGGAGTCCACCTCTCCGCAGACAAA
GCCTTGCTGCAGGTGGACAGAGCCGATGTGTGGGATGCGGGCCATTACACCTGTGAGGCACTGAACC
AGGCCGGCCACTCAGAGAAACACTACAATCTGAACGTCTGGGGTCAACCCCTCCCCGGGGAGGGGGC
TGGCCTCCAGCACGTGTCGGCTGTGGGGAGGCTGTTGTACCTGGGACAGGCCCAGCTGGCTCAGGAA
GGAACATACACCTGTGAATGCAGCAACGTGGTGGGGAACAGCAGCCAGGACCTGCAGCTGGAGGTGC
ACGTTCCCCCTCAGATTGCCGGTCCCCGGGAGCCTCCCACACAAGTCTCTGTGGTCCAGGATGGAGT
GGCCACTCTGGAGTGCAACGCCACAGGGAAACCCCCTCCGACAGTGACATGGGAGCGGGACGGCCAG
CCCGTGGGGGCTGAACTGGGCCTGCAGCTGCAGAACCAGGGTCAGAGCCTGCATGTGGAGCGGGCCC
AGGCTGCCCACACTGGACGCTACAGCTGTGTGGCCGAGAACCTGGCTGGGAGGGCAGAGAGGAAGTT
TGAGCTCTCCGTACTGGTGCCCCCAGAGCTCATTGGAGACTTGGACCCGCTGACCAACATCACTGCT
GCCTTGCACAGCCCCTTAACTCTGCTCTGTGAAGCCATGGGGATCCCACCTCCAGCCATCCGCTGGT
TCCGAGGGGAGGAGCCTGTCAGCCCCGGGGAGGACACCTACCTGCTGGCAGGTGGCTGGATGCTGAA
GATGACTCAGACACAGGAGCAAGACAGTGGCCTCTACTCATGCCTGGCAAGCAACGAGGCTGGGGAG
GCACGGAGGAACTTCAGTGTGGAGGTGCTGGTTCCTCCCAGTATTGAGAACGAGGACTTGGAGGAGG
TGATCAAGGTCCTTGATGGACAGACTGCCCATCTTATGTGCAACGTCACAGGCCACCCACAGCCCAA
GCTCACATGGTTCAAAGATGGCCGGCCTCTGGCTAGGGGAGATGCTCACCACATCTCCCCAGACGGA
GTCCTCCTGCAGGTCCTCCAGGCAAACCTGTCCAGTGCTGGCCACTACTCCTGCATTGCAGCCAACG
CTGTTGGGGAGAAGACCAAACACTTCCAGCTCAGTGTCCTGTTGGCTCCCACCATCCTGGGAGGGGC
CGAGGACAGTGCAGATGAGGAGGTGACCGTGACTGTCAACAACCCCATCTCTCTGATCTGCGAGGCC
CTGGCCTTCCCTTCCCCCAACATCACCTGGATGAAGGACGGGGCCCCGTTTGAGGCCTCCAGGAACA
TCCAGCTGCTCCCAGGTACCCACGGGCTGCAGATCCTGAATGCCCAGAAGGAAGATGCTGGCCAGTA
CACCTGCGTGGTCACCAATGAGCTCGGGGAGGCCGTGAAAAACTACCATGTGGAAGTGCTCATCCCC
CCTTCCATCTCCAAAGACGACCCCTTGGCGGAGGTCGGCGTGAAGGAGGTGAAGACCAAGGTCAACA
GCACCTTGACCTTGGAGTGTGAGAGCTGGGCTGTGCCCCCGCCCACCATCCGCTGGTACAAGGATGG
ACAGCCCGTGACCCCCAGCTCGCGGCTGCAGGTCCTGGGTGAAGGGCGACTGCTCCAGATCCAGCCC
ACACAGGTCTCAGACTCGGGGCGGTACCTGTGTGTGGCCACCAATGTGGCTGGCGAGGACGACCAGG
ACTTCAACGTGCTCATCCAGGTGCCCCCCATGTTCCAGAAGGTGGGTGATTTCAGTGCAGCCTTCGA
GATCCTGTCCCGGGAGGAGGAGGCCCGGGGCGGAGTCACGGAATACAGGGAGATCGTGGAGAACAAC
CCAGCCTACCTGTACTGCGACACCAACGCGATCCCACCCCCGGACCTCACCTGGTACAGAGAGGATC
AGCCCCTCTCGGCCGGGGATGAGGTGTCTGTGCTGCAAGGAGGCCGGGTCCTGCAGATCCCCCTGGT
GCGGGCAGAGAACGCCGGGAGGTACTCGTGCAAGGCCTCCAACGAGGTGGGCGAGGACTGGCTGCAC
TACGAGCTGCTGGTGCTGACCCCACCTGTGATCCTGGGTGACACAGAGGAGCTGGTGGAAGAGGTGA
CAGTCAATGCCAGCAGCACCGTCAGCCTGCAGTGCCCGGCCCTGGGAAACCCCGTGCCCACCATCTC
ATGGCTCCAGAATGGGCTGCCTTTCTCCCCGAGCCCACGGCTGCAGGTCCTGGAGGACGGGCAAGTC
TTGCAGGTTTCCACGGCAGAGGTGGCCGACGCCGCCAGCTACATGTGTGTGGCCGAGAACCAGGCGG
GCTCCGCTGAGAAGCTCTTCACCCTCAGGGTTCAAGGCCTGGACTTGGAGCAGGTCACTGCCATCCT
CAACAGCAGCGTCTCCCTCCCTTGCGACGTCCACGCTCACCCAAACCCCGAGGTCACGTGGTACAAG
GACAGCCAGGCCCTCTCCCTGGGTGAAGAGGTCTTCCTCCTGCCTGGCACCCACACGCTGCAGCTGG
GGAGAGCACGGCTGTCGGACTCCGGGATGTACACATGCGAAGCCCTCAATGCTGCCGGCCGAGACCA
GAAGCTGGTGCAGCTCAGTGTTCTGGTTCCCCCGGCCTTCAGGCAGGCTCCCAGAGGTCCCCAGGAT
GCGGTCCTGGTGAGGGTCGGGGACAAAGCTGTCCTGAGCTGCGAGACAGATGCGCTCCCTGAGCCAA
CTGTGACCTGGTACAAGGATGGGCAGCCCCTGGTCCTGGCACAGCGGACCCAGGCTCTGCGGGGTGG
GCAGAGGCTGGAGATCCAGGAAGCCCAGGTATCGGATAAAGGTTTATACAGCTGTAAAGTCAGCAAC
GTGGCTGGGGAGGCCGTGCCGACCTTCACCCTCACCGTCCAGGTGCCCCCAACATTTGAGAACCCCA
AGACAGAGACAGTGACCCAGGTGGCTGGGAGCCCCCTGGTCCTGACCTGTGATGTGTCCGGGGTCCC
TGCACCCACGGTCACTTGGCTGAAGGACAGGATGCCTGTGGAGAGCAGCGCGGTGCACGGTGTGGTC
TCCCGGGGGGGCCGCCTCCAGCTGAGCCGCCTGCAACCGGCCCAGGCGGGCACCTACACGTGCGTGG
CTGAGAACACCCAGGCTGAGGCCCGCAAGGACTTCGTGGTAGCAGTGCTGGTGGCCCCCCGGATCCG
GAGCTCGGGCGTGGCGCGGGAGCACCATGTCTTGGAAGGGCAGGAGGTGCGGCTGGACTGTGAGGCC
GATGGGCAGCCGCCGCCGGACGTGGCCTGGCTGAAGGACGGCAGCCCGCTGGGCCAGGACATGGGCC
CCCACCTCCGGTTCTACCTGGACGGCGGCTCCCTGGTGCTAAAAGGCCTGAGGGCCTCGGACGCGGG
TGCCTACACCTGCGTGGCCCACAACCCAGCCGGGGAGGACGCCAGGCTGCACACGGTGAATGTGCTG
GTTCCTCCCACCATCAAGCAGGGAGCAGACGGCTCGGGGACCCTGGTGAGCAGGCCTGGGGAGCTGG
TGACCATGGTGTGCCCTGTGCGGGGCTCCCCGCCCATCCACGTGAGCTGGCTCAAGGACGGCCTGCC
CCTCCCGCTCTCCCAGCGCACCCTCCTCCACGGCTCTGGCCACACCCTCAGGATTTCCAAGGTGCAA
TTGGCAGACGCTGGCATCTTCACCTGTGTGGCCGCAAGCCCAGCTGGCGTGGCGGACAGGAACTTCA
CCTTGCAGGTGCAGGTGCCCCCTGTCCTGGAGCCGGTGGAGTTCCAGAATGACGTGGTGGTGGTTCG
TGGCTCCCTGGTGGAACTCCCGTGCGAGGCCCGGGGCGTTCCCCTGCCTCTCGTGTCGTGGATGAAG
GATGGGGAACCCTTGTTGTCCCAGAGCCTCGAGCAGGGGCCCAGCCTGCAGCTGGAGGCAGTGGGAG
CTGGTGACTCGGGGACCTACTCCTGTGTGGCCGTGAGCGAGGCGGGGGAAGCCAGGAGGCATTTCCA
GCTGACCGTCATGGAGCCCCCTCACATTGAGGACTCAGGCCAGCCTACAGAGCTGTCGCTGACCCCC
GGCGCCCCCATGGAGCTCCTCTGTGATGCCCAGGGCACCCCCCAGCCCAACATCACCTGGCATAAGG
ACGGGCAGGCCCTGACCAGGCTGGAGAACAACAGCAGAGCCACACGGGTGCTCCGGGTGGAGAATGT
GCAGGTTAGGGATGCTGGGCTGTACACTTGTCTGGCTGAAAGCCCTGCAGGTGCAATTGAGAAGAGC
TTCCGGGTCAGGGTTCAAGCCCCTCCAAACATTGTTGGGCCCCGAGGCCCCCGCTTTGTGGTCGGCC
TGGCCCCAGGGCAGCTGGTCCTGGAGTGTTCGGTGGAGGCAGAGCCAGCGCCCAAGATCACGTGGCA
CCGAGACGGCATTGTGCTGCAGGAGGACGCCCACACACAATTCCCGGAGCGGGGCAGGTTCCTCCAG
CTGCAGGCCCTGAGCACGGCTGACAGCGGCGACTACAGCTGCACAGCCCGCAACGCCGCAGGCAGCA
CTAGTGTCGCCTTCCGCGTGGAGATCCACACGGTGCCCACCATCCGGTCAGGACCACCTGCAGTGAA
CGTCTCAGTGAACCAGACAGCCCTGCTGCCTTGCCAGGCCGACGGCGTGCCCGCACCCCTCGTGAGC
TGGCGGAAGGACAGGGTCCCCCTGGATCCCAGGAGCCCCAGGGCAACCCCCATCCATTCTAGGTTTG
AAATTCTGCCTGAGGGTTCCCTGAGAATCCAGCCAGTCCTTGCCCAGGACGCCGGCCACTACCTCTG
CCTGGCATCCAACTCTGCTGGCTCCGATCGTCAAGGCCGTGACCTACGGGTCTTGGAGCCTCCAGCC
ATCGCCCCCAGCCCCTCCAACCTGACCCTGACCGCCCACACCCCAGCCTTGCTGCCCTGCGAGGCCA
GCGGCTCCCCTAAGCCCCTGGTGGTCTGGTGGAAGGACGGACAGAAGCTGGACTTCCGCCTGCAGCA
GGGCGCCTACCGCCTCCTGCCCTCCAACGCCCTGCTCCTCACGGCCCCCGGCCCCCAGGACTCAGCC
CAGTTTGAATGCGTGGTGAGCAATGAGGTGGGCGAGGCCCACAGGCTCTACCAGGTGACCGTCCATG
TGCCTCCCACCATTGCCGATGACCAGACAGACTTCACCGTGACCATGATGGCACCTGTGGTCCTCAC
ATGTCACAGCACGGGTATACCAGCTCCGACCGTGTCCTGGAGCAAGGCAGGCGCCCAGCTAGGAGCT
CGGGGGAGTGGCTATCGTGTCTCACCATCGGGCGCCCTGGAGATCGCGCAGGCCCTCCCCATCCACG
CAGGCCGCTACACCTGCTCAGCCCGCAACTCTGCCGGCGTAGCCCACAAGCACGTCTTCCTCACTGT
GCAAGCCTCCCCGGTGGTGAAGCCGCTGCCCAGCGTGGTTCGGGCAGTGGCAGAGGAGGAGGTGCTG
CTGCCCTGCGAGGCCTCAGGCATCCCCCGGCCGACCATCACCTGGCAGAAGGAAGGGCTCAACGTCG
CTACTGGAGTGAGTACCCAGGTCCTACCAGCCGGACAGCTGCGGATTGCCCATGCCAGCCCAGAGGA
TGCTGGAAACTATCTCTGCATCGCTAAGAACAGTGCGGGCAGTGCCATGGGGAAGACGCGGCTGGTG
GTGCAAGTCCCACCAGTGATCGAGAATGGCCTCCCAGACCTGTCCACCACCGAAGGCTCCCACGCCT
TCTTGCCTTGCAAGGCGAGGGGCAGTCCTGAGCCCAACATCACCTGGGACAAAGATGGCCAGCCTGT
GTCGGGCGCCGAGGGGAAGTTCACCATCCAGCCTTCTGGGGAGTTGCTGGTGAAGAACTTGGAGGGC
CAGGACGCAGGCACCTATACCTGTACCGCTGAGAACGCCGTGGGCCGGGCCCGCCGCCGCGTGCACC
TCACCATCCTGGTACTGCCTGTGTTCACCACCCTGCCTGGGGACCGCAGCCTGCGCCTTGGGGACAG
GCTGTGGCTTCGCTGTGCAGCCCGGGGCAGCCCCACCCCTCGCATTGGCTGGACTGTCAACGACCGG
CCAGTCACAGAAGGGGTGTCTGAGCAGGATGGAGGCAGCACGCTGCAGCGGGCCGCTGTCTCCAGAG
AAGACAGCGGGACCTATGTCTGCTGGGCGGAGAACAGAGTGGGCCGCACGCAGGCGGTCAGCTTCGT
CCACGTGAAGGAGGCTCCTGTCCTACAAGGGGAGGCTTTCTCCTACCTGGTGGAACCTGTAGGAGGC
AGCATTCAGCTAGACTGTGTGGTGCGTGGAGACCCAGTGCCGGACATCCACTGGATCAAAGATGGCC
TTCCACTGCGGGGCAGCCACCTCCGGCACCAGCTGCAGAATGGCTCGCTGACCATCCGCAGGACTGA
GGCAAGGCGGGGCCTGGCACCTTGGAGGGACGATGCGGGACGGTACCAGTGCCTGGCAGAGAATGAG
ATGGGCGTGGCGAAGAAAGTGGTGATCCTCGTCCTGCAGACCAGGATGGTGCCAGCAGAGCCCCACT
TGAAGCGCCAACTCCCACCGATCCCCAGCAATAATGAGGCACCCTCCCTGTTCCCGGGTGTCCATGG
AGGCCACGTGGGGAACCCGGACTTCCACTCTCATCTAGCAGAAGTTCTCGCCGTTCAGTTGCTGGCT
GGGTCCCTGCTCTTCTCAGCCAGGGCCATGCCGCAGGCCAGCACAGCAGCCATTTCCCTTTTGGCTC
CTACCAGTTTTGCCCCTTTTCCTGATGATATTTCTCAGGGCATACTTTCATCCTCTACTGCACATCA
AGGCAGCCCCCAGGGGTGGCAAAAGCTGCTGTTTTTCACAGCCATCCCTAATAAAACCACTGTGATG
GTCACGGTGGAGCCCCAGGACATGACAGTGAGATCTGGGGATGACGTGGCCCTGCGGTGCCAGGCCA
CTGGAGAGCCCACACCCACCATTGAATGGCTACAGGCGGGTCAACCCTTGCGGGCCAGCCGGCGGCT
CCGGACCCTGCCCGATGGGAGCCTGTGCCTGGAGAACGTGGAGACTGGGGATGCAGGCACCTACGAC
TGCGTCGCTCACAACCTCCTGGGCTCTGCCACAGCCCGGGCGTTCCTGGTCTGTGCCAGCCACGCCA
TCGTGGGCTCCCGGCATTTCAGAGACCCACAGGTCTTCTGTGAGTTTGTGGTCCCGCCTCCTCATTT
TACAGGGGAGCCCCAGGGGAGCTGGGGCAGCATGACTGGGGTGATAAATGGCCGGAAATTTGGCGTG
GCCACACTCAACACCAGCGTGATGCAGGAGGCACACTCCGGGGTCAGCAGCATCCACAGCAGCATCC
GCCATGTCCCAGCAAACGTGGGGCCTCTGATGCGGGTGCTCGTGGTCACCATCGCCCCCATCTACTG
GGCCCTGGCCAGAGAGAGTGGGGAAGCCCTGAATGGCCACTCTCTGACTGGGGGCAGGTTCCGGCAG
GAGTCACACGTGGAGTTTGCTACAGGGGAGCTGCTCACGATGACCCAGGTGGCCCGGGGTCTGGATC
CCGATGGCCTCCTGCTCCTCGACGTGGTGGTCAATGGCGTTGTCCCCGAGAGCCTGGCTGACGCAGA
TCTTCAAGTGCAGGACTTTGAGGAGCACTACGTGCAAACAGGGCCTGGCCAGCTGTTCGTGGGCTCC
ACACAGCGCTTCTTCCAGGGCGGCCTCCCCTCGTTCCTACGCTGCAACCACAGCATCCAGTACAACG
CGGCCCGGGGCCCCCAGCCCCAGCTGGTGCAGCACCTGCGGGCCTCAGCTATCAGCTCGGCCTTTGA
TCCAGAGGCCGAGGCCCTGCGCTTCCAGCTCGCTACAGCCCTGCAGGCGGAGGAGAACGAGGTCGGC
TGCCCCGAGGGCTTTGAGCTGGACTCCCAGGGAGCGTTTTGTGTGGACAGGGACGAGTGCTCAGGAG
GCCCTAGCCCCTGCTCCCATGCCTGCCTTAATGCACCCGGCCGCTTCTCCTGCACCTGCCCCACTGG
CTTCGCCCTGGCCTGGGATGACAGGAACTGCAGAGATGTGGACGAGTGTGCGTGGGATGCTCACCTC
TGCCGAGAGGGACAGCGCTGTGTGAACCTGCTCGGGTCCTACCGCTGCCTCCCCGACTGTGGGCCTG
GCTTCCGGGTGGCTGATGGGGCCGGCTGTGAAGATGTGGACGAATGCCTGGAGGGGTTGGACGACTG
TCACTACAACCAGCTCTGCGAGAACACCCCAGGCGGTCACCGCTGCAGCTGCCCCAGGGGTTACCGG
ATGCAGGGCCCCAGCCTGCCCTGCCTAGATGTCAATGAGTGCCTGCAGCTGCCCAAGGCCTGCGCCT
ACCAGTGCCACAACCTCCAGGGCAGCTACCGCTGCCTGTGCCCCCCAGGCCAGACCCTCCTTCGCGA
CGGCAAGGCCTGCACCTCACTGGAGCGGAATGGACAAAATGTGACCACCGTCAGCCACCGAGGCCCT
CTATTGCCCTGGCTGCGGCCCTGGGCCTCGATCCCCGGTACCTCCTACCACGCCTGGGTCTCTCTCC
GTCCGGGTCCCATGGCCCTGAGCAGTGTGGGCCGGGCCTGGTGCCCTCCTGGTTTCATCAGGCAGAA
CGGAGTCTGCACAGACCTTGACGAGTGCCGCGTGAGGAACCTGTGTCAGCACGCCTGCCGCAACACT
GAGGGCAGCTACCAGTGCCTGTGCCCCGCCGGCTACCGTCTGCTCCCCAGCGGGAAGAACTGCCAGG
ACATCAACGAGTGCGAGGAGGAGAGCATCGAGTGTGGACCCGGCCAGATGTGCTTCAACACCCGTGG
CAGCTACCAGTGTGTGGACACACCCTGTCCTGCCACCTACCGGCAGGGCCCCAGCCCTGGGACGTGC
TTCCGCCGCTGCTCGCAGGACTGCGGCACGGGCGGCCCCTCTACGCTGCAGTACCGGCTGCTGCCGC
TGCCCCTGGGCGTGCGCGCCCACCACGACGTGGCCCGCCTCACCGCCTTCTCCGAGGTCGGCGTCCC
CGCCAACCGCACCGAGCTCAGCATGCTGGAGCCCGACCCCCGCAGCCCCTTCGCGCTGCGTCCGCTG
CGCGCGGGCCTTGGCGCGGTCTACACCCGTCGCGCGCTCACCCGCGCCGGCCTCTACCGGCTCACCG
TGCGTGCTGCGGCACCGCGCCACCAAAGCGTCTTCGTCTTGCTCATCGCCGTGTCCCCCTACCCCTA
CTAA

Variant sequences of NOV9 are included in Example 3, Table 21. A variant sequence can include a single nucleotide polymorphism (SNP). A SNP can, in some instances, be referred to as a “cSNP” to denote that the nucleotide sequence containing the SNP originates as a cDNA.

The NOV9 protein (SEQ ID NO:18) encoded by SEQ ID NO:17 is 3931 amino acid residues in length and is presented using the one-letter amino acid code in Table 9B. Psort analysis predicts the NOV9 protein of the invention to be localized at the plasma membrane with a certainty of 0.7300.

TABLE 9B
Encoded NOV9 protein sequence

(SEQ ID NO:18)

MSALFAAVYQMLKPRLVHNSPHPVTYQIEASLKPEQPGVTLVSIPVFLAPSWHKASELIPTQSFR
AQGAGKQLLGSPCPQVPPSIREDGRKANVSGMAGQSLTLECDANGFPVPEIVWLKDAQLIPKVGG
HRLLDEGQSLHFPRIQEGDSGLYSCRAENQAGTAQRDFHLLVLTPPSVLGAGAAQEVLGLAGADV
ELQCWTSGVPTPQVEWTKDRQPVLPGGPHLQVQEDGQVLRITGSHVGDEGRYQCVAFSPAGQQAR
DFQLRVHAPPTIWGSNETGEVAVMEDHLVQLLCEARGVPTPNITWFKDGALLPTSTKVVYTRGGR
QLQLGRAQSSDAGVYTCKASNAVGAAEKATRLDVYVPPTIEGAGGRPYVVKAVAGRPVALECVAR
GHPSPTLSWHHEGLPVAESNESRLETDGSVLRLESPGEASSGLYSCVASSPAGEAVLQYSVEVQV
PPQLLVAEGLGQVTTIVGQPLELPCQASGSPVPTIQWLQNGRPAEELAGVQVASQGTTLHIDHVE
LDHSGLFACQATNEAGTAGAEVEVSVHEFPSVSIIGGENITAPFLQPVTLQCIGDGVPTPSLRWW
KDGVALAAFGGNLQIEKVDLRDEGIYTCAATNLAGESKREVALKVLVPPNIEPGPVNKAVLENAS
VTLECLASGVPPPDVSWFKGHQPVSSWMGVTVSVDGRVLRIEQAQLSDAGSYRCVASNVAGSTEL
RYGLRVNVPPRITLPPSLPGPVLVNTPVRLTCNATGAPSPTLMWLKDGNPVSPAGTPGLQVFPGG
RVLTLASARASDSGRYSCVAVSAVGEDRQDVVLQVHMPPSILGEELNVSVVANESVALECQSHAM
PPPVLSWWKDGRPLEPRPGVHLSADKALLQVDRADVWDAGHYTCEALNQAGHSEKHYNLNVWGQP
LPGEGAGLQHVSAVGRLLYLGQAQLAQEGTYTCECSNVVGNSSQDLQLEVHVPPQIAGPREPPTQ
VSVVQDGVATLECNATGKPPPTVTWERDGQPVGAELGLQLQNQGQSLHVERAQAAHTGRYSCVAE
NLAGRAERKFELSVLVPPELIGDLDPLTNITAALHSPLTLLCEAMGIPPPAIRWFRGEEPVSPGE
DTYLLAGGWMLKMTQTQEQDSGLYSCLASNEAGEARRNFSVEVLVPPSIENEDLEEVIKVLDGQT
AHLMCNVTGHPQPKLTWFKDGRPLARGDAHHISPDGVLLQVLQANLSSAGHYSCIAANAVGEKTK
HFQLSVLLAPTILGGAEDSADEEVTVTVNNPISLICEALAFPSPNITWMKDGAPFEASRNIQLLP
GTHGLQILNAQKEDAGQYTCVVTNELGEAVKNYHVEVLIPPSISKDDPLAEVGVKEVKTKVNSTL
TLECESWAVPPPTIRWYKDGQPVTPSSRLQVLGEGRLLQIQPTQVSDSGRYLCVATNVAGEDDQD
FNVLIQVPPMFQKVGDFSAAFEILSREEEARGGVTEYREIVENNPAYLYCDTNAIPPPDLTWYRE
DQPLSAGDEVSVLQGGRVLQIPLVRAENAGRYSCKASNEVGEDWLHYELLVLTPPVILGDTEELV
EEVTVNASSTVSLQCPALGNPVPTISWLQNGLPFSPSPRLQVLEDGQVLQVSTAEVADAASYMCV
AENQAGSAEKLFTLRVQGLDLEQVTAILNSSVSLPCDVHAHPNPEVTWYKDSQALSLGEEVFLLP
GTHTLQLGRARLSDSGMYTCEALNAAGRDQKLVQLSVLVPPAFRQAPRGPQDAVLVRVGDKAVLS
CETDALPEPTVTWYKDGQPLVLAQRTQALRGGQRLEIQEAQVSDKGLYSCKVSNVAGEAVRTFTL
TVQVPPTFENPKTETVSQVAGSPLVLTCDVSGVPAPTVTWLKDRMPVESSAVHGVVSRGGRLQLS
RLQPAQAGTYTCVAENTQAEARKDFVVAVLVAPRIRSSGVAREHHVLEGQEVRLDCEADGQPPPD
VAWLKDGSPLGQDMGPHLRFYLDCCSLVLKGLRASDAGAYTCVAHNPAGEDARLHTVNVLVPPTI
KQGADGSGTLVSRPGELVTMVCPVRGSPPIHVSWLKDGLPLPLSQRTLLHGSGHTLRISKVQLAD
AGIFTCVAASPAGVADRNFTLQVQVPPVLEPVEFQNDVVVVRGSLVELPCEARGVPLPLVSWMKD
GEPLLSQSLEQGPSLQLEAVGAGDSGTYSCVAVSEAGEARRHFQLTVMEPPHIEDSGQPTELSLT
PGAPMELLCDAQGTPQPNITWHKDGQALTRLENNSRATRVLRVENVQVRDAGLYTCLAESPAGAI
EKSFRVRVQAPPNIVGPRGPRFVVGLAPGQLVLECSVEAEPAPKITWHRDGIVLQEDAHTQFPER
GRFLQLQALSTADSGDYSCTARNAAGSTSVAFRVEIHTVPTIRSGPPAVNVSVNQTALLPCQADG
VPAPLVSWRKDRVPLDPRSPRATPIHSRFEILPEGSLRIQPVLAQDAGHYLCLASNSAGSDRQGR
DLRVLEPPAIAPSPSNLTLTAHTPALLPCEASGSPKPLVVWWKDGQKLDFRLQQGAYRLLPSNAL
LLTAPGPQDSAQFECVVSNEVGEAHRLYQVTVHVPPTIADDQTDFTVTMMAPVVLTCHSTGIPAP
TVSWSKAGAQLGARGSGYRVSPSGALEIGQALPIHAGRYTCSARNSAGVAHKHVFLTVQASPVVK
PLPSVVRAVAEEEVLLPCEASGIPRPTITWQKEGLNVATGVSTQVLPGGQLRIAHASPEDAGNYL
CIAKNSAGSAMGKTRLVVQVPPVIENGLPDLSTTEGSHAFLPCKARGSPEPNITWDKDGQPVSGA
EGKFTIQPSGELLVKNLEGQDAGTYTCTAENAVGRARRRVHLTILVLPVFTTLPGDRSLRLGDRL
WLRCAARGSPTPRIGWTVNDRPVTEGVSEQDGGSTLQRAAVSREDSGTYVCWAENRVGRTQAVSF
VHVKEAPVLQGEAFSYLVEPVGGSIQLDCVVRGDPVPDIHWIKDGLPLRGSHLRHQLQNGSLTIR
RTEARRGLAPWRDDAGRYQCLAENEMGVAKKVVILVLQTRMVPAEPHLKRQLPPIPSNNEAPSLF
PGVHGGHVGNPDFHSHLAEVLAVQLLAGSLLFSARAMPQASTAAISLLAPTSFAPFPDDISQGIL
SSSTAHQGSPQGWQKLLFFTAIPNKTTVMVTVEPQDMTVRSGDDVALRCQATGEPTPTIEWLQAG
QPLRASRRLRTLPDGSLWLENVETGDAGTYDCVAHNLLGSATARAFLVCASHAIVGSRHFRDPQV
FCEFVVPPPHFTGEPQGSWGSMTGVINGRKFGVATLNTSVMQEAHSGVSSIHSSIRHVPANVGPL
MRVLVVTIAPIYWALARESGEALNGHSLTGGRFRQESHVEFATGELLTMTQVARGLDPDGLLLLD
VVVNGVVPESLADADLQVQDFEEHYVQTGPGQLFVGSTQRFFQGGLPSFLRCNHSIQYNAARGPQ
PQLVQHLRASAISSAFDPEAEALRFQLATALQAEENEVGCPEGFELDSQGAFCVDRDECSGGPSP
CSHACLNAPGRFSCTCPTGFALAWDDRNCRDVDECAWDAHLCREGQRCVNLLGSYRCLPDCGPGF
RVADGAGCEDVDECLEGLDDCHYNQLCENTPGGHRCSCPRGYRMQGPSLPCLDVNECLQLPKACA
YQCHNLQGSYRCLCPPGQTLLRDGKACTSLERNGQNVTTVSHRGPLLPWLRPWASIPGTSYHAWV
SLRPGPMALSSVGRAWCPPGFIRQNGVCTDLDECRVRNLCQHACRNTEGSYQCLCPAGYRLLPSG
KNCQDINECEEESIECGPGQMCFNTRGSYQCVDTPCPATYRQGPSPGTCFRRCSQDCGTGGPSTL
QYRLLPLPLGVRAHHDVARLTAFSEVGVPANRTELSMLEPDPRSPFALRPLRAGLGAVYTRRALT
RAGLYRLTVRAAAPRHQSVFVLLIAVSPYPY

A search against the Patp database, a proprietary database that contains sequences published in patents and patent publications, yielded several homologous proteins shown in Table 9C.

TABLE 9C
Patp results for NOV9

| | | | Smallest |
|---|---|---|---|
| | | | Sum |
| Sequences producing High-scoring | Reading | High | Prob |
| Segment Pairs: | Frame | Score | P (N) |
| >patp:AAY53667 Sequence gi/3328186 | +1 | 1529 | 6.5e−244 |
| >patp:AAY87206 Human secreted protein | +1 | 2235 | 6.4e−230 |
| sequence ID NO: 245 | | | |
| >patp:AAE06183 Human gene 57 encoded | +1 | 2235 | 6.4e−230 |
| secreted protein | | | |
| >patp:AAY87120 Human secreted protein | +1 | 2235 | 6.4e−230 |
| sequence SEQ ID: 159 | | | |
| >patp:AAE06097 Human gene 57 secreted | +1 | 2235 | 6.4e−230 |
| protein HRACD80 | | | |

In a BLAST search of public sequence databases, it was found, for example, that the nucleic acid sequence of this invention has 625 of 1067 bases (58%) identical to a gb:GENBANK-ID:HSLTGFBP4|acc:Y13622.1 mRNA from Homo sapiens (mRNA for latent transforming growth factor-beta binding protein-4). The full amino acid sequence of the protein of the invention was found to have 502 of 1665 amino acid residues (30%) identical to, and 767 of 1665 amino acid residues (46%) similar to, the 5198 amino acid residue ptnr:SPTREMBL-ACC:076518 protein from Caenorhabditis elegans (HEMICENTIN PRECURSOR).

NOV9 also has homology to the proteins shown in the BLASTP data in Table 9D.

TABLE 9D
BLAST results for NOV9

| Gene Index/ | | | | | |
|---|---|---|---|---|---|
| Identifier | Protein/Organism | Length (aa) | Identity (%) | Positives (%) | Expect |
| gi\|14575679\|gb\| | hemicentin | 5636 | 1230/3017 | 1785/3017 | 0.0 |
| AAK68690.1\|AF156100_1 | [Homo sapiens] | | (40%) | (58%) | |
| (AF156100) | | | | | |
| gi\|18547943\|ref\|XP_{—} | hemicentrin | 3645 | 979/2379 | 1413/2379 | 0.0 |
| 053531.3\|(XM_053531) | [Homo sapiens] | | (41%) | (59%) | |
| gi\|17568539\|ref\|NP_{—} | Ig superfamily | 5175 | 857/3077 | 1348/3077 | 0.0 |
| 509636.1\|(NM_077235) | repeats (I-type) | | (27%) | (42%) | |
| | [Caenorhabditis | | | | |
| | elegans] | | | | |
| gi\|17568541\|ref\|NP_{—} | IG | 5198 | 857/3077 | 1348/3077 | 0.0 |
| 509635.1\|(NM_077234) | (immunoglobulin) | | (27%) | (42%) | |
| | superfamily (47 | | | | |
| | domains) | | | | |
| | [Caenorhabditis | | | | |
| | elegans] | | | | |
| gi\|13872813\|emb\| | fibulin-6 | 2673 | 552/1399 | 796/1399 | 0.0 |
| CAC37630.1\|(AJ306906) | [Homo sapiens] | | (39%) | (56%) | |

A multiple sequence alignment is given in Table 9E, with the NOV9 protein being shown-on line 1 in Table 9E in a ClustalW analysis, and comparing the NOV9 protein with the related protein sequences shown in Table 9D. This BLASTP data is displayed graphically in the ClustalW in Table 9E.

[image]

[image]

[image]

[image]

[image]

The NOV9 Clustal W alignment shown in Table 9E was modified to begin at amino residue 4080. The data in Table 9E includes all of the regions overlapping with the NOV9 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 9F lists the domain description from DOMAIN analysis results against NOV9.

TABLE 9F
Domain Analysis of NOV9

| | Region of | | |
|---|---|---|---|
| Model | Homology | Score (bits) | E value |
| ig | 99-157 | 46.50 | 6.1e−10 |
| ig | 192-251 | 38.00 | 2.1e−07 |
| ig | 286-344 | 41.90 | 1.4e−08 |
| ig | 380-438 | 32.10 | 1.3e−05 |
| ig | 473-531 | 45.90 | 8.9e−10 |
| ig | 565-615 | 35.50 | 1.2e−06 |
| ig | 648-706 | 43.80 | 4e−09 |
| ig | 740-800 | 39.10 | 1e−07 |
| ig | 833-891 | 27.10 | 0.00041 |
| ig | 981-1039 | 42.90 | 7.1e−09 |
| ig | 1075-1133 | 28.90 | 0.00012 |
| ig | 1168-1226 | 30.80 | 3.2e−05 |
| ig | 1264-1322 | 39.60 | 7.2e−08 |
| ig | 1362-1420 | 43.40 | 5.2e−09 |
| ig | 1473-1531 | 29.40 | 8.2e−05 |
| ig | 1568-1626 | 35.70 | 1e−06 |
| ig | 1654-1712 | 45.20 | 1.5e−09 |
| ig | 1749-1807 | 43.80 | 3.8e−09 |
| ig | 1841-1899 | 43.00 | 6.8e−09 |
| ig | 1934-1994 | 41.40 | 2e−08 |
| ig | 2030-2088 | 49.90 | 5.8e−11 |
| ig | 2123-2177 | 49.60 | 6.8e−11 |
| ig | 2212-2268 | 37.50 | 3e−07 |
| ig | 2303-2361 | 28.60 | 0.00014 |
| ig | 2394-2459 | 28.20 | 0.00019 |
| ig | 2492-2552 | 32.90 | 7.2e−06 |
| ig | 2585-2643 | 22.80 | 0.0081 |
| ig | 2676-2733 | 37.50 | 3.1e−07 |
| ig | 2766-2824 | 43.10 | 6.3e−09 |
| ig | 2857-2913 | 44.10 | 3e−09 |
| ig | 2947-3012 | 18.00 | 0.22 |
| ig | 3162-3219 | 34.70 | 2.1e−06 |
| EGF | 3504-3539 | 38.30 | 1.8e−07 |
| EGF | 3589-3626 | 13.80 | 1.3 |
| EGF | 3632-3667 | 33.30 | 5.4e−06 |
| EGF | 3739-3773 | 38.40 | 1.6e−07 |

Consistent with other known members of the Hemicentin Precursor-like family of proteins, NOV9 has, for example, thirty-three immunoglobulin (ig) signature sequences and four epidermal growth factor (EGF) signature sequences, as well as homology to other members of the Hemicentin Precursor-like Protein Family. NOV9 nucleic acids, and the encoded polypeptides, according to the invention are useful in a variety of applications and contexts. For example, NOV9 nucleic acids and polypeptides can be used to identify proteins that are members of the Hemicentin Precursor-like Protein Family. The NOV9 nucleic acids and polypeptides can also be used to screen for molecules, which inhibit or enhance NOV9 activity or function. Specifically, the nucleic acids and polypeptides according to the invention may be used as targets for the identification of small molecules that modulate or inhibit, e.g., cellular activation, cellular differentiation, and signal transduction. These molecules can be used to treat, e.g., Cardiovascular diseases, Hyperparathyroidism, Hypoparathyroidism, Lymphedema, Allergies as well as other diseases, disorders and conditions.

In addition, various NOV9 nucleic acids and polypeptides according to the invention are useful, inter alia, as novel members of the protein families according to the presence of domains and sequence relatedness to previously described proteins. For example, the NOV9 nucleic acids and their encoded polypeptides include structural motifs that are characteristic of proteins belonging to the Hemicentin Precursor-like Protein Family.

Hemicentrin is an extracellular matrix protein with a modular sturcture. Like NOV9, the hemicentrin structure includes many immunoglobulin domains flanked by EGF domains. The protein is likely involved in cellular differentiation of epithelial tissue.

The NOV9 nucleic acids and polypeptides, antibodies and related compounds according to the invention will be useful in therapeutic and diagnostic applications in the mediation of cardiac, immune and endocrine physiology. As such, the NOV9 nucleic acids and polypeptides, antibodies and related compounds according to the invention may be used to treat cardiovascular, immune, and endocrine disorders, e.g., Cardiovascular diseases, Hyperparathyroidism, Hypoparathyroidism, Lymphedema, Allergies as well as other diseases, disorders and conditions.

The NOV9 nucleic acids and polypeptides are useful for detecting specific cell types. For example, expression analysis has demonstrated that a NOV9 nucleic acid is expressed in Adipose, Thyroid, Colon, Lymph node, Bone, Myometrium, Prostate, Testis, Aorta, Vein.

Additional utilities for NOV9 nucleic acids and polypeptides according to the invention are disclosed herein.

NOV10

A NOV10 polypeptide has been identified as a Selectin-like protein. The novel NOV10 nucleic acid sequences maps to the chromosome 9. Two alternative novel NOV10, NOV10a and NOV10b, nucleic acids and encoded polypeptides are provided.

NOV10a

A NOV10 variant is NOV10a (alternatively referred to herein as CG94661-01), which includes the 1268 nucleotide sequence (SEQ ID NO:19) shown in Table 10A. A NOV10a ORF begins with a ATG initiation codon at nucleotides 145-147 and ends with a TGA codon at nucleotides 871-873. Putative untranslated regions upstream from the initiation codon and downstream from the termination codon are underlined in Table 10A, and the start and stop codons are in bold letters.

TABLE 10A
NOV10a Nucleotide Sequence

(SEQ ID NO:19)

GCGGCCGCCACCCTCCGTGGCAAGGCGAGGCCCCGGGGGCGGGCCGGGGTCACCACGCCTGTCCCAG
GGAACCGCACAGACGGTACTCACCCTTCTTGCGATGATGTGAGATGATAAAATGCCTACATGATGAG
ATGAAGTGAGATGAAAAACATAGGCCTTGTGATGGAATGGGAAATTCCAGAGATAATTTGCACGTGC
GCTAAGCTGCGGCTACCCCCGCAAGCAACCTTCCAAGTCCTTCGTGGCAATGGTGCTTCCGTGGGGA
CCGTGCTCATGTTCCGCTGCCCCTCCAACCACCAGATGGTGGGGTCTGGGCTCCTCACCTGCACCTG
GAAGGGGAGCATCGCTGAGTGGTCTTCAGGGTCCCCAGTGTGCAAACTGGTGCCACCACACGAGACC
TTTGGCTTCAAGGTGGCCGTGATCGCCTCCATTGTGAGCTGTGCCATCATCCTGCTCATGTCCATGG
CCTTCCTCACCTGCTGCCTCCTCAAGTGCGTGAAGAAGAGCAAGCGGCGGCGCTCCAACAGGTCAGC
CCAGCTGTGGTCCCAGCTGAAAGATGAGGACTTGGAGACGGTGCAGCCCGCATACCTTGGCCTCAAG
CACTTCAACAAACCCGTGAGCGGGCCCAGCCAGGCGCACGACAACCACAGCTTCACCACAGACCATG
GTGAGAGCACCAGCAAGCTGGCCAGTGTGACCCGCAGCGTGGACAAGGACCCTGGGATCCCCAGAGC
TCTAAGCCTCAGTGGCTCCTCCAGCTCACCCCAAGCCCAGGTGATGGTGCACATGGCAAACCCCAGA
CAGCCCCTGCCTGCCTCTGGGCTGGCCACAGGAATGCCACAACAGCCCGCAGCATATGCCCTAGGGT
GACCACGCAGTGAGGCTGGTGCCCATGCTCCACACTGGGAGGCCAGGCTGACCCCACCAGCCAGTCA
GCTACAACTCCACATCAACTCCACATGCGCCCAGCTCGAGACTGATGAGTGGAATCAGCTTCCAGGT
GTAGGGACCCCTTGAGGGGCCGAGCTGACATCCAAGGCTGAGGACCCCAGTGGGGAGTGTTCTGTTC
CGGCATATCCTGGCCGTAACGATTTTTATAGTTATGGACTACTTGAAACCACTACTGAGGGTAATTT
ACTAGCTGTGGCCTCCCACTAACTAGCATTCCTTTAAAGAGACTGGGAAATGTTTTAAGCAAATCTA
GTTTTGTATAATAAAATAAGAAAATAGCAATAAACTTCTTTTCAGCAACTACAAAAAAAAA

The NOV10a polypeptide (SEQ ID NO:20) encoded by SEQ ID NO:19 is 242 amino acid residues in length and is presented using the one-letter amino acid code in Table 10B. The Psort profile for the NOV10a predicts that this peptide is likely to be localized at the plasma membrane with a certainty of 0.7000.

TABLE 10B
NOV10a protein sequence

(SEQ ID NO:20)

MKNIGLVMEWEIPEIICTCAKLRLPPQATFQVLRGNGASVGTVLMFRCPSNHQMVGSGLLTCTWKGS
IAEWSSGSPVCKLVPPHETFGFKVAVIASIVSCAIILLMSMAFLTCCLLKCVKKSKRRRSNRSAQLW
SQLKDEDLETVQAAYLGLKHFNKPVSGPSQAHDNHSFTTDHGESTSKLASVTRSVDKDPGIPRALSL
SGSSSSPQAQVMVHMANPRQPLPASGLATGMPQQPAAYALG

NOV10b

Alternatively, a NOV10 variant is the novel NOV10b (alternatively referred to herein as CG94661-02), which includes the 887 nucleotide sequence (SEQ ID NO:21) shown in Table 10C. NOV10b was created by polymerase chain reaction (PCR) using the primers detailed in Example 1, Table 17. Primers were designed based on in silico predictions of the full length or some portion (one or more exons) of the cDNA/protein sequence of the invention. The PCR product derived by exon linking, covering the entire open reading frame, was cloned into the pCR2.1 vector from Invitrogen to provide clone 143260::COR 100348691_extn.698976.C20.

The NOV10b ORF begins with a Kozak consensus ATG initiation codon at nucleotides 72-74 and ends with a TGA codon at nucleotides 1958-1960. Putative untranslated regions upstream from the initiation codon and downstream from the termination codon are underlined in Table 10C, and the start and stop codons are in bold letters.

TABLE 10C
NOV10b Nucleotide Sequence

(SEQ ID NO:21)

GCACAGACGGTACTCACCCTTCTTGCGATGATGTGAGATGATAAAATGCCTACATGATGAGATGAAG
TGAGATGAAAAACATAGGCCTTGTGATGGAATGGGAAATTCCAGAGATAATTTGCATGTGCGCTAAG
CTGCGGCTACCCCCGCAAGCAACCTTCCAAGTCCTTCGTGGCAATGGTGCTTCCGTGGGGACCGTGC
TCATGTTCCGCTGCCCCCCCAACCACCAGATGGTGGGGTCTGGGCTCCTCACCTGCACCTGGAAGGG
GAGCATCGCTGAGTGGTCTTCAGGGTCCCCAGTGTGCAAACTGGTGCCACCACACGAGACCTTTGGC
TTCAAGGTGGCCGTGATCGCCTCCATTGTGAGCTGTGCCATCATCCTGCTCATGTCCATGGCCTTCC
TCACCTGCTGCCTCCTCAAGTGCGTGAAGAAGAGCAAGCGGCGGCGCTCCAACAGGTCAGCCCAGCT
GTGGTCCCAGCTGAAAGATGAGGACTTGGAGACGGTGCAGGCCGCATACCTTGGCCTCAAGCACTTC
AACAAACCCGTGAGCGGGCCCAGCCAGGCGCACGACAACCACAGCTTCACCACAGACCATGGTGAGA
GCACCAGCAAGCTGGCCAGTGTGACCCGCAGCGTGGACAAGGACCCTGGGATCCCCAGAGCTCTAAG
CCTCAGTGGCTCCTCCAGCTCACCCCAAGCCCAGGTGATGGTGCACATGGCAAACCCCAGACAGCCC
CTGCCTGCCTCTGGGCTGGCCACAGGAATGCCACAACAGCCCGCAGCATATGCCCTAGGGTGACCAC
GCAGTGAGCCTGGTGCCCATGCTCCACACTGGGAGGCCAGGCTGACCCCACCAGCCAGTCAGCTACA
ACTCCACATCAACTCC

Variant sequences of NOV10b are included in Example 3, Table 22. A variant sequence can include a single nucleotide polymorphism (SNP). A SNP can, in some instances, be referred to as a “cSNP” to denote that the nucleotide sequence containing the SNP originates as a cDNA.

The NOV10b protein (SEQ ID NO:22) encoded by SEQ ID NO:21 is 242 amino acid residues in length and is presented using the one-letter code in Table 10D. The Psort profile for NOV10b predicts that this sequence is likely to be localized at the plasma membrane with a certainty of 0.7000.

TABLE 10D
NOV10b protein sequence

(SEQ ID NO:22)

MKNIGLVMEWEIPEIICMCAKLRLPPQATFQVLRGNGASVGTVLMFRCPPNHQMVGSGLLTCTWKGS
IAEWSSGSPVCKLVPPHETFGFKVAVIASIVSCAIILLMSMAFLTCCLLKCVKKSKRRRSNRSAQLW
SQLKDEDLETVQAAYLGLKHFNKPVSGPSQAHDNHSFTTDHGESTSKLASVTRSVDKDPGIPRALSL
SGSSSSPQAQVMVHMANPRQPLPASGLATGMPQQPAAYALG

NOV10 Clones

Unless specifically addressed as NOV10a or NOV10b, any reference to NOV10 is assumed to encompass all variants. NOV10a differs from NOV10b at amino acid position 18 (T>M) and amino acid position 50 (S>P) as shown in Tables 100B and 10D.

A search against the Patp database, a proprietary database that contains sequences published in patents and patent publications, yielded several homologous proteins shown in Table 10E.

TABLE 10E
Patp results for NOV10

| | | | Smallest |
|---|---|---|---|
| | | | Sum |
| Sequences producing High-scoring | Reading | High | Prob |
| Segment Pairs: | Frame | Score | P (N) |
| >patp:AAM93054 Human digestive system | +1 | 210 | 7.2e−17 |
| antigen | | | |
| >patp:AAR05494 Endothelial leukocyte | +1 | 113 | 0.0016 |
| adhesion molecule-1 | | | |
| >patp:AAR08116 Endothelial leucocyte | +1 | 113 | 0.0016 |
| adhesion molecule-1 | | | |
| >patp:AAW18839 E-selectin | +1 | 113 | 0.0016 |
| >patp:AAW46733 Endothelial leukocyte | +1 | 113 | 0.0016 |
| adhesion molecule-1 | | | |

In a BLAST search of public sequence databases, it was found, for example, that the NOV10a nucleic acid sequence of this invention has 438 of 447 bases (97%) identical to a gb:GENBANK-ID:HSM802384|acc:AL137623.1 mRNA from Homo sapiens (cDNA DKFZp434J1812 (from clone DKFZp434J1812)). The full amino acid sequence of the protein of the invention was found to have 110 of 139 amino acid residues (79%) identical to, and 123 of 139 amino acid residues (88%) similar to, the 269 amino acid residue ptnr:SPTREMBL-ACC:Q9D176 protein from Mus musculus (1700017111 RIK PROTEIN).

Similarly, it was found, for example, the NOV10b nucleic acid sequence of this invention has 438 of 447 bases (97%) identical to a gb:GENBANK-ID:HSM802384|acc:AL137623.1 mRNA from Homo sapiens (cDNA DKFZp434J1812 (from clone DKFZp434J1812)). The full amino acid sequence of the protein of the invention was found to have 108 of 138 amino acid residues (78%) identical to, and 121 of 138 amino acid residues (87%) similar to, the 269 amino acid residue ptnr:SPTREMBL-ACC:Q9D176 protein from Mus musculus (1700017111 RIK PROTEIN).

Additional BLAST results are shown in Table 10F.

TABLE 10F
BLAST results for NOV10

| Gene Index/ | | | | | |
|---|---|---|---|---|---|
| Identifier | Protein/Organism | Length (aa) | Identity (%) | Positives (%) | Expect |
| >gi\|15779059\|gb\| | Similar to RIKEN | 255 | 192/225 | 192/225 | e−101 |
| AAH14601.1\|AAH14601 | cDNA 1700017I11 | | (85%) | (85%) | |
| (BC014601) | gene | | | | |
| | [Homo sapiens] | | | | |
| >gi\|12834785\|dbj\| | Sushi domain | 269 | 130/246 | 142/246 | 8e−56 |
| BAB23043.1\| | (SCR repeat) | | (52%) | (56%) | |
| (AK003860) | containing | | | | |
| | protein˜data | | | | |
| | source: Pfam, | | | | |
| | source | | | | |
| | key: PF00084, | | | | |
| | evidence: ISS˜ | | | | |
| | putative | | | | |
| | [Mus musculus] | | | | |
| >gi\|12850544\|dbj\| | Sushi domain | 170 | 71/102 | 77/102 | 6e−35 |
| BAB28764.1\| | (SCR repeat) | | (69%) | (74%) | |
| (AK013276) | containing | | | | |
| | protein˜data | | | | |
| | source: Pfam, | | | | |
| | source | | | | |
| | key: PF00084, | | | | |
| | evidence: ISS˜ | | | | |
| | putative | | | | |
| | [Mus musculus] | | | | |
| >gi\|12838976\|dbj\| | Sushi domain | 149 | 55/73 | 61/73 | 2e−26 |
| BAB24394.1\| | (SCR repeat) | | (75%) | (83%) | |
| (AK006068) | containing | | | | |
| | protein˜data | | | | |
| | source: Pfam, | | | | |
| | source | | | | |
| | key: PF00084, | | | | |
| | evidence: ISS˜ | | | | |
| | putative | | | | |
| | [Mus musculus] | | | | |
| >gi\|7494498\|pir\|\| | scavenger | 2043 | 30/87 | 42/87 | 2e−04 |
| T18524 | receptor | | (34%) | (47%) | |
| | cysteine-rich | | | | |
| | protein homolog | | | | |
| | srcrm2 - Geodia | | | | |
| | cydonium | | | | |

A multiple sequence alignment is given in Table 10G, with the NOV10 protein of the invention being shown on line 1, in a ClustalW analysis comparing NOV10 with related protein sequences disclosed in Table 1° F.

[image]

[image]

The NOV10 Clustal W alignment shown in Table 10F was modified to begin at amino residue 1600 and end at amino acid residue 2000. The data in Table 10F includes all of the regions overlapping with the NOV10 protein sequences.

The NOV10 Clustal W alignment shown in Table 10G was modified to begin at amino residue 1601. The data in Table 10G includes all of the regions overlapping with the NOV10 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 1 OH lists the domain description from DOMAIN analysis results against NOV10.

TABLE 10H
Domain Analysis of NOV10

| | Region of | | |
|---|---|---|---|
| Model | Homology | Score (bits) | E value |
| Sushi domain | 19-78 | 15.8 | 0.0075 |
| (SCR repeat) | | | |

Consistent with other known members of the Selectin-like family of proteins, NOV10 has, for example, a Sushi domain (SCR repeat) signature sequences and homology to other members of the Selectin-like Protein Family. NOV10 nucleic acids, and the encoded polypeptides, according to the invention are useful in a variety of applications and contexts. For example, NOV10 nucleic acids and polypeptides can be used to identify proteins that are members of the Selectin-like Protein Family. The NOV10 nucleic acids and polypeptides can also be used to screen for molecules, which inhibit or enhance NOV10 activity or function. Specifically, the nucleic acids and polypeptides according to the invention may be used as targets for the identification of small molecules that modulate or inhibit, e.g., cellular adhesion and signal transduction. These molecules can be used to treat, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Von Hippel-Lindau (VHL) syndrome, Cirrhosis, Transplantation, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Systemic lupus erythematosus, Autoimmune disease, Asthma, Emphysema, Scleroderma, allergy as well as other diseases, disorders and conditions.

In addition, various NOV10 nucleic acids and polypeptides according to the invention are useful, inter alia, as novel members of the protein families according to the presence of domains and sequence relatedness to previously described proteins. For example, the NOV10 nucleic acids and their encoded polypeptides include structural motifs that are characteristic of proteins belonging to the Selectin-like Protein Family.

The NOV10 nucleic acids and polypeptides, antibodies and related compounds according to the invention will be useful in therapeutic and diagnostic applications in the mediation of cardiac and immune physiology. As such, the NOV10 nucleic acids and polypeptides, antibodies and related compounds according to the invention may be used to treat cardiovascular and immune disorders, e.g., Cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderna, Obesity, Transplantation, Von Hippel-Lindau (VHL) syndrome, Cirrhosis, Transplantation, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Systemic lupus erythematosus, Autoimmune disease, Asthma, Emphysema, Scieroderma, allergy as well as other diseases, disorders and conditions.

The NOV10 nucleic acids and polypeptides are useful for detecting specific cell types. For example, expression analysis has demonstrated that a NOV10 nucleic acid is expressed in Heart, Thyroid, Parotid Salivary glands, Liver, Colon, Ascending Colon, Bone Marrow, Peripheral Blood, Lymphoid tissue, Spleen, Lymph node, Tonsils, Thymus, Cerebellum, Spinal Chord, Cervix, Mammary gland/Breast, Ovary, Placenta, Uterus, Oviduct/Uterine Tube/Fallopian tube, Vulva, Prostate, Testis, Lung, Kidney, Kidney Cortex, Retina, Skin.

Additional utilities for NOV10 nucleic acids and polypeptides according to the invention are disclosed herein.

NOV11

A NOV11 polypeptide has been identified as a Nuclear Protein-like protein (also referred to as CG94325-01). The disclosed novel NOV11 nucleic acid (SEQ ID NO:23) of 8670 nucleotides is shown in Table 11A. The novel NOV11 nucleic acid sequences maps to the chromosome 15.

An ORF begins with an ATG initiation codon at nucleotides 204-206 and ends with a TAA codon at nucleotides 7152-7154. A putative untranslated region and/or downstream from the termination codon is underlined in Table 11A, and the start and stop codons are in bold letters.

TABLE 11A
NOV11 Nucleotide Sequence

(SEQ ID NO:23)

ACGCGTAGAGCCGCTTTGCGCGTGCGCATCACCTAGGCGGTTAGATTTGAATACTTCACTGAGGCGA
GCCGGGCGTTGTGAGCGGACTGCTAGAGGCGGCTGTCTGTTTCCGCTCTAAGGAAACTCAGAGCGTG
TGGACCCCAAACAAGTCTGCGCAAAATTTGTCGAGGAGGTTTGCCGCGGCAGAAAAGTTTTCTTCAA
AAATGGATGGGGTGTCTTCAGAGGCTAATGAAGAAAATGACAATATAGAGAGACCTGTTAGAAGACG
GCATTCTTCAATATTGAAACCCCCAAGGAGTCCTCTTCAGGACCTCAGAGGTGGGAATGAAAGAGTT
CAGGAATCCAATGCTTTGAGAAATAAGAAAAACTCTCGTCGAGTCAGCTTTGCAGATACTATAAAGG
TATTCCAGACGGAGTCTCATATGAAAATAGTGAGAAAGTCAGAAATGGAAGAAACAGAAACAGGAGA
AAATCTTCTTTTGATACAGAATAAGAAATTAGAAGATAATTACTGTGAAATTACTGGGATGAACACA
TTGCTTTCTGCTCCCATTCATACCCAGATGCAACAGAAGGAGTTTTCAATTATAGAACATACCCGTG
AAAGGAAACATGCAAATGACCAGACAGTCATTTTTTCAGATGAAAACCAGATGGACCTGACATCAAG
TCACACTGTAATGATTACCAAAGGCCTTTTAGATAATCCCATAAGTGAAAAGTCCACCAAGATAGAT
ACCACATCATTTCTAGCTAATTTAAAGCTTCACACCGAGGACTCAAGAATGAAAAAAGAAGTAAATT
TTTCCGTGGATCAAAACACTTCTTCAGAAAATAAAATAGATTTCAATGACTTCATAAAAAGATTGAA
AACAGGAAAATGTAGTGCTTTTCCTGATGTGCCTGATAAAGAAAATTTTGAGATACCTATTTATTCC
AAGGAACCGAACAGTGCCTCTTCTACACATCAAATGCATGTATCTCTTAAGGAAGATGAAAATAACA
GTAATATTACTAGGCTCTTTAGAGAAAAAGATGATGGGATGAATTTCACCCAGTGTCATACAGCCAA
TATTCAGACATTGATTCCCACATCCAGTGAGACCAACTCACGGGAATCTAAAGGTAATGATATTACA
ATTTATGGCAATGACTTTATGGACTTGACATTTAACCACACTTTGCAGATCTTACCTGCAACAGGTA
ATTTTTCTGAAATAGAAAATCAAACTCAGAATGCCATGGATGTAACAACAGGTTATGGAACTAAAGC
TTCAGGAAATAAAACAGTTTTTAAGAGTAAACAAAATACTGCTTTTCAAGACCTTTCCATAAACTCT
GCAGACAAAATACATATTACCAGAAGTCATATTATGGGGGCAGAAACTCACATAGTCTCACAGACTT
GTAATCAGGATGCCAGAATATTAGCCATGACCCCAGAATCTATATATTCTAATCCATCTATTCAAGG
TTGTAAGACTGTTTTCTATTCTAGTTGTAATGATGCCATGGAAATGACCAAATGTCTCTCAAATATG
AGAGAGGAGAAAAATTTGCTAAAGCATGACAGTAATTATTCTAAAATGTATTGCAATCCAGATGCTA
TGTCTTCTCTCACAGAGAAAACTATTTATTCCGGAGAGGAGAACATGGACATTACCAAGAGTCATAC
AGTTGCAATAGATAATCAAATTTTTAAACAAGATCAATCAAATGTGCAAATAGCAGCTGCACCAACA
CCCGAAAAAGAAATGATGCTCCAAAATCTTATGACCACATCAGAAGATGGGAAAATGAATGTAAATT
GTAACTCAGTTCCTCATGTATCTAAGGAAAGAATACAGCAGAGCCTGTCAAATCCTTTGTCTATTTC
ATTGACTGATAGAAAGACTGAACTCTTATCAGGTGAAAATACGGATTTGACTGAAAGTCACACAAGT
AACTTAGCAAGTCAGGTTCCTCTTGCAGCTTATAATCTAGCACCGGAGAGTACCAGTGAATCTCACT
CTCAGAGCAAAAGCTCTTCAGATGAATGTGAAGAAATTACCAAAAGTCGTAATGAACCATTTCAGCG
ATCAGACATAATAGCCAAAAACAGCTTAACCGACACCTGGAACAAAGACAAAGATTGGGTTTTGAAG
ATTTTGCCCTACCTTGATAAAGATTCTCCTCAGTCAGCTGATTGTAATCAGGAGATAGCAACAAGCC
ATAATATAGTCTACTGTGGTGGAGTTCTTGATAAACAAATAACTAATAGAAATACAGTATCATGGGA
ACAATCTTTGTTTTCTACCACAAAGCCATTATTTTCATCAGGACAGTTCTCTATGAAAAATCATGAT
ACTGCTATAAGTAGTCATACAGTGAAATCTGTACTAGGCCAGAATTCTAAACTGGCTGAGCCACTGA
GGAAAAGTTTAAGCAATCCCACACCTGACTATTGCCATGACAAGATGATTATATGTTCAGAGGAAGA
GCAAAATATGGATCTAACAAAGAGCCACACTGTCGTCATTGGATTTGGTCCTTCTGAACTACAAGAA
CTTGGTAAAACTAATTTAGAACACACTACTGGCCAGCTAACAACAATGAACAGACAGATAGCTGTAA
AAGTTGAAAAATGTGGTAAAAGTCCCATAGAAAAAAGTGGAGTGCTTAAATCTAACTGTATTATGGA
TGTGTTAGAGGACGAAAGTGTACAGAAACCTAAATTTCCAAAGGAAAAGCAAAATGTCAAAATTTGG
GGAAGGAAAAGTGTTGGTGGACCAAAAATTGATAAGACTATTGTATTTTCAGAAGACGATAAGAATG
ATATGGATATCACTAAGAGTTATACAATAGAAATAAACCATAGACCTTTATTAGAGAAACGTGATTG
TCATTTGGTGCCATTGGCAGGAACTTCTGAAACTATTTTATATACATGTGGGCAGGATGACATGGAG
ATCACTAGAAGTCACACAACTGCCTTAGAATGTAAAACTGTCTCACCAGATGAAATAACTACTAGGC
CTATGGACAAAACTGTAGTGTTTGTAGATAATCATGTTGAACTAGAAATGACAGAGTCCCATACTGT
TTTCATTGACTACCAAGAAAAGGAAAGAACAGACAGACCTAACTTTGAACTATCCCAAAGGAAAAGC
CTAGGAACACCAACAGTGATATGTACTCCTACTGAGGAGAGTGTTTTCTTTCCAGGAAATGGTGAAA
GTGACCGTCTAGTAGCAAATGACAGCCAGCTAACCCCTCTGGAGGAATGGTCTAATAATAGGGGCCC
TGTAGAGGTAGCTGATAACATGGAATTGTCTAAATCAGCCACTTGCAAAAACATCAAAGATGTACAA
AGTCCTGGATTTCTGAATGAACCTCTATCAAGCAAAAGTCAGAGAAGAAAAAGCCTTAAGCTAAAAA
ATGACAAGACCATTGTATTTTCAGAGAATCATAAAAATGATATGGATATTACCCAGAGTTGTATGGT
GGAAATAGATAACGAAAGTGCCCTGGAGGATAAAGAGGACTTCCATTTGGCAGGGGCTTCTAAAACT
ATTTTGTATTCATGTGGGCAGGATGACATGGAGATCACTAGGAGTCACACAACTGCCTTAGAATGTA
AAACTCTCCTGCCAAACGAAATAGCTATTAGGCCCATGGACAAAACCGTATTGTTCACAGATAATTA
CAGTGATCTGGAAGTCACCGATTCCCATACTGTTTTCATTGACTGTCAAGCCACAGAGAAAATACTT
GAAGAAAACCCTAAATTTGGAATAGGAAAAGGAAAAAACTTGGGTGTTTCCTTTCCTAAGGATAATA
GCTGTGTTCAAGAAATCGCTGAAAAACAAGCACTGGCTGTAGGAAACAAAATAGTTCTTCACACCGA
GCAAAAGCAACAACTCTTTGCTGCTACTAATAGAACTACTAATGAAATCATCAAATTTCATAGTGCT
GCTATGGATGAAAAGGTCATAGGGAAAGTTGTAGACCAGGCCTGTACATTGGAAAAAGCGCAAGTTG
AAAGCTGTCAGTTAAATAATAGAGATAGAAGAAATGTGGACTTTACAAGTAGTCATGCAACTGCTGT
TTGTGGATCCAGTGATAATTATTCCTGTTTACCAAATGTTATTTCCTGTACTGATAATTTGGAGGGT
AGTGCCATGCTCTTATGTGATAAAGATGAGGAAAAAGCCAATTATTGCCCAGTGCAAAATGATCTTG
CTTATGCAAATGATTTTGCCAGTGAATATTACTTGGAATCTGAGGGACAGCCTCTCTCTGCTCCTTG
TCCTTTGTTAGAGAAGGAAGAAGTTATTCAAACCAGTACCAAAGGACAGTTAGACTGTGTTATAACA
CTGCACAAAGATCAAGATCTGATTAAGGATCCACGAAATCTATTGGCTAATCAAACTTTAGTATATA
GTCAAGATCTGGGGGAGATGACTAAACTTAATTCAAAGCGAGTATCTTTTAAGCTTCCAAAGGATCA
AATGAAAGTCTATGTTGATGACATTTATGTTATTCCTCAGCCTCATTTCTCAACCGACCAACCTCCA
TTACCTAAAAAAGGACAGAGTAGTATCAATAAAGAAGAAGTAATACTGTCTAAAGCTGGAAATAAGA
GTTTAAATATTATAGAAAATTCCTCTGCACCCATATGTGAAAACAAGCCCAAAATACTCAATAGTGA
GGAATGGTTTGCTGCAGCCTGTAAAAAAGAACTGAAGGAAAATATTCAAACAACTAACTATAATACA
GCTCTAGATTTCCACAGTAACTCAGACGTAACTAAGCAAGTCATTCAAACTCATGTCAATGCTGGAG
AAGCACCAGATCCTGTAATTACATCTAATGTTCCATGTTTTCATAGTATCAAACCAAATCTGAATAA
TTTGAATGGAAAAACTGGAGAGTTTTTAGCCTTTCAAACTGTTCATCTACCACCCCTTCCAGAGCAA
TTACTTGAATTAGGAAATAAGGCACACAATGATATGCATATAGTGCAAGCTACAGAAATACATAATA
TTAACATAATCTCCAGCAATGCTAAAGATAGTAGAGATGAGGAAAATAAAAAGTCTCATAATGGAGC
TGAAACCACCTCTCTACCGCCAAAGACAGTTTTTAAAGATAAAGTAAGGAGATGTTCTTTGGGAATC
TTTTTGCCTAGATTGCCCAACAAGACAAATTGTAGTGTCACTGGTATTGATGACCTGGAACAGATTC
CAGCAGACACAACTGATATAAATCACTTAGAAACTCAGCCGGTCTCTAGCAAAGATTCAGGCATTGG
ATCTGTTGCAGGTAAACTGAACCTAAGTCCTTCTCAATATATAAATGAGGAAAATCTTCCTGTATAT
CCTGATGAGATCAATTCTTCAGACTCTATTAACATAGAAACTGAGGAAAAGGCCTTGATTGAGACAT
ACCAAAAAGAGATTTCACCATATGAAAATAAAATGGGAAAAACTTGCAATAGCCAAAAAAGAACGTG
GGTACAAGAAGAAGAAGATATTCATAAGGAGAAAAAAATCAGAAAAAATGAGATTAAGTTTAGTGAT
ACGACACAAGATCGGGAGATTTTTGATCACCATACTGAAGAGGATATAGATAAAAGTGCTAACAGTG
TATTGATAAAAAACCTGAGCAGGACCCCATCTAGTTGCAGCAGCTCTCTGGATTCAATCAAGGCTGA
TGGGACCTCTCTGGACTTCAGCACTTACCGCAGTAGTCAAATGGAATCACAGTTTCTCAGAGATACT
ATTTGTGAAGAGAGCTTGAGGGAGAAACTCCAAGATGGGAGAATAACAATAAGGGAGTTCTTTATAC
TTCTCCAGGTCCACATCTTGATACAGAAACCCCGACAGAGCAATCTCCCAGGCAATTTTACTGTAAA
CACACCACCTACTCCAGAAGACCTGATGTTAAGTCAATATGTTTACCGACCCAAGATACAGATTTAT
AGAGAAGATTGTGAGGCTCGTCGCCAAAAGATTGAAGAATTAAAGCTTTCTGCATCGAACCAAGATA
AGCTGTTGGTTGATATAAATAAGAACCTGTGGGAAAAAATGAGACACTGCTCTGACAAAGAGCTGAA
GGCCTTTGGAATTTATCTTAACAAAATAAAGTCATGTTTTACCAAGATGACTAAAGTCTTCACTCAC
CAAGGAAAAGTGGCTCTGTATGGCAAGCTGGTGCAGTCAGCTCAGAATGAGAGGGAGAAACTTCAAA
TAAAGATAGATGAGATGGATAAAATACTTAAGAAGATCGATAACTGCCTCACTGAGATGGAAACAGA
AACTAAGAATTTGGAGGATGAAGAGAAAAACAATCCTGTGGAAGAATGGGATTCTGAAATGAGAGCT
GCAGAAAAAGAATTGGAACAGCTGAAAACTGAAGAAGAGGAGCTTCAAAGAAATCTCTTAGAACTGG
AGGTACAAAAAGAGCAGACCCTTGCTCAAATAGACTTTATGCAAAAACAAAGAAATAGAACTGAAGA
GCTACTGGATCAGTTGAGCTTGTCTGAGTGGGATGTCGTTGAGTGGAGTGATGATCAAGCTGTATTC
ACCTTTGTTTATGACACGATACAACTCACCATCACCTTTGAAGAGTCAGTTGTTGGTTTCCCTTTCC
TGGACAAGCGTTATAGGAAGATTGTTGATGTCAATTTTCAATCTCTGTTAGATGAGGATCAAGCTCC
TCCTTCCTCCCTTTTAGTTCATAAGCTTATTTTCCAGTACGTTGAAGAAAAGGAATCCTGGAAGAAG
ACATGTACAACCCAGCATCAGTTACCCAAGATGCTTGAAGAATTCTCACTGGTAGTGCACCATTGCA
GACTCCTTGGAGAGGAGATTGAGTATTTAAAGAGATGGGGACCAAATTATAACCTAATGAACATAGA
TATTAATAATAATGAATTGAGACTTTTATTCTCTAGCTCCGCAGCATTTGCAAAGTTTGAAATAACT
TTGTTTCTCTCAGCCTATTATCCATCTGTACCATTACCTTCCACCATTCAGAATCACGTTGGGAACA
CTAGCCAAGATGATATTGCTACCATTCTATCTAAAGTGCCACTGGAGAACAACTACCTGAAGAATGT
AGTCAAGCAAATTTACCAAGATCTGTTTCAGGACTGCCATTTCTACCACTAGACCCTTGGACCACCA
TTGGAACAACCAAGCAGAATGTACTTGATATTATTTCAGCGTCCCATTGCTGTTCAGCCTTTGTTTT
TACGTCATTACAAGCTGAGTAAAATTCCTTCTGATGATGTTATAGTTAATCTGTATGTTTTTTATAT
CTCTGCAGAATGATGGTGATGAAGTCTGGATGGTAGGCCTCATAGCCTACTATCAACTTACTCATCT
TTGTACCAAAGGTTTAAGTAATAGGACACTTAGGAAAAATGTCTCCTAACTAAACTAGTGCTTTCTG
CTTTAGTACAAGCCCTAAGGATTAACTTAAGTATAAGAAGTGTTATCACTGACAAGAACATTAGCCA
TTTTCCCATAACTAGATAGAGCTATGATTTTTTAGGTTGCCTGGCTTCTGCCTAGCAGATATTTCTG
GAGTAGAAATGTATCTGTCTACAAACTATTATCCTTTTTCTCCGTTACTAAAATGCTATTAAGAGAA
AGTAGGGCTGGGTGTGAGCCACCACACCCAGCAATGTTTTCTTAATAAGTATAGTTTTTCTAGGGAA
AGTTAATTCATTTTTGTCTAGTACATATATGTAAATATATTAATGTTGTTTTTGTGTTTGTGATGTA
GTAAGGAGATGTACATAGAAATTCATTGAGGTATATAGATACTCATCTGTCTAGGCAGTTCCCAATT
TTCTGAAGAATGTTTTACAGCAAAATTTTCTATTTTCTTTTATTAAATAGTGACACGTCAAACAATG
TCACATCCAAAACACTAGTTTCATCAATTTCTAGCAGTAATAATAGACTTGCTGTAAGTATTGTTTT
CTGATGCCATACCCTTGTCATACATATTATTAAATGACCAATATTATGTATGAAGTAGACAAAAAAA
TTTACTCAAACTTCATTCAAATCCTAATTGTGATAATTTTTGTTTTATATTTAATTATAAACCAAAA
TACATTTGCATTTTTAAGCTAATTTGTCTCAAAATTTTGCTTTATATTTTTGGATCAGGTTAAAGTC
CTGTGGATCCCCTGAATGTTATTGTCCCTCTTGATTGGTTTTTACTTCTGAGCTATACGTCAAAAGA
CACATAAGCTTCAAAAGTCAAGACAAACCTCATTTGCCATAAAAATCAAGATATAGATGTTCTGTTC
CGTAAACTCCTTGAAAAACATTTTAAAGTCATCAATATGATCTGTTTCCCATGAAACTTAAGTTAGC
TTTCTTATTGGAGTTATTTCTTTTCTGTAAGTCTGAAAAGTAGAGATTTTGTTTTACGCATTTTAGT
AACCTGCAACAACCAACTCTAAAAAAGATTTGGCTTGTAATGACGGTCTCTGCTTTTTTGGGTTTGG
AGTACACAATTGTAATATTTACTTAGTTATTTGTGTTTTTCTTTGTTCAAGGTATTGACTAGTTTCA
TAAATTTTTTGCAAGTTTTTCTTTCATTGGTTGGAAAGCAGATTACATTTTGCACTATTAAAATAAG
TTTATTACTTTAAAAAAAAAGTCGACG

Variant sequences of NOV11 are included in Example 3, Table 23. A variant sequence can include a single nucleotide polymorphism (SNP). A SNP can, in some instances, be referred to as a “cSNP” to denote that the nucleotide sequence containing the SNP originates as a cDNA.

The NOV11 protein (SEQ ID NO:24) encoded by SEQ ID NO:23 is 2316 amino acid residues in length and is presented using the one-letter amino acid code in Table 11B. Psort analysis predicts the NOV11 protein of the invention to be localized at the nucleus with a certainty of 0.8800.

TABLE 11B
Encoded NOV11 protein sequence

(SEQ ID NO:24)

MDGVSSEANEENDNIERPVRRRHSSILKPPRSPLQDLRGGNERVQESNALRNKKNSRRVSFADTI
KVFQTESHMKIVRKSEMEETETGENLLLIQNKKLEDNYCEITGMNTLLSAPIHTQMQQKEFSIIE
HTRERKHANDQTVIFSDENQMDLTSSHTVMITKGLLDNPISEKSTKIDTTSFLANLKLHTEDSRM
KKEVNFSVDQNTSSENKIDFNDFIKRLKTGKCSAFPDVPDKENFEIPIYSKEPNSASSTHQMHVS
LKEDENNSNITRLFREKDDGMNFTQCHTANIQTLIPTSSETNSRESKGNDITIYGNDFMDLTFNH
TLQILPATGNFSEIENQTQNAMDVTTGYGTKASGNKTVFKSKQNTAFQDLSINSADKIHITRSHI
MGAETHIVSQTCNQDARILAMTPESIYSNPSIQGCKTVFYSSCNDAMEMTKCLSNMREEKNLLKH
DSNYSKMYCNPDAMSSLTEKTIYSGEENMDITKSHTVAIDNQIFKQDQSNVQIAAAPTPEKEMML
QNLMTTSEDGKMNVNCNSVPHVSKERIQQSLSNPLSISLTDRKTELLSGENTDLTESHTSNLGSQ
VPLAAYNLAPESTSESHSQSKSSSDECEEITKSRNEPFQRSDIIAKNSLTDTWNKDKDWVLKILP
YLDKDSPQSADCNQEIATSHNIVYCGGVLDKQITNRNTVSWEQSLFSTTKPLFSSGQFSMKNHDT
AISSHTVKSVLGQNSKLAEPLRKSLSNPTPDYCHDKMIICSEEEQNMDLTKSHTVVIGFGPSELQ
ELGKTNLEHTTGQLTTMNRQIAVKVEKCGKSPIEKSGVLKSNCIMDVLEDESVQKPKFPKEKQNV
KIWGRKSVGGPKIDKTIVFSEDDKNDMDITKSYTIEINHRPLLEKRDCHLVPLAGTSETILYTCG
QDDMEITRSHTTALECKTVSPDEITTRPMDKTVVFVDNHVELEMTESHTVFIDYQEKERTDRPNF
ELSQRKSLGTPTVICTPTEESVFFPGNGESDRLVANDSQLTPLEEWSNNRGPVEVADNMELSKSA
TCKNIKDVQSPGFLNEPLSSKSQRRKSLKLKNDKTIVFSENHKNDMDITQSCMVEIDNESALEDK
EDFHLAGASKTILYSCGQDDMEITRSHTTALECKTLLPNEIAIRPMDKTVLFTDNYSDLEVTDSH
TVFIDCQATEKILEENPKFGIGKGKNLGVSFPKDNSCVQEIAEKQALAVGNKIVLHTEQKQQLFA
ATNRTTNEIIKFHSAAMDEKVIGKVVDQACTLEKAQVESCQLNNRDRRNVDFTSSHATAVCGSSD
NYSCLPNVISCTDNLEGSAMLLCDKDEEKANYCPVQNDLAYANDFASEYYLESEGQPLSAPCPLL
EKEEVIQTSTKGQLDCVITLHKDQDLIKDPRNLLANQTLVYSQDLGEMTKLNSKRVSFKLPKDQM
KVYVDDIYVIPQPHFSTDQPPLPKKGQSSINKEEVILSKAGNKSLNIIENSSAPICENKPKILNS
EEWFAAACKKELKENIQTTNYNTALDFHSNSDVTKQVIQTHVNAGEAPDPVITSNVPCFHSIKPN
LNNLNGKTGEFLAFQTVHLPPLPEQLLELGNKAHNDMHIVQATEIHNINIISSNAKDSRDEENKK
SHNGAETTSLPPKTVFKDKVRRCSLGIFLPRLPNKRNCSVTGIDDLEQIPADTTDINHLETQPVS
SKDSGIGSVAGKLNLSPSQYINEENLPVYPDEINSSDSINIETEEKALIETYQKEISPYENKMGK
TCNSQKRTWVQEEEDIHKEKKIRKNEIKFSDTTQDREIFDHHTEEDIDKSANSVLIKNLSRTPSS
CSSSLDSIKADGTSLDFSTYRSSQMESQFLRDTICEESLREKLQDGRITIREFFILLQVHILIQK
PRQSNLPGNFTVNTPPTPEDLMLSQYVYRPKIQIYREDCEARRQKIEELKLSASNQDKLLVDINK
NLWEKMRHCSDKELKAFGIYLNKIKSCFTKMTKVFTHQGKVALYGKLVQSAQNEREKLQIKIDEM
DKILKKIDNCLTEMETETKNLEDEEKNNPVEEWDSEMRAAEKELEQLKTEEEELQRNLLELEVQK
EQTLAQIDFMQKQRNRTEELLDQLSLSEWDVVEWSDDQAVFTFVYDTIQLTITFEESVVGFPFLD
KRYRKIVDVNFQSLLDEDQAPPSSLLVHKLIFQYVEEKESWKKTCTTQHQLPKMLEEFSLVVHHC
RLLGEEIEYLKRWGPNYNLMNIDINNNELRLLFSSSAAFAKFEITLFLSAYYPSVPLPSTIQNHV
GNTSQDDIATILSKVPLENNYLKNVVKQIYQDLFQDCHFYH

A search against the Patp database, a proprietary database that contains sequences published in patents and patent publications, yielded several homologous proteins shown in Table 11C.

TABLE 11C
Patp results for NOV11

| | | | Smallest |
|---|---|---|---|
| | | | Sum |
| Sequences producing High-scoring | Reading | High | Prob |
| Segment Pairs: | Frame | Score | P (N) |
| >patp:AAW88398 Human testis secreted | +1 | 2444 | 1.7e−253 |
| protein do15_4 | | | |
| >patp:AAU71933 Human bone marrow | +1 | 2444 | 1.7e−253 |
| tissue polypeptide #11 | | | |
| >patp:AAU71961 Human bone marrow | +1 | 2444 | 1.7e−253 |
| tissue polypeptide #39 | | | |
| >patp:AAU71933 Human bone marrow | +1 | 2444 | 1.7e−253 |
| tissue polypeptide #11 | | | |
| >patp:AAU71961 Human bone marrow | +1 | 2444 | 1.7e−253 |
| tissue polypeptide #39 | | | |

In a BLAST search of public sequence databases, it was found, for example, that the nucleic acid sequence of this invention has 5584 of 5584 bases (100%) identical to a gb:GENBANK-ID:AB046790|acc:AB046790.1 mRNA from Homo sapiens (mRNA for KIAA1570 protein, partial cds). The full amino acid sequence of the protein of the invention was found to have 1790 of 1793 amino acid residues (99%) identical to, and 1792 of 1793 amino acid residues (99%) similar to, the 1833 amino acid residue ptnr:SPTREMBL-ACC:Q9NR92 protein from Homo sapiens (AF] 5Q14 PROTEIN).

NOV11 also has homology to the proteins shown in the BLASTP data in Table 11D.

TABLE 11D
BLAST results for NOV11

| Gene Index/ | | | | | |
|---|---|---|---|---|---|
| Identifier | Protein/Organism | Length (aa) | Identity (%) | Positives (%) | Expect |
| gi\|18308012\|gb\| | AF15q14 isoform 2 | 2316 | 2316/2316 | 2316/2316 | 0.0 |
| AAL67803.1\|AF461041_1 | [Homo sapiens] | | (100%) | (100%) | |
| (AF461041) | | | | | |
| gi\|9966807\|ref\|NP_{—} | AF15q14 protein | 1833 | 1790/1793 | 1792/1793 | 0.0 |
| 065113.1\|(NM_020380) | [Homo sapiens] | | (99%) | (99%) | |
| gi\|14749154\|ref\|XP_{—} | AF15q14 protein | 1833 | 1789/1793 | 1791/1793 | 0.0 |
| 031524.1\|(XM_031524) | [Homo sapiens] | | (99%) | (99%) | |
| gi\|10047205\|dbj\| | KIAA1570 protein | 1360 | 1360/1360 | 1360/1360 | 0.0 |
| BAB13396.1\|(AB046790) | [Homo sapiens] | | (100%) | (100%) | |
| gi\|14749150\|ref\|XP_{—} | similar to | 915 | 900/900 | 900/900 | 0.0 |
| 012461.3\|(XM_012461) | KIAA1570 protein | | (100%) | (100%) | |
| | [Homo sapiens] | | | | |

A multiple sequence alignment is given in Table 11E, with the NOV11 protein being shown on line 1 in Table 11E in a ClustalW analysis, and comparing the NOV11 protein with the related protein sequences shown in Table 11D. This BLASTP data is displayed graphically in the ClustalW in Table 11E.

[image]

[image]

[image]

[image]

[image]

[image]

[image]

NOV11 nucleic acids, and the encoded polypeptides, according to the invention are useful in a variety of applications and contexts. For example, NOV11 nucleic acids and polypeptides can be used to identify proteins that are members of the Nuclear Protein-like Protein Family. The NOV11 nucleic acids and polypeptides can also-be used to screen for molecules, which inhibit or enhance NOV11 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., Von Hippel-Lindau (VHL) syndrome, Cirrhosis, Transplantation, Hemophilia, hypercoagulation, Idiopathic thrombocytopenic purpura, autoimmume disease, allergies, immunodeficiencies, transplantation, Graft vesus host, Cardiovascular diseases, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, as well as other diseases, disorders and conditions.

In addition, various NOV11 nucleic acids and polypeptides according to the invention are useful, inter alia, as novel members of the protein families according to the presence of sequence relatedness to previously described proteins. The NOV11 nucleic acids and polypeptides, antibodies and related compounds according to the invention will be useful in therapeutic and diagnostic applications in the mediation of cardiac, immune, and nerve physiology. As such, the NOV11 nucleic acids and polypeptides, antibodies and related compounds according to the invention may be used to treat cardiovascular, immune, and nervous system disorders, e.g., Von Hippel-Lindau (VHL) syndrome, Cirrhosis, Transplantation, Hemophilia, hypercoagulation, Idiopathic thrombocytopenic purpura, autoimmume disease, allergies, immunodeficiencies, transplantation, Graft vesus host, Cardiovascular diseases, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, as well as other diseases, disorders and conditions.

The NOV11 nucleic acids and polypeptides are useful for detecting specific cell types. For example, expression analysis has demonstrated that a NOV11 nucleic acid is expressed in Adipose, Aorta, Artery, Coronary Artery, Umbilical Vein, Thyroid, Liver, Small Intestine, Duodenum, Colon, Ascending Colon, Bone Marrow, Lymph node, Tonsils, Thymus, Cartilage, Muscle, Brain, Cervix, Uterus, Vulva, Prostate, Testis, Lung, Bronchus, Urinary Bladder, Kidney, Skin, Epidermis, Dermis.

Additional utilities for NOV11 nucleic acids and polypeptides according to the invention are disclosed herein.

NOV12

A NOV12 polypeptide has been identified as a Plasma Membrane Protein-like protein (also referred to as CG94282-01). The disclosed novel NOV12 nucleic acid (SEQ ID NO:25) of 8811 nucleotides is shown in Table 12A. The novel NOV12 nucleic acid sequences maps to the chromosome 12.

An ORF begins with an ATG initiation codon at nucleotides 1-3 and ends with a TAG codon at nucleotides 4378-4380. A putative untranslated region and/or downstream from the termination codon is underlined in Table 12A, and the start and stop codons are in bold letters.

TABLE 12A
NOV12 Nucleotide Sequence

(SEQ ID NO:25)

ATGCTGTTCAAGCTCCTGCAGAGACAAACCTATACCTGCCTGCCCACAGGTATGGGCTCTACGTGTGCTTCTT
GGGCGTCGTTGTCACCATCGTCTCCGCCTTCCAGTTCGGAGAGTGGGTAGAAGCCAGGGATCCTGCCAAACATC
CTATAGTGCACAGGACAGCCCCTACAACAAAGAATCATCCAGCCCAAAATGTCGATAGTGCTGAAGTTGAGAAA
TCCGGAATTAGAAGGGGCAAGAATGGCTGCAGGGCAGTTAGTCTACAGGACTGGCCTGGGACTAGAGGATGTGC
CAATTTCACCTTCGCCTTCTGCCATGATTGTAAGTTTTCTGAGGTCTCCCAGAAACGCTTCCTGTACATCCTGC
AGAACTGTCATTGGTTAACTGATTGGGGTTGGACTTGGTTGGCTCTGCTCCACGGGTCTCTCATCCTCCAGGGA
CCAGCCAGCGAACCTGGTTGTGTTCTTCTCAAGGCAAAGGTGGTTCTGGAATGGAGCCGAGATCAATACCATGT
TTTGTTTGATTCCTATAGAGACAATATTGCTGGAAAGTCCTTTCAGAATCGGCTTTGTCTGCCCATGCCGATTG
ACGTTGTTTACACCTGGGTGAATGGCACAGATCTTGAACTACTGAAGGAACTACAGCAGGTCAGAGAACAGATG
GAGGAGGAGCAGAAAGCAATGAGAGAAATCCTTGGGAAAAACACAACGGAACCTACTAAGAAGAGTGAGAAGCA
GTTAGAGTGTTTGCTAACACACTGCATTAAGGTGCCAATGCTTGTCCTGGACCCAGCCCTGCCAGCCAACATCA
CCCTGAAGGACCTGCCATCTCTTTATCCTTCTTTTCATTCTGCCAGTGACATTTTCAATGTTGCAAAACCAAAA
AACCCTTCTACCAATGTCTCAGTTGTTGTTTTTGACAGTACTAAGGATGGGACATTGCTCACTCAGAAGGTGAC
TTTTGAGTGGAAATGTGAAGAAGGTGAGGTAGCCAGCAATGCGAATATCTGGGGAAAGACTGATCTGGGTTCCC
CCAGGAGGCCTTTGCCATGGCCTGTGGCCCTGGAGCCACCTAGGGCTCAGCTCAGCTCTGCCCTACAGATTCTC
ACTAGGCCACGGGTATCTCAGGACAGAGCCAACACAAGTTATGAAATTAAACTAGACACACCCCTTCTTCGAGG
TTACGCCAAGCCAGTGCCTGGGCCTGAAACTGGCCTGCAGCCCCTCAGCTTCGCCCACTGCCTTCCGACCCTGG
ACCTTCGCAAAGTGAACGAGCTTCGGGACTTCGTGAAAATGTATAAGCAGGATCCGAGCATTCTGCATACCAAG
GAAACGTGCTTTCTGAGGGAGCAGGTGGAGAGCATGGGGGAAAGCTATTATAAATCAGAAGAAAATATCAAGGA
ATTAAAAACAGGTAGTAAGAAGGTGGAGGAAAACATAAGCACAGACGAACTATCAAGTGAGGAAAGTGATCTAG
AAATTGATAACGAAGCTGTGATTGAACCAGACACTGATTCCCCTCAAGAAATGGGAGATGGAGAGGCCAGTGTA
GCGCTTCTAAAACTGAATAACCCCAAGGATTTTCAAGAATTGAATAAGCAAACTAAGAAGAACATGACCATTGA
TGGAAAAGAACTGACCATAAGTCCTGCATATTTATTATGGGATCTGAGCGCCATCAGCCAGTCTAAGCAGGATG
AAGACATCTCTGCCAGTCGTTTTGAAGATAACGAAGAACTGAGGTACTCATTGCGATCTATCGAGAGGCATGCA
CCATGGGTTCGGAATATTTTCATTGTCACCAACGGGCAGATTCCATCCTGGCTGAACCTTGACAATCCTCGAGT
GACAATAGTAACACACCAGGATGTTTTTCGAAATTTGAGCCACTTGCCTACCTTTAGTTCACCTGCTATTGAAA
GTCACATTCATCGCATCGAAGGGCTGTCCCAGAAGTTTATTTACCTAAATGATGATGTCATGTTTGGGAAGGAT
GTCTGGCCAGATGATTTTTACAGTCACTCCAAAGGCCAGAAGGTTTATTTGACATGGCCTGTGCCAAACTGTGC
CGAGGGCTGCCCAGGTTCCTGGATTAAGGATGGCTATTGTGACAAGGCTTGTAATAATTCAGCCTGCGATTGGG
ATGGTGGGGATTGCTCTGGAAACAGTGGAGGGAGTCGCTATATTGCAGGAGGTGGAGGTACTGGGAGTATTGGA
GTTGGACAGCCCTGGCAGTTTGGTGGAGGAATAAACAGTGTCTCTTACTGTAATCAGGGATGTGCGAATTCCTG
GCTCGCTGATAAGTTCTGTGACCAAGCATGCAATGTCTTGTCCTGTGGGTTTGATGCTGGCGACTGTGGGCAAG
AAAACTCAGACTCAAAGAATAGGAAAACAGAGGAAAAATGCCCAGTTAAAAAAAAAAAAATCATGTTTCTGTTT
TTTCCTCTAGATCATTTTCATGAATTGTATAAAGTGATCCTTCTCCCAAACCAGACTCACTATATTATTCCAAA
AGGTGAATGCCTGCCTTATTTCAGCTTTGCAGAAGTAGCCAAAAGAGGAGTTGAAGGTGCCTATAGTGACAATC
CAATAATTCGACATGCTTCTATTGCCAACAAGTGGAAAACCATCCACCTCATAATGCACAGTGGAATGAATGCC
ACCACAATACATTTTAATCTCACGTTTCAAAATACAAACGATGAAGAGTTCAAAATGCAGATAACAGTGGAGGT
GGACACAAGGGAGGGACCAAAACTGAATTCTACAGCCCAGAAGGGTTACGAAAATTTAGTTAGTCCCATAACAC
TTCTTCCAGAGGCGGAAATCCTTTTTGAGGATATTCCCAAAGAAAAACGCTTCCCGAAGTTTAAGAGACATGAT
GTTAACTCAACAAGGAGAGCCCAGGAAGAGGTGAAAATTCCCCTGGTAAATATTTCACTCCTTCCAAAAGACGC
CCAGTTGAGTCTCAATACCTTGGATTTGCAACTGGAACATGGAGACATCACTTTGAAAGGATACAATTTGTCCA
AGTCAGCCTTGCTGAGATCATTTCTGATGAACTCACAGCATGCTAAAATAAAAAATCAAGCTATAATAACAGAT
GAAACAAATGACAGTTTGGTGGCTCCACAGGAAAAACAGGTTCATAAAAGCATCTTGCCAAACAGCTTAGGAGT
GTCTGAAAGATTGCAGAGGTTGACTTTTCCTGCAGTGAGTGTAAAAGTGAATGGTCATGACCAGGGTCAGAATC
CACCCCTGGACTTGGAGACCACAGCAAGATTTAGAGTGGAAACTCACACCCAAAAAACCATAGGCGGAAATGTG
ACAAAAGAAAAGCCCCCATCTCTGATTGTTCCACTGGAAAGCCAGATGACAAAAGAAAAGAAAATCACAGGGAA
AGAAAAAGAGAACAGTAGAATGGAGGAAAATGCTGAAAATCACATAGGCGTTACTGAAGTGTTACTTGGAAGAA
AGCTGCAGCATTACACAGATAGTTACTTGGGCTTTTTGCCATGGGAGAAAAAAAAGTATTTCCAAGATCTTCTC
GACGAAGAAGAGTCATTGAAGACACAATTGGCATACTTCACTGATAGCAAAAATACTGGGAGGCAACTAAAAGA
TACATTTGCAGATTCCCTCAGATATGTAAATAAAATTCTAAATAGCAAGTTTGGATTCACATCGCGGAAAGTCC
CTGCTCACATGCCTCACATGATTGACCGGATTGTTATGCAAGAACTGCAAGATATGTTCCCTGAAGAATTTGAC
AAGACGTCATTTCACAAAGTGCGCCATTCTGAGGATATGCAGTTTGCCTTCTCTTATTTTTATTATCTCATGAG
TGCAGTGCAGCCACTGAATATATCTCAAGTCTTTGATGAAGTTGATACAGATCAATCTGGTGTCTTGTCTGACA
GAGAAATCCGAACACTGGCTACCAGAATTCACGAACTGCCGTTAAGTTTGCAGGATTTGACAGGTCTGGAACAC
ATGCTAATAAATTGCTCAAAAATGCTTCCTGCTGATATCACGCAGCTAAATAATATTCCACCAACTCAGGAATC
CTACTATGATCCCAACCTGCCACCGGTCACTAAAAGTCTAGTAACAAACTGTAAACCAGTAACTGACAAAATCC
ACAAAGCATATAAGGACAAAAACAAATATAGGTTTGAAATCATGGGAGAAGAAGAAATCGCTTTTAAAATGATT
CGTACCAACGTTTCTCATGTGGTTGGCCAGTTGGATGACATAAGAAAAAACCCTAGGATCTCACTCTGTTGTCC
AAGCTGGAATGCAGTAATGCAAACATGGCTCACTGTAGCCTCGACCTCGTGGGCTCAAGCAATCCTCCCACCTC
AGCCTCCTGACTAGTGGAACCACAGACATGAGCTGCTGCACCCAGCTAAAATGGAGTATTTTTAATTTCTGGGT
CTTTTAAATGCATTTGGAGGTCTTTAGTTTTACCTCACTGAAATTAGGATTTTAATTATAAATAATCAAAGATG
TGAACCTTACAGACATTTTAAAGCCATTATATTTTTTCTATAAACCCTGTTCTCGTTTGGAGGAGAAAGAAATT
GGAATTTTCAAAAAAAATAAAAATACCTTTAACACCTATTTAGTGTCTTTAGTAATCCAGTAAAATACTTGATT
TTTTACTAAATGTTTCCCACAAGCCAAGCAAACCATAAGCTACAATAATAATTACCTAGCGTACAGCCCTCTTT
GCATATGCTGTTCCCTCCACTTGAAGTGTACTGTTTAATTTCTTAAAATAACTTTAGCTTTTAAGAACCAATTT
TGATGGGAGTACAGACTTCCCCCATTTTCTTGATGAGTTCTCTCCGTCATGTGTAGTAATAATGTGAGAATTTG
CAGTTTTTAGTTGTAGCCTATACTTTTAGGTCTTTGTGCCAATTTGAAAGTTATTGGGTTAGAGTATTCATAGA
CATTTTCATGGTACTTAAAGGGACAGGGGTTTAGTAAAAAGACACATGGCAAGCCAGGCTTTTTCCACAGTTTG
CCAGGCCCAGCTGCCTCTTGTGTACCTGAACAGATTTTATCATTAACCCTTGTTTATGTTGTTTTGTTTTATTT
CGACGAAGGCTTATTTTAAGTCAGGCATGGAAAACTAGACTTCAGACTGACTTCAGCTTTAAGGACATGTTTAT
CCCGTTAACAGGGAGTCTGGGATAGACAATCTCCAGGCTTTGTTTTTCTCTGAATTTCTTAGCTCTGCTTGTGA
TGGCTTCATCATCAGGCCACAGACCATTAACACATTCTAGAACTTTAACATTGGTTAAATAATACCATCTAATA
GCCTGTCTTCAGCATTTCCCCAGTTGCCTCCAAATGCCCTTCATAGCTGTTCTCTGCCTCTGTTTGTTTTTAAT
CCAAGATACACTCAAGGCTCATATATTAGGTTGACATAGCTCTTTAGTATCCTTTAATTTAAAGCAGTCTCCAG
GTTTAGAGAAAGATGAATGAGCTTTCACATACCCCTCACTTGTCTTCTTCAGAAGTGTAGGCTACAACTAAAAC
TTCCTTCTTCAGAAGGAAGACAAGTGATTTATATTTATTTACTTCCATTTCTATTTGACCTTGTTTCATCTAAA
CACACCCACTCCACCACTGCTACCTGATTAATATTAAGTGAATCTCAAACATTGTATCATTTTAGCTCCACGTT
TTTTGTATGTATCTCCAAAATATAAAGATTCTTAAAAATATAACCACAATACCATTATCACCCTAAAAAAATCA
ATAATGATTCCTTAATATGACCAACTACTTTGTCAATGTACACCTTTCACTCCTCTTAACTTTCATAAAGACTT
ATGTGTTTTTTTGGTTTTTAAGTTTGTTGGTTTGAAGTTAAATCTATGGGTTTTCCCTCCATCTCTCTTTTTTT
AACCGTATAATTTTTTGCATGTGTATATGAATAAATCTGATTATAGATTCTATAGCTATCTTACACTTTGTCCC
TCTCTGATTGAATCCTAGTTAACAAGTTTCTATGTCTCTTGTATTTCCCATAAATTGGTAGTTGGATCTGAAGG
CTTTATCAGGTTTGTTTGATTTTTTTTTTTTTAATTTTGGCGAATCTACTTCAAAAGTATTGGCCTACCTACAA
GCCACTTTAATCGGCCCTTAGTTTAGTGACCTTTGCCTTGAAAGGAACTTGAAACAAGCAAGGAAGCACCACTG
TAATCTGCTTTTTTGCCAGAACTGTAGCATCTTACAGCTTGGTTAGAGACATAGTAAGCAGAAATTATCAAATT
CATATAATCTGTAGCTATAAGGCACTGTCTCTCTCTCTCAATTATTTACATGATTTTTCTTTGTAATATAACTA
TCATTTCAGAGAACTTGGTTTTGATTTTTTTTTTTTAATCTTTTTGAGACAGAGTCTCGCTTTATCACCCAGGC
TGGAGTGCAGTGGTGCAATCTAAAGATTGCTGACTGCAACCTCTGCCTCCCGAGTTCAGCAATTCTAGTGCCTC
AGCCTCTCGAGTAGCTGGGATTACAGGCATGCCACCACACCCGGCTAATTTTTTTGTATTTTTAGTAAAGACAG
GGTTTCACCATGTTGGCTAGGCTGGTCTCAAATTTTTGACCTCAAGTAATCAGCCTACCTTGATCTCCCAAAGT
GCTGGGATTACAGGCATGAGCCACCATGCATGGCCTTCAGAGAACTTGGTTTTAGGTACTTACGGATTGTCTTT
CTTTTTTTTCCTCACTGCAGCCTCTCCCTCCCAGGTTCAAGCGATTCTCCTACCTCAGCTTCCTGAAGAGCTGG
GACCACAGGAAGTTTGTTTGCCTGAATGACAACATTGACCACAATCATAAAGATGCTCAGACAGTGAAGGCTGT
TCTCAGGGACTTCTATGAATCCATGTTCCCCATACCTTCCCAATTTGAACTGCCAAGAGAGTATCGAAACCGTT
TCCTTCATATGCATGAGCTGCAGGAATGGAGGGCTTATCGAGACAAATTGAAGTTTTGGACCCATTGTGTACTA
GCAACATTGATTATGTTTACTATATTCTCATTTTTTGCTGAGCAGTTAATTGCACTTAAGCGGAAGATATTTCC
CAGAAGGAGGATACACAAAGAAGCTAGTCCCAATCGAATCAGAGTATAGAAGATCTTCATTTGAAAACCATCTA
CCTCAGCATTTACTGAGCATTTTAAAACTCAGCTTCACAGAGATGTCTTTGTGATGTGATGCTTAGCAGTTTGG
CCCGAAGAAGGAAAATATCCAGTACCATGCTGTTTTGTGGCATGAATATAGCCCACTGACCAGGAATTATTTAA
CCAACCCACTGAAAACTTGTGTGTTGAGCAGCTCTGAACTGATTTTACTTTTAAAGAATTTGCTCATGGACCTG
TCATCCTTTTTATAAAAAGGCTCACTGACAAGAGACAGCTGTTAATTTCCCACAGCAATCATTGCAGACTAACT
TTATTAGGAGAACCCTATGCCAGCTGGGAGTGATTGCTAAGAGGCTCCAGTCTTTGCATTCCAAAGCCTTTTGC
TAAAGTTTTGCACTTTTTTTTTTTCATTTCCCATTTTTAAGTAGTTACTAAGTTAACTAGTTATTCTTGCTTCT
GAGTATAACGAATTGGGATGTCTAAACCTATTTTTATAGATGTTATTTAAATAATGCAGCAATATCACCTCTTA
TTGACAATACCTAAATTATGAGTTTTATTAATATTTAAGACTGTAAATGGTCTTAAACCACTAACTACTGAAGA
GCTCAATGATTGACATCTGAAATGCTTTGTAATTATTGACTTCAGCCCCTAAGAATGCTATGATTTCACGTGCA
GGTCTAATTTCAAAGGGCTAGAGTTAGTACTACTTACCAGATGTAATTATGTTTTGGAAATGTACATATTCAAA
CAGAAGTGCCTCATTTTAGAAATGAGTAGTGCTGATGGCACTGGCACATTACAGTGGTGTCTTGTTTAATACTC
ATTGGTATATTCCAGTAGCTATCTCTCTCAGTTGGTTTTTGATAGAACAGAGGCCAGCAAACTTTCTTTGTAAA
AGGCTGGTTAGTAAATTATTGCAGGCCACCTGTGTCTTTGTCATACATTCTTCTTGCTGTTGTTTAGTTTGTTT
TTTTTCAAACAACCCTCTAAAAATGTAAAAACCATGTTTAGCTTGCAGCTGTACAAAAACTGCCCACCAGCCAG
ATGTGACCCTCAGGCCATCATTTGCCAATCACTGAGAATTAGTTTTTGTTGTTGTTGTTGTTGTTGTTTTTGAG
ACAGAGTCTCTCTCTGTTGCCCAGGCTGGAGTGCAGTGGCGCAATCTCAGCTCACTGCAACCTCCGCCTCCCGG
GTTCAAGCAGTTCTGTCTCAGCCTTCTGAGTAGCTGGGACTACAGGTGCATGCCACCACACCCTGCTAATTTTT
GTATTTTTAGTAGAGACGGGGGTTCCACCATATTGGTCAGGCTTATCTTGAACTCCTGACCTCAGGTGATCCAC
CTGCCTCTGCCTCCCAAAGTGCTGAGATTACAGGCATAAGCCAGTGCACCCAGCCGAGAATTAGTATTTTTATG
TATGGTTAAACCTTGGCGTCTAGCCATATTTTATGTCATAATACAATGGATTTGTGAAGAGCAGATTCCATGAG
TAACTCTGACAGGTATTTTAGATCATGATCTCAACAATATTCTTCCAAAATGGCATACATCTTTTGTACAAAGA
ACTTGAAATGTAAATACTGTGTTTGTGCTGTAAGAGTTGTGTATTTCAAAAACTGAAATCTCATAAAAAGTTAA
ATTTT

Variant sequences of NOV12 are included in Example 3, Table 24. A variant sequence can include a single nucleotide polymorphism (SNP). A SNP can, in some instances, be referred to as a “cSNP” to denote that the nucleotide sequence containing the SNP originates as a cDNA.

The NOV12 protein (SEQ ID NO:26) encoded by SEQ ID NO:25 is 1459 amino acid residues in length and is presented using the one-letter amino acid code in Table 12B. Psort analysis predicts the NOV12 protein of the invention to be localized at the plasma membrane with a certainty of 0.6500.

TABLE 12B
Encoded NOV12 protein sequence

(SEQ ID NO:26)

MLFKLLQRQTYTCLSHRYGLYVCFLGVVVTIVSAFQFGEWVEARDPAKHPIVHRTAPTTKNHPAQ
NVDSAEVEKSGIRRGKNGCRAVSLQDWPGTRGCANFTFAFCHDCKFSEVSQKRFLYILQNCHWLT
DWGWTWLALLHGSLILQGPASEPGCVLLKAKVVLEWSRDQYHVLFDSYRDNIAGKSFQNRLCLPM
PIDVVYTWVNGTDLELLKELQQVREQMEEEQKAMREILGKNTTEPTKKSEKQLECLLTHCIKVPM
LVLDPALPANITLKDLPSLYPSFHSASDIFNVAKPKNPSTNVSVVVFDSTKDGTLLTQKVTFEWK
CEEGEVASNANIWGKTDLGSPRRPLPWPVALEPPRAQLSSALQILTRPRVSQDRANTSYEIKLDT
PLLRGYAKPVPGPETGLQPLSFAHCLPTLDLRKVNELRDFVKMYKQDPSILHTKETCFLREQVES
MGESYYKSEENIKELKTGSKKVEENISTDELSSEESDLEIDNEAVIEPDTDSPQEMGDGEASVAL
LKLNNPKDFQELNKQTKKNMTIDGKELTISPAYLLWDLSAISQSKQDEDISASRFEDNEELRYSL
RSIERHAPWVRNIFIVTNGQIPSWLNLDNPRVTIVTHQDVFRNLSHLPTFSSPAIESHIHRIEGL
SQKFIYLNDDVMFGKDVWPDDFYSHSKGQKVYLTWPVPNCAEGCPGSWIKDGYCDKACNNSACDW
DGGDCSGNSGGSRYIAGGGGTGSIGVGQPWQFGGGINSVSYCNQGCANSWLADKFCDQACNVLSC
GFDAGDCGQENSDSKNRKTEEKCPVKKKKIMFLFFPLDHFHELYKVILLPNQTHYIIPKGECLPY
FSFAEVAKRGVEGAYSDNPIIRHASIANKWKTIHLIMHSGMNATTIHFNLTFQNTNDEEFKMQIT
VEVDTREGPKLNSTAQKGYENLVSPITLLPEAEILFEDIPKEKRFPKFKRHDVNSTRRAQEEVKI
PLVNISLLPKDAQLSLNTLDLQLEHGDITLKGYNLSKSALLRSFLMNSQHAKIKNQAIITDETND
SLVAPQEKQVHKSILPNSLGVSERLQRLTFPAVSVKVNGHDQGQNPPLDLETTARFRVETHTQKT
IGGNVTKEKPPSLIVPLESQMTKEKKITGKEKENSRMEENAENHIGVTEVLLGRKLQHYTDSYLG
FLPWEKKKYFQDLLDEEESLKTQLAYFTDSKNTGRQLKDTFADSLRYVNKILNSKFGFTSRKVPA
HMPHMIDRIVMQELQDMFPEEFDKTSFHKVRHSEDMQFAFSYFYYLMSAVQPLNISQVFDEVDTD
QSGVLSDREIRTLATRIHELPLSLQDLTGLEHMLINCSKMLPADITQLNNIPPTQESYYDPNLPP
VTKSLVTNCKPVTDKIHKAYKDKNKYRFEIMGEEEIAFKMIRTNVSHVVGQLDDIRKNPRISLCC
PSWNAVMQTWLTVASTSWAQAILPPQPPD

A search against the Patp database, a proprietary database that contains sequences published in patents and patent publications, yielded several homologous proteins shown in Table 12C.

TABLE 12C
Patp results for NOV12

| | | | Smallest |
|---|---|---|---|
| | | | Sum |
| Sequences producing High-scoring | Reading | High | Prob |
| Segment Pairs: | Frame | Score | P (N) |
| >patp:ABB30279 Peptide #2930 | +1 | 1900 | 7.5e−196 |
| encoded by breast cell | | | |
| >patp:AAM56268 Human brain expressed | +1 | 1900 | 7.5e−196 |
| single exon probe | | | |
| >patp:AAM16457 Peptide #2891 | +1 | 1900 | 7.5e−196 |
| encoded by probe | | | |
| >patp:AAM28952 Peptide #2989 | +1 | 1900 | 7.5e−196 |
| encoded by probe | | | |
| >patp:AAM04186 Peptide #2868 | +1 | 1900 | 7.5e−196 |
| encoded by probe | | | |

In a BLAST search of public sequence databases, it was found, for example, that the nucleic acid sequence of this invention has 6444 of 6447 bases (99%) identical to a gb:GENBANK-ID:AB033034|acc:AB033034.1 mRNA from Homo sapiens (mRNA for KIAA1208 protein, partial cds). The full amino acid sequence of the protein of the invention was found to have 663 of 663 amino acid residues (100%) identical to, and 663 of 663 amino acid residues (100%) similar to, the 663 amino acid residue ptnr:SPTREMBL-ACC:Q9ULL2 protein from Homo sapiens (KIAA1208 PROTEIN).

NOV12 also has homology to the proteins shown in the BLASTP data in Table 12D.

TABLE 12D
BLAST results for NOV12

| Gene Index/ | | | | | |
|---|---|---|---|---|---|
| Identifier | Protein/Organism | Length (aa) | Identity (%) | Positives (%) | Expect |
| gi\|6382022\|dbj\| | KIAA1208 protein | 663 | 663/663 | 663/663 | 0.0 |
| BAA86522.1\|(AB033034) | [Homo sapiens] | | (100%) | (100%) | |
| gi\|16551459\|dbj\| | unnamed protein | 847 | 585/613 | 585/613 | 0.0 |
| BAB71102.1\|(AK056137) | product | | (95%) | (95%) | |
| | [Homo sapiens] | | | | |
| gi\|2137411\|pir\|\| | hypothetical | 384 | 277/400 | 307/400 | e−142 |
| I49528 | protein | | (69%) | (76%) | |
| | [Mus musculus] | | | | |
| gi\|11360271\|pir\|\| | hypothetical | 248 | 134/137 | 135/137 | 2e−73 |
| T50618 | protein | | (97%) | (97%) | |
| | DKFZp762B226.1 | | | | |
| | [Homo sapiens] | | | | |
| gi\|7303923\|gb\| | CG8027 gene | 652 | 84/155 | 114/155 | 9e−49 |
| AAF58967.1\|(AE003834) | product | | (54%) | (73%) | |
| | [Drosophila | | | | |
| | melanogaster] | | | | |

A multiple sequence alignment is given in Table 12E, with the NOV12 protein being shown on line 1 in Table 12E in a ClustalW analysis, and comparing the NOV12 protein with the related protein sequences shown in Table 12D. This BLASTP data is displayed graphically in the ClustalW in Table 12E.

[image]

[image]

[image]

[image]

The NOV12 Clustal W alignment shown in Table 12E was modified to end at amino residue 1200. The data in Table 1E includes all of the regions overlapping with the NOV12 protein sequences.

NOV12 nucleic acids, and the encoded polypeptides, according to the invention are useful in a variety of applications and contexts. For example, NOV12 nucleic acids and polypeptides can be used to identify proteins that are members of the Plasma Membrane Protein-like Protein Family. The NOV12 nucleic acids and polypeptides can also be used to screen for molecules, which inhibit or enhance NOV12 activity or function. Specifically, the nucleic acids and polypeptides according to the invention may be used as targets for the identification of small molecules that modulate or inhibit, e.g., cellular activation and signal transduction. These molecules can be used to treat, e.g., Diabetes, Von Hippel-Lindau (VHL) syndrome, Pancreatitis, Obesity, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Von Hippel-Lindau (VHL) syndrome, Cirrhosis, Transplantation, Von Htippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection as well as other diseases, disorders and conditions.

In addition, various NOV12 nucleic acids and polypeptides according to the invention are useful, inter alia, as novel members of the protein families according to the sequence relatedness to previously described proteins. The NOV12 nucleic acids and polypeptides, antibodies and related compounds according to the invention will be useful in therapeutic and diagnostic applications in the mediation of cardiac, nerve, and immune physiology. As such, the NOV12 nucleic acids and polypeptides, antibodies and related compounds according to the invention may be used to treat cardiovascular, immune, and nervous system disorders, e.g., Diabetes, Von Hippel-Lindau (VHL) syndrome, Pancreatitis, Obesity, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Von Hippel-Lindau (VHL) syndrome, Cirrhosis, Transplantation, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection as well as other diseases, disorders and conditions.

The NOV12 nucleic acids and polypeptides are useful for detecting specific cell types. For example, expression analysis has demonstrated that a NOV12 nucleic acid is expressed in Pancreas, Uterus, Epidermis, Heart, Coronary Artery, Adrenal Gland/Suprarenal gland, Pancreas, Parathyroid Gland, Salivary Glands, Liver, Small Intestine, Bone Marrow, Peripheral Blood, Lymphoid tissue, Lymph node, Cartilage, Brain, Hypothalamus, Spinal Chord, Mammary gland/Breast, Uterus, Prostate, Testis, Lung, Kidney, Epidermis, Hair Follicle.

Additional utilities for NOV12 nucleic acids and polypeptides according to the invention are disclosed herein.

NOV13

A NOV13 polypeptide has been identified as a BHLH Factor MATH6-like protein (also referred to as CG94399-01). The disclosed novel NOV13 nucleic acid (SEQ ID NO:27) of 2244 nucleotides is shown in Table 13A. The novel NOV13 nucleic acid sequences maps to the chromosome 2.

An ORF begins with an ATG initiation codon at nucleotides 105-107 and ends with a TGA codon at nucleotides 1062-1064. A putative untranslated region and/or downstream from the termination codon is underlined in Table 13A, and the start and stop codons are in bold letters.

TABLE 13A
NOV13 Nucleotide Sequence

(SEQ ID NO:27)

ACGCGTGAAGGGCGGCCGAAGCGGGAGAGCCAGAGACTCCTCGGCGCTGAGCGCGGCGGCGGCCCGG
GCAGCCCCACGCCCCTGCCTCGCGCGCCGCCCGCGCCATGAAGCACATCCCGGTCCTCGAGGACGGG
CCGTGGAAGACCGTGTGCGTGAAGGAGCTGAACGGCCTTAAGAAGCTCAAGCGGAAAGGCAAGGAGC
CGGCGCGGCGCGCGAACGGCTATAAAACTTTCCGACTGGACTTGGAAGCGCCCGAGCCCCGCGCCGT
AGCCACCAACGGGCTGCGGGACAGGACCCATCGGCTGCAGCCGGTCCCGGTACCGGTCCGGTGCCAG
TCCCAGTGGCGCCGGCCGTTCCCCCAAGAGGGGGCACGGACACAGCCGGGGAGCGCGGGGGCTCTCG
GGCGCCCGAGGTCTCCGACGCGCGGAAACGTGCTTCGCCCTAGGCGCAGTGGGGCCAGGACTCCCCA
CGCCGCCGCCGCCGCCGCCTCCTGCGCCCCAGAGCCAGGCACCTGGGGGCCCAGAGGCACAGCCTTT
CGGGAGCCGGGTCTGCGTCCTCGCATCTTGCTGTGCGCACCGCCCGCGCCCCGCGCCGTCAGCACCC
CCAGCACCGCCAGCGCCCCCGGAGTCCACTGTGCGCCCTGGCCCCCGACGCGCCCCGGGGAAAGTTC
CTACTCGTCAATTTCACACGTAATTTACAATAACCACCAGGATTCCTCCGCGTCGCCTAGGAAACGA
CCGGGCGAAGCGACTGCCGCCTCCTCCGAGATCAAAGCCCTGCAGCAGACCCGGAGGCTCCTGGCGA
ACGCCAGGGAGCGGACGCGGGTGCACACCATCAGCGCAGCCTTCGAGGCGCTCAGGAAGCAGGTGCC
GTGCTACTCATATGGGCAGAAGCTGTCCAAACTGGCCATCCTGAGGATCGCCTGTAACTACATCCTG
TCCCTGGCGCGGCTGGCTGACCTTGACTACAGTGCCGACCACAGCAACCTCAGCTTCTCCGAGTGTG
TGCAGCGCTGCACCCGCACCCTGCAGGCCGAGGGACGTGCCAAGAAGCGCAAGGAGTGACTGGCTGC
AGGCAAGACCAAGGCCACCACTGTGGGCCCTCCTTCCAGTCAGGCCTGAGGACAAGGTGAGCTCGCT
GAGTCCAGCCTCGTGGTCTTCTCCAAGATGGCGCCCCACTTGGAGCCTACAGCCTCTCAGGGTCGGA
TCGGAGCACGCCTGCCTCCCTCTCCCCTCCGCCCTCACCCAGCCAATCCGAGGCTGCTTCGCACGTT
GCCCTCTGCCTGGTGGGGAGGGGAGAGCTCAGCCCCCGACTCACTCAGACCCCAAGGCCCACTGTCC
AGCTGCAGAAATTCGTTGCCAAAGATTGGACAGAGACACCGAAGGAAATGGGGTGGTGAAACCCCAC
AGCGAAAAGCCACACCGTTGCTCTGTGACTTTTGCTCCTCCTGTTGCCTGAGCCCCATCTCAAGCCA
AAGATGAGTCAGTGGTTCTGCTAGGAACTCATGGAATGGATGGGCATTTGATGACCCCTGGGGGTCA
TCTTGGCCCTCTGACCTGGTGCTCTCTCTCCACTGGGCCTTGTGCTGGTTGAGTGCAAGACAAGCCT
TAGGGGCTGTGAGAGGGAGGCTGGGGTGCCTGGGCGGGGCTGGGAGTGGGACCTGAGATCCCTGCCC
ACTCTCTCCCCTTCATTGGCTTGCCCAGGCCACTGGCCCCAGTTCTCAGTGTCCCTTGGGGTCCAGG
CTCCTTGGGCCCTAAGCATCACCAGAAGGGAGTAAGCAGGGAGAGAAGCAATATTACTCCCTCCCCT
ACACCAGGGACTTGCCCCAGGGCGGCTACCTATGGGTCTTTGCTTCCCCAGCCAGCCTCTCCTCACT
GTGACCCACCCCCATGGGCCCCCGTCCCAGGCAGCCAGCACCATGGGCAGGCCCTGCCATGGACAGA
AAAAGACTTTTTCTCTTGTTCAGCCTGCACGTGGCCTGAGGAAGGAGTAGAGGCTGGGTTGGCTGGA
GCCGTCCTACTGGGCAAGATGGCGCCCCACTTGGAGGGCGGTGGTCTGTTACAGGGTGTGCAGGGGC
AGAGAAGGAAGGGACCAGGGGACTGGGCCAGTATGTGGAGGATGGGGCCTGCGTGTTCAAAGCCAAG
GCCCGCCCCTTCCTTGTGCTCAAATGGCCAAAGCTGTTCACGTCTGTGCTCAACCATCTGCTTCAAA
TTGAAGTAAAAGCCCCAAAATGTCAAGAAAAAA

Variant sequences of NOV13 are included in Example 3, Table 25. A variant sequence can include a single nucleotide polymorphism (SNP). A SNP can, in some instances, be referred to as a “cSNP” to denote that the nucleotide sequence containing the SNP originates as a cDNA.

The NOV13 protein (SEQ ID NO:28) encoded by SEQ ID NO:27 is 319 amino acid residues in length and is presented using the one-letter amino acid code in Table 13B. Psort analysis predicts the NOV13 protein of the invention to be localized at the nucleus with a certainty of 0.7000.

TABLE 13B
Encoded NOV13 protein sequence
(SEQ ID NO:28)

MKHIPVLEDGPWKTVCVKELNGLKKLKRKGKEPARRANGYKTFRLDLEAPEPRAVATNGLRDRTH
RLQPVPVPVRCQSQWRRPFPQEGARTQPGSAGALGRPRSPTRGNVLRPRRSGARTPHAAAAAASC
APEPGTWGPRGTAFREPGLRPRILLCAPPAPRAVSTPSTASAPGVHCAPWPPTRPGESSYSSISH
VIYNNHQDSSASPRKRPGEATAASSEIKALQQTRRLLANARERTRVHTISAAFEALRKQVPCYSY
GQKLSKLAILRIACNYILSLARLADLDYSADHSNLSFSECVQRCTRTLQAEGRAKKRKE

A search against the Patp database, a proprietary database that contains sequences published in patents and patent publications, yielded several homologous proteins shown in Table 13C.

TABLE 13C
Patp results for NOV13

| | | | Smallest |
|---|---|---|---|
| | | | Sum |
| Sequences producing High-scoring | Reading | High | Prob |
| Segment Pairs: | Frame | Score | P (N) |
| >patp:AAM93743 Human polypeptide, SEQ | +1 | 1197 | 2.3e−121 |
| ID NO: 3717 | | | |
| >patp:AAB95274 Human protein sequence | +1 | 1197 | 2.3e−121 |
| SEQ ID NO: 17476 | | | |
| >patp:AAU16607 Human novel secreted | +1 | 534 | 4.2e−51 |
| protein, Seq ID 1560 | | | |
| >patp:ABG00300 Novel human diagnostic | +1 | 334 | 6.8e−33 |
| protein #291 | | | |
| >patp:ABG00300 Novel human diagnostic | +1 | 334 | 6.8e−33 |
| protein #291 | | | |

In a BLAST search of public sequence databases, it was found, for example, that the nucleic acid sequence of this invention has 372 of 657 bases (56%) identical to a gb:GENBANK-ID:HSBBICP4A|acc:L14320.1 mRNA from Bovine herpesvirus 1 (Bovine herpesvirus type 1 early-intermediate transcription control protein (BICP4) gene, complete cds). The full amino acid sequence of the protein of the invention was found to have 238 of 322 amino acid residues (73%) identical to, and 244 of 322 amino acid residues (75%) similar to, the 322 amino acid residue ptnr:TREMBLNEW-ACC:BAB39468 protein from Mus musculus (BHLH FACTOR MATH6).

NOV13 also has homology to the proteins shown in the BLASTP data in Table 13D.

TABLE 13D
BLAST results for NOV13

| Gene Index/ | | | | | |
|---|---|---|---|---|---|
| Identifier | Protein/Organism | Length (aa) | Identity (%) | Positives (%) | Expect |
| gi\|14249530\|ref\|NP_{—} | hypothetical | 321 | 246/321 | 246/321 | 2e−93 |
| 116216.1\|(NM_032827) | protein FLJ14708 | | (76%) | (76%) | |
| | [Homo sapiens] | | | | |
| gi\|13383235\|dbj\| | bHLH factor Math6 | 322 | 233/329 | 240/329 | 3e−86 |
| BAB39468.1\|(AB049066) | [Mus musculus] | | (70%) | (72%) | |
| gi\|17864454\|ref\|NP_{—} | net[ Drosophila | 365 | 55/97 | 76/97 | 5e−21 |
| 524820.1\|(NM_080081) | melanogaster] | | (56%) | (77%) | |
| gi\|7296271\|gb\| | CG11450 gene | 261 | 55/97 | 76/97 | 2e−20 |
| AAF51562.1\|(AE003590) | product | | (56%) | (77%) | |
| | [Drosophila | | | | |
| | melanogaster] | | | | |
| gi\|18858289\|ref\|NP_{—} | atonal homolog 2a | 325 | 36/82 | 51/82 | 2e−10 |
| 571891.1\|(NM_131816) | [Danio rerio] | | (43%) | (61%) | |

A multiple sequence alignment is given in Table 13E, with the NOV13 protein being shown on line 1 in Table 13E in a ClustalW analysis, and comparing the NOV13 protein with the related protein sequences shown in Table 13D. This BLASTP data is displayed graphically in the ClustalW in Table 13E.

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 13F lists the domain description from DOMAIN analysis results against NOV13.

TABLE 13F
Domain Analysis of NOV13

| | Region of | | |
|---|---|---|---|
| Model | Homology | Score (bits) | E value |
| Helix-loop- | 234-280 | 55.8 | 4.0e−09 |
| helix domain | | | |
| Helix-loop- | 229-281 | 61.4 | 1.9e−14 |
| helix DNA- | | | |
| binding domain | | | |
| (HLH) | | | |

Consistent with other known members of the BHLH Factor MATH6-like family of proteins, NOV13 has, for example, a Helix-loop-helix domain and a Helix-loop-helix DNA binding domain (HLH) signature sequence as well as homology to other members of the BHLH Factor MATH6-like Protein Family. NOV13 nucleic acids, and the encoded polypeptides, according to the invention are useful in a variety of applications and contexts. For example, NOV13 nucleic acids and polypeptides can be used to identify proteins that are members of the BHLH Factor MATH6-like Protein Family. The NOV13 nucleic acids and polypeptides can also be used to screen for molecules, which inhibit or enhance NOV13 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., Diabetes, Von Hippel-Lindau (VHL) syndrome, Pancreatitis, Obesity, Inflammatory bowel disease, Diverticular disease, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy as well as other diseases, disorders and conditions.

In addition, various NOV13 nucleic acids and polypeptides according to the invention are useful, inter alia, as novel members of the protein families according to the presence of domains and sequence relatedness to previously described proteins. For example, the NOV13 nucleic acids and their encoded polypeptides include structural motifs that are characteristic of proteins belonging to the BHLH Factor MATH6-like Protein Family.

A number of eukaryotic proteins, probably sequence specific DNA-binding proteins that act as transcription factors belong to this family. They share a conserved domain that is formed of two amphipathic helices joined by a variable length linker region that could form a loop (Littlewood and Evan, Protein Prof 2: 621-702 (1995).) This ‘helix-loop-helix’ (HLH) domain mediates protein dimerization and has been found in a large variety of proteins (Garrell and Campuzano, Bioessays 13:,493-498 (1991); Kato and Dang, FASEB J. 6: 3065-72 (1992).) Most of these proteins have an short basic region adjacent to the HLH domain that specifically binds to DNA. They are referred as basic helix-loop-helix proteins (bHLH), and are classified in two groups: class A (ubiquitous) and class B (tissue-specific). The HLH proteins lacking the basic domain (Einc, Id) function as negative regulators since they form heterodimers, but fail to bind DNA. The hairy-related proteins (hairy, E(spl), deadpan) also repress transcription although they can bind DNA. The proteins of this subfamily act together with co-repressor proteins, like groucho, through their C-terminal motif WRPW.

MATH6 (Inoue, et al., Genes to Cells 6: 977-86 (2001)) is a distant homolog of Drosophila proneuronal gene Atonal. Murine expression is higest in developing nervous system (ventricular zone and mantle layer, spinal cord, dorsal root ganglia). MATH6 is expressed by neuronal precursor cells and designated neurons, e.g., cerebellar Purkinje cells.

The closest mammalian homolog to MATH6 is NeuroD. NeuroD point mutations and NeuroD gene knockout animals have severe diabetes and die perinatally. The NeuroD knockout animals lack beta-Islet cells and could not be rescued with insulin administration. Also, the NeuroD knockout animals are deaf due to a loss of inner ear sensory neurons.

The NOV13 nucleic acids and polypeptides, antibodies and related compounds according to the invention will be useful in therapeutic and diagnostic applications in the mediation of metabolism as well as nerve and immune physiology. As such, the NOV13 nucleic acids and polypeptides, antibodies and related compounds according to the invention may be used to treat metabolic, hearing, nervous system, immune disorders, e.g., Diabetes, Von Hippel-Lindau (VHL) syndrome, Pancreatitis, Obesity, Inflammatory bowel disease, Diverticular disease, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy as well as other diseases, disorders and conditions.

The NOV13 nucleic acids and polypeptides are useful for detecting specific cell types. For example, expression analysis has demonstrated that a NOV13 nucleic acid is expressed in Pancreas, Umbilical Vein, Small Intestine, Cartilage, Synovium/Synovial membrane, Brain, Placenta, Oviduct/Uterine Tube/Fallopian tube, Lung, Brain, Uterus.

Additional utilities for NOV13 nucleic acids and polypeptides according to the invention are disclosed herein.

NOV14

A NOV14 polypeptide has been identified as a Putative Protein-Tyrosine Phosphatase-like protein (also referred to as CG94366-01). The disclosed novel NOV14 nucleic acid (SEQ ID NO:29) of nucleotides is shown in Table 14A. The novel NOV14 nucleic acid sequences maps to the chromosome 22.

An ORF begins with an ATG initiation codon at nucleotides 248-250 and ends with a TAA codon at nucleotides 1679-1681. A putative untranslated region and/or downstream from the termination codon is underlined in Table 14A, and the start and stop codons are in bold letters.

TABLE 14A
NOV14 Nucleotide Sequence

(SEQ ID NO:29)

ATTGAGTTTGAAATAACTGCCACCACAAAGTCTGTCACACATTGAGACTGAGGTCATAATAAAGAGG
TTTACTTAAATAGGGAAGCATTACTATTTTCCCCCGCCTAAGATTTTGGTTGTCGCCATATAAATCC
TCATTTCTAATAAAGAGAAAAAGACATTCCAGGTTCCAATAGTGCTATACACATGAATAGTCAGAAA
TTAATTGGTTTCTGTCTAGAATAATGAAAAGTAATTTTTCCAAAATATGAATTCAGAATTAAGTCTC
CTCTCTGACTGTTTTCTCTTATCATCCGCTAGTCCACAGACAAACGAATTTAAAGGAGCAACCGAGG
AGGCACCTGCGAAAGAAAGCCCACACACAGGTGAATTTAAAGGAGCAGCCCTGGTGTCACCTATCAG
TAAAAGAATGTTAGAACGACTTTCCAAGTTTGAAGTTGGAGATGCTGAAAATGTTGCTTCATATGAC
AGCAAGATTAAGAAAATTGTTCATTCAATTGTGTCATCCTTTGCAGTTGGGATATTTGGAGTTTTCC
TGATCTTGTTGGATGTGGCTCTGATCTTTGCTGACCTAATTTTCACTGATAGCAAAGTTTATATTCC
TTTGGAGTATCGTTCTATTTCTCTAGCTATTGCTTTATTTTTTCTCATGGATGTTCTTCTTCGAGTA
TTTGTAGAAAGGAGACAGCATTATTTTTCTGATTTACTTAACATTTTAGATACTGCCGTTACTGTGA
TTATTCTGCTGGTTGATGTCGTTTACATTTTTTTTGACGTTAAGTTTCTTAAGGATATTCCCAGATG
GACACGTTTATTTCGACTTCTACGACTTATCATTCTGATAAGAGTTTTTCGTCTGGCTCATCTAAAA
AGACAACTTGGAAAGCTGATAAGAAGGCTGGTAAGTAGGNGATACGAAAGGGATGGATTTGACCTAG
ACCTCACTTATATTACAGAACGTATTGTCGCTATGTCATTTCCATCTTCGGGAGGCCAGTCTTTCTA
TCGGAATCCAATTAAGGAAGTCGTACAGTTTCTAGACAAGAAACATCCAAACCACTATCGAGTCTAC
AATCTATGCAGTGAAAGAGCTTATGATCCTAAGCACTTCCATAATAGGGTCAGTAGAATCATGATCG
ATGATCATAATGTCCCCACTCTAAGGGAGATGGTAGCATTCTCCAAGGAAGTGTTGGAGTGGATGGC
TCAAGATTCTGAAAACATCGTAGTGATTCACTGTAAAGGAGGCAAAGGTAGAACCGGAACTATGGTT
TGTGCCTGCCTGATTGCCAGTGAAATATTTTTAACTGCAGAGGAAAGATTGTACTATTTTGGAGAAC
GGCGAACAGATAAAACCAATGGCACTAAATATCAGGGAGTAGAAACTCCTTCTCAGAATAGATATGT
TGGATATTTTGCACAAGTGAAACATAGCTACAACTGGAATCTCCCTCCAAGAAAAACACTGTTTATA
AAAAGATTAGTTATTTATTCGATTCATGGTAAGTGTTTAGATCTAAAAGTCCAAATAGTAATGAAGA
AAAAGATTGTCTTTTCCTGCACTTCCTTAAACAGTTGTCGGGTAAGAGAAAACATGGAAACAGACAG
GGTAATAATTGATGTGTTCAACTGTCCACCTCTGTATGATGATGTGAAAGTGCAATTTTTTTTTTCT
TTTTAGGATTTTCCTAAATACTATCACAACTACCCTTTTTTCTTCTGGTTTAACACATCTTTAATAC
AAAATAACAGGCTTTATCTACAAAGAAATGAATTGGATAATCTTCATAAACAAAAAACATGGAAAAT
TTATCAACCAGAATATGCAGTAGAGATATATTTTGATGAGAAATGACTTAAGTTATGTTGTAACTGG
TAGCTGATTAAGTATAGTTCCCTGCACCCCTTCTGGGAAAGAATTATGTTCTTTCTAACCCTGCCAC
ATAGTTATATGTTCTAAATCTTCCTTGCTGGTACATCTATATTGATATATGTATACACATGTTCTTT
ATAAATCTATTAAATATATACAGATAAA

The NOV14 protein (SEQ ID NO:30) encoded by SEQ ID NO:29 is 477 amino acid residues in length and is presented using the one-letter amino acid code in Table 14B. Psort analysis predicts the NOV14 protein of the invention to be localized at the plasma membrane with a certainty of 0.6000.

TABLE 14B
Encoded NOV14 protein sequence

(SEQ ID NO:30)

MNSELSLLSDCFLLSSASPQTNEFKGATEEAPAKESPHTGEFKGAALVSPISKRMLERLSKFEVG
DAENVASYDSKIKKIVHSIVSSFAVGIFGVFLILLDVALIFADLIFTDSKVYIPLEYRSISLAIA
LFFLMDVLLRVFVERRQHYFSDLLNILDTAVTVIILLVDVVYIFFDVKFLKDIPRWTRLFRLLRL
IILIRVFRLAHLKRQLGKLIRRLVSRXYERDGFDLDLTYITERIVAMSFPSSGGQSFYRNPIKEV
VQFLDKKHPNHYRVYNLCSERAYDPKHFHNRVSRIMIDDHNVPTLREMVAFSKEVLEWMAQDSEN
IVVIHCKGGKGRTGTMVCACLIASEIFLTAEERLYYFGERRTDKTNGTKYQGVETPSQNRYVGYF
AQVKHSYNWNLPPRKTLFIKRLVIYSIHGKCLDLKVQIVMKKKIVFSCTSLNSCRVRENMETDRV
IIDVFNCPPLYDDVKVQFFFSF

A search against the Patp database, a proprietary database that contains sequences published in patents and patent publications, yielded several homologous proteins shown in Table 14C.

TABLE 14C
Patp results for NOV14

| | | | Smallest |
|---|---|---|---|
| | | | Sum |
| Sequences producing High-scoring | Reading | High | Prob |
| Segment Pairs: | Frame | Score | P (N) |
| >patp:AAG67459 Amino acid sequence of | +1 | 1895 | 2.5e−195 |
| a human polypeptide | | | |
| >patp:AAG67638 Amino acid sequence of | +1 | 1895 | 2.5e−195 |
| a human protein | | | |
| >patp:AAB73230 Human phosphatase | +1 | 574 | 1.8e−111 |
| AA493915_h | | | |
| >patp:AAW34402 Protein encoded by | +1 | 473 | 1.2e−44 |
| gene IMAGE clone 264611 | | | |
| >patp:AAY07450 Human TS10q23.3 gene | +1 | 473 | 1.2e−44 |
| bases 453-2243 | | | |

In a BLAST search of public sequence databases, it was found, for example, that the nucleic acid sequence of this invention has 1105 of 1427 bases (77%) identical to a gb:GENBANK-ID:AF007118|acc:AF007118.1 mRNA from Homo sapiens (putative tyrosine phosphatase mRNA, complete cds). The full amino acid sequence of the protein of the invention was found to have 369 of 462 amino acid residues (79%) identical to, and 402 of 462 amino acid residues (87%) similar to, the 551 amino acid residue ptnr:SWISSNEW-ACC:P56180 protein from Homo sapiens (PUTATIVE PROTEIN-TYROSINE PHOSPHATASE TPTE (EC 3.1.3.48)).

NOV14 also has homology to the proteins shown in the BLASTP data in Table 14D.

TABLE 14D
BLAST results for NOV14

| Gene Index/ | | | | | |
|---|---|---|---|---|---|
| Identifier | Protein/Organism | Length (aa) | Identity (%) | Positives (%) | Expect |
| gi\|7019559\|ref\|NP_{—} | transmembrane | 551 | 369/462 | 402/462 | 0.0 |
| 037447.1\|(NM_013315) | phosphatase with | | (79%) | (86%) | |
| | tensin homology; | | | | |
| | tensin, putative | | | | |
| | protein-tyrosine | | | | |
| | phosphatase | | | | |
| | [Homo sapiens] | | | | |
| gi\|16166555\|ref\|XP_{—} | similar to | 551 | 367/462 | 401/462 | 0.0 |
| 055073.1\|(XM_055073) | transmembrane | | (79%) | (86%) | |
| | phosphatase with | | | | |
| | tensin homology | | | | |
| | [Homo sapiens] | | | | |
| gi\|18640756\|ref\|NP_{—} | similar to | 445 | 316/462 | 343/462 | e−156 |
| 570141.1\|(NM_130785) | PUTATIVE PROTEIN- | | (68%) | (73%) | |
| | TYROSINE | | | | |
| | PHOSPHATASE TPTE | | | | |
| | [Homo sapien] | | | | |
| gi\|14787415\|emb\| | tyrosine | 664 | 275/432 | 336/432 | e−141 |
| CAC44243.1\|(AJ311311) | phosphatase | | (63%) | (77%) | |
| | isoform A [Mus | | | | |
| | musculus] | | | | |
| gi\|14787417\|emb\| | tyrosine | 645 | 275/432 | 336/432 | e−141 |
| CAC44244.1\|(AJ311312) | phosphatase | | (63%) | (77%) | |
| | isoform B [Mus | | | | |
| | musculus] | | | | |

A multiple sequence alignment is given in Table 14E, with the NOV14 protein being shown on line 1 in Table 14E in a ClustalW analysis, and comparing the NOV14 protein with the related protein sequences shown in Table 14D. This BLASTP data is displayed graphically in the ClustalW in Table 14E.

[image]

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 14F lists the domain description from DOMAIN analysis results against NOV14.

TABLE 14F
Domain Analysis of NOV14

| | Region of | | |
|---|---|---|---|
| Model | Homology | Score (bits) | E value |
| Dual | 231-378 | −48.8 | 3.2 |
| specificity | | | |
| phosphatase, | | | |
| catalytic doma | | | |

Consistent with other known members of the Putative Protein-Tyrosine Phosphatase-like family of proteins, NOV14 has, for example, a dual specificity protein phosphatase signature sequence and homology to other members of the Putative Protein-Tyrosine Phosphatase-like Protein Family. NOV14 nucleic acids, and the encoded polypeptides, according to the invention are useful in a variety of applications and contexts. For example, NOV14 nucleic acids and polypeptides can be used to identify proteins that are members of the Putative Protein-Tyrosine Phosphatase-like Protein Family. The NOV14 nucleic acids and polypeptides can also be used to screen for molecules, which inhibit or enhance NOV14 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., Cardiovascular diseases, cystitis, incontinence as well as other diseases, disorders and conditions

In addition, various NOV14 nucleic acids and polypeptides according to the invention are useful, inter alia, as novel members of the protein families according to the presence of domains and sequence relatedness to previously described proteins. For example, the NOV14 nucleic acids and their encoded polypeptides include structural motifs that are characteristic of proteins belonging to the Putative Protein-Tyrosine Phosphatase-like Protein Family.

Cellular processes involving growth, differentiation, transformation and metabolism are often regulated in part by protein phosphorylation and dephosphorylation. The protein tyrosine phosphatases (PTPs), which hydrolyze the phosphate monoesters of tyrosine residues, all share a common active site motif and are classified into 3 groups. These include the receptor-like PTPs, the intracellular PTPs, and the dual-specificity PTPs, which can dephosphorylate at serine and threonine residues as well as at tyrosines. Diamond et al. (1994) described a PTP from regenerating rat liver that is a member of a fourth class. The gene, which they designated Prl1, was one of many immediate-early genes. Overexpression of Prl1 in stably transfected cells resulted in a transformed phenotype, which suggested that it may play some role in tumorigenesis. By using an in vitro prenylation screen, Cates et al. (1996) isolated 2 human cDNAs encoding PRL1 homologs, designated PTP(CAAX1) and PTP(CAAX2)(PRL2), that are farnesylated in vitro by mammalian farnesyl:protein transferase. Overexpression of these PTPs in epithelial cells caused a transformed phenotype in cultured cells and tumor growth in nude mice. The authors concluded that PTP(CAAX1) and PTP(CAAX2) represent a novel class of isoprenylated, oncogenic PTPs. Peng et al. (1998) reported that the human PTP(CAAX1) gene, or PRL1, is composed of 6 exons and contains 2 promoters. The predicted mouse, rat, and human PRL1 proteins are identical. Zeng et al. (1998) determined that the human PRL1 and PRL2 proteins share 87% amino acid sequence identity.

The NOV14 nucleic acids and polypeptides, antibodies and related compounds according to the invention will be useful in therapeutic and diagnostic applications in the mediation of cardiac and renal physiology. As such, the NOV14 nucleic acids and polypeptides, antibodies and related compounds according to the invention may be used to treat cardiovascular and urogenital system disorders, e.g., Cardiovascular diseases, cystitis, incontinence as well as other diseases, disorders and conditions. The NOV14 nucleic acids and polypeptides are useful for detecting specific cell types. For example, expression analysis has demonstrated that a NOV14 nucleic acid is expressed in Urinary bladder.

Additional utilities for NOV14 nucleic acids and polypeptides according to the invention are disclosed herein.

NOV15

A NOV15 polypeptide has been identified as a Leucine Rich Repeat (LRR)-like protein (also referred to as CG95387-02). The disclosed novel NOV15 nucleic acid (SEQ ID NO:31) of 3136 nucleotides is shown in Table 15A. The novel NOV15 nucleic acid sequences maps to the chromosome 19.

An ORF begins with an ATG initiation codon at nucleotides 330-332 and ends with a TAA codon at nucleotides 2331-2333. A putative untranslated region and/or downstream from the termination codon is underlined in Table 15A, and the start and stop codons are in bold letters.

TABLE 15A
NOV15 Nucleotide Sequence

(SEQ ID NO:31)

ACTCCTGACCTAAAGTGATCCACTCGCCTTGGCCTCCCAAAGTGCTACGATTACAGCCCTCATTCTC
TTTTGCTCCTCAGCTGACACAGGACAAGATCATCTGTCTACCCAATCATGAGCTCCAGGAGAACTTA
TCAGAGGCCCCGTGCCAGCAATTCCTCCCTCGCGCGATCCCTGAGCACATTGGCGCCCTGCAGGAGG
TTAAACCCCTTAAGAACAATTTGGACCTGCAGCAATACAGCTTTATTAACCAGCTGTGTTATGAGAC
GGCCCTGCACTGGTATGCCAAGTACTTCCCTTACCTCGTGGTCATTCACACACTCATCTTCATGGTC
TGCACCAGTTTCTGGTTCAAGTTCCCTGGCACCAGCTCCAACATTGAACACTTCATCTCCATCCTCG
GCAACTGTTTCGACTCTCCATGGACCACCAGGGCCCTATCCGAGGTCTCCGGGGAGAACCAGAAGGG
CCCAGCAGCCACCGAACGGGCTCCGGCATCCATAGTGCCCATCGCAGGCACCGGCCCGGGGAAGGCA
CGGGAGGGTGAGAAGGAGAAAGTGCTCGCGCAACCCGAGAAGGTGGTGACCGAGCCTCCAGTTGTCA
CCCTGTTGGACAAGAAGGAGGGTGAGCAAGCCAAAGCCCTGTTTGAGAAGGTGAACAAGTTCCGCAT
GCACCTGGAAGAGGCCGACATCCTGTACACCATGTACATCCGACAGACGGTCCTGAAAGTGTGTAAG
TTCCTGGCCATCCTGGTCTACAACCTGGTCTATGTGGAGAAGATCAGTTTCCTGGTGGCCTGTAGCG
TGGAGACGTCAGAGGTCACGGGCTACGCCAGCTTCTGCTGCAACCACACCAAGGCCCACCTCTTCTC
CAAGCTGGCCTTCTGTTACATCTCCTTTGTGTGCATCTACGCACTTACCTGCATCTACACGCTCTAC
TGGCTCTTCCACCGGCCCCTCAAGGAGTACTCCTTCCGTTCCGTGCGGGAGGAGACTGGCATGGGGG
ACATTCCTGACGTCAAGAATGACTTCGCCTTCATGCTGCACCTCATCGATCAGTACGACTCCCTCTA
CTCCAAGCGCTTCGCCGTCTTCCTGTCCGAGGTCAGCGAAAGCCGTCTAAAGCAGCTCAATCTCAAC
CACGAGTGGACCCCCCAGAAGCTTCGACACAAGCTGCACCGCAATGCCGCGGCCCGGCTGGAGCTAA
CCCTCTGCATGCTGCCGGGTCTGCCCGACACCGTCTTTGAGCTCAGTGAGGTGGAGTCACTCAGGCT
GGAGGCCATCTGCGATATCACCTTCCCCCCGGGGCTGTCACAGCTCGTCCACTTGCAGCAGCTCAGC
TTGCTCCACTCGCCCGCCAGGCTACCCTTCTCCTTGCAGGTCTTCCTGCGGCACCACCTGAAGGTGA
TGCGCGTCAAATGCGAGCAGCTCCGCGAGCTGCCGCTTTGGGTGTTTGGGCTGCGGGGCTTGGAGGA
GCTGCACCTGGAGGGCCTTTTCCCCCACGAGCTAGCTCGGGCAGCCACCCTGGAGACCCTCCGGGAG
CTGAACCAGCTCAAGGTGTTGTCCCTCCGGAGCAACCCCGGGAAGGTCCCACCCAGTGTGACCGACG
TTGCTGCCCACCTGCAGACGCTCAGCCTGCACAACGATGGGGCCCGTCTGGTTGCCCTGAACAGCCT
CAAGAAGCTGGCGGCATTGCGGGAGCTGGAGCTGGTGGCCTGCGGGCTGGAGCGCATCCCCCATGCA
GTGTTCACCCTGGGTGCGCTGCAGGAACTTGACCTCAAGGACAACCACCTGCGCTCCATCGAGGAAA
TCCTCAGCTTCCAGCACTGCCGGAAGCTGGTCACGCTCAGGCTGTGGCACAACCAGATCCCCTACGT
CCCTGAGCACGTGCGGAAGCTCAGCAGCCTGGAGCAGCTCTACCTCAGCTACAACAACCTGGAGACC
CTGCCCTCCCAGCTCGGCCTGTGCTCAGGCCTCCGTCTGCTGGATGTGTCCCACAATGGGCTACACT
CCCTGCCACCCGAGGTGGGCCTCCTGCAGAACCTACAGCACCTGGCCCTCTCCTACAATGCCCTGGA
GGCCCTGCCCGAAGAGCTCTTCTTCTGCCGCAACCTGCGCACGTTGCTTCTCGGCGACAACCAACTG
AGCCAGCTCTCGCCCCACGTGGGTGCCCTCAGAGCCCTCAGCCGCCTGGAGCTCAAAGGCAACCGCT
TAGAGGCGCTGCCAGAAGAACTTGGCAACTGTGGGGCGCTCAAGAACGCGGAACTCCTGGTGGAACA
CACGCTTTACCAGGGTCTGCCGGCAGAAGTGCGGGACAAGATGGAGGAGGAATGAAGCTGGGGTCGG
GCCGTTTTAGGTAGAGCCTTAAAAATGCTTCTGCCCTGGAATCTCAACCATTATCTTCCAAGATAGC
AAGCCAAGTGGGTCTAGGCCAGGACATGCGGGGGGGCGGGGCCAGCTGTGTCATCTTTCTGGGGCCC
AGGACGATCTCGGCTGGTTTGTCTGGGGAGACAGACAGGATGTTGTGGAGGTGCGGTGGAACCTGGT
ATGGACGGATTAACTCAGTCATGGCATTCTCCGACCAAAACCACACCTGTGTCTCTGGCAGGCTGCC
TGGCCTTGCTCCCATCCCTAGAACTGCTGCCTCTCCCTGCATATTCCAGCTCAATTAGTGCCACATA
TGGGGGAAACGACACATCCCAGTGGCATTTCCAACACTCCCCCTCCCCATGCAACAAAGCAACTTAC
TTCTGGAGTTCTCTCCCAAGGAGAGGACACAGACACAGTTGTTTCCTGTGTTATATGTTAGCTCCGA
ACAATGGTTCTCATTTGGCTAAGCATCAAAATCACCTAGCGAGCCGGTCCAAAACAAAATATCCCAG
TCCCCTCCCCTGAAACACTGACTCAGGAGGTTTGGTTGGGGGCCAGGAGTCTGTTCCTAAATATTCC
AGGTAGTTCTCGTGCAGGTAAGTGGCCCTGAGACAGTATGTTGGGAAATGCTGACGTAAAGGTATCA
GGGCCGGGCGCTGTGGCTCATGACTATAATCCCAGCTGTTTGAGAGCCCAATGCAGGAGGATCGTTG
AGCTCAGCAGTTCGAGATCAGCCTCGGTAACATAGCGACACCCCACCTCTGCCA

Variant sequences of NOV15 are included in Example 3, Table 26. A variant sequence can include a single nucleotide polymorphism (SNP). A SNP can, in some instances, be referred to as a “cSNP” to denote that the nucleotide sequence containing the SNP originates as a cDNA.

The NOV15 protein (SEQ ID NO:32) encoded by SEQ ID NO:31 is 667 amino acid residues in length and is presented using the one-letter amino acid code in Table 15B. Psort analysis predicts the NOV15 protein of the invention to be localized at the plasma membrane with a certainty of 0.7900.

TABLE 15B
Encoded NOV15 protein sequence

(SEQ ID NO:32)

MVCTSFWFKFPGTSSKIEHFISILGKCFDSPWTTRALSEVSGENQKGPAATERAAASIVAMAGTG
PGKAGEGEKEKVLAEPEKVVTEPPVVTLLDKKEGEQAKALFEKVKKFRMHVEEGDILYTMYIRQT
VLKVCKFLAILVYNLVYVEKISFLVACRVETSEVTGYASFCCNHTKAHLFSKLAFCYISFVCIYG
LTCIYTLYWLFHRPLKEYSFRSVREETGMGDIPDVKNDFAFMLHLIDQYDSLYSKRFAVFLSEVS
ESRLKQLNLNHEWTPEKLRQKLQRNAAGRLELALCMLPGLPDTVFELSEVESLRLEAICDITFPP
GLSQLVHLQELSLLHSPARLPFSLQVFLRDHLKVMRVKCEELREVPLWVFGLRCLEELHLEGLFP
QELARAATLESLRELKQLKVLSLRSNAGKVPASVTDVAGHLQRLSLHNDGARLVALNSLKKLAAL
RELELVACGLERIPHAVFSLCALQELDLKDNHLRSIEEILSFQHCRKLVTLRLWHNQIAYVPEHV
RKLRSLEQLYLSYNKLETLPSQLGLCSGLRLLDVSRNGLHSLPPEVGLLQNLQHLALSYNALEAL
PEELFFCRKLRTLLLGDNQLSQLSPHVGALRALSRLELKCNRLEALPEELGNCGGLKKAGLLVED
TLYQGLPAEVRDKMEEE

A search against the Patp database, a proprietary database that contains sequences published in patents and patent publications, yielded several homologous proteins shown in Table 15C.

TABLE 15C
Patp results for NOV15

| | | | Smallest |
|---|---|---|---|
| | | | Sum |
| Sequences producing High-scoring | Reading | High | Prob |
| Segment Pairs: | Frame | Score | P (N) |
| >patp:AAY70473 Human CNAP-1 | +1 | 2147 | 5.0e−222 |
| >patp:AAG75413 Human colon cancer | +1 | 1882 | 6.0e−194 |
| antigen protein | | | |
| >patp:AAM41692 Human polypeptide SEQ | +1 | 1878 | 1.6e−193 |
| ID NO 6623 | | | |
| >patp:AAU20426 Human secreted protein, | +1 | 1835 | 5.8e−189 |
| Seq ID No 418 | | | |
| >patp:AAB92855 Human protein sequence | +1 | 1795 | 1.0e−184 |
| SEQ ID NO: 11424 | | | |

In a BLAST search of public sequence databases, it was found, for example, that the nucleic acid sequence of this invention has 2227 of 2228 bases (99%) identical to a gb:GENBANK-ID:AK027073|acc:AK027073.1 mRNA from Homo sapiens (cDNA: FLJ23420 fis, clone HEP22352). The full amino acid sequence of the protein of the invention was found to have 444 of 444 amino acid residues (100%) identical to, and 444 of 444 amino acid residues (100%) similar to, the 444 amino acid residue ptnr:SPTREMBL-ACC:Q9H5H8 protein from Homo sapiens (cDNA: FLJ23420 FIS, CLONE HEP22352).

NOV15 also has homology to the proteins shown in the BLASTP data in Table 15D.

TABLE 15D
BLAST results for NOV15

| Gene Index/ | | | | | |
|---|---|---|---|---|---|
| Identifier | Protein/Organism | Length (aa) | Identity (%) | Positives (%) | Expect |
| gi\|13376597\|ref\|NP_{—} | hypothetical | 444 | 444/444 | 444/444 | 0.0 |
| 079337.1\|(NM_025061) | protein FLJ23420 | | (100%) | (100%) | |
| | [Homo sapiens] | | | | |
| gi\|14150009\|ref\|NP_{—} | hypothetical | 708 | 404/667 | 520/667 | 0.0 |
| 115646.1\|(NM_032270) | protein | | (60%) | (77%) | |
| | DKFZp586J1119 | | | | |
| | [Homo sapiens] | | | | |
| gi\|19343671\|gb\| | Similar to | 708 | 404/671 | 526/671 | 0.0 |
| AAH25473.1\|(BC025473) | hypothetical | | (60%) | (78%) | |
| | protein | | | | |
| | DKFZp586J1119 | | | | |
| | [Mus musculus] | | | | |
| gi\|7243272\|dbj\| | KIAA1437 protein | 811 | 367/666 | 84/666 | 0.0 |
| BAA92675.1\|(AB037858) | [Homo sapiens] | | (55%) | (72%) | |
| gi\|8922442\|ref\|NP_{—} | hypothetical | 682 | 345/673 | 470/673 | 0.0 |
| 060573.1\|(NM_018103) | protein FLJ10470 | | (51%) | (69%) | |
| | [Homo sapiens] | | | | |

A multiple sequence alignment is given in Table 15E, with the NOV15 protein being shown on line 1 in Table 15E in a ClustalW analysis, and comparing the NOV15 protein with the related protein sequences shown in Table 15D. This BLASTP data is displayed graphically in the ClustalW in Table 15E.

[image]

[image]

The NOV15 Clustal W alignment shown in Table 15E was modified to begin at amino residue 121. The data in Table 15E includes all of the regions overlapping with the NOV15 protein sequences.

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 15F lists the domain description from DOMAIN analysis results against NOV15.

TABLE 15F
Domain Analysis of NOV15

| | Region of | | |
|---|---|---|---|
| Model | Homology | Score (bits) | E value |
| LRR | 454-476 | 5.6 | 1.5e+02 |
| LRR | 477-498 | 10.8 | 26 |
| LRR | 502-524 | 11.9 | 16 |
| LRR | 525-547 | 18.8 | 0.13 |
| LRR | 548-570 | 15.9 | 0.99 |
| LRR | 571-593 | 13.9 | 4 |
| LRR | 594-616 | 12.3 | 12 |
| LRR | 617-639 | 9.4 | 42 |

Consistent with other known members of the LRR-like family of proteins, NOV15 has, for example, eight Leucine Rich Repeat (LRR) signature sequences and homology to other members of the LRR-like Protein Family. NOV15 nucleic acids, and the encoded polypeptides, according to the invention are useful in a variety of applications and contexts. For example, NOV15 nucleic acids and polypeptides can be used to identify proteins that are members of the LRR-like Protein Fainmily. The NOV15 nucleic acids and polypeptides can also be used to screen for molecules, which inhibit or enhance NOV15 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., cancer, trauma, regeneration (in vitro and in vivo), viral/bacterial/parasitic infections, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, Von Hippel-Lindau (VHL) syndrome, cirrhosis, transplantation, Hirschsprung's disease, Crohn's Disease, appendicitis, osteoporosis, hypercalceimia, arthritis, ankylosing spondylitis, scoliosis, systemic lupus erythematosus, autoimmune disease, xerostomia as well as other diseases, disorders and conditions.

In addition, various NOV15 nucleic acids and polypeptides according to the invention are useful, inter alia, as novel members of the protein families according to the presence of domains and sequence relatedness to previously described proteins. For example, the NOV15 nucleic acids and their encoded polypeptides include structural motifs that are characteristic of proteins belonging to the LRR-like Protein Family.

LRR Proteins are a family of proteins characterized by a structural motif rich in leucine residues. They are either transmembrane or secreted proteins and are involved in protein-protein interactions. Members of this family have been implicated in extracellular matrix assembly and cellular growth. In addition, several proteins belonging to this family, such as slit, Toll and robo have been shown to mediate key roles in central nervous system development and organogenesis in Drosophila. Vertebrate orthologs of these proteins have also been shown to have similar roles in the CNS as well as other organ systems like kidney.

LRRs are relatively short motifs (22-28 residues in length) found in a variety of cytoplasmic, membrane and extracellular proteins. Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments (Packman et al. FEBS Lett. 1991; 291: 87-91). These results are consistent with the idea that LRRs mediate protein-protein interactions and cellular adhesion. Other functions of LRR-containing proteins include, for example, binding to enzymes and vascular repair.

The NOV15 nucleic acids and polypeptides, antibodies and related compounds according to the invention will be useful in therapeutic and diagnostic applications in the mediation of cardiac, immune, and nerve physiology. As such, the NOV15 nucleic acids and polypeptides, antibodies and related compounds according to the invention may be used to treat cardiovascular, nervous, and immune system disorders, e.g., cancer, trauma, regeneration (in vitro and in vivo), viral/bacterial/parasitic infections, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, Von Hippel-Lindau (VHL) syndrome, cirrhosis, transplantation, Hirschsprung's disease, Crohn's Disease, appendicitis, osteoporosis, hypercalceimia, arthritis, ankylosing spondylitis, scoliosis, systemic lupus erythematosus, autoimmune disease, xerostomia as well as other diseases, disorders and conditions.

The NOV15 nucleic acids and polypeptides are useful for detecting specific cell types. For example, expression analysis has demonstrated that a NOV15 nucleic acid is expressed in Coronary Artery, Parotid Salivary glands, Liver, Colon, Bone, Synovium/Synovial membrane, and Brain.

Additional utilities for NOV15 nucleic acids and polypeptides according to the invention are disclosed herein.

NOV16

A NOV16 polypeptide has been identified as a RhoGEF-like protein (also referred to as CG95419-02). The disclosed novel NOV16 nucleic acid (SEQ ID NO:33) of 5372 nucleotides is shown in Table 16A. The novel NOV16 nucleic acid sequences maps to the chromosome 5.

An ORF begins with an ATG initiation codon at nucleotides 61-63 and ends with a TAA codon at nucleotides 5179-5181. A putative untranslated region and/or downstream from the termination codon is underlined in Table 16A, and the start and stop codons are in bold letters.

TABLE 16A
NOV16 Nucleotide Sequence

(SEQ ID NO:33)

CCATGGGGCCTCCTGCAATAACTTCTCTTGTTTATTATTTTCATTGCAGATGCGAAAGCCATGGAGT
TGAGCTGCACCGAAGCACCTCTTTACCAGCGGCAGATGATGATCTATGCGAAGTTTGACAAAAATGT
GTATCTTCCTGAAGATGCTGAGTTTTACTTTACTTATGACGGATCTCATCACCGACATCTCATGATT
GCAGAGCGCATCGAGGATAACGTTCTCCAGTCCAGCGTCCCAGCCCATGGGCTTCAGGAGACGGTGA
CGGTATCTGTGTGCCTCTGCTCCGAAGCTTACTCTCCGGTGACCATGGGCTCTGGCTCAGTGACCTA
CGTCGACAACATGCCTTGCAGGCTGCCTCGTCTGCTGGTGACGCACGCCAATCGCCTCACAGCCTGC
AGCCACCAGACCCTGCTGACCCCATTTGCCTTGACGGCAGGACCACTGCCTGCCTTGCATGAGGAGC
TCGTGCTGGCTCTGACCCATCTGGAATTGCCTCTAGAGTGGACTGTGTTCGGAAGTTCTTCACTTGA
AGTATCTTCTCACAGAGAATCTCTTCTACACCTGGCTATGAGATCGGGCCTGGCTAAACTTTCCCAG
TTCTTCTTGTGTCTCCCGGGGCGAGTCCAGGCCTTCGCTTTACCCAACGAAGAGGGTGCCACACCAT
TAGACTTAGCTTTACGTGAAGGACACTCCAAGCTGGTCGAAGACGTCACAAGTTTTCAGGGCAGATG
GTCCCCAAGCTTCTCCCGAGTGCAGCTCAGTGAAGAAGCCTCCTTGCATTACATTCACTCATCCGAA
ACGCTGACCCTGACCCTGAACCACACAGCCGAGCATTTGTTGGAGGCAGATATTAAACTCTTCCGGA
AATACTTTTGGGATAGAGCCTTTCTTGTCAAGGCCTTTGAGCAACAAGCCACGCCAGAGCAAAGAAC
AGCTATCCCCTCCAGCGGTCCAGAAACTGAAGAAGAGATTAAGAATTCAGTGTCCAGCAGATCACCA
GCCGAAAAGGAAGATATAAAGCGTCTCAAAAGCCTGGTGGTTCAACACAATGAACATCAAGACCAGC
ACAGCCTAGATTCTAGATCGCTCCTTCCATATCCTAAAAAATCCAACCCGCCCTCGACATTGCTTGC
TCCAGGCCGGCTTTCACACATGCTGAATGGAGGTGATGAAGTCTACGCTAACTGTATGGTGATTGAT
CAGGTTGGTGATTTGGATATCAGCTATATTAATATAGAGGCAATCACTGCCACTACCAGCCCTGAAT
CCAGAGCTTGCACTCTGTGGCCTCAGAGCAGCAAACACACCCTTCCTACAGAAACCAGTCCCAGTGT
GTACCCACTTAGTGAAAATGTCCAAGCGACAGCACACACTGAAGCCCAGCAGTCCTTCATGTCACCA
TCAAGTTCGTGTGCTTCCAACTTGAATCTTTCTTTTGGTTGGCATGGATTTGAAAAGGAACAAAGTC
ATCTAAAGAAAAGAAGTTCTAGCCTTGATGCCTTCGACGCCGACAGTGAAGGGGAAGGGCATTCTGA
GCCATCCCACATCTGTTACACTCCAGGGTCTCAGACCTCCTCAAGAACTGGCATTCCTAGTGGGGAT
GAATTGGACTCTTTTGAGACTAACACTGAACCGGATTTTAATATCTCCAGGGCTGAATCCCTTCCTC
TATCAAGTAATCTACAGTTGAAGGAATCACTGCTTTCTGGAGTTCGCTCACGTTCTTATTCTTGCTC
GTCACCCAAAATTTCTTTAGGAAAAACTCCTTTCGTGCGTGAATTAACAGTATGCAGTTCAAGTGAA
GAGCAAAAAGCTTACACCTTATCCGAGCCACCAACAGAAAACAGGATTCAGGAAGAAGAATGGGATA
AATACATCATACCTGCCAAATCAGAGTCTGAAAAATATAAAGTGAGTCGAACTTTCAGTTTCCTCAT
GAATAGGATGACTAGCCCTCGGAATAAATCAAAGACAAAAAGCAAGGATGCCAAAGATAAAGAGAAG
CTGAATCGACATCAGTTTGCCCCAGGAACATTCTCTGGGGTTCTGCAGTGTTTGGTTTGTGATAAAA
CACTCCTGGGGAAAGAGTCACTGCAGTGTTCTAGTTGTAATGCAAATGTGCACAAAGGTTGTAAAGA
TGCTCCGCCTGCATCCACCAAGAAATTCCAAGAGAAATATAACAAGAACAAACCACAGACCATCCTT
GGAAGTTCTTCATTTAGAGACATCCCACAGCCTGGTCTCTCCTTGCACCCTTCTTCCTCCGTGCCTG
TTGGATTGCCGACTGGAAGGAGGGAGACTGTGGGACACGTCCATCCATTGTCCAGAAGTGTTCCAGG
TACCACCTTGGAAAGCTTCAGGAGGTCAGCCACATCCTTGGAGTCTGAGAGTGACAATAACAGCTGC
AGAAGCAGGTCTCATTCTGATGAGCTGCTACAGTCCATCGGCTCTTCTCCCTCTACAGAGTCTTTCA
TAATGGAAGATGTTGTGGATTCTTCTCTGTGGAGTGACCTCAGCAGTGATGCCCAGCAGTTTGAAGC
AGAATCTTGGAGTCTTGTGGTGGATCCCTCATTTTGTAATAGGCAGGAGAAGGATGTCATCAAAAGA
CAGGATGTCATTTTTGAGCTAATGCAAACAGAGATGCATCACATCCAGACCCTGTTCATCATGTCTG
AGATCTTCAGGAAAGGCATGAAAGAGGAGCTGCAGCTGGACCACAGCACCCTGGATAAAATTTTCCC
CTGTTTAGATGAGTTGCTTGAAATCCACAGCCATTTCTTCTACAGTATGAAGGAACGAAGGCAGGAA
TCAACTGCTGGCAGCGACAGGAATTTTGTGATCGACCGAATTGGAGATATTTTGGTACAACAGTTTT
CAGAAGAAAATCCAAGTAAAATGAAGAAAATATATGGAGAATTCTGTTCCCATCATAAAGAAGCTGT
TAACCTCTTTAAAGAACTCCAGCAGAATAAAAAGTTTCAGAATTTTATTAAGCTCCGAAATAGTAAT
CTTTTGGCTCGACGCCCAGGAATTCCAGAATGCATTCTGTTGGTCACTCAGCGTATTACAAAATACC
CTGTCTTGGTGCAAAGGATATTCCAGTACACAAAGGAAAGAACTGAGGAACATAAACACTTACGCAA
ACCCCTTTGCTTAATTAAACACATGATTGCAACAGTCGATTTAAAAGTCAATGAATATGAGAAAAAC
CAAAAATGGCTTGAGATCCTAAATAAGATTGAAAACAAAACATACACCAAGCTCAAAAATGGACATG
TGTTTAGGAAGCAGGCACTCATGAGTGAAGAAAGGACTCTGTTATATGATGGCCTTGTTTACTGGAA
ACTGCTACAGGTCGTTTCAAAGATATCCTAGCTCTACTTCTAACTGATGTGCTAACTCTTTTTACAA
GAAAAAGACCAGAAATACATCTTTGCAGCCGTTGATCAGAAGCCATCAGTTATTTCCCTTCAAAAGC
TTATTGCTAGAGAAGTTGCTAATGAGGAGAGACCAATGTTTCTGATCAGTGCTTCATCTGCTGGTCC
TGAGATGTATGAAATTCACACCAATTCCAAGGAGGAACGCAATAACTGGATGACACGGATCCACCAG
GCTGTAGAAAGTTGTCCTGAAGAAAAAGGGGGAAGGACAAGTGAATCTGATGAAGACAACAGGAAAG
CTGAAGCCAGAGTGGCCAAAATTCAGCAATGTCAAGAAATACTCACTAACCAAGACCAACAAATTTC
TGCCTATTTCGAGGAGAAGCTCCATATCTATGCTGAACTTGGACAACTGAGCGGATTTGAGGACGTC
CATCTAGAGCCCCACCTCCTTATTAAACCTGACCCAGGCGAGCCTCCCCACGCACCCTCATTACTGG
CAGCAGCACTGAAACAAGCTGAGAGCCTACAAGTTGCAGTGAAGGCCTCACAGATGGGCGCCGTGAG
TCAATCATGTGAGGACAGTTGTGGAGACTCTCTCTTGGCCGACACACTCAGTTCTCATGATGTACCA
GGATCACCGACTGCCTCATTAGTCACAGGAGGGAGAGAAGGAAGAGGCTCTTCGGATGTGGATCCCG
GGATCCAGGGTGTGGTAACCGACTTGGCCGTCTCTGATGCAGGGGAGAAGGTCCAATGTAGAAATTT
TCCAGGTTCTTCACAATCAGAGATTATACAAGCCATACAGAATTTAACCCGTCTCTTATACAGCCTT
CACGCCGCCTTGACCATTCAGGACAGCCACATTGAGATCCACAGGCTGCTTCTCCAGCACCACCAGG
GCCTCTCTCTCCGCCACTCTATCCTCCGAGGCGGCCCCTTGCACCACCAGAAGTCTCGCGACCCGGA
CAGGCACCATGAGGAGCTGGCCAATGTGCACCAGCTTCAGCACCACCTCCACCAGGACCAGCGGCGC
TGGCTGCGCAGGTGTGACCAGCACCAGCGGGCGCAGGCGACCAGGGAGAGCTGGCTGCAGGAGCGGG
AGCGCGAGTGCCAGTCGCAGGAGGAGCTGCTGCTGCGGAGCCGGGOCGAGCTGGACCTCCAGCTCCA
GGAGTACCAGCACACCCTGCAGCGCCTGAGCGAGGGCCAGCCCCTGGTGGAGAGGGAGCAGGCGAGG
ATGCGGCCCCACCAGAGCCTGCTGGGCCACTGGAAGCACGGCCGCCAGAGGAGCCTCCCCGCCGTCC
TCCTTCCGGCTGGCCCCGAGGTAATGGAACTTAATCGATCTGAGACTTTATGTCATCAAAACTCATT
CTTCATCAATGAAGCTTTAGTACAAATGTCATTTAACACTTTCAACAAACTGAATCCGTCAGTTATC
CATCAGGATGCCACTTACCCTACAACTCAATCTCATTCTGACTTGGTGAGGACTAGTGAACATCAAG
TACACCTCAAGGTGGACCCTTCTCAGCCTTCGAATGTCAGTCACAAACTGTGGACACCCGCTGGTTC
CGGCCATCAGATACTTCCTTTCCAAGAAAGCAGCAAGGATTCTTGTAAAAATGATTTGGACACCTCC
CACACTCAGTCCCCAACCCCCCATGACTCAAATTCACACCGCCCTCAACTGCAGGCGTTTATAACAG
AAGCAAAGCTAAATCTACCGACAAGGACAATGACCAGACAAGATGGGGAAACTGCAGATCGAGCCAA
AGAAAATATTGTTTACCTCTAATTGTGTTGTCATTTTTCCAAACAAAACAAAACACTGGCACTTTTG
GGAGAAACTTTTTGTCTCCATTCCTTATGTATGTGTGATTCTCTGTGTCCAAATTGCTTTAAGAATA
ATATTTAATATTTCCTGGAACCTCATTTTTTTCCCATGACTCTAATTAAATTATTCAAAGCCAAAAA
AAAAAAAAAAAA

The NOV16 protein (SEQ ID NO:34) encoded by SEQ ID NO:33 is 1706 amino acid residues in length and is presented using the one-letter amino acid code in Table 16B. Psort analysis predicts the NOV16 protein of the invention to be localized in the cytoplasm with a certainty of 0.4500.

TABLE 16B
Encoded NOV16 protein sequence

(SEQ ID NO:34)

MELSCSEAPLYQGQMMIYAKFDKNVYLPEDAEFYFTYDGSHQRHVMTAERIEDNVLQSSVPGHGL
QETVTVSVCLCSEGYSPVTMGSGSVTYVDNMACRLARLLVTQANRLTACSHQTLLTPFALTAGAL
PALDEELVLALTHLELPLEWTVLGSSSLEVSSHRESLLHLAMRWGLAKLSQFFLCLPGGVQALAL
PNEEGATPLDLALREGHSKLVEDVTSFQGRWSPSFSRVQLSEEASLHYIHSSETLTLTLNHTAEH
LLEADIKLFRKYFWDRAFLVKAFEQEARPEERTAMPSSGAETEEEIKNSVSSRSAAEKEDIKRVK
SLVVQHNEHEDQHSLDSRSLLRYPKKSKPPSTLLAAGRLSDMLNGGDEVYANCMVIDQVGDLDIS
YTNIECITATTSPESRGCTLWPQSSKHTLPTETSPSVYPLSENVEGTAHTEAQQSFMSPSSSCAS
NLNLSFGWHGFEKEQSHLKKRSSSLDALDADSEGEGHSEPSHICYTPGSQSSSRTGTPSGDELDS
FETNTEPDFNISRAESLPLSSNLQLKESLLSGVRSRSYSCSSPKISLGKTRLVRELTVCSSSEEQ
KAYSLSEPPRENRIQEEEWDKYIIPAKSESEKYKVSRTFSFLMNRMTSPRNKSKTKSKDAKDKEK
LNRHQPAPGTFSGVLQCLVCDKTLLGKESLQCSSCNANVHKGCKDAAPACTKKFQEKYNKNKPQT
ILGSSSFRDIPQPGLSLHPSSSVPVGLPTGRRETVGQVHPLSRSVPGTTLESFRRSATSLESESD
NNSCRSRSHSDELLQSMGSSPSTESFIMEDVVDSSLWSDLSSDAQEFEAESWSLVVDPSFCNRQE
KDVIKRQDVIFELMQTEMHHIQTLFIMSEIFRKGMKEELQLDHSTVDKIFPCLDELLEIHRHFFY
SMKERRQESSAGSDRNFVIDRIGDILVQQFSEENASKMKKIYGEFCCHHKEAAALFKELQQNKKF
QNFIKLRNSNLLARRRCIPECILLVTQRTTKYPVLVERILQYTKERTEEHKDLRKALCLIKDMIA
TVDLKVNEYEKNQKWLEILNKIENKTYTKLKNGHVFRKQALMSEERTLLYDCLVYWKTATGRFKD
ILALLLTDVLLFLQEKDQKYIFAAVDQKPSVISLQKLIAREVANEERGMFLISASSAGPEMYEIH
TNSKEERNNWMRRIQQAVESCPEEKGGRTSESDEDKRKAEARVAKIQQCQEILTNQDQQTCAYLE
EKLHIYAELGELSGFEDVHLEPHLLIKPDPGEPPQAASLLAAALKEAESLQVAVKASQMGAVSQS
CEDSCGDSVLADTLSSHDVPGSPTASLVTGGREGRGCSDVDPGIQGVVTDLAVSDAGEKVECRNF
PGSSQSEIIQAIQNLTRLLYSLQAALTIQDSHIEIHRLVLQQQEGLSLGHSILRGCPLQDQKSRD
ADRQHEELANVHQLQHQLQQEQRRWLRRCEQQQRAQATRESWLQERERECQSQEELLLRSRCELD
LQLQEYQHSLERLREGQRLVEREQARMRAQQSLLGHWKHGRQRSLPAVLLPGGPEVMELNRSESL
CHENSFFTNEALVQMSFNTFNKLNPSVIHQDATYPTTQSHSDLVRTSEHQVDLKVDPSQPSNVSH
KLWTAAGSGHQILPFQESSKDSCKNDLDTSHTESPTPHDSNSHRPQLQAFITEAKLNLPTRTMTR
QDCETGDGAKENIVYL

A search against the Patp database, a proprietary database that contains sequences published in patents and patent publications, yielded several homologous proteins shown in Table 16C.

TABLE 16C
Patp results for NOV16

| | | | Smallest |
|---|---|---|---|
| | | | Sum |
| Sequences producing High-scoring | Reading | High | Prob |
| Segment Pairs: | Frame | Score | P (N) |
| >patp:ABB44551 Human wound healing | +1 | 1470 | 2.8e−150 |
| related polypeptide | | | |
| >patp:AAW93941 Human brx protein | +1 | 1436 | 1.5e−148 |
| >patp:ABG05537 Novel human diagnostic | +1 | 1436 | 1.5e−148 |
| protein #5528 | | | |
| >patp:ABG05537 Novel human diagnostic | +1 | 1436 | 1.5e−148 |
| protein #5528 | | | |
| >patp:ABG15870 Novel human diagnostic | +1 | 1447 | 7.5e−148 |
| protein #15861 | | | |

In a BLAST search of public sequence databases, it was found, for example, that the nucleic acid sequence of this invention has 4339 of 5274 bases (82%) identical to a gb:GENBANK-ID:MMU73199|acc:U73199.1 mRNA from Mus musculus (Rho-guanine nucleotide exchange factor mRNA, complete cds). The full amino acid sequence of the protein of the invention was found to have 1350 of 1670 amino acid residues (80%) identical to, and 1460 of 1670 amino acid residues (87%) similar to, the 1693 amino acid residue ptnr:SWISSPROT-ACC:P97433 protein from Mus musculus (RHO-GUANINE NUCLEOTIDE EXCHANGE FACTOR(RHOGEF) (RIP2)).

NOV16 also has homology to the proteins shown in the BLASTP data in Table 16D.

TABLE 16D
BLAST results for NOV16

| Gene Index/ | | | | | |
|---|---|---|---|---|---|
| Identifier | Protein/Organism | Length (aa) | Identity (%) | Positives (%) | Expect |
| gi\|7106395\|ref\|NP_{—} | Rho interacting | 1693 | 1350/1674 | 1460/1674 | 0.0 |
| 036156.1\|(NM_012026) | protein 2; Rho | | (80%) | (86%) | |
| | specific exchange | | | | |
| | factor | | | | |
| | [Mus musculus] | | | | |
| gi\|18602674\|ref\|XP_{—} | hypothetical | 669 | 668/669 | 668/669 | 0.0 |
| 016989.5\|(XM_016989) | protein FLJ21817 | | (99%) | (99%) | |
| | similar to Rhoip2 | | | | |
| | [Homo sapiens] | | | | |
| gi\|10438441\|dbj\| | unnamed protein | 669 | 669/669 | 669/669 | 0.0 |
| BAB15243.1\|(AK025816) | product | | (100%) | (100%) | |
| | [Homo sapiens] | | | | |
| gi\|15341761\|gb\| | hypothetical | 615 | 609/613 | 609/613 | 0.0 |
| AAH12946.1\|AAH12946 | protein FLJ21817 | | (99%) | (99%) | |
| (BC012946) | similar to Rhoip2 | | | | |
| | [Homo sapiens] | | | | |
| gi\|17437752\|ref\|XP_{—} | similar to Rho | 590 | 290/292 | 291/292 | e−170 |
| 068710.1\|(XM_068710) | interacting | | (99%) | (99%) | |
| | protein 2; Rho | | | | |
| | specific exchange | | | | |
| | factor | | | | |
| | [Homo sapiens] | | | | |

A multiple sequence alignment is given in Table 16E, with the NOV16 protein being shown on line 1 in Table 16E in a ClustalW analysis, and comparing the NOV16 protein with the related protein sequences shown in Table 16D. This BLASTP data is displayed graphically in the ClustalW in Table 16E.

[image]

[image]

[image]

[image]

[image]

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 16F lists the domain description from DOMAIN analysis results against NOV16.

TABLE 16F
Domain Analysis of NOV16

| | Region of | | |
|---|---|---|---|
| Model | Homology | Score (bits) | E value |
| Phorbol | 654-700 | 40.1 | 4.9e−08 |
| esters/diacylglycerol | | | |
| binding dom (DAG_PE- | | | |
| bind) | | | |
| PHD-finger (PHD) | 666-703 | 1.4 | 0.06 |
| Phorbol | 654-698 | 46.2 | 2.0e−05 |
| esters/diacylglycerol | | | |
| binding domain (DAG | | | |
| PE-bind) | | | |
| C1, Protein kinase C | 654-698 | 45.8 | 2.0e−05 |
| conserved region 1 | | | |
| RhoGEF domain | 854-1044 | 78.4 | 1.5e−19 |
| (RhoGEF) | | | |
| bZIP transcription | 1022-1048 | 5.0 | 7.1 |
| factor (bZIP) | | | |
| PH domain (PH) | 1088-1189 | 40.9 | 4.5e−10 |

Consistent with other known members of the subunit family of proteins, NOV16 has, for example, a RhoGEF signature sequence and homology to other members of the RhoGEF-like Protein Family. NOV16 nucleic acids, and the encoded polypeptides, according to the invention are useful in a variety of applications and contexts. For example, NOV16 nucleic acids and polypeptides can be used to identify proteins that are members of the RhoGEF-like Protein Family. The NOV16 nucleic acids and polypeptides can also be used to screen for molecules, which inhibit or enhance NOV16 activity or function. Specifically, the nucleic acids and polypeptides according to the invention may be used as targets for the identification of small molecules that modulate or inhibit, e.g., cellular activation, cellular replication, and signal transduction. These molecules can be used to treat, e.g., cancer, trauma, regeneration (in vitro and in vivo), viral/bacterial/parasitic infections, diabetes, Von Hippel-Lindau (VHL) syndrome, pancreatitis, obesity, anemia, bleeding disorders, scleroderma, transplantation, hemophilia, hypercoagulation, idiopathic thrombocytopenic purpura, immunodeficiencies, graft versus host disease, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cirrhosis, transplantation, adrenoleukodystrophy, congenital adrenal hyperplasia, hemophilia, hypercoagulation, idiopathic thrombocytopenic purpura, autoimmune disease, allergies, immunodeficiencies, transplantation, graft versus host disease, lymphedema, allergies, immunodeficiencies, osteoporosis, hypercalceimia, arthritis, ankylosing spondylitis, scoliosis, tendinitis, systemic lupus erythematosus, autoimmune disease, asthma, emphysema, scleroderma, ARDS, endometriosis, fertility, hyperthyroidism, hypothyroidism, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, psoriasis, actinic keratosis, tuberous sclerosis, acne, hair growth/loss, alopecia, pigmentation disorders, endocrine disorders as well as other diseases, disorders and conditions.

In addition, various NOV16 nucleic acids and polypeptides according to the invention are useful, inter alia, as novel members of the protein families according to the presence of domains and sequence relatedness to previously described proteins. For example, the NOV16 nucleic acids and their encoded polypeptides include structural motifs that are characteristic of proteins belonging to the RhoGEF-like Protein Family.

GEF (Guanine nucleotide exchange factor) for Rho/Rac/Cdc42-like GTPases is also called Dbl-homologous (DH) domain. It appears that PH (pleckstrin homology) domains invariably occur C-terminal to RhoGEF/DH domains. Although the exact function of PH domains is unclear, several choices include binding to the beta/gamma subunit of heterotrimeric G proteins, binding to lipids, e.g. phosphatidylinositol-4,5-bisphosphate, binding to phosphorylated Ser/Thr residues, attachment to membranes by an unknown mechanism. The DAG_PE-binding domain binds two zinc ions; the ligands of these metal ions are probably the six cysteines and two histidines that are conserved in this domain and can regulate signal transduction by the PKC family of kinases.

NOV16 belongs to the guanine nucleotide exchange factor family of proteins which play a significant role in signal transduction. The guanine nucleotide exchange factor (GEF) domain that regulates GTP binding protein signaling. The GEF domain regulates positively the signaling cascades that utilize GTP-binding proteins (such as those of the ras superfamily) that function as molecular switches in fundamental events such as signal transduction, cytoskeleton dynamics and intracellular trafficking. An example of a protein containing GEF and PH domains is FGDI (faciogenital dyplasia protein) Experiments have shown that the GEF and (PH) domains of FGDI can bind specifically to the Rho family GTPase Cdc42Hs and stimulates the GDP-GTP exchange of the isoprenylated form of Cdc42Hs. The GEF domain of FGDI has also been shown to activate 2 kinases involved in cell proliferation; the Jun NH2-terminal kinase and the p70 S6 kinase (Zheng et al.; J. Biol. Chem Dec. 27, 1996;271(52):33169-72). Thus, NOV16 polypeptide may play an important role in normal development as well as disease. This class of molecules (GEFs) is also being considered as a good drug target as the guanine nucleotide exchange factor RasGRP is a high-affinity target for diacylglycerol and phorbol esters and is bound by bryostatin 1, a compound currently in clinical trials (Lorenzo et al.; Mol. Pharmacol 2000 May; 57(5):840-6). The homolog of RhoGEF, DIhoGEF2 fail to gastrulate due to a defect in cell shape changes required for tissue invagination and the mRNA is found throughout oogenesis and embryogenesis (Barrett et al.; Cell 1997; 91(7):905-15; Werneretal.; Gene 1997; 187(1):107-14). RhoGEF also interacts with c-Jun amino-terminal kinase (JNK) interacting protein-1 (JIP-1). JIP-1 might function as a scaffold protein by complexing specific components of the JNK signaling pathway, namely JNK, mitogen-activated protein kinase kinase 7, and mixed lineage kinase 3 (Meyer et al.; J Biol Chem 1999; 274(49):35113-8).

The NOV16 nucleic acids and polypeptides, antibodies and related compounds according to the invention will be useful in therapeutic and diagnostic applications in the mediation of blood and nerve physiology. As such, the NOV16 nucleic acids and polypeptides, antibodies and related compounds according to the invention may be used to treat blood and nervous system disorders, e.g., cancer, trauma, regeneration (in vitro and in vivo), viral/bacterial/parasitic infections, diabetes, Von Hippel-Lindau (VHL) syndrome, pancreatitis, obesity, anemia, bleeding disorders, scleroderma, transplantation, hemophilia, hypercoagulation, idiopathic thrombocytopenic purpura, immunodeficiencies, graft versus host disease, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cirrhosis, transplantation, adrenoleukodystrophy, congenital adrenal hyperplasia, hemophilia, hypercoagulation, idiopathic thrombocytopenic purpura, autoimmune disease, allergies, immunodeficiencies, transplantation, graft versus host disease, lymphedema, allergies, immunodeficiencies, osteoporosis, hypercalceimia, arthritis, ankylosing spondylitis, scoliosis, tendinitis, systemic lupus erythematosus, autoimmune disease, asthma, emphysema, scleroderma, ARDS, endometriosis, fertility, hyperthyroidism, hypothyroidism, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, psoriasis, actinic keratosis, tuberous sclerosis, acne, hair growth/loss, alopecia, pigmentation disorders, endocrine disorders as well as other diseases, disorders and conditions.

The NOV16 nucleic acids and polypeptides are useful for detecting specific cell types. For example, expression analysis has demonstrated that a NOV16 nucleic acid is expressed in Adipose, Umbilical Vein, Pancreas, Thymus, Brain, Lung, Kidney, Adrenal Gland/Suprarenal gland, Peripheral Blood, Lymph node, Cartilage, Mammary gland/Breast, Uterus, Prostate, Trachea, Cochlea, Dermis, Heart, Aorta, Coronary Artery, Thyroid, Liver, Bone, Bone Marrow, Spinal Cord, Cervix, and Retina.

Additional utilities for NOV16 nucleic acids and polypeptides according to the invention are disclosed herein.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them. The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELLISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31, and 33, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, and 33 as a hybridization probe, NOVX molecules can be isolated using standard hybridization and cloning techniques (e.g., as described in Sambrook, et al., (eds.), Molecular Cloning: a Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, 31, and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, or 33, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29, 31, or 33 is one that is sufficiently complementary to the nucleotide sequence shown SEQ ID NOS:1, 3, 5, 7, 9, 11, 13, 15, 17, 19, 21, 23, 25, 27, 29, 31, or 33 that it can hydrogen bond with little or no mismatches to the nucleotide sequence shown SEQ ID NOS:1, 3, 5, 7, 9, 11, 13, 15, 17, 19, 21, 23, 25, 27, 29, 31, or 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species. Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31, and 33, as well as a polypeptide possessing NOVX biological activity. Various biological activities of the NOVX proteins are described below.

An NOVX polypeptide is encoded by the open reading frame (“OPF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31, or 33; or an anti-sense strand nucleotide sequence of SEQ ID NOS:1, 3, 5, 7, 9, 11, 13, 15, 17, 19, 21, 23, 25, 27, 29, 31, or 33; or of a naturally occurring mutant of SEQ ID NOS:1, 3, 5, 7, 9, 11, 13, 15, 17, 19, 21, 23, 25, 27, 29, 31, and 33.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, 31, or 33, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31, and 33 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31, and 33. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, or 34.

In addition to the human NOVX nucleotide sequences shown in SEQ ID NOS:1, 3, 5, 7, 9, 11, 13, 15, 17, 19, 21, 23, 25, 27, 29, 31, and 33, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, 31, and 33 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31, and 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31, and 33, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31, and 33, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, a Laboratory Manual, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, 31, and 33, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, a Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31, and 33, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32, or 34. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2, 4, 6, 8, 10, 12, 14, 16, 18, 20, 22, 24, 26, 28, 30, 32, and 34 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, and 34. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32, and 34; more preferably at least about 70% homologous SEQ ID NOS:2, 4, 6, 8, 10, 12, 14, 16, 18, 20, 22, 24, 26, 28, 30, 32, or 34; still more preferably at least about 80% homologous to SEQ ID NOS:2, 4, 6, 8, 10, 12, 14, 16, 18, 20, 22, 24, 26, 28, 30, 32, or 34; even more preferably at least about 90% homologous to SEQ ID NOS:2, 4, 6, 8, 10, 12, 14, 16, 18, 20, 22, 24, 26, 28, 30, 32, or 34; and most preferably at least about 95% homologous to SEQ ID NOS:2, 4, 6, 8, 10, 12, 14, 16, 18, 20, 22, 24, 26, 28, 30, 32, or 34.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, or 34 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, and 33, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31, and 33 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagetnesis, and the resultant mutants can be screened for NOVX biological activity to identify mutants that retain activity. Following mutagenesis of SEQ ID NOS:1, 3, 5, 7, 9, 11, 13, 15, 17, 19, 21, 23, 25, 27, 29, 31, and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, or 34, or antisense nucleic acids complementary to an NOVX nucleic acid sequence of SEQ ID NOS:1, 3, 5, 7, 9, 11, 13, 15, 17, 19, 21, 23, 25, 27, 29, 31, and 33, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methyl inosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in sit such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an x-anomeric nucleic acid molecule. An u-anomeric nucleic acid molecule forms specific double-stranded hybrids with complementary RNA in which, contrary to the usual P-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, 31, and 33). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N. Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposoines or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Left. 5: 119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence-of NOVX polypeptides whose sequences are provided in SEQ ID NOS:2, 4, 6, 8, 10, 12, 14, 16, 18, 20, 22, 24, 26, 28, 30, 32, or 34. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, or 34 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32, or 34) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, or 34. In other embodiments, the NOVX protein is substantially homologous to SEQ ID NOS:2, 4, 6, 8, 10, 12, 14, 16, 18, 20, 22, 24, 26, 28, 30, 32, or 34, and retains the functional activity of the protein of SEQ ID NOS:2, 4, 6, 8, 10, 12, 14, 16, 18, 20, 22, 24, 26, 28, 30, 32, or 34, yet differs in amino acid sequence due to natural allelic variation or mutagenesis, as described in detail, below.

Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, or 34, and retains the functional activity of the NOVX proteins of SEQ ID NOS:2, 4, 6, 8, 10, 12, 14, 16, 18, 20, 22, 24, 26, 28, 30, 32, or 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 31, and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, or 34), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fuised in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs hoinologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol, 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al, Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Strict. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylaimine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fe receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), d iisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids.

In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: a Laboratory Manual. 2nd ed., Cold Spring Harbor Laboratory, Cold Spring Harbor Laboratory Press, Cold Spring Harbor, N.Y., 1989. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the F-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the tern as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: a Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1,3, 5, 7, 9, 11, 13, 15, 17, 19, 21, 23, 25, 27, 29, 31, and 33 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31, and 33), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31, and 33 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: a Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilinut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein. In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Tritono X-114, Thesit®, lsotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO). In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway. The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, 31, and 33, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: a Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057). Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, and 33 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials. These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, 31, and 33, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking), a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Nail. Acad. Sci. USA 86: 1173-1177); Qu Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Muitation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039. In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl.Acids Res 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification. The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug)-of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide Imniimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. As shown in Table 17, PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 17

PCR Primers for Exon Linking

| | | | | |
|---|---|---|---|---|
| | | SEQ | | SEQ |
| NOVX | | ID | | ID |
| Clone | Primer 1 (5′ - 3′) | NO | Primer 2 (5′ - 3′) | NO |

| | | | | |
|---|---|---|---|---|
| 10b | GCACAGACGGTACTCACCCTTCTT | 112 | GGAGTTGATGTGGAGTTGTAGCTGACT | 113 |

### Example 2

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosysteins ABI PRISM® 7700 or an ABI PRISM (T 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing norinal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:128s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No.4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMang Universal Master mix (Applied Biosystems; catalog No.4324020), following the manufacturer's instructions.

Probes and primers were designed for each assay according to Applied Biosystems Primer Express Software package (version 1 for Apple Computer's Macintosh Power PC) or a similar algorithm using the target sequence as input. Default settings were used for reaction conditions and the following parameters were set before selecting primers: primer concentration=250 nM, primer melting temperature (Tm) range 58°-60° C., primer optimal Tm=59° C., maximum primer difference=2° C., probe does not have 5′G, probe Tm must be 10° C. greater than primer Tin, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMang One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pi. eff=pi effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 M non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 1001 M non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 1001 M non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-1L4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP, at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCDI 106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7μ RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al, Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodinan Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirn diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheiiner's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodeoeneration_VI.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV2 (CG93210-01: Plasma Membrane Ring Finger Protein)

Expression of NOV2 gene (CG93210-01) was assessed using the primer-probe set Ag3845, described in Table AA. Results of the RTQ-PCR runs are shown in Tables AB, AC, AD and AE.

TABLE AA

Probe Name Ag3845

| | | | |
|---|---|---|---|
| Primers | Sequences | Length | Start Position |

| | | | | |
|---|---|---|---|---|
| Forward | 5′-ccaatatcgagtggaagttgac-3′ | (Seq ID NO:114) | 22 | 651 |
| Probe | TET-5′-cttgtggaccacctgtggcctct-3′-TAMRA | (Seq ID NO:115) | 23 | 673 |
| Reverse | 5′-agtcctgccatcctccatag-3′ | (Seq ID NO:116) | 20 | 706 |

TABLE AB
CNS_neurodegeneration_v1.0

| | Rel. Exp. | | Rel. Exp. |
|---|---|---|---|
| | (%) Ag3845, | | (%) Ag3845, |
| | Run | | Run |
| Tissue Name | 206878154 | Tissue Name | 206878154 |
| AD 1 Hippo | 20.9 | Control (Path) 3 | 23.8 |
| | | Temporal Ctx | |
| AD 2 Hippo | 50.7 | Control (Path) 4 | 61.6 |
| | | Temporal Ctx | |
| AD 3 Hippo | 16.8 | AD 1 Occipital Ctx | 27.0 |
| AD 4 Hippo | 31.0 | AD 2 Occipital Ctx | 0.2 |
| | | (Missing) | |
| AD 5 hippo | 84.7 | AD 3 Occipital Ctx | 14.6 |
| AD 6 Hippo | 48.3 | AD 4 Occipital Ctx | 33.0 |
| Control 2 Hippo | 53.2 | AD 5 Occipital Ctx | 15.6 |
| Control 4 Hippo | 27.5 | AD 6 Occipital Ctx | 67.4 |
| Control (Path) 3 | 19.6 | Control 1 Occipital | 7.6 |
| Hippo | | Ctx | |
| AD 1 Temporal | 26.4 | Control 2 Occipital | 98.6 |
| Ctx | | Ctx | |
| AD 2 Temporal | 45.7 | Control 3 Occipital | 50.7 |
| Ctx | | Ctx | |
| AD 3 Temporal | 28.7 | Control 4 Occipital | 18.9 |
| Ctx | | Ctx | |
| AD 4 Temporal | 30.4 | Control (Path) 1 | 100.0 |
| Ctx | | Occipital Ctx | |
| AD 5 Inf | 81.8 | Control (Path) 2 | 17.9 |
| Temporal Ctx | | Occipital Ctx | |
| AD 5 Sup | 39.8 | Control (Path) 3 | 8.0 |
| Temporal Ctx | | Occipital Ctx | |
| AD 6 Inf | 62.9 | Control (Path) 4 | 31.0 |
| Temporal Ctx | | Occipital Ctx | |
| AD 6 Sup | 47.0 | Control 1 Parietal | 21.9 |
| Temporal Ctx | | Ctx | |
| Control 1 | 10.9 | Control 2 Parietal | 65.5 |
| Temporal Ctx | | Ctx | |
| Control 2 | 63.7 | Control 3 Parietal | 30.4 |
| Temporal Ctx | | Ctx | |
| Control 3 | 34.6 | Control (Path) 1 | 86.5 |
| Temporal Ctx | | Parietal Ctx | |
| Control 4 | 21.3 | Control (Path) 2 | 30.4 |
| Temporal Ctx | | Parietal Ctx | |
| Control (Path) 1 | 95.3 | Control (Path) 3 | 15.9 |
| Temporal Ctx | | Parietal Ctx | |
| Control (Path) 2 | 54.0 | Control (Path) 4 | 57.4 |
| Temporal Ctx | | Parietal Ctx | |

TABLE AC
General_screening_panel_v1.4

| | Rel. Exp. | | Rel. Exp. |
|---|---|---|---|
| | (%) Ag3845, | | (%) Ag3845, |
| | Run | | Run |
| Tissue Name | 213608497 | Tissue Name | 213608497 |
| Adipose | 1.1 | Renal ca. TK-10 | 6.1 |
| Melanoma* | 3.5 | Bladder | 6.6 |
| Hs688(A).T | | | |
| Melanoma* | 3.7 | Gastric ca. (liver | 14.5 |
| Hs688(B).T | | met.) NCI-N87 | |
| Melanoma* M14 | 3.1 | Gastric ca. KATO | 6.3 |
| | | III | |
| Melanoma* | 4.0 | Colon ca. SW-948 | 2.6 |
| LOXIMVI | | | |
| Melanoma* | 3.6 | Colon ca. SW480 | 7.0 |
| SK-MEL-5 | | | |
| Squamous cell | 0.9 | Colon ca.* | 3.5 |
| carcinoma SCC-4 | | (SW480 met) SW620 | |
| Testis Pool | 1.7 | Colon ca. HT29 | 1.7 |
| Prostate ca.* | 9.4 | Colon ca. HCT-116 | 9.4 |
| (bone met) PC-3 | | | |
| Prostate Pool | 1.6 | Colon ca. CaCo-2 | 6.1 |
| Placenta | 2.0 | Colon cancer | 3.5 |
| | | tissue | |
| Uterus Pool | 0.8 | Colon ca. SW1116 | 2.9 |
| Ovarian ca. | 8.7 | Colon ca. Colo- | 0.4 |
| OVCAR-3 | | 205 | |
| Ovarian ca. | 20.9 | Colon ca. SW-48 | 1.1 |
| SK-OV-3 | | | |
| Ovarian ca. | 3.3 | Colon Pool | 5.4 |
| OVCAR-4 | | | |
| Ovarian ca. | 39.0 | Small Intestine | 3.2 |
| OVCAR-5 | | Pool | |
| Ovarian ca. | 13.7 | Stomach Pool | 2.2 |
| IGROV-1 | | | |
| Ovarian ca. | 8.6 | Bone Marrow Pool | 1.3 |
| OVCAR-8 | | | |
| Ovary | 1.4 | Fetal Heart | 1.9 |
| Breast ca. | 3.2 | Heart Pool | 3.1 |
| MCF-7 | | | |
| Breast ca. | 9.2 | Lymph Node Pool | 5.4 |
| MDA-MB-231 | | | |
| Breast ca. | 11.0 | Fetal Skeletal | 0.9 |
| BT 549 | | Muscle | |
| Breast ca. | 100.0 | Skeletal Muscle | 1.3 |
| T47D | | Pool | |
| Breast ca. | 2.0 | Spleen Pool | 1.6 |
| MDA-N | | | |
| Breast Pool | 3.7 | Thymus Pool | 3.1 |
| Trachea | 1.1 | CNS cancer | 12.0 |
| | | (glio/astro) U87- | |
| | | MG | |
| Lung | 0.5 | CNS cancer | 20.2 |
| | | (glio/astro) U- | |
| | | 118-MG | |
| Fetal Lung | 4.2 | CNS cancer | 6.7 |
| | | (neuro; met) SK- | |
| | | N-AS | |
| Lung ca. NCI-N417 | 1.2 | CNS cancer (astro) | 4.6 |
| | | SF-539 | |
| Lung ca. LX-1 | 2.9 | CNS cancer (astro) | 10.7 |
| | | SNB-75 | |
| Lung ca. NCI-H146 | 1.8 | CNS cancer (glio) | 14.6 |
| | | SNB-19 | |
| Lung ca. SHP-77 | 5.0 | CNS cancer (glio) | 21.8 |
| | | SF-295 | |
| Lung ca. A549 | 6.4 | Brain (Amygdala) | 3.1 |
| | | Pool | |
| Lung ca. NCI-H526 | 1.5 | Brain (cerebellum) | 9.9 |
| Lung ca. NCI-H23 | 24.3 | Brain (fetal) | 6.8 |
| Lung ca. NCI-H460 | 8.4 | Brain | 3.3 |
| | | (Hippocampus) Pool | |
| Lung ca. HOP-62 | 6.7 | Cerebral Cortex | 6.0 |
| | | Pool | |
| Lung ca. NC1-H522 | 8.8 | Brain (Substantia | 7.0 |
| | | nigra) Pool | |
| Liver | 0.4 | Brain (Thalamus) | 4.5 |
| | | Pool | |
| Fetal Liver | 3.4 | Brain (whole) | 3.3 |
| Liver ca. HepG2 | 2.0 | Spinal Cord Pool | 3.0 |
| Kidney Pool | 10.2 | Adrenal Gland | 1.4 |
| Fetal Kidney | 5.9 | Pituitary gland | 1.1 |
| | | Pool | |
| Renal ca. 786-0 | 6.5 | Salivary Gland | 0.7 |
| Renal ca. A498 | 2.7 | Thyroid (female) | 2.5 |
| Renal ca. ACHN | 5.1 | Pancreatic ca. | 2.8 |
| | | CAPAN2 | |
| Renal ca. UO-31 | 5.8 | Pancreas Pool | 5.7 |

TABLE AD
Panel 2.1

| | Rel. Exp. | | Rel. Exp. |
|---|---|---|---|
| | (%) Ag3845, | | (%) Ag3845, |
| | Run | | Run |
| Tissue Name | 170686165 | Tissue Name | 170686165 |
| Normal Colon | 8.4 | Kidney Cancer | 23.3 |
| | | 9010320 | |
| Colon cancer | 1.3 | Kidney margin | 100.0 |
| (OD06064) | | 9010321 | |
| Colon cancer | 1.3 | Kidney Cancer | 25.7 |
| margin (OD06064) | | 8120607 | |
| Colon cancer | 1.1 | Kidney margin | 16.4 |
| (OD06159) | | 8120608 | |
| Colon cancer | 0.3 | Normal Uterus | 19.9 |
| margin (OD06159) | | | |
| Colon cancer | 24.0 | Uterus Cancer | 20.7 |
| (OD06298-08) | | | |
| Colon cancer | 14.4 | Normal Thyroid | 3.1 |
| margin | | | |
| (OD06298-018) | | | |
| Colon Cancer Gr.2 | 6.3 | Thyroid Cancer | 12.7 |
| ascend colon | | | |
| (ODO3921) | | | |
| Colon Cancer | 16.4 | Thyroid Cancer | 4.5 |
| margin (ODO3921) | | A302152 | |
| Colon cancer | 1.5 | Thyroid margin | 13.4 |
| metastasis | | A302153 | |
| (OD06104) | | | |
| Lung margin | 8.9 | Normal Breast | 12.2 |
| (OD06104) | | | |
| Colon mets to | 36.3 | Breast Cancer | 14.2 |
| lung (OD04451-01) | | | |
| Lung margin | 2.0 | Breast Cancer | 20.6 |
| (OD04451-02) | | | |
| Normal Prostate | 7.4 | Breast Cancer | 26.4 |
| | | (OD04590-01) | |
| Prostate Cancer | 5.5 | Breast Cancer Mets | 20.6 |
| (OD04410) | | (OD04590-03) | |
| Prostate margin | 27.5 | Breast Cancer | 51.4 |
| (OD04410) | | Metastasis | |
| Normal Lung | 20.3 | Breast Cancer | 0.0 |
| Invasive poor | 10.2 | Breast Cancer | 10.4 |
| diff. lung adeno | | 9100266 | |
| 1 (ODO4945-01) | | | |
| Lung margin | 14.3 | Breast margin | 7.0 |
| (ODO4945-03) | | 9100265 | |
| Lung Malignant | 10.9 | Breast Cancer | 8.5 |
| Cancer (OD03126) | | A209073 | |
| Lung margin | 7.7 | Breast margin | 34.6 |
| (OD03126) | | A2090734 | |
| Lung Cancer | 12.2 | Normal Liver | 17.7 |
| (OD05014A) | | | |
| Lung margin | 4.5 | Liver Cancer | 9.0 |
| (OD05014B) | | 1026 | |
| Lung Cancer | 10.2 | Liver Cancer | 12.9 |
| (OD04237-01) | | 1025 | |
| Lung margin | 14.2 | Liver Cancer | 13.7 |
| (OD04237-02) | | 6004-T | |
| Ocular Mel Met to | 12.2 | Liver Tissue | 4.8 |
| Liver (ODO4310) | | 6004-N | |
| Liver margin | 9.3 | Liver Cancer | 44.4 |
| (ODO4310) | | 6005-T | |
| Melanoma Mets | 12.8 | Liver Tissue | 29.3 |
| to Lung (OD04321) | | 6005-N | |
| Lung margin | 10.2 | Liver Cancer | 26.1 |
| (OD04321) | | | |
| Normal Kidney | 14.0 | Normal Bladder | 13.0 |
| Kidney Ca, Nuclear | 27.5 | Bladder Cancer | 6.8 |
| grade 2 (OD04338) | | | |
| Kidney margin | 13.0 | Bladder Cancer | 25.9 |
| (OD04338) | | | |
| Kidney Ca Nuclear | 17.1 | Normal Ovary | 18.4 |
| grade ½ | | | |
| (OD04339) | | | |
| Kidney margin | 21.3 | Ovarian Cancer | 13.1 |
| (OD04339) | | | |
| Kidney Ca, Clear | 15.6 | Ovarian cancer | 3.0 |
| cell type | | (OD06145) | |
| (OD04340) | | | |
| Kidney margin | 19.2 | Ovarian cancer | 20.7 |
| (OD04340) | | margin (OD06145) | |
| Kidney Ca, Nuclear | 10.7 | Normal Stomach | 15.2 |
| grade 3 (OD04348) | | | |
| Kidney margin | 14.8 | Gastric Cancer | 10.1 |
| (OD04348) | | 9060397 | |
| Kidney Cancer | 53.6 | Stomach margin | 7.0 |
| (OD04450-01) | | 9060396 | |
| Kidney margin | 8.4 | Gastric Cancer | 14.7 |
| (OD04450-03) | | 9060395 | |
| Kidney Cancer | 1.1 | Stomach margin | 7.4 |
| 8120613 | | 9060394 | |
| Kidney margin | 4.4 | Gastric Cancer | 12.2 |
| 8120614 | | 064005 | |

TABLE AE
Panel 4.1D

| | Rel. Exp. | | Rel. Exp. |
|---|---|---|---|
| | (%) Ag3845, | | (%) Ag3845, |
| | Run | | Run |
| Tissue Name | 169960518 | Tissue Name | 169960518 |
| Secondary Th1 | 15.6 | HUVEC IL-1beta | 54.7 |
| act | | | |
| Secondary Th2 | 23.7 | HUVEC IFN gamma | 40.3 |
| act | | | |
| Secondary Tr1 | 35.1 | HUVEC TNF alpha + | 19.6 |
| act | | IFN gamma | |
| Secondary Th1 | 21.8 | HUVEC TNF alpha + | 24.0 |
| rest | | IL4 | |
| Secondary Th2 | 29.7 | HUVEC IL-11 | 29.5 |
| rest | | | |
| Secondary Tr1 | 29.3 | Lung | 100.0 |
| rest | | Microvascular | |
| | | EC none | |
| Primary Th1 act | 21.6 | Lung | 75.8 |
| | | Microvascular | |
| | | EC TNFalpha + | |
| | | IL-1beta | |
| Primary Th2 act | 24.5 | Microvascular | 34.2 |
| | | Dermal EC none | |
| Primary Tr1 act | 22.4 | Microsvasular | 46.7 |
| | | Dermal EC | |
| | | TNFalpha + | |
| | | IL-1beta | |
| Primary Th1 rest | 14.4 | Bronchial | 16.2 |
| | | epithelium | |
| | | TNFalpha + | |
| | | IL1beta | |
| Primary Th2 | 28.5 | Small airway | 13.6 |
| rest | | epithelium none | |
| Primary Tr1 | 29.7 | Small airway | 37.4 |
| rest | | epithelium | |
| | | TNFalpha + | |
| | | IL-1beta | |
| CD45RA CD4 | 8.2 | Coronery artery | 41.5 |
| lymphocyte act | | SMC rest | |
| CD45RO CD4 | 14.3 | Coronery artery | 50.3 |
| lymphocyte act | | SMC TNFalpha + | |
| | | IL-1beta | |
| CD8 lymphocyte | 17.2 | Astrocytes rest | 45.1 |
| act | | | |
| Secondary CD8 | 13.4 | Astrocytes | 59.0 |
| lymphocyte rest | | TNFalpha + | |
| | | IL-1beta | |
| Secondary CD8 | 34.4 | KU-812 (Basophil) | 19.6 |
| lymphocyte act | | rest | |
| CD4 lymphocyte | 0.9 | KU-812 (Basophil) | 24.8 |
| none | | PMA/ionomycin | |
| 2ry Th1/Th2/ | 25.7 | CCD1106 | 34.6 |
| Tr1_anti-CD95 | | (Keratinocytes) | |
| CH11 | | none | |
| LAK cells rest | 12.9 | CCD1106 | 29.5 |
| | | (Keratinocytes) | |
| | | TNFalpha + IL- | |
| | | 1beta | |
| LAK cells IL-2 | 15.6 | Liver cirrhosis | 14.9 |
| LAK cells IL- | 12.4 | NCI-H292 none | 15.8 |
| 2 + IL-12 | | | |
| LAK cells IL- | 14.5 | NCI-H292 IL-4 | 17.9 |
| 2 + IFN gamma | | | |
| LAK cells IL- | 20.9 | NCI-H292 IL-9 | 22.4 |
| 2 + IL-18 | | | |
| LAK cells PMA/ | 5.0 | NCI-H292 IL-13 | 15.4 |
| ionomycin | | | |
| NK Cells IL-2 | 23.8 | NCI-H292 IFN | 14.6 |
| rest | | gamma | |
| Two Way MLR 3 | 20.6 | HPAEC none | 47.0 |
| day | | | |
| Two Way MLR 5 | 11.2 | HPAEC TNF alpha + | 55.1 |
| day | | IL-1 beta | |
| Two Way MLR 7 | 15.1 | Lung fibroblast | 43.2 |
| day | | none | |
| PBMC rest | 9.9 | Lung fibroblast | 44.8 |
| | | TNF alpha + | |
| | | IL-1 beta | |
| PBMC PWM | 10.6 | Lung fibroblast | 41.5 |
| | | IL-4 | |
| PBMC PHA-L | 26.8 | Lung fibroblast | 51.4 |
| | | IL-9 | |
| Ramos (B cell) | 5.6 | Lung fibroblast | 52.1 |
| none | | IL-13 | |
| Ramos (B cell) | 7.5 | Lung fibroblast | 46.7 |
| ionomycin | | IFN gamma | |
| B lymphocytes | 12.2 | Dermal fibroblast | 29.3 |
| PWM | | CCD1070 rest | |
| B lymphocytes | 35.1 | Dermal fibroblast | 47.6 |
| CD40L and IL-4 | | CCD1070 TNF alpha | |
| EOL-1 dbcAMP | 12.8 | Dermal fibroblast | 15.6 |
| | | CCD1070 IL-1 beta | |
| EOL-1 dbcAMP | 10.6 | Dermal fibroblast | 17.7 |
| PMA/ionomycin | | IFN gamma | |
| Dendritic cells | 25.5 | Dermal fibroblast | 28.3 |
| none | | IL-4 | |
| Dendritic cells | 17.4 | Dermal | 18.8 |
| LPS | | Fibroblasts rest | |
| Dendritic cells | 14.8 | Neutrophils TNFa + | 1.3 |
| anti-CD40 | | LPS | |
| Monocytes rest | 35.4 | Neutrophils rest | 3.4 |
| Monocytes LPS | 26.8 | Colon | 6.7 |
| Macrophages | 14.5 | Lung | 15.1 |
| rest | | | |
| Macrophages | 6.7 | Thymus | 12.5 |
| LPS | | | |
| HUVEC none | 40.9 | Kidney | 22.8 |
| HUVEC starved | 47.3 | | |

CNS_neurodegeneration_v1.0 Summary: Ag3845 This panel does not show differential expression of the CG93210-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3845 Highest expression of the CG93210-01 gene is seen in a breast cancer cell line (CT=26.1). In addition, significant levels of expression are seen in a cluster of samples derived from brain, ovarian, breast and lung cancer cell lines. Thus, expression of this gene could be used to differentiate between these samples and other samples on this panel and as a marker to detect the presence of these cancers. This protein contains a domain that is homologous to a ring finger domain that is though to have intrinsic function as a ubiquituin ligase. Ubiquituin ligase activity of BRCA1 is thought to be important in the prevention of breast and ovarian cancers. Therefore, therapeutic modulation of the expression or function of this gene may be effective in the treatment of ovarian, brain, breast and lung cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, Such as obesity and diabetes. In addition, this gene is expressed at much higher levels in fetal liver (CT=29) when compared to expression in adult liver (CT=34). Thus, expression of this gene may be used to differentiate between the fetal and adult source of these tissue.

This molecule is also expressed at moderate levels in the CNS, including the hippocampus, thalainus, substantia nigra and cerebral cortex. Therefore, therapeutic modulation of the expression or function of this gene may be useful in the treatment of neurologic disorders, such as Alzheiiner's disease, Parkinson's disease, schizophrenia, multiple sclerosis, stroke and epilepsy.

Overall, the ubiquitous expression of this gene suggests a wider role for the corresponding protein product in cell function.

References:

Hashizume R, Fukuda M, Maeda T, Nishikawa H, Oyake D, Yabuki Y, Ogata H, Ohta T. The RING heterodimer BRCA1-BARD1 is a ubiquitin ligase inactivated by a breast cancer-derived mutation. J Biol Chem May 4, 2001;276(18):14537-40

BRCA1-BARD1 constitutes a heterodimeric RING finger complex associated through its N-terminal regions. Here we demonstrate that the BRCA1-BARD1 heterodimeric RING finger complex contains significant ubiquitin ligase activity that can be disrupted by a breast cancer-derived RING finger mutation in BRCA1. Whereas individually BRCA1 and BARD1 have very low ubiquitin ligase activities in vitro, BRCA1 combined with BARD1 exhibits dramatically higher activity. Bacterially purified RING finger domains comprising residues 1-304 of BRCA1 and residues 25-189 of BARD1 are capable of polymerizing ubiquitin. The steady-state level of transfected BRCA1 in vivo was increased by co-transfection of BARD1, and reciprocally that of transfected BARD1 was increased by BRCA1 in a dose-dependent manner. The breast cancer-derived BARD1-interaction-deficient mutant, BRCA1(C61G), does not exhibit ubiquitin ligase activity in vitro. These results suggest that the BRCA1-BARD1 complex contains a ubiquitin ligase activity that is important in prevention of breast and ovarian cancer development.

PMID: 11278247

Panel 2.1 Summary: Ag3845 Highest expression of the CG93210-01 gene is seen in normal kidney (CT=30.3). There is also higher expression in a kidney tumor when compared to the corresponding matched normal tissue. Overall, this gene is widely expressed in this panel, consistent with expression in the previous panels. Thus, expression of this gene could be used to differentiate these samples from other samples on this panel. Furthermore, therapeutic modulation of the expression or function of this gene may be effective in the treatment of kidney cancer.

Panel 4.1D Summary: Ag3845 This gene is expressed in most of the samples on this panel, with highest expression of the CG93210-10 gene in untreated lung microvascular endothelial cells. This gene is also expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

B. NOV4 (CG93187-01): Protocadherin Alpha C2 Short Form Protein

Expression of gene CG93187-01 was assessed using the primer-probe set Ag3844, described in Table BA. Results of the RTQ-PCR runs are shown in Tables BB, BC and BD.

TABLE BA

Probe Name Ag3844

| | | | |
|---|---|---|---|
| Primers | Sequences | Length | Start Position |

| | | | | |
|---|---|---|---|---|
| Forward | 5′-aagtcctgtctcaggcagaata-3′ | (Seq ID NO:117) | 22 | 2294 |
| Probe | TET-5′-tattccaagcaccttccctgggatct-3′-TAMRA | (Seq ID NO:118) | 26 | 2324 |
| Reverse | 5′-ctgtcccactggttctcagtat-3′ | (Seq ID NO:119) | 22 | 2357 |

TABLE BB
CNS_neurodegeneration_v1.0

| | Rel. Exp. | | Rel. Exp. |
|---|---|---|---|
| | (%) Ag3844, | | (%) Ag3844, |
| | Run | | Run |
| Tissue Name | 206878153 | Tissue Name | 206878153 |
| AD 1 Hippo | 3.8 | Control (Path) 3 | 9.2 |
| | | Temporal Ctx | |
| AD 2 Hippo | 17.7 | Control (Path) 4 | 22.5 |
| | | Temporal Ctx | |
| AD 3 Hippo | 4.2 | AD 1 Occipital | 3.1 |
| | | Ctx | |
| AD 4 Hippo | 8.2 | AD 2 Occipital | 0.0 |
| | | Ctx (Missing) | |
| AD 5 Hippo | 100.0 | AD 3 Occipital | 1.7 |
| | | Ctx | |
| AD 6 Hippo | 23.8 | AD 4 Occipital | 10.7 |
| | | Ctx | |
| Control 2 Hippo | 11.7 | AD 5 Occipital | 5.3 |
| | | Ctx | |
| Control 4 Hippo | 5.8 | AD 6 Occipital | 3.1 |
| | | Ctx | |
| Control (Path) | 3.2 | Control 1 | 0.0 |
| 3 Hippo | | Occipital Ctx | |
| AD 1 Temporal | 2.6 | Control 2 | 12.7 |
| Ctx | | Occipital Ctx | |
| AD 2 Temporal | 6.7 | Control 3 | 26.8 |
| Ctx | | Occipital Ctx | |
| AD 3 Temporal | 0.0 | Control 4 | 2.9 |
| Ctx | | Occipital Ctx | |
| AD 4 Temporal | 30.8 | Control (Path) 1 | 92.0 |
| Ctx | | Occipital Ctx | |
| AD 5 Inf Temporal | 84.1 | Control (Path) 2 | 23.2 |
| Ctx | | Occipital Ctx | |
| AD 5 Sup Temporal | 25.2 | Control (Path) 3 | 2.5 |
| Ctx | | Occipital Ctx | |
| AD 6 Inf Temporal | 12.0 | Control (Path) 4 | 16.2 |
| Ctx | | Occipital Ctx | |
| AD 6 Sup Temporal | 9.7 | Control 1 | 4.9 |
| Ctx | | Parietal Ctx | |
| Control 1 | 3.2 | Control 2 | 18.6 |
| Temporal Ctx | | Parietal Ctx | |
| Control 2 | 19.3 | Control 3 | 12.8 |
| Temporal Ctx | | Parietal Ctx | |
| Control 3 | 23.2 | Control (Path) 1 | 49.3 |
| Temporal Ctx | | Parietal Ctx | |
| Control 3 | 4.7 | Control (Path) 2 | 14.7 |
| Temporal Ctx | | Parietal Ctx | |
| Control (Path) 1 | 40.1 | Control (Path) 3 | 9.4 |
| Temporal Ctx | | Parietal Ctx | |
| Control (Path) 2 | 49.3 | Control (Path) 4 | 54.7 |
| Temporal Ctx | | Parietal Ctx | |

TABLE BC
Panel 2.1

| | Rel. Exp. | | Rel. Exp. |
|---|---|---|---|
| | (%) Ag3844, | | (%) Ag3844, |
| | Run | | Run |
| Tissue Name | 170686164 | Tissue Name | 170686164 |
| Normal Colon | 0.0 | Kidney Cancer | 11.1 |
| | | 9010320 | |
| Colon cancer | 0.0 | Kidney margin | 0.0 |
| (OD06064) | | 9010321 | |
| Colon cancer | 7.4 | Kidney Cancer | 0.0 |
| margin (OD06064) | | 8120607 | |
| Colon cancer | 0.0 | Kidney margin | 12.2 |
| (OD06159) | | 8120608 | |
| Colon cancer | 12.9 | Normal Uterus | 39.2 |
| margin (OD06159) | | | |
| Colon cancer | 9.7 | Uterus Cancer | 36.6 |
| (OD06298-08) | | | |
| Colon cancer | 0.0 | Normal Thyroid | 0.0 |
| margin (OD06298- | | | |
| 018) | | | |
| Colon Cancer Gr.2 | 5.8 | Thyroid Cancer | 25.2 |
| ascend colon | | | |
| (ODO3921) | | | |
| Colon Cancer | 13.3 | Thyroid Cancer | 0.0 |
| margin (ODO3921) | | A302152 | |
| Colon cancer | 6.9 | Thyroid margin | 31.9 |
| metastasis | | A302153 | |
| (OD06104) | | | |
| Lung margin | 78.5 | Normal Breast | 64.6 |
| (OD06104) | | | |
| Colon mets to | 0.0 | Breast Cancer | 0.0 |
| lung (OD04451-01) | | | |
| Lung margin | 19.6 | Breast Cancer | 0.0 |
| (OD04451-02) | | | |
| Normal Prostate | 17.1 | Breast Cancer | 0.0 |
| | | (OD04590-01) | |
| Prostate Cancer | 15.9 | Breast Cancer | 32.8 |
| (OD04410) | | Mets (OD04590-03) | |
| Prostate margin | 0.0 | Breast Cancer | 47.3 |
| (OD04410) | | Metastasis | |
| Normal Lung | 92.7 | Breast Cancer | 21.8 |
| Invasive poor | 12.0 | Breast Cancer | 0.0 |
| diff. lung adeno 1 | | 9100266 | |
| (ODO4945-01) | | | |
| Lung margin | 79.0 | Breast margin | 0.0 |
| (ODO4945-03) | | 9100265 | |
| Lung Malignant | 10.2 | Breast Cancer | 0.0 |
| Cancer (OD03126) | | A209073 | |
| Lung margin | 31.4 | Breast margin | 21.5 |
| (OD03126) | | A2090734 | |
| Lung Cancer | 18.8 | Normal Liver | 43.2 |
| (OD05014A) | | | |
| Lung margin | 22.8 | Liver Cancer | 0.0 |
| (OD05014B) | | 1026 | |
| Lung Cancer | 20.7 | Liver Cancer | 25.3 |
| (OD04237-01) | | 1025 | |
| Lung margin | 8.6 | Liver Cancer | 0.0 |
| (OD04237-02) | | 6004-T | |
| Ocular Mel Met to | 0.0 | Liver Tissue | 0.0 |
| Liver (ODO4310) | | 6004-N | |
| Liver margin | 12.9 | Liver Cancer | 0.0 |
| (ODO4310) | | 6005-T | |
| Melanoma Mets to | 30.4 | Liver Tissue | 0.0 |
| Lung (OD04321) | | 6005-N | |
| Lung margin | 100.0 | Liver Cancer | 19.6 |
| (OD04321) | | | |
| Normal Kidney | 14.3 | Normal Bladder | 15.7 |
| Kidney Ca, | 12.6 | Bladder Cancer | 0.0 |
| Nuclear grade 2 | | | |
| (OD04338) | | | |
| Kidney margin | 15.3 | Bladder Cancer | 0.0 |
| (OD04338) | | | |
| Kidney Ca Nuclear | 16.8 | Normal Ovary | 0.0 |
| grade ½ | | | |
| (OD04339) | | | |
| Kidney margin | 0.0 | Ovarian Cancer | 0.0 |
| (OD04339) | | | |
| Kidney Ca, Clear | 26.4 | Ovarian cancer | 0.0 |
| cell type | | (OD06145) | |
| (OD04340) | | | |
| Kidney margin | 9.6 | Ovarian cancer | 16.8 |
| (OD04340) | | margin (OD06145) | |
| Kidney Ca, | 8.8 | Normal Stomach | 35.6 |
| Nuclear grade 3 | | | |
| (OD04348) | | | |
| Kidney margin | 20.0 | Gastric Cancer | 0.0 |
| (OD04348) | | 9060397 | |
| Kidney Cancer | 29.7 | Stomach margin | 0.0 |
| (OD04450-01) | | 9060396 | |
| Kidney margin | 0.0 | Gastric Cancer | 9.7 |
| (OD04450-03) | | 9060395 | |
| Kidney Cancer | 0.0 | Stomach margin | 23.2 |
| 8120613 | | 9060394 | |
| Kidney margin | 0.0 | Gastric Cancer | 19.9 |
| 8120614 | | 064005 | |

TABLE BD
Panel 4.1D

| | Rel. Exp. | | Rel. Exp. |
|---|---|---|---|
| | (%) Ag3844, | | (%) Ag3844, |
| | Run | | Run |
| Tissue Name | 169960434 | Tissue Name | 169960434 |
| Secondary Th1 | 30.8 | HUVEC IL-1beta | 3.5 |
| act | | | |
| Secondary Th2 | 94.0 | HUVEC IFN gamma | 19.8 |
| act | | | |
| Secondary Tr1 | 64.6 | HUVEC TNF alpha + | 15.3 |
| act | | IFN gamma | |
| Secondary Th1 | 35.6 | HUVEC TNF alpha + | 2.8 |
| rest | | IL4 | |
| Secondary Th2 | 66.0 | HUVEC IL-11 | 3.4 |
| rest | | | |
| Secondary Tr1 | 67.8 | Lung | 14.9 |
| rest | | Microvascular EC | |
| | | none | |
| Primary Th1 act | 18.7 | Lung | 20.7 |
| | | Microvascular EC | |
| | | TNFalpha + IL- | |
| | | 1beta | |
| Primary Th2 act | 25.9 | Microvascular | 4.0 |
| | | Dermal EC none | |
| Primary Tr1 act | 28.5 | Microsvasular | 4.0 |
| | | Dermal EC | |
| | | TNFalpha + | |
| | | IL-1beta | |
| Primary Th1 rest | 68.3 | Bronchial | 0.0 |
| | | epithelium | |
| | | TNFalpha + | |
| | | IL1beta | |
| Primary Th2 rest | 89.5 | Small airway | 0.0 |
| | | epithelium none | |
| Primary Tr1 rest | 71.2 | Small airway | 0.6 |
| | | epithelium | |
| | | TNFalpha + | |
| | | IL-1beta | |
| CD45RA CD4 | 27.4 | Coronery artery | 1.9 |
| lymphocyte act | | SMC rest | |
| CD45RO CD4 | 50.3 | Coronery artery | 0.9 |
| lymphocyte act | | SMC TNFalpha + | |
| | | IL-1beta | |
| CD8 lymphocyte | 35.4 | Astrocytes rest | 3.8 |
| act | | | |
| Secondary CD8 | 29.7 | Astrocytes | 0.5 |
| lymphocyte rest | | TNFalpha + | |
| | | IL-1beta | |
| Secondary CD8 | 30.4 | KU-812 (Basophil) | 0.6 |
| lymphocyte act | | rest | |
| CD4 lymphocyte | 15.5 | KU-812 (Basophil) | 3.8 |
| none | | PMA/ionomycin | |
| 2ry Th1/Th2/Tr1_{—} | 100.0 | CCD1106 | 2.2 |
| anti-CD95 CH11 | | (Keratinocytes) | |
| | | none | |
| LAK cells rest | 34.2 | CCD1106 | 0.7 |
| | | (Keratinocytes) | |
| | | TNFalpha + | |
| | | IL-1beta | |
| LAK cells IL-2 | 60.7 | Liver cirrhosis | 2.5 |
| LAK cells IL-2 + | 43.8 | NCI-H292 none | 10.7 |
| IL-12 | | | |
| LAK cells IL-2 + | 79.0 | NCI-H292 IL-4 | 6.5 |
| IFN gamma | | | |
| LAK cells IL-2 + | 66.0 | NCI-H292 IL-9 | 8.2 |
| IL-18 | | | |
| LAK cells | 15.9 | NCI-H292 IL-13 | 9.7 |
| PMA/ionomycin | | | |
| NK Cells IL-2 | 61.6 | NCI-H292 IFN | 7.2 |
| rest | | gamma | |
| Two Way MLR 3 | 68.3 | HPAEC none | 3.5 |
| day | | | |
| Two Way MLR 5 | 26.4 | HPAEC TNF alpha + | 21.9 |
| day | | IL-1 beta | |
| Two Way MLR 7 | 17.2 | Lung fibroblast | 2.2 |
| day | | none | |
| PBMC rest | 4.4 | Lung fibroblast | 0.7 |
| | | TNF alpha + | |
| | | IL-1 beta | |
| PBMC PWM | 29.7 | Lung fibroblast | 2.2 |
| | | IL-4 | |
| PBMC PHA-L | 16.3 | Lung fibroblast | 8.2 |
| | | IL-9 | |
| Ramos (B cell) | 23.2 | Lung fibroblast | 0.0 |
| none | | IL-13 | |
| Ramos (B cell) | 8.4 | Lung fibroblast | 2.1 |
| ionomycin | | IFN gamma | |
| B lymphocytes | 10.5 | Dermal fibroblast | 9.0 |
| PWM | | CCD1070 rest | |
| B lymphocytes | 30.8 | Dermal fibroblast | 60.7 |
| CD40L and IL-4 | | CCD1070 TNF alpha | |
| EOL-1 dbcAMP | 0.0 | Dermal fibroblast | 5.1 |
| | | CCD1070 IL-1 beta | |
| EOL-1 dbcAMP | 0.0 | Dermal fibroblast | 10.6 |
| PMA/ionomycin | | IFN gamma | |
| Dendritic cells | 17.2 | Dermal fibroblast | 9.0 |
| none | | IL-4 | |
| Dendritic cells | 4.9 | Dermal | 7.3 |
| LPS | | Fibroblasts rest | |
| Dendritic cells | 3.2 | Neutrophils | 0.0 |
| anti-CD40 | | TNFa + LPS | |
| Monocytes rest | 10.4 | Neutrophils rest | 6.2 |
| Monocytes LPS | 10.5 | Colon | 5.8 |
| Macrophages rest | 12.8 | Lung | 3.5 |
| Macrophages LPS | 7.9 | Thymus | 41.5 |
| HUVEC none | 5.2 | Kidney | 1.9 |
| HUVEC starved | 4.4 | | |

CNS_neurodegeneration_v1.0 Summary: Ag3844 The CG93187-01 gene, a protocadherin homolog, is detected at low levels in the CNS, with highest expression in the hippocampus of an Alzheimer's patient. While this gene shows no differential expression between the brains of Alzheimer's patients and controls, this expression profile suggests a role for this gene in the CNS. The cadherins have been shown to be critical for CNS development, specifically for the guidance of axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Since protocadherins play an important role in synaptogenesis this gene product may also be involved in depression, schizophrenia, which also involve synaptogeneisis.

References:

Hilschmann N, Barnikol H U, Barnikol-Watanabe S, Gotz H, Kratzin H, Thinnes F P. The immunoglobulin-like genetic predetermination of the brain: the protocadherins, blueprint of the neuronal network. Naturwissenschaften 2001 January;88(1):2-12

The morphogenesis of the brain is governed by synaptogenesis. Synaptogenesis in turn is determined by cell adhesion molecules, which bridge the synaptic cleft and, by homophilic contact, decide which neurons are connected and which are not. Because of their enormous diversification in specificities, protocadherins (pcdh alpha, pcdh beta, pcdh gamma), a new class of cadherins, play a decisive role. Surprisingly, the genetic control of the protocadherins is very similar to that of the immunoglobulins. There are three sets of variable (V) genes followed by a corresponding constant (C) gene. Applying the rules of the immunoglobulin genes to the protocadherin genes leads, despite of this similarity, to quite different results in the central nervous system. The lymphocyte expresses one single receptor molecule specifically directed against an outside stimulus. In contrast, there are three specific recognition sites in each neuron, each expressing a different protocadherin. In this way, 4,950 different neurons arising from one stem cell form a neuronal network, in which homophilic contacts can be formed in 52 layers, permitting an enormous number of different connections and restraints between neurons. This network is one module of the central computer of the brain. Since the V-genes are generated during evolution and V-gene translocation during embryogenesis, outside stimuli have no influence on this network. The network is an inborn property of the protocadherin genes. Every circuit produced, as well as learning and memory, has to be based on this genetically predetermined network. This network is so universal that it can cope with everything, even the unexpected. In this respect the neuronal network resembles the recognition sites of the immunoglobulins.

General_screening_panel_v1.4 Summary: Ag3844 Results from one experiment with the CG93187-01 gene are not included. The amp plot indicates that there were experimental difficulties with this run.

Panel 2.1 Summary: Ag3844 Significant expression of the CG93187-01 gene is restricted to the lung in this panel (CTs=34.5-35). Thus, expression of this gene could be used to differentiate between lung derived tissue and other samples on this panel and as a marker to detect the presence of lung cancer. Furthermore, therapeutic modulation of the expression or function of this gene may be effective in the treatment of lung cancer.

Panel 4.1D Summary: Ag3844 Highest expression of the CG93187-01 gene, a protocadherin alpha homolog, is seen in secondary Th1/TH2/Tr1 cells treated with anti-CD95 (CT=30.5). Overall, expression appears to be higher in hematopoietically derived samples when compared to expression in fibroblasts and endothelial cells. Detection in LAK cells suggests that modulation of the function of this gene product may also lead to improvement of symptoms associated with tumor immunology and tumor cell clearance, as well as removal of virally and bacterial infected cells. In addition, the gene product could also potentially be used therapeutically in the treatment of asthma, emphysema, IBD, lupus or arthritis and in other diseases in which T cells and B cells are activated.

C. NOV5 (CG95083-01): Novel Nuclear Protein

Expression of gene CG95083-01 was assessed using the primer-probe set Ag3918, described in Table CA. Results of the RTQ-PCR runs are shown in Tables CB, CC and CD.

TABLE CA

Probe Name Ag3918

| | | | |
|---|---|---|---|
| Primers | Sequences | Length | Start Position |

| | | | | |
|---|---|---|---|---|
| Forward | 5′-aagctctggtttgtcatcaaag-3′ | (Seq ID NO:120) | 22 | 2086 |
| Probe | TET-5′-tctctacacctacatggccagtgagg-3′-TAMRA | (Seq ID NO:121) | 26 | 2115 |
| Reverse | 5′-atactctccaaqgccactttgt-3′ | (Seq ID NO: 122) | 22 | 2141 |

TABLE CB
CNS_neurodegeneration_v1.0

| | Rel. Exp. | | Rel. Exp. |
|---|---|---|---|
| | (%) Ag3918, | | (%) Ag3918, |
| | Run | | Run |
| Tissue Name | 212248493 | Tissue Name | 212248493 |
| AD 1 Hippo | 21.8 | Control (Path) | 28.3 |
| | | 3 Temporal Ctx | |
| AD 2 Hippo | 21.8 | Control (Path) | 44.4 |
| | | 4 Temporal Ctx | |
| AD 3 Hippo | 24.7 | AD 1 Occipital | 27.7 |
| | | Ctx | |
| AD 4 Hippo | 11.7 | AD 2 Occipital | 0.0 |
| | | Ctx (Missing) | |
| AD 5 hippo | 100.0 | AD 3 Occipital | 36.9 |
| | | Ctx | |
| AD 6 Hippo | 62.4 | AD 4 Occipital | 19.9 |
| | | Ctx | |
| Control 2 Hippo | 22.1 | AD 5 Occipital | 27.0 |
| | | Ctx | |
| Control 4 Hippo | 20.9 | AD 6 Occipital | 55.5 |
| | | Ctx | |
| Control (Path) | 22.8 | Control 1 | 28.5 |
| 3 Hippo | | Occipital Ctx | |
| AD 1 Temporal | 35.8 | Control 2 | 61.6 |
| Ctx | | Occipital Ctx | |
| AD 2 Temporal | 35.6 | Control 3 | 25.3 |
| Ctx | | Occipital Ctx | |
| AD 3 Temporal | 34.2 | Control 4 | 18.0 |
| Ctx | | Occipital Ctx | |
| AD 4 Temporal | 33.4 | Control (Path) 1 | 69.7 |
| Ctx | | Occipital Ctx | |
| AD 5 Inf | 92.7 | Control (Path) 2 | 21.8 |
| Temporal Ctx | | Occipital Ctx | |
| AD 5 Sup | 79.0 | Control (Path) 3 | 34.9 |
| Temporal Ctx | | Occipital Ctx | |
| AD 6 Inf | 71.7 | Control (Path) 4 | 39.0 |
| Temporal Ctx | | Occipital Ctx | |
| AD 6 Sup | 85.9 | Control 1 | 18.0 |
| Temporal Ctx | | Parietal Ctx | |
| Control 1 | 19.5 | Control 2 | 42.9 |
| Temporal Ctx | | Parietal Ctx | |
| Control 2 | 22.8 | Control 3 | 19.6 |
| Temporal Ctx | | Parietal Ctx | |
| Control 3 | 28.5 | Control (Path) 1 | 66.9 |
| Temporal Ctx | | Parietal Ctx | |
| Control 4 | 11.2 | Control (Path) 2 | 31.6 |
| Temporal Ctx | | Parietal Ctx | |
| Control (Path) | 57.0 | Control (Path) 3 | 28.3 |
| 1 Temporal Ctx | | Parietal Ctx | |
| Control (Path) | 22.1 | Control (Path) 4 | 81.8 |
| 2 Temporal Ctx | | Parietal Ctx | |

TABLE CC
General_screening_panel_v1.4

| | Rel. Exp. | | Rel. Exp. |
|---|---|---|---|
| | (%) Ag3918, | | (%) Ag3918, |
| | Run | | Run |
| Tissue Name | 219824451 | Tissue Name | 219824451 |
| Adipose | 15.2 | Renal ca. | 0.0 |
| | | TK-10 | |
| Melanoma* | 0.0 | Bladder | 4.2 |
| Hs688(A).T | | | |
| Melanoma* | 0.0 | Gastric ca. | 0.2 |
| Hs688(B).T | | (liver met.) | |
| | | NCI-N87 | |
| Melanoma* M14 | 0.0 | Gastric ca. | 0.0 |
| | | KATO III | |
| Melanoma* | 0.0 | Colon ca. | 0.0 |
| LOXIMVI | | SW-948 | |
| Melanoma* | 0.0 | Colon ca. | 68.8 |
| SK-MEL-5 | | SW480 | |
| Squamous cell | 0.1 | Colon ca.* | 29.9 |
| carcinoma SCC-4 | | (SW480 met) | |
| | | SW620 | |
| Testis Pool | 6.6 | Colon ca. | 0.0 |
| | | HT29 | |
| Prostate ca.* | 0.1 | Colon ca. | 0.0 |
| (bone met) PC-3 | | HCT-116 | |
| Prostate Pool | 3.3 | Colon ca. | 0.2 |
| | | CaCo-2 | |
| Placenta | 14.3 | Colon cancer | 8.3 |
| | | tissue | |
| Uterus Pool | 8.4 | Colon ca. | 0.0 |
| | | SW1116 | |
| Ovarian ca. | 0.2 | Colon ca. | 13.7 |
| OVCAR-3 | | Colo-205 | |
| Ovarian ca. | 0.0 | Colon ca. | 0.0 |
| SK-OV-3 | | SW-48 | |
| Ovarian ca. | 0.0 | Colon Pool | 16.0 |
| OVCAR-4 | | | |
| Ovarian ca. | 0.3 | Small Intestine | 7.7 |
| OVCAR-5 | | Pool | |
| Ovarian ca. | 0.0 | Stomach Pool | 6.7 |
| IGROV-1 | | | |
| Ovarian ca. | 0.0 | Bone Marrow | 9.2 |
| OVCAR-8 | | Pool | |
| Ovary | 7.9 | Fetal Heart | 14.1 |
| Breast ca. | 0.5 | Heart Pool | 7.4 |
| MCF-7 | | | |
| Breast ca. | 0.0 | Lymph Node Pool | 16.8 |
| MDA-MB-231 | | | |
| Breast ca. | 0.0 | Fetal Skeletal | 17.8 |
| BT 549 | | Muscle | |
| Breast ca. | 0.9 | Skeletal Muscle | 4.9 |
| T47D | | Pool | |
| Breast ca. | 0.0 | Spleen Pool | 20.4 |
| MDA-N | | | |
| Breast Pool | 13.5 | Thymus Pool | 5.7 |
| Trachea | 8.3 | CNS cancer | 0.1 |
| | | (glio/astro) | |
| | | U87-MG | |
| Lung | 3.7 | CNS cancer | 0.0 |
| | | (glio/astro) | |
| | | U-118-MG | |
| Fetal Lung | 100.0 | CNS cancer | 0.1 |
| | | (neuro; met) | |
| | | SK-N-AS | |
| Lung ca NCI-N417 | 0.0 | CNS cancer | 0.0 |
| | | (astro) SF-539 | |
| Lung ca. LX-1 | 69.7 | CNS cancer | 0.0 |
| | | (astro) SNB-75 | |
| Lung ca. NCI- | 0.0 | CNS cancer | 0.0 |
| H146 | | (glio) SNB-19 | |
| Lung ca. SHP-77 | 0.0 | CNS cancer | 0.0 |
| | | (glio) SF-295 | |
| Lung ca. A549 | 0.4 | Brain | 1.1 |
| | | (Amygdala) Pool | |
| Lung ca. NCI- | 0.4 | Brain | 4.8 |
| H526 | | (cerebellum) | |
| Lung ca. NCI-H23 | 0.1 | Brain (fetal) | 4.3 |
| Lung ca. NCI- | 0.3 | Brain | 2.0 |
| H460 | | (Hippocampus) | |
| | | Pool | |
| Lung ca. HOP-62 | 0.0 | Cerebral Cortex | 2.1 |
| | | Pool | |
| Lung ca. NCI- | 0.1 | Brain | 1.2 |
| H522 | | (Substantia | |
| | | nigra) Pool | |
| Liver | 1.5 | Brain | 1.8 |
| | | (Thalamus) Pool | |
| Fetal Liver | 7.7 | Brain (whole) | 3.5 |
| Liver ca. HepG2 | 0.0 | Spinal Cord | 1.5 |
| | | Pool | |
| Kidney Pool | 18.8 | Adrenal Gland | 7.7 |
| Fetal Kidney | 14.8 | Pituitary gland | 1.4 |
| | | Pool | |
| Renal ca. 786-0 | 0.0 | Salivary Gland | 1.7 |
| Renal ca. A498 | 0.0 | Thyroid | 3.4 |
| | | (female) | |
| Renal ca. ACHN | 0.0 | Pancreatic ca. | 1.5 |
| | | CAPAN2 | |
| Renal ca. UO-31 | 0.1 | Pancreas Pool | 12.4 |

TABLE CD
Panel 4.1D

| | Rel. Exp. | | Rel. Exp. |
|---|---|---|---|
| | (%) Ag3918, | | (%) Ag3918, |
| | Run | | Run |
| Tissue Name | 170128260 | Tissue Name | 170128260 |
| Secondary Th1 | 0.0 | HUVEC IL-1beta | 58.6 |
| act | | | |
| Secondary Th2 | 0.0 | HUVEC IFN gamma | 24.0 |
| act | | | |
| Secondary Tr1 | 0.0 | HUVEC TNF alpha + | 35.4 |
| act | | IFN gamma | |
| Secondary Th1 | 0.0 | HUVEC TNF alpha + | 32.3 |
| rest | | IL4 | |
| Secondary Th2 | 0.0 | HUVEC IL-11 | 44.1 |
| rest | | | |
| Secondary Tr1 | 0.0 | Lung Microvascular | 100.0 |
| rest | | EC none | |
| Primary Th1 act | 0.0 | Lung Microvascular | 34.9 |
| | | EC TNFalpha + | |
| | | IL-1beta | |
| Primary Th2 act | 0.0 | Microvascular | 72.2 |
| | | Dermal EC none | |
| Primary Tr1 act | 0.0 | Microsvasular | 33.4 |
| | | Dermal EC | |
| | | TNFalpha + IL- | |
| | | 1beta | |
| Primary Th1 rest | 0.0 | Bronchial | 0.0 |
| | | epithelium | |
| | | TNFalpha + IL1beta | |
| Primary Th2 rest | 0.0 | Small airway | 0.2 |
| | | epithelium none | |
| Primary Tr1 rest | 0.0 | Small airway | 0.0 |
| | | epithelium | |
| | | TNFalpha + IL- | |
| | | 1beta | |
| CD45RA CD4 | 0.0 | Coronery artery | 0.9 |
| lymphocyte act | | SMC rest | |
| CD45RO CD4 | 0.1 | Coronery artery | 1.5 |
| lymphocyte act | | SMC TNFalpha + IL- | |
| | | 1beta | |
| CD8 lymphocyte | 0.0 | Astrocytes rest | 0.0 |
| act | | | |
| Secondary CD8 | 0.0 | Astrocytes | 0.0 |
| lymphocyte rest | | TNFalpha + IL- | |
| | | 1beta | |
| Secondary CD8 | 0.0 | KU-812 (Basophil) | 0.0 |
| lymphocyte act | | rest | |
| CD4 lymphocyte | 0.0 | KU-812 (Basophil) | 0.0 |
| none | | PMA/ionomycin | |
| 2ry Th1/Th2/Tr1_{—} | 0.0 | CCD1106 | 0.0 |
| anti-CD95 CH11 | | (Keratinocytes) | |
| | | none | |
| LAK cells rest | 0.8 | CCD1106 | 0.0 |
| | | (Keratinocytes) | |
| | | TNFalpha + IL- | |
| | | 1beta | |
| LAK cells IL-2 | 0.0 | Liver cirrhosis | 4.2 |
| LAK cells IL- | 0.0 | NCI-H292 none | 0.0 |
| 2 + IL-12 | | | |
| LAK cells IL- | 0.0 | NCI-H292 IL-4 | 0.0 |
| 2 + IFN gamma | | | |
| LAK cells IL- | 0.0 | NCI-H292 IL-9 | 0.0 |
| 2 + IL-18 | | | |
| LAK cells | 0.2 | NCI-H292 IL-13 | 0.2 |
| PMA/ionomycin | | | |
| NK Cells IL-2 | 0.0 | NCI-H292 IFN | 0.0 |
| rest | | gamma | |
| Two Way MLR 3 | 0.0 | HPAEC none | 55.9 |
| day | | | |
| Two Way MLR 5 | 0.0 | HPAEC TNF alpha + | 69.3 |
| day | | IL-1 beta | |
| Two Way MLR 7 | 0.0 | Lung fibroblast | 0.1 |
| day | | none | |
| PBMC rest | 0.1 | Lung fibroblast | 0.1 |
| | | TNF alpha + IL-1 | |
| | | beta | |
| PBMC PWM | 0.0 | Lung fibroblast | 0.1 |
| | | IL-4 | |
| PBMC PHA-L | 0.0 | Lung fibroblast | 0.1 |
| | | IL-9 | |
| Ramos (B cell) | 0.0 | Lung fibroblast | 0.0 |
| none | | IL-13 | |
| Ramos (B cell) | 0.0 | Lung fibroblast | 0.1 |
| ionomycin | | IFN gamma | |
| B lymphocytes | 0.0 | Dermal fibroblast | 0.0 |
| PWM | | CCD1070 rest | |
| B lymphocytes | 0.0 | Dermal fibroblast | 0.0 |
| CD40L and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.1 | Dermal fibroblast | 0.5 |
| PMA/ionomycin | | IFN gamma | |
| Dendritic cells | 9.0 | Dermal fibroblast | 1.5 |
| none | | IL-4 | |
| Dendritic cells | 1.4 | Dermal | 0.3 |
| LPS | | Fibroblasts rest | |
| Dendritic cells | 18.7 | Neutrophils | 0.0 |
| anti-CD40 | | TNFa + LPS | |
| Monocytes rest | 0.0 | Neutrophils rest | 0.0 |
| Monocytes LPS | 0.0 | Colon | 2.1 |
| Macrophages | 4.2 | Lung | 24.8 |
| rest | | | |
| Macrophages | 0.4 | Thymus | 4.0 |
| LPS | | | |
| HUVEC none | 46.3 | Kidney | 23.3 |
| HUVEC starved | 62.9 | | |

CNS_neurodegeneration_v1.0 Summary: Ag3918 This panel does not show differential expression of the CG95083-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3918 Highest expression of the CG95083-01 gene is seen in the fetal lung (CT=26.5). In addition, this gene is expressed at much higher levels in fetal lung when compared to expression in the adult counterpart (CT=31.3). Thus, expression of this gene may be used to differentiate between the fetal and adult source of this tissue.

In addition, significant levels of expression are seen in a samples derived from colon and lung cancer cell lines. Thus, expression of this gene could be used to differentiate between these samples and other samples on this panel and as a marker to detect the presence of these cancers. Furthermore, therapeutic modulation of the expression or function of this gene may be effective in the treatment of colon and lung cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at low levels in the CNS, including the hippocampus, amygdala, cerebellum, thalamus, substantia nigra and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3918 Highest expression of the CG95083-01 gene is seen in untreated lung microvascular endothelial cells (CT=26.6). This gene is expressed consistently in endothelium samples including HPAEC, HUVEC and lung and dermal microvascular EC.

Therefore, expression of this gene could be used as a marker of endothelial cells. Furthermore, therapies designed with the protein encoded by this transcript could be important in the regulating endothelium function including leukocyte extravasation, a major component of inflammation during asthma, IBD, and psoriasis.

D. NOV6 (CG94989-01): Novel Secretory Protein

Expression of gene CG94989-01 was assessed using the primer-probe set Ag3980, described in Table DA. Results of the RTQ-PCR runs are shown in Tables DB, DC, DD and DE.

TABLE DA

Probe Name Ag3980

| | | | |
|---|---|---|---|
| Primers | Sequences | Length | Start |

| | | | | |
|---|---|---|---|---|
| Forward | 5′-ctaccagacacctgtgcaagac-3′ | (Seq ID NO:123) | 22 | 974 |
| Probe | TET-5′-caggaggttcctgcctaacaaatcca-3′-TAMRA | (Seq ID NO: | 26 | 1010 |
| Reverse | 5′-cttgacaaagccacaaacactt-3′ | (Seq ID NO:125) | 22 | 1036 |

TABLE DB
CNS_neurodegeneration_v1.0

| | Rel. Exp. | | Rel. Exp. |
|---|---|---|---|
| | (%) Ag3980, | | (%) Ag3980, |
| | Run | | Run |
| Tissue Name | 206880051 | Tissue Name | 206880051 |
| AD 1 Hippo | 27.9 | Control (Path) 3 | 13.8 |
| | | Temporal Ctx | |
| AD 2 Hippo | 29.9 | Control (Path) 4 | 25.5 |
| | | Temporal Ctx | |
| AD 3 Hippo | 25.9 | AD 1 Occipital Ctx | 28.3 |
| AD 4 Hippo | 9.3 | AD 2 Occipital Ctx | 0.0 |
| | | (Missing) | |
| AD 5 hippo | 84.1 | AD 3 Occipital Ctx | 25.3 |
| AD 6 Hippo | 45.4 | AD 4 Occipital Ctx | 18.7 |
| Control 2 Hippo | 32.3 | AD 5 Occipital Ctx | 20.6 |
| Control 4 Hippo | 18.2 | AD 6 Occipital Ctx | 31.2 |
| Control (Path) | 13.4 | Control 1 | 9.0 |
| 3 Hippo | | Occipital Ctx | |
| AD 1 Temporal | 37.6 | Control 2 | 49.3 |
| Ctx | | Occipital Ctx | |
| AD 2 Temporal | 30.8 | Control 3 | 22.8 |
| Ctx | | Occipital Ctx | |
| AD 3 Temporal | 19.8 | Control 4 | 18.8 |
| Ctx | | Occipital Ctx | |
| AD 4 Temporal | 18.8 | Control (Path) 1 | 74.7 |
| Ctx | | Occipital Ctx | |
| AD 5 Inf | 100.0 | Control (Path) 2 | 8.8 |
| Temporal Ctx | | Occipital Ctx | |
| AD 5 SupTemporal | 73.7 | Control (Path) 3 | 12.6 |
| Ctx | | Occipital Ctx | |
| AD 6 Inf | 42.0 | Control (Path) 4 | 19.5 |
| Temporal Ctx | | Occipital Ctx | |
| AD 6 Sup | 37.9 | Control 1 Parietal | 15.2 |
| Temporal Ctx | | Ctx | |
| Control 1 | 17.4 | Control 2 Parietal | 53.6 |
| Temporal Ctx | | Ctx | |
| Control 2 | 30.8 | Control 3 Parietal | 16.4 |
| Temporal Ctx | | Ctx | |
| Control 3 | 14.3 | Control (Path) 1 | 46.3 |
| Temporal Ctx | | Parietal Ctx | |
| Control 4 | 16.5 | Control (Path) 2 | 26.8 |
| Temporal Ctx | | Parietal Ctx | |
| Control (Path) 1 | 46.7 | Control (Path) 3 | 11.8 |
| Temporal Ctx | | Parietal Ctx | |
| Control (Path) 2 | 22.7 | Control (Path) 4 | 36.1 |
| Temporal Ctx | | Parietal Ctx | |

TABLE DC
General_screening_panel_v1.4

| | Rel. Exp. | | Rel. Exp. |
|---|---|---|---|
| | (%) Ag3980, | | (%) Ag3980, |
| | Run | | Run |
| Tissue Name | 217534219 | Tissue Name | 217534219 |
| Adipose | 0.7 | Renal ca. TK-10 | 6.8 |
| Melanoma* | 0.0 | Bladder | 0.6 |
| Hs688(A).T | | | |
| Melanoma* | 0.0 | Gastric ca. (liver | 0.0 |
| Hs688(B).T | | met.) NCI-N87 | |
| Melanoma* M14 | 0.2 | Gastric ca. KATO | 0.0 |
| | | III | |
| Melanoma* | 0.0 | Colon ca. SW-948 | 6.3 |
| LOXIMVI | | | |
| Melanoma* SK- | 6.6 | Colon ca. SW480 | 0.5 |
| MEL-5 | | | |
| Squamous cell | 0.0 | Colon ca.* (SW480 | 0.7 |
| carcinoma SCC-4 | | met) SW620 | |
| Testis Pool | 4.5 | Colon ca. HT29 | 0.0 |
| Prostate ca.* | 60.7 | Colon ca. HCT-116 | 0.0 |
| (bone met) PC-3 | | | |
| Prostate Pool | 0.3 | Colon ca. CaCo-2 | 0.0 |
| Placenta | 5.5 | Colon cancer | 2.6 |
| | | tissue | |
| Uterus Pool | 0.9 | Colon ca. SW1116 | 0.0 |
| Ovarian ca. | 0.0 | Colon ca. Colo- | 0.0 |
| OVCAR-3 | | 205 | |
| Ovarian ca. | 0.0 | Colon ca. SW-48 | 0.0 |
| SK-OV-3 | | | |
| Ovarian ca. | 0.1 | Colon Pool | 1.9 |
| OVCAR-4 | | | |
| Ovarian ca. | 1.3 | Small Intestine | 1.9 |
| OVCAR-5 | | Pool | |
| Ovarian ca. | 7.7 | Stomach Pool | 1.1 |
| IGROV-1 | | | |
| Ovarian ca. | 0.6 | Bone Marrow Pool | 0.8 |
| OVCAR-8 | | | |
| Ovary | 0.4 | Fetal Heart | 1.4 |
| Breast ca. | 4.7 | Heart Pool | 0.9 |
| MCF-7 | | | |
| Breast ca. | 0.0 | Lymph Node Pool | 0.9 |
| MDA-MB-231 | | | |
| Breast ca. BT | 0.0 | Fetal Skeletal | 10.4 |
| 549 | | Muscle | |
| Breast ca. T47D | 1.9 | Skeletal Muscle | 9.5 |
| | | Pool | |
| Breast ca. MDA- | 0.1 | Spleen Pool | 16.2 |
| N | | | |
| Breast Pool | 1.1 | Thymus Pool | 0.8 |
| Trachea | 1.0 | CNS cancer | 0.0 |
| | | (glio/astro) | |
| | | U87-MG | |
| Lung | 0.2 | CNS cancer | 0.0 |
| | | (glio/astro) | |
| | | U-118-MG | |
| Fetal Lung | 3.1 | CNS cancer | 0.0 |
| | | (neuro; met) SK-N- | |
| | | AS | |
| Lung ca. NCI- | 0.7 | CNS cancer (astro) | 0.0 |
| N417 | | SF-539 | |
| Lung ca. LX-1 | 0.1 | CNS cancer (astro) | 0.1 |
| | | SNB-75 | |
| Lung ca. NCI- | 10.7 | CNS cancer (glio) | 5.6 |
| H146 | | SNB-19 | |
| Lung ca. SHP-77 | 0.5 | CNS cancer (glio) | 0.1 |
| | | SF-295 | |
| Lung ca. A549 | 5.0 | Brain (Amygdala) | 14.6 |
| | | Pool | |
| Lung ca. NCI- | 9.3 | Brain (cerebellum) | 100.0 |
| H526 | | | |
| Lung ca. NCI- | 6.5 | Brain (fetal) | 38.2 |
| H23 | | | |
| Lung ca. NCI- | 4.2 | Brain | 11.6 |
| H460 | | (Hippocampus) Pool | |
| Lung ca. HOP-62 | 1.7 | Cerebral Cortex | 12.9 |
| | | Pool | |
| Lung ca. NCI- | 3.0 | Brain (Substantia | 18.4 |
| H522 | | nigra) Pool | |
| Liver | 0.1 | Brain (Thalamus) | 20.4 |
| | | Pool | |
| Fetal Liver | 4.3 | Brain (whole) | 17.1 |
| Liver ca. HepG2 | 0.2 | Spinal Cord Pool | 21.5 |
| Kidney Pool | 3.1 | Adrenal Gland | 5.3 |
| Fetal Kidney | 4.0 | Pituitary gland | 2.0 |
| | | Pool | |
| Renal ca. 786-0 | 1.5 | Salivary Gland | 0.8 |
| Renal ca. A498 | 0.0 | Thyroid (female) | 1.0 |
| Renal ca. ACHN | 12.9 | Pancreatic ca. | 0.1 |
| | | CAPAN2 | |
| Renal ca. UO-31 | 20.6 | Pancreas Pool | 0.8 |

TABLE DD
Panel 2.1

| | Rel. Exp. | | Rel. Exp. |
|---|---|---|---|
| | (%) Ag3980, | | (%) Ag3980, |
| | Run | | Run |
| Tissue Name | 170721076 | Tissue Name | 170721076 |
| Normal Colon | 35.8 | Kidney Cancer | 5.0 |
| | | 9010320 | |
| Colon cancer | 0.0 | Kidney margin | 40.1 |
| (OD06064) | | 9010321 | |
| Colon cancer | 27.9 | Kidney Cancer | 41.8 |
| margin (OD06064) | | 8120607 | |
| Colon cancer | 6.2 | Kidney margin | 28.5 |
| (OD06159) | | 8120608 | |
| Colon cancer | 27.9 | Normal Uterus | 4.9 |
| margin (OD06159) | | | |
| Colon cancer | 14.0 | Uterus Cancer | 0.9 |
| (OD06298-08) | | | |
| Colon cancer | 90.1 | Normal Thyroid | 0.0 |
| margin | | | |
| (OD06298-018) | | | |
| Colon Cancer | 5.6 | Thyroid Cancer | 6.2 |
| Gr.2 ascend | | | |
| colon (ODO3921) | | | |
| Colon Cancer | 9.7 | Thyroid Cancer | 1.3 |
| margin (ODO3921) | | A302152 | |
| Colon cancer | 2.6 | Thyroid margin | 3.7 |
| metastasis | | A302153 | |
| (OD06104) | | | |
| Lung margin | 7.1 | Normal Breast | 6.9 |
| (OD06104) | | | |
| Colon mets to | 0.8 | Breast Cancer | 0.2 |
| lung (OD04451- | | | |
| 01) | | | |
| Lung margin | 1.8 | Breast Cancer | 1.8 |
| (OD04451-02) | | | |
| Normal Prostate | 0.0 | Breast Cancer | 0.6 |
| | | (OD04590-01) | |
| Prostate Cancer | 0.0 | Breast Cancer | 3.4 |
| (OD04410) | | Mets (OD04590-03) | |
| Prostate margin | 0.4 | Breast Cancer | 3.8 |
| (OD04410) | | Metastasis | |
| Normal Lung | 2.3 | Breast Cancer | 0.0 |
| Invasive poor | 0.0 | Breast Cancer | 3.4 |
| diff. lung | | 9100266 | |
| adeno 1 | | | |
| (ODO4945-01) | | | |
| Lung margin | 2.3 | Breast margin | 7.3 |
| (ODO4945-03) | | 9100265 | |
| Lung Malignant | 3.5 | Breast Cancer | 0.0 |
| Cancer (OD03126) | | A209073 | |
| Lung margin | 3.4 | Breast margin | 3.9 |
| (OD03126) | | A2090734 | |
| Lung Cancer | 1.5 | Normal Liver | 1.2 |
| (OD05014A) | | | |
| Lung margin | 0.6 | Liver Cancer 1026 | 5.5 |
| (OD05014B) | | | |
| Lung Cancer | 53.2 | Liver Cancer 1025 | 1.8 |
| (OD04237-01) | | | |
| Lung margin | 4.0 | Liver Cancer | 0.9 |
| (OD04237-02) | | 6004-T | |
| Ocular Mel Met | 3.4 | Liver Tissue | 0.9 |
| to Liver | | 6004-N | |
| (ODO4310) | | | |
| Liver margin | 1.6 | Liver Cancer | 29.7 |
| (ODO4310) | | 6005-T | |
| Melanoma Mets | 0.0 | Liver Tissue | 11.0 |
| to Lung | | 6005-N | |
| (OD04321) | | | |
| Lung margin | 4.9 | Liver Cancer | 1.1 |
| (OD04321) | | | |
| Normal Kidney | 12.2 | Normal Bladder | 0.8 |
| Kidney Ca, | 29.9 | Bladder Cancer | 0.8 |
| Nuclear grade 2 | | | |
| (OD04338) | | | |
| Kidney margin | 17.6 | Bladder Cancer | 1.2 |
| (OD04338) | | | |
| Kidney Ca | 4.9 | Normal Ovary | 6.3 |
| Nuclear grade | | | |
| ½ (OD04339) | | | |
| Kidney margin | 10.3 | Ovarian Cancer | 0.4 |
| (OD04339) | | | |
| Kidney Ca, Clear | 25.0 | Ovarian cancer | 0.0 |
| cell type | | (OD06145) | |
| (OD04340) | | | |
| Kidney margin | 13.5 | Ovarian cancer | 3.1 |
| (OD04340) | | margin (OD06145) | |
| Kidney Ca, | 2.2 | Normal Stomach | 6.2 |
| Nuclear grade | | | |
| 3 (OD04348) | | | |
| Kidney margin | 9.2 | Gastric Cancer | 1.9 |
| (OD04348) | | 9060397 | |
| Kidney Cancer | 100.0 | Stomach margin | 1.3 |
| (OD04450-01) | | 9060396 | |
| Kidney margin | 25.3 | Gastric Cancer | 9.7 |
| (OD04450-03) | | 9060395 | |
| Kidney Cancer | 5.7 | Stomach margin | 3.6 |
| 8120613 | | 9060394 | |
| Kidney margin | 5.6 | Gastric Cancer | 3.4 |
| 8120614 | | 064005 | |

TABLE DE
Panel 4.1D

| | Rel. Exp. | | Rel. Exp. |
|---|---|---|---|
| | (%) Ag3980, | | (%) Ag3980, |
| | Run | | Run |
| Tissue Name | 170729091 | Tissue Name | 170729091 |
| Secondary Th1 | 0.0 | HUVEC IL-1beta | 62.0 |
| act | | | |
| Secondary Th2 | 0.3 | HUVEC IFN gamma | 97.9 |
| act | | | |
| Secondary Tr1 | 0.0 | HUVEC TNF alpha + | 23.3 |
| act | | IFN gamma | |
| Secondary Th1 | 0.3 | HUVEC TNF alpha + | 61.6 |
| rest | | IL4 | |
| Secondary Th2 | 0.0 | HUVEC IL-11 | 82.4 |
| rest | | | |
| Secondary Tr1 | 0.0 | Lung Microvascular | 3.2 |
| rest | | EC none | |
| Primary Th1 act | 0.0 | Lung Microvascular | 0.9 |
| | | EC TNFalpha + IL- | |
| | | 1beta | |
| Primary Th2 act | 0.0 | Microvascular | 11.1 |
| | | Dermal EC none | |
| Primary Tr1 act | 0.0 | Microsvasular | 8.0 |
| | | Dermal EC | |
| | | TNFalpha + IL- | |
| | | 1beta | |
| Primary Th1 | 0.0 | Bronchial | 0.0 |
| rest | | epithelium | |
| | | TNFalpha + | |
| | | IL1beta | |
| Primary Th2 | 0.0 | Small airway | 0.4 |
| rest | | epithelium none | |
| Primary Tr1 | 0.0 | Small airway | 0.0 |
| rest | | epithelium | |
| | | TNFalpha + IL- | |
| | | 1beta | |
| CD45RA CD4 | 1.7 | Coronery artery | 0.7 |
| lymphocyte act | | SMC rest | |
| CD45RO CD4 | 0.0 | Coronery artery | 1.4 |
| lymphocyte act | | SMC TNFalpha + | |
| | | IL-1beta | |
| CD8 lymphocyte | 0.4 | Astrocytes rest | 12.4 |
| act | | | |
| Secondary CD8 | 0.3 | Astrocytes | 4.6 |
| lymphocyte rest | | TNFalpha + IL- | |
| | | 1beta | |
| Secondary CD8 | 0.0 | KU-812 (Basophil) | 0.0 |
| lymphocyte act | | rest | |
| CD4 lymphocyte | 0.0 | KU-812 (Basophil) | 0.7 |
| none | | PMA/ionomycin | |
| 2ry Th1/Th2/ | 0.0 | CCD1106 | 0.0 |
| Tr1_anti-CD95 | | (Keratinocytes) | |
| CH11 | | none | |
| LAK cells rest | 0.3 | CCD1106 | 0.0 |
| | | (Keratinocytes) | |
| | | TNFalpha + IL- | |
| | | 1beta | |
| LAK cells IL-2 | 0.3 | Liver cirrhosis | 1.1 |
| LAK cells IL-2 + | 0.0 | NCI-H292 none | 0.0 |
| IL-12 | | | |
| LAK cells IL-2 + | 0.0 | NCI-H292 IL-4 | 0.0 |
| IFN gamma | | | |
| LAK cells IL-2 + | 0.0 | NCI-H292 IL-9 | 0.0 |
| IL-18 | | | |
| LAK cells | 0.0 | NCI-H292 IL-13 | 0.0 |
| PMA/ionomycin | | | |
| NK Cells IL-2 | 4.3 | NCI-H292 IFN | 1.8 |
| rest | | gamma | |
| Two Way MLR 3 | 0.0 | HPAEC none | 100.0 |
| day | | | |
| Two Way MLR 5 | 0.0 | HPAEC TNF alpha + | 26.8 |
| day | | IL-1 beta | |
| Two Way MLR 7 | 0.3 | Lung fibroblast | 3.7 |
| day | | none | |
| PBMC rest | 0.0 | Lung fibroblast | 0.0 |
| | | TNF alpha + IL- | |
| | | 1 beta | |
| PBMC PWM | 0.0 | Lung fibroblast | 2.0 |
| | | IL-4 | |
| PBMC PHA-L | 0.0 | Lung fibroblast | 0.9 |
| | | IL-9 | |
| Ramos (B cell) | 0.0 | Lung fibroblast | 1.2 |
| none | | IL-13 | |
| Ramos (B cell) | 0.0 | Lung fibroblast | 0.8 |
| ionomycin | | IFN gamma | |
| B lymphocytes | 0.0 | Dermal fibroblast | 1.9 |
| PWM | | CCD1070 rest | |
| B lymphocytes | 0.3 | Dermal fibroblast | 3.4 |
| CD40L and IL-4 | | CCD1070 TNF alpha | |
| EOL-1 dbcAMP | 0.0 | Dermal fibroblast | 3.2 |
| | | CCD1070 IL-1 beta | |
| EOL-1 dbcAMP | 0.0 | Dermal fibroblast | 5.7 |
| PMA/ionomycin | | IFN gamma | |
| Dendritic cells | 0.0 | Dermal fibroblast | 52.5 |
| none | | IL-4 | |
| Dendritic cells | 0.0 | Dermal Fibroblasts | 1.5 |
| LPS | | rest | |
| Dendritic cells | 0.0 | Neutrophils TNFa + | 1.6 |
| anti-CD40 | | LPS | |
| Monocytes rest | 0.0 | Neutrophils rest | 0.0 |
| Monocytes LPS | 0.0 | Colon | 15.7 |
| Macrophages rest | 0.0 | Lung | 70.2 |
| Macrophages LPS | 0.0 | Thymus | 12.3 |
| HUVEC none | 61.1 | Kidney | 26.8 |
| HUVEC starved | 68.8 | | |

CNS_neurodegeneration_v1.0 Summary: Ag3980 This panel does not show differential expression of the CG94989-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3980 Highest expression of the CG94989-01 gene is seen in the cerebellum (CT=25.8). In addition, expression of this gene appears to be highly brain preferential, with high to moderate levels of expression in all regions of the CNS examined. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among tissues with metabolic function, this gene is expressed at moderate to low levels in pituitary, adipose, adrenal gland, pancreas, thyroid, and adult and fetal skeletal muscle and heart, and fetal liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal liver (CT=31) when compared to expression in the adult counterpart (CT=35). Thus, expression of this gene may be used to differentiate between the fetal and adult source of these tissue. Furthermore, the higher levels of expression in fetal liver suggest a role for this protein product in the development of the organ. Therefore, therapeutic modulation of the expression or function of this gene may help in the regeneration of the liver in the adult and in the treatment of diseases that affect the liver such as Von Hippel-Lindau (VHL) syndrome and cirrhosis.

In addition, there is significant expression in a sample derived from a prostate cancer cell line (CT=26.5) and a cluster of renal cancer cell lines. Thus, expression of this gene could be used to differentiate between the prostate cancer cell line sample and other samples on this panel and as a marker to detect the presence of prostate cancer. Furthermore, therapeutic modulation of the expression or function of this gene may be effective in the treatment of prostate and kidney cancers.

Panel 2.1 Summary: Ag3980 Highest expression of the CG94989-01 gene is seen in a kidney cancer (CT=30.7). Futhermore, expression is higher in kidney, lung and liver cancers when compared to expression in normal adjacent tissue. Conversely, expression is higher in colon tissue than in the corresponding tumor samples. Thus, expression of this gene could be used to differentiate between these samples and other samples on this panel and as a marker to detect the presence of these cancers. Furtherinore, therapeutic modulation of the expression or function of this gene may be effective in the treatment of colon, kidney, lung and liver cancers.

Panel 4.1D Summary: Ag3980 Highest expression of the CG94989-01 gene is seen in untreated pulmonary aortic endothelial cells (CT=30.6). Significant expression is also seen in a cluster of samples derived from HUVEC endothelial cells. Thus, expression of this gene could be used to differentiate these endothelial cells from other samples on this panel. Furthermore, this expression profile suggests that this gene product may be involved in endothelial cell function. Therefore, therapeutic modulation of the gene product may reduce or eliminate the symptoms in patients with autoimmune and inflammatory diseases in which endothelial cells are involved, such as lupus erythematosus, asthma, emphysema, Crohn's disease, ulcerative colitis, rheumatoid arthritis, osteoarthritis, and psoriasis.

E. NOV7 (CG94978-01): Transmission-Blocking Target Antigen S230 Precursor

Expression of gene CG94978-01 was assessed using the primer-probe set Ag3977, described in Table AA. Results of the RTQ-PCR runs are shown in Tables EB, EC and ED.

TABLE EA

Probe Name Ag3977

| | | | |
|---|---|---|---|
| Primers | Sequences | Length | Start Position |

| | | | | |
|---|---|---|---|---|
| Forward | 5′-aaataccccaaggaacctacct-3′ | (Seq ID NO:126) | 22 | 773 |
| Probe | TET-5′-atataccagccagcccctgtggagag-3′-TAMRA | (Seq ID NO:127) | 26 | 800 |
| Reverse | 5′-ctcttccttccccacttcttc-3′ | (Seq ID NO: 128) | 21 | 832 |

TABLE EB
CNS_neurodegeneration_v1.0

| | Rel. Exp. | | Rel. Exp. |
|---|---|---|---|
| | (%) Ag3977, | | (%) Ag3977, |
| | Run | | Run |
| Tissue Name | 206879692 | Tissue Name | 206879692 |
| AD 1 Hippo | 17.3 | Control (Path) 3 | 7.5 |
| | | Temporal Ctx | |
| AD 2 Hippo | 30.6 | Control (Path) 4 | 43.8 |
| | | Temporal Ctx | |
| AD 3 Hippo | 8.6 | AD 1 Occipital | 31.4 |
| | | Ctx | |
| AD 4 Hippo | 17.6 | AD 2 Occipital | 0.0 |
| | | Ctx (Missing) | |
| AD 5 hippo | 100.0 | AD 3 Occipital | 8.5 |
| | | Ctx | |
| AD 6 Hippo | 73.7 | AD 4 Occipital | 30.8 |
| | | Ctx | |
| Control 2 Hippo | 47.6 | AD 5 Occipital | 29.9 |
| | | Ctx | |
| Control 4 Hippo | 23.3 | AD 6 Occipital | 54.3 |
| | | Ctx | |
| Control (Path) | 10.2 | Control 1 | 8.8 |
| 3 Hippo | | Occipital Ctx | |
| AD 1 Temporal | 32.1 | Control 2 | 72.2 |
| Ctx | | Occipital Ctx | |
| AD 2 Temporal | 45.4 | Control 3 | 23.0 |
| Ctx | | Occipital Ctx | |
| AD 3 Temporal | 12.8 | Control 4 | 10.2 |
| Ctx | | Occipital Ctx | |
| AD 4 Temporal | 31.6 | Control (Path) 1 | 73.7 |
| Ctx | | Occipital Ctx | |
| AD 5 Inf | 94.6 | Control (Path) 2 | 15.1 |
| Temporal Ctx | | Occipital Ctx | |
| AD 5 SupTemporal | 53.2 | Control (Path) 3 | 7.1 |
| Ctx | | Occipital Ctx | |
| AD 6 Inf | 94.6 | Control (Path) 4 | 20.0 |
| Temporal Ctx | | Occipital Ctx | |
| AD 6 Sup | 72.2 | Control 1 Parietal | 14.1 |
| Temporal Ctx | | Ctx | |
| Control 1 | 8.5 | Control 2 Parietal | 67.8 |
| Temporal Ctx | | Ctx | |
| Control 2 | 61.6 | Control 3 Parietal | 28.9 |
| Temporal Ctx | | Ctx | |
| Control 3 | 26.6 | Control (Path) 1 | 67.8 |
| Temporal Ctx | | Parietal Ctx | |
| Control 4 | 18.8 | Control (Path) 2 | 27.9 |
| Temporal Ctx | | Parietal Ctx | |
| Control (Path) 1 | 76.3 | Control (Path) 3 | 10.4 |
| Temporal Ctx | | Parietal Ctx | |
| Control (Path) 2 | 29.7 | Control (Path) 4 | 59.5 |
| Temporal Ctx | | Parietal Ctx | |

TABLE EC
General_screening_panel_v1.4

| | Rel. Exp. | | Rel. Exp. |
|---|---|---|---|
| | (%) Ag3977, | | (%) Ag3977, |
| | Run | | Run |
| Tissue Name | 217525330 | Tissue Name | 217525330 |
| Adipose | 6.6 | Renal ca. TK-10 | 50.7 |
| Melanoma* | 18.3 | Bladder | 19.3 |
| Hs688(A).T | | | |
| Melanoma* | 13.1 | Gastric ca. (liver | 100.0 |
| Hs688(B).T | | met.) NCI-N87 | |
| Melanoma* M14 | 42.0 | Gastric ca. KATO | 34.9 |
| | | III | |
| Melanoma* | 47.0 | Colon ca. SW-948 | 15.0 |
| LOXIMVI | | | |
| Melanoma* SK- | 39.2 | Colon ca. SW480 | 41.2 |
| MEL-5 | | | |
| Squamous cell | 26.4 | Colon ca.* (SW480 | 29.5 |
| carcinoma SCC-4 | | met) SW620 | |
| Testis Pool | 16.5 | Colon ca. HT29 | 30.4 |
| Prostate ca.* | 41.5 | Colon ca. HCT-116 | 39.0 |
| (bone met) PC-3 | | | |
| Prostate Pool | 6.9 | Colon ca. CaCo-2 | 39.5 |
| Placenta | 17.3 | Colon cancer | 14.0 |
| | | tissue | |
| Uterus Pool | 6.8 | Colon ca. SW1116 | 19.2 |
| Ovarian ca. | 17.8 | Colon ca. Colo- | 12.5 |
| OVCAR-3 | | 205 | |
| Ovarian ca. | 72.7 | Colon ca. SW-48 | 7.6 |
| SK-OV-3 | | | |
| Ovarian ca. | 15.8 | Colon Pool | 17.8 |
| OVCAR-4 | | | |
| Ovarian ca. | 30.8 | Small Intestine | 16.8 |
| OVCAR-5 | | Pool | |
| Ovarian ca. | 31.4 | Stomach Pool | 11.8 |
| IGROV-1 | | | |
| Ovarian ca. | 25.2 | Bone Marrow Pool | 6.2 |
| OVCAR-8 | | | |
| Ovary | 9.0 | Fetal Heart | 9.6 |
| Breast ca. | 26.1 | Heart Pool | 8.8 |
| MCF-7 | | | |
| Breast ca. | 27.2 | Lymph Node Pool | 21.3 |
| MDA-MB-231 | | | |
| Breast ca. BT | 58.6 | Fetal Skeletal | 7.0 |
| 549 | | Muscle | |
| Breast ca. T47D | 60.3 | Skeletal Muscle | 21.5 |
| | | Pool | |
| Breast ca. MDA- | 7.3 | Spleen Pool | 10.4 |
| N | | | |
| Breast Pool | 17.9 | Thymus Pool | 18.2 |
| Trachea | 9.8 | CNS cancer (glio/ | 40.3 |
| | | astro) U87-MG | |
| Lung | 3.7 | CNS cancer (glio/ | 47.6 |
| | | astro) U-118-MG | |
| Fetal Lung | 13.9 | CNS cancer | 40.6 |
| | | (neuro; met) SK-N- | |
| | | AS | |
| Lung ca. NCI- | 14.0 | CNS cancer (astro) | 31.4 |
| N417 | | SF-539 | |
| Lung ca. LX-1 | 27.4 | CNS cancer (astro) | 48.0 |
| | | SNB-75 | |
| Lung ca. NCI- | 11.0 | CNS cancer (glio) | 15.2 |
| H146 | | SNB-19 | |
| Lung ca. SHP-77 | 35.6 | CNS cancer (glio) | 35.1 |
| | | SF-295 | |
| Lung ca. A549 | 24.1 | Brain (Amygdala) | 28.7 |
| | | Pool | |
| Lung ca. NCI- | 17.1 | Brain (cerebellum) | 49.0 |
| H526 | | | |
| Lung ca. NCI- | 37.9 | Brain (fetal) | 16.0 |
| H23 | | | |
| Lung ca. NCI- | 24.1 | Brain | 15.1 |
| H460 | | (Hippocampus) Pool | |
| Lung ca. HOP-62 | 15.0 | Cerebral Cortex | 21.3 |
| | | Pool | |
| Lung ca. NCI- | 30.8 | Brain (Substantia | 14.3 |
| H522 | | nigra) Pool | |
| Liver | 4.9 | Brain (Thalamus) | 25.5 |
| | | Pool | |
| Fetal Liver | 15.1 | Brain (whole) | 35.8 |
| Liver ca. HepG2 | 21.6 | Spinal Cord Pool | 18.2 |
| Kidney Pool | 16.0 | Adrenal Gland | 15.3 |
| Fetal Kidney | 12.1 | Pituitary gland | 7.5 |
| | | Pool | |
| Renal ca. 786-0 | 28.3 | Salivary Gland | 6.7 |
| Renal ca. A498 | 7.1 | Thyroid (female) | 6.1 |
| Renal ca. ACHN | 16.3 | Pancreatic ca. | 37.4 |
| | | CAPAN2 | |
| Renal ca. UO-31 | 27.2 | Pancreas Pool | 22.2 |

TABLE ED
Panel 4.1D

| | Rel. Exp. | | Rel. Exp. |
|---|---|---|---|
| | (%) Ag3977, | | (%) Ag3977, |
| | Run | | Run |
| Tissue Name | 170737276 | Tissue Name | 170737276 |
| Secondary Th1 | 37.9 | HUVEC IL-1beta | 33.7 |
| act | | | |
| Secondary Th2 | 61.1 | HUVEC IFN gamma | 30.6 |
| act | | | |
| Secondary Tr1 | 36.6 | HUVEC TNF alpha + | 25.7 |
| act | | IFN gamma | |
| Secondary Th1 | 21.5 | HUVEC TNF alpha + | 34.9 |
| rest | | IL4 | |
| Secondary Th2 | 15.8 | HUVEC IL-11 | 25.9 |
| rest | | | |
| Secondary Tr1 | 27.2 | Lung Microvascular | 42.0 |
| rest | | EC none | |
| Primary Th1 act | 33.4 | Lung Microvascular | 28.5 |
| | | EC TNFalpha + IL- | |
| | | 1beta | |
| Primary Th2 act | 52.5 | Microvascular | 24.8 |
| | | Dermal EC none | |
| Primary Tr1 act | 39.5 | Microsvasular | 22.5 |
| | | Dermal EC | |
| | | TNFalpha + IL- | |
| | | 1beta | |
| Primary Th1 | 10.9 | Bronchial | 17.4 |
| rest | | epithelium | |
| | | TNFalpha + | |
| | | IL1beta | |
| Primary Th2 | 11.5 | Small airway | 13.7 |
| rest | | epithelium none | |
| Primary Tr1 | 17.3 | Small airway | 21.6 |
| rest | | epithelium | |
| | | TNFalpha + IL- | |
| | | 1beta | |
| CD45RA CD4 | 35.4 | Coronery artery | 13.0 |
| lymphocyte act | | SMC rest | |
| CD45RO CD4 | 40.3 | Coronery artery | 12.7 |
| lymphocyte act | | SMC TNFalpha + IL- | |
| | | 1beta | |
| CD8 lymphocyte | 35.1 | Astrocytes rest | 2.9 |
| act | | | |
| Secondary CD8 | 32.1 | Astrocytes | 9.8 |
| lymphocyte rest | | TNFalpha + IL- | |
| | | 1beta | |
| Secondary CD8 | 16.3 | KU-812 (Basophil) | 41.5 |
| lymphocyte act | | rest | |
| CD4 lymphocyte | 13.9 | KU-812 (Basophil) | 46.3 |
| none | | PMA/ionomycin | |
| 2ry Th1/Th2/ | 45.4 | CCD1106 | 51.8 |
| Tr1_anti-CD95 | | (Keratinocytes) | |
| CH11 | | none | |
| LAK cells rest | 34.2 | CCD1106 | 30.1 |
| | | (Keratinocytes) | |
| | | TNFalpha + IL- | |
| | | 1beta | |
| LAK cells IL-2 | 28.1 | Liver cirrhosis | 10.7 |
| LAK cells IL-2 + | 17.0 | NCI-H292 none | 15.3 |
| IL-12 | | | |
| LAK cells IL-2 + | 20.6 | NCI-H292 IL-4 | 14.6 |
| IFN gamma | | | |
| LAK cells IL-2 + | 30.4 | NCI-H292 IL-9 | 18.2 |
| IL-18 | | | |
| LAK cells | 22.7 | NCI-H292 IL-13 | 18.8 |
| PMA/ionomycin | | | |
| NK Cells IL-2 | 56.6 | NCI-H292 IFN | 19.2 |
| rest | | gamma | |
| Two Way MLR 3 | 33.9 | HPAEC none | 25.5 |
| day | | | |
| Two Way MLR 5 | 26.8 | HPAEC TNF alpha + | 36.3 |
| day | | IL-1 beta | |
| Two Way MLR 7 | 23.2 | Lung fibroblast | 32.1 |
| day | | none | |
| PBMC rest | 19.1 | Lung fibroblast | 20.4 |
| | | TNF alpha + IL-1 | |
| | | beta | |
| PBMC PWM | 27.9 | Lung fibroblast | 24.7 |
| | | IL-4 | |
| PBMC PHA-L | 36.9 | Lung fibroblast | 28.1 |
| | | IL-9 | |
| Ramos (B cell) | 38.7 | Lung fibroblast | 22.4 |
| none | | IL-13 | |
| Ramos (B cell) | 37.1 | Lung fibroblast | 27.0 |
| ionomycin | | IFN gamma | |
| B lymphocytes | 28.9 | Dermal fibroblast | 28.5 |
| PWM | | CCD1070 rest | |
| B lymphocytes | 54.3 | Dermal fibroblast | 39.0 |
| CD40L and IL-4 | | CCD1070 TNF alpha | |
| EOL-1 dbcAMP | 36.9 | Dermal fibroblast | 22.7 |
| | | CCD1070 IL-1 beta | |
| EOL-1 dbcAMP | 20.6 | Dermal fibroblast | 18.6 |
| PMA/ionomycin | | IFN gamma | |
| Dendritic cells | 29.1 | Dermal fibroblast | 31.6 |
| none | | IL-4 | |
| Dendritic cells | 32.3 | Dermal Fibroblasts | 14.9 |
| LPS | | rest | |
| Dendritic cells | 31.9 | Neutrophils TNFa + | 16.7 |
| anti-CD40 | | LPS | |
| Monocytes rest | 34.6 | Neutrophils rest | 17.1 |
| Monocytes LPS | 100.0 | Colon | 11.0 |
| Macrophages rest | 37.6 | Lung | 15.8 |
| Macrophages LPS | 18.0 | Thymus | 29.7 |
| HUVEC none | 19.1 | Kidney | 40.6 |
| HUVEC starved | 33.7 | | |

CNS_neurodegeneration_v1.0 Summary: Ag3977 This panel does not show differential expression of the CG94978-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3977 Highest expression of the CG94978-01 gene is seen in a gastric cancer cell line (CT=25.3). Overall, this gene appears to show a moderate association with cancer cell lines, when compared to expression in normal tissue samples. Thus, expression of this gene could be used as a marker for the presence of cancer. Furthermore, therapeutic modulation of the expression or function of this gene may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at high to moderate levels in the CNS, including the hippocampus, amygdala, cerebellum, thalamus, substantia nigra and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3977 Highest expression of the CG94978-01 gene is seen in LPS stimulated monocytes (CT=29.7). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

F. NOV8 (CG94713-01): Nuclear Protein

Expression of gene CG94713-01 was assessed using the primer-probe sets Ag3945 and Ag4790, described in Tables FA and FB. Results of the RTQ-PCR runs are shown in Tables FC, FD, FE and FF.

TABLE FA

Probe Name Ag3945

| | | | |
|---|---|---|---|
| Primers | Sequences | Length | Start Position |

| | | | | |
|---|---|---|---|---|
| Forward | 5′-tttgataagagcgaagctcaag-3′ | (Seq ID NO:129) | 22 | 1051 |
| Probe | TET-5′-tgctgaacccagagcataaagtcactg-3′-TAMRA | (Seq ID NO:130) | 27 | 1073 |
| Reverse | 5′-atcctttcctttggagaccat-3′ | (Seq ID NO:131) | 21 | 1117 |

TABLE FB

Probe Name Ag4790

| | | | |
|---|---|---|---|
| Primers | Sequences | Length | Start Position |

| | | | | |
|---|---|---|---|---|
| Forward | 5′-ttatcaaaagctggcaccat-3′ | (Seq ID NO:132) | 20 | 1039 |
| Probe | TET-5′-aagggcattcatcatcaataagacaa-3′-TAMRA | (Seq ID NO:133) | 26 | 1011 |
| Reverse | 5′-aaggaaatgggccaaaatc-3′ | (Seq ID NO:134) | 19 | 973 |

TABLE FC
CNS_neurodegeneration_v1.0

| | Rel. Exp. | | Rel. Exp. |
|---|---|---|---|
| | (%) Ag3945, | | (%) Ag3945, |
| | Run | | Run |
| Tissue Name | 212343352 | Tissue Name | 212343352 |
| AD 1 Hippo | 31.9 | Control (Path) 3 | 20.3 |
| | | Temporal Ctx | |
| AD 2 Hippo | 37.4 | Control (Path) 4 | 39.8 |
| | | Temporal Ctx | |
| AD 3 Hippo | 20.9 | AD 1 Occipital | 37.1 |
| | | Ctx | |
| AD 4 Hippo | 7.2 | AD 2 Occipital | 0.0 |
| | | Ctx (Missing) | |
| AD 5 hippo | 74.2 | AD 3 Occipital | 26.6 |
| | | Ctx | |
| AD 6 Hippo | 70.2 | AD 4 Occipital | 24.1 |
| | | Ctx | |
| Control 2 Hippo | 14.1 | AD 5 Occipital | 30.6 |
| | | Ctx | |
| Control 4 Hippo | 22.8 | AD 6 Occipital | 29.9 |
| | | Ctx | |
| Control (Path) | 37.4 | Control 1 | 6.9 |
| 3 Hippo | | Occipital Ctx | |
| AD 1 Temporal | 87.1 | Control 2 | 28.7 |
| Ctx | | Occipital Ctx | |
| AD 2 Temporal | 45.7 | Control 3 | 23.5 |
| Ctx | | Occipital Ctx | |
| AD 3 Temporal | 28.9 | Control 4 | 21.3 |
| Ctx | | Occipital Ctx | |
| AD 4 Temporal | 25.5 | Control (Path) 1 | 85.9 |
| Ctx | | Occipital Ctx | |
| AD 5 Inf | 100.0 | Control (Path) 2 | 13.2 |
| Temporal Ctx | | Occipital Ctx | |
| AD 5 SupTemporal | 90.8 | Control (Path) 3 | 10.6 |
| Ctx | | Occipital Ctx | |
| AD 6 Inf | 92.0 | Control (Path) 4 | 13.6 |
| Temporal Ctx | | Occipital Ctx | |
| AD 6 Sup | 67.8 | Control 1 | 14.8 |
| Temporal Ctx | | Parietal Ctx | |
| Control 1 | 27.0 | Control 2 | 71.7 |
| Temporal Ctx | | Parietal Ctx | |
| Control 2 | 33.2 | Control 3 | 15.3 |
| Temporal Ctx | | Parietal Ctx | |
| Control 3 | 24.3 | Control (Path) 1 | 71.2 |
| Temporal Ctx | | Parietal Ctx | |
| Control 4 | 11.3 | Control (Path) 2 | 33.0 |
| Temporal Ctx | | Parietal Ctx | |
| Control (Path) 1 | 53.6 | Control (Path) 3 | 15.0 |
| Temporal Ctx | | Parietal Ctx | |
| Control (Path) 2 | 28.3 | Control (Path) 4 | 27.7 |
| Temporal Ctx | | Parietal Ctx | |

TABLE FD
General_screening_panel_v1.4

| | Rel. Exp. (%) | Rel. Exp. (%) | | Rel. Exp. (%) | Rel. Exp. (%) |
|---|---|---|---|---|---|
| | Ag3945, Run | Ag4790, Run | | Ag3945, Run | Ag4790, Run |
| Tissue Name | 219275408 | 223203132 | Tissue Name | 219275408 | 223203132 |
| Adipose | 0.6 | 3.1 | Renal ca. TK-10 | 9.9 | 15.9 |
| Melanoma* | 0.0 | 0.1 | Bladder | 32.5 | 55.1 |
| Hs688(A).T | | | | | |
| Melanoma* | 0.0 | 0.0 | Gastric ca. (liver | 37.1 | 50.0 |
| Hs688(B).T | | | met.) NCI-N87 | | |
| Melanoma* | 3.7 | 9.4 | Gastric ca. | 92.7 | 98.6 |
| M14 | | | KATO III | | |
| Melanoma* | 0.0 | 0.0 | Colon ca. SW- | 2.3 | 4.5 |
| LOXIMVI | | | 948 | | |
| Melanoma* | 0.0 | 25.0 | Colon ca. SW480 | 2.0 | 2.4 |
| SK-MEL-5 | | | | | |
| Squamous cell | 2.8 | 5.5 | Colon ca.* | 0.0 | 0.1 |
| carcinoma | | | (SW480 met) | | |
| SCC-4 | | | SW620 | | |
| Testis Pool | 1.6 | 3.9 | Colon ca. HT29 | 3.6 | 4.6 |
| Prostate ca.* | 0.5 | 1.3 | Colon ca. HCT- | 31.4 | 41.8 |
| (bone met) | | | 116 | | |
| PC-3 | | | | | |
| Prostate Pool | 2.5 | 8.1 | Colon ca. CaCo-2 | 36.3 | 77.9 |
| Placenta | 0.4 | 1.4 | Colon cancer | 5.6 | 10.2 |
| | | | tissue | | |
| Uterus Pool | 0.5 | 3.6 | Colon ca. | 6.5 | 11.6 |
| | | | SW1116 | | |
| Ovarian ca. | 19.8 | 57.8 | Colon ca. Colo- | 0.5 | 1.4 |
| OVCAR-3 | | | 205 | | |
| Ovarian ca. | 5.0 | 10.5 | Colon ca. SW-48 | 1.8 | 7.3 |
| SK-OV-3 | | | | | |
| Ovarian ca. | 1.5 | 4.0 | Colon Pool | 4.0 | 14.2 |
| OVCAR-4 | | | | | |
| Ovarian ca. | 9.7 | 17.9 | Small Intestine | 8.9 | 20.3 |
| OVCAR-5 | | | Pool | | |
| Ovarian ca. | 0.2 | 1.0 | Stomach Pool | 4.0 | 11.2 |
| IGROV-1 | | | | | |
| Ovarian ca. | 0.0 | 0.6 | Bone Marrow | 1.3 | 4.5 |
| OVCAR-8 | | | Pool | | |
| Ovary | 3.6 | 9.5 | Fetal Heart | 7.3 | 31.0 |
| Breast ca. | 4.2 | 6.2 | Heart Pool | 4.3 | 16.4 |
| MCF-7 | | | | | |
| Breast ca. | 3.8 | 4.6 | Lymph Node | 5.2 | 23.8 |
| MDA-MB- | | | Pool | | |
| 231 | | | | | |
| Breast ca. BT | 0.0 | 0.0 | Fetal Skeletal | 3.1 | 11.2 |
| 549 | | | Muscle | | |
| Breast ca. | 14.8 | 25.3 | Skeletal | 5.6 | 13.6 |
| T47D | | | Muscle Pool | | |
| Breast ca. | 3.7 | 8.0 | Spleen Pool | 1.6 | 7.0 |
| MDA-N | | | | | |
| Breast Pool | 4.3 | 10.8 | Thymus Pool | 2.7 | 5.4 |
| Trachea | 9.2 | 18.6 | CNS cancer | 0.0 | 0.0 |
| | | | (glio/astro) U87- | | |
| | | | MG | | |
| Lung | 3.1 | 10.1 | CNS cancer | 0.0 | 0.3 |
| | | | (glio/astro) U- | | |
| | | | 118-MG | | |
| Fetal Lung | 31.0 | 60.3 | CNS cancer | 2.7 | 6.3 |
| | | | (neuro;met) SK- | | |
| | | | N-AS | | |
| Lung ca. NCI- | 0.4 | 0.7 | CNS cancer | 0.0 | 0.3 |
| N417 | | | (astro) SF-539 | | |
| Lung ca. LX-1 | 0.0 | 0.5 | CNS cancer | 0.4 | 0.7 |
| | | | (astro) SNB-75 | | |
| Lung ca. NCI- | 0.4 | 1.0 | CNS cancer | 0.3 | 0.4 |
| H146 | | | (glio) SNB-19 | | |
| Lung ca. | 21.3 | 38.2 | CNS cancer | 23.5 | 32.8 |
| SHP-77 | | | (glio) SF-295 | | |
| Lung ca. | 100.0 | 100.0 | Brain | 0.5 | 2.5 |
| A549 | | | (Amygdala) Pool | | |
| Lung ca. NCI- | 0.0 | 0.0 | Brain | 1.2 | 3.4 |
| H526 | | | (cerebellum) | | |
| Lung ca. NCI- | 1.1 | 3.4 | Brain (fetal) | 0.0 | 0.3 |
| H23 | | | | | |
| Lung ca. NCI- | 0.0 | 0.4 | Brain | 1.8 | 4.0 |
| H460 | | | (Hippocampus) | | |
| | | | Pool | | |
| Lung ca. | 0.3 | 1.6 | Cerebral Cortex | 1.9 | 5.1 |
| HOP-62 | | | Pool | | |
| Lung ca. NCI- | 0.0 | 0.0 | Brain (Substantia | 1.5 | 3.5 |
| H522 | | | nigra) Pool | | |
| Liver | 0.2 | 1.1 | Brain (Thalamus) | 2.1 | 11.3 |
| | | | Pool | | |
| Fetal Liver | 10.6 | 17.6 | Brain (whole) | 0.8 | 1.0 |
| Liver ca. | 13.3 | 19.2 | Spinal Cord Pool | 3.9 | 7.9 |
| HepG2 | | | | | |
| Kidney Pool | 15.8 | 23.7 | Adrenal Gland | 3.4 | 9.7 |
| Fetal Kidney | 2.3 | 13.4 | Pituitary gland | 1.4 | 4.3 |
| | | | Pool | | |
| Renal ca. 786- | 12.5 | 11.1 | Salivary Gland | 1.7 | 3.4 |
| 0 | | | | | |
| Renal ca. | 4.7 | 12.2 | Thyroid (female) | 0.7 | 1.2 |
| A498 | | | | | |
| Renal ca. | 0.0 | 0.0 | Pancreatic ca. | 1.3 | 2.9 |
| ACHN | | | CAPAN2 | | |
| Renal ca. UO-31 | 4.0 | 6.2 | Pancreas Pool | 12.3 | 27.9 |

TABLE FE
Panel 2.1

| | Rel. Exp. | | Rel. Exp. |
|---|---|---|---|
| | (%) Ag3945, | | (%) Ag3945, |
| | Run | | Run |
| Tissue Name | 170686173 | Tissue Name | 170686173 |
| Normal Colon | 52.1 | Kidney Cancer | 6.1 |
| | | 9010320 | |
| Colon cancer | 74.2 | Kidney margin | 94.0 |
| (OD06064) | | 9010321 | |
| Colon cancer | 4.0 | Kidney Cancer | 4.8 |
| margin (OD06064) | | 8120607 | |
| Colon cancer | 8.0 | Kidney margin | 2.3 |
| (OD06159) | | 8120608 | |
| Colon cancer | 25.5 | Normal Uterus | 14.9 |
| margin (OD06159) | | | |
| Colon cancer | 69.7 | Uterus Cancer | 63.3 |
| (OD06298-08) | | | |
| Colon cancer | 29.3 | Normal Thyroid | 2.0 |
| margin (OD06298- | | | |
| 018) | | | |
| Colon Cancer | 8.5 | Thyroid Cancer | 2.6 |
| Gr.2 ascend | | | |
| colon (ODO3921) | | | |
| Colon Cancer | 17.2 | Thyroid Cancer | 9.0 |
| margin (ODO3921) | | A302152 | |
| Colon cancer | 5.5 | Thyroid margin | 5.9 |
| metastasis | | A302153 | |
| (OD06104) | | | |
| Lung margin | 28.7 | Normal Breast | 59.0 |
| (OD06104) | | | |
| Colon mets to | 11.9 | Breast Cancer | 2.5 |
| lung (OD04451- | | | |
| 01) | | | |
| Lung margin | 79.6 | Breast Cancer | 6.4 |
| (OD04451-02) | | | |
| Normal Prostate | 11.6 | Breast Cancer | 1.7 |
| | | (OD04590-01) | |
| Prostate Cancer | 4.6 | Breast Cancer | 24.0 |
| (OD04410) | | Mets (OD04590- | |
| | | 03) | |
| Prostate margin | 41.5 | Breast Cancer | 13.4 |
| (OD04410) | | Metastasis | |
| Normal Lung | 35.4 | Breast Cancer | 4.3 |
| Invasive poor | 64.6 | Breast Cancer | 11.7 |
| diff. lung | | 9100266 | |
| adeno 1 | | | |
| (ODO4945-01) | | | |
| Lung margin | 19.8 | Breast margin | 1.6 |
| (ODO4945-03) | | 9100265 | |
| Lung Malignant | 26.6 | Breast Cancer | 11.8 |
| Cancer (OD03126) | | A209073 | |
| Lung margin | 57.4 | Breast margin | 29.9 |
| (OD03126) | | A2090734 | |
| Lung Cancer | 19.8 | Normal Liver | 74.7 |
| (OD05014A) | | | |
| Lung margin | 19.9 | Liver Cancer | 0.8 |
| (OD05014B) | | 1026 | |
| Lung Cancer | 4.5 | Liver Cancer | 10.2 |
| (OD04237-01) | | 1025 | |
| Lung margin | 35.8 | Liver Cancer | 12.7 |
| (OD04237-02) | | 6004-T | |
| Ocular Mel Met | 37.4 | Liver Tissue | 4.8 |
| to Liver | | 6004-N | |
| (ODO4310) | | | |
| Liver margin | 55.5 | Liver Cancer | 9.9 |
| (ODO4310) | | 6005-T | |
| Melanoma Mets | 16.7 | Liver Tissue | 5.9 |
| to Lung | | 6005-N | |
| (OD04321) | | | |
| Lung margin | 78.5 | Liver Cancer | 12.7 |
| (OD04321) | | | |
| Normal Kidney | 28.3 | Normal Bladder | 65.5 |
| Kidney Ca, | 43.5 | Bladder Cancer | 0.0 |
| Nuclear grade 2 | | | |
| (OD04338) | | | |
| Kidney margin | 11.4 | Bladder Cancer | 4.4 |
| (OD04338) | | | |
| Kidney Ca | 6.4 | Normal Ovary | 3.5 |
| Nuclear grade | | | |
| ½ (OD04339) | | | |
| Kidney margin | 28.1 | Ovarian Cancer | 3.5 |
| (OD04339) | | | |
| Kidney Ca, Clear | 4.2 | Ovarian cancer | 0.0 |
| cell type | | (OD06145) | |
| (OD04340) | | | |
| Kidney margin | 42.3 | Ovarian cancer | 23.8 |
| (OD04340) | | margin (OD06145) | |
| Kidney Ca, | 1.1 | Normal Stomach | 74.7 |
| Nuclear grade 3 | | | |
| (OD04348) | | | |
| Kidney margin | 21.9 | Gastric Cancer | 2.2 |
| (OD04348) | | 9060397 | |
| Kidney Cancer | 100.0 | Stomach margin | 1.8 |
| (OD04450-01) | | 9060396 | |
| Kidney margin | 41.8 | Gastric Cancer | 40.9 |
| (OD04450-03) | | 9060395 | |
| Kidney Cancer | 0.0 | Stomach margin | 14.0 |
| 8120613 | | 9060394 | |
| Kidney margin | 0.6 | Gastric Cancer | 30.8 |
| 8120614 | | 064005 | |

TABLE FF
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3945, | Ag4790, | | Ag3945, | Ag4790, |
| | Run | Run | | Run | Run |
| Tissue Name | 170701960 | 223235418 | Tissue Name | 170701960 | 223235418 |
| Secondary Th1 act | 0.0 | 0.0 | HUVEC IL-1beta | 0.0 | 0.5 |
| Secondary Th2 act | 0.0 | 0.1 | HUVEC IFN | 0.0 | 0.2 |
| | | | gamma | | |
| Secondary Tr1 act | 0.0 | 0.0 | HUVEC TNF | 0.0 | 0.1 |
| | | | alpha + IFN | | |
| | | | gamma | | |
| Secondary Th1 rest | 0.0 | 0.1 | HUVEC TNF | 0.0 | 0.4 |
| | | | alpha + IL4 | | |
| Secondary Th2 rest | 0.8 | 1.0 | HUVEC IL-11 | 0.0 | 0.1 |
| Secondary Tr1 rest | 1.4 | 0.0 | Lung | 0.7 | 0.4 |
| | | | Microvascular EC | | |
| | | | none | | |
| Primary Th1 act | 0.0 | 0.6 | Lung | 0.0 | 0.6 |
| | | | Microvascular EC | | |
| | | | TNFalpha + IL- | | |
| | | | 1beta | | |
| Primary Th2 act | 1.3 | 1.6 | Microvascular | 4.0 | 3.1 |
| | | | Dermal EC none | | |
| Primary Tr1 act | 0.5 | 1.1 | Microsvasular | 1.7 | 2.3 |
| | | | Dermal EC | | |
| | | | TNFalpha + IL- | | |
| | | | 1beta | | |
| Primary Th1 rest | 0.0 | 0.0 | Bronchial | 21.0 | 25.3 |
| | | | epithelium | | |
| | | | TNFalpha + | | |
| | | | IL1beta | | |
| Primary Th2 rest | 0.0 | 0.1 | Small airway | 4.5 | 5.3 |
| | | | epithelium none | | |
| Primary Tr1 rest | 0.0 | 0.0 | Small airway | 6.7 | 8.4 |
| | | | epithelium | | |
| | | | TNFalpha + IL- | | |
| | | | 1beta | | |
| CD45RA CD4 | 0.0 | 0.0 | Coronery artery | 0.5 | 0.0 |
| lymphocyte act | | | SMC rest | | |
| CD45RO CD4 | 0.0 | 0.0 | Coronery artery | 0.0 | 0.3 |
| lymphocyte act | | | SMC TNFalpha + | | |
| | | | IL-1beta | | |
| CD8 lymphocyte | 0.0 | 0.1 | Astrocytes rest | 0.0 | 0.1 |
| act | | | | | |
| Secondary CD8 | 0.0 | 0.0 | Astrocytes | 0.5 | 0.5 |
| lymphocyte rest | | | TNFalpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 19.5 | 19.3 |
| Th1/Th2/Tr1_anti- | | | (Keratinocytes) | | |
| CD95 CH11 | | | none | | |
| LAK cells rest | 0.7 | 1.1 | CCD1106 | 12.6 | 11.0 |
| | | | (Keratinocytes) | | |
| | | | TNFalpha + IL- | | |
| | | | 1beta | | |
| LAK cells IL-2 | 0.0 | 0.0 | Liver cirrhosis | 29.7 | 46.0 |
| LAK cells IL-2 + IL- | 0.0 | 0.4 | NCI-H292 none | 40.3 | 52.9 |
| 12 | | | | | |
| LAK cells IL- | 0.0 | 0.1 | NCI-H292 IL-4 | 51.1 | 86.5 |
| 2 + IFN gamma | | | | | |
| LAK cells IL-2 + | 0.4 | 0.4 | NCI-H292 IL-9 | 91.4 | 100.0 |
| IL-18 | | | | | |
| LAK cells | 0.0 | 0.2 | NCI-H292 IL-13 | 73.7 | 47.0 |
| PMA/ionomycin | | | | | |
| NK Cells IL-2 rest | 0.0 | 0.0 | NCI-H292 IFN | 100.0 | 55.5 |
| | | | gamma | | |
| Two Way MLR 3 | 0.0 | 0.0 | HPAEC none | 0.9 | 0.0 |
| day | | | | | |
| Two Way MLR 5 | 0.0 | 0.2 | HPAEC TNF | 0.0 | 0.1 |
| day | | | alpha + IL-1 beta | | |
| Two Way MLR 7 | 0.0 | 0.0 | Lung fibroblast | 1.2 | 1.0 |
| day | | | none | | |
| PBMC rest | 0.0 | 0.0 | Lung fibroblast | 0.0 | 0.3 |
| | | | TNF alpha + IL-1 beta | | |
| PBMC PWM | 0.0 | 0.0 | Lung fibroblast | 0.9 | 0.5 |
| | | | IL-4 | | |
| PBMC PHA-L | 0.0 | 0.0 | Lung fibroblast | 1.4 | 0.7 |
| | | | IL-9 | | |
| Ramos (B cell) | 0.0 | 0.0 | Lung fibroblast | 1.0 | 0.5 |
| none | | | IL-13 | | |
| Ramos (B cell) | 0.0 | 0.0 | Lung fibroblast | 1.4 | 0.6 |
| ionomycin | | | IFN gamma | | |
| B lymphocytes | 0.0 | 0.1 | Dermal fibroblast | 0.8 | 0.0 |
| PWM | | | CCD1070 rest | | |
| B lymphocytes | 0.0 | 0.2 | Dermal fibroblast | 0.0 | 0.3 |
| CD40L and IL-4 | | | CCD1070 TNF | | |
| | | | alpha | | |
| EOL-1 dbcAMP | 0.0 | 0.0 | Dermal fibroblast | 0.0 | 0.0 |
| | | | CCD1070 IL-1 | | |
| | | | beta | | |
| EOL-1 dbcAMP | 0.0 | 0.0 | Dermal fibroblast | 2.9 | 1.3 |
| PMA/ionomycin | | | IFN gamma | | |
| Dendritic cells | 3.4 | 3.2 | Dermal fibroblast | 1.0 | 1.0 |
| none | | | IL-4 | | |
| Dendritic cells | 1.9 | 3.6 | Dermal | 5.5 | 3.7 |
| LPS | | | Fibroblasts rest | | |
| Dendritic cells anti- | 13.6 | 10.4 | Neutrophils | 0.6 | 0.0 |
| CD40 | | | TNFa + LPS | | |
| Monocytes rest | 0.0 | 0.1 | Neutrophils rest | 0.0 | 0.1 |
| Monocytes LPS | 0.0 | 0.0 | Colon | 22.2 | 10.3 |
| Macrophages rest | 7.4 | 8.7 | Lung | 32.5 | 34.9 |
| Macrophages LPS | 0.8 | 0.7 | Thymus | 25.9 | 9.3 |
| HUVEC none | 0.0 | 0.3 | Kidney | 56.3 | 51.8 |
| HUVEC starved | 0.0 | 0.7 | | | |

CNS_neurodegeneration_v1.0 Summary: Ag3945 This panel does not show differential expression of the CG94713-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3945/Ag4790 Two experiments with two different probe and primer sets produce results that are in excellent agreement, with highest expression of the CG94713-01 gene in a lung cancer cell line (CTs=24-30). In addition, significant levels of expression are seen in a cluster of samples derived from brain, colon, gastric, ovarian, and melanoma cancer cell lines. Thus, expression of this gene could be used to differentiate between these samples and other samples on this panel and as a marker to detect the presence of these cancers. Furthermore, therapeutic modulation of the expression or function of this gene may be effective in the treatment of brain, colon, gastric, ovarian, mclanoma, and lung cancers.

Among tissues with metabolic function, the region of the gene corresponding to the Ag4890 probe and primer set is seen at high to moderate in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes. In contrast, the Ag3945 probe and primer set showed lower levels of expression of this gene in pancreas, fetal heart and fetal liver (CTs=33-35). Expression in the other metabolic samples is low/undetectable.

The gene is also expressed at moderate to low levels in all regions of the CNS examined, including the hippocampus, amygdala, cerebellum, thalamus, substantia nigra and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 2.1 Summary: Ag3945 Highest expression of the CG94713-01 gene is seen in a kidney cancer sample (CT=32.1). Significant expression is also seen in normal uterine, breast, stomach and bladder. Thus, expression of this gene could be used to as a marker to detect the presence of these cancers. Furthermore, therapeutiQmodulation of the expression or function of this gene may be effective in the treatment of uterine, breast, stomach and bladder cancers.

Panel 4.1D Summary: Ag3945/Ag4790 Two experiments with two different probe and primer sets produce results that are in excellent agreement, with highest expression of the CG94713-01 gene in IL-9 and IFN-gamma treated NCI-H292 cells (CTs=28-32). The gene is also expressed in a cluster of treated and untreated samples derived from the NCI-H292 cell line, a human airway epithelial cell line that produces mucins. Mucus overproduction is an important feature of bronchial asthma and chronic obstructive pulmonary disease samples. The transcript is also expressed at lower but still significant levels in small airway epithelium treated with IL-1 beta and TNF-alpha. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

G. NOV9 (CG94702-01): Hemicentin Precursor

Expression of gene CG94702-01 was assessed using the primer-probe sets Ag3944 and Ag986, described in Tables GA and GB. Results of the RTQ-PCR runs are shown in Tables GC, GD, GE and GF.

TABLE GA

Probe Name Ag3944

| | | | |
|---|---|---|---|
| Primers | Sequences | Length | Start Position |

| | | | | |
|---|---|---|---|---|
| Forward | 5′-acagctgtaaagtcagcaacgt-3′ | (Seq ID NO:135) | 22 | 5408 |
| Probe | TET-5′-accttcaccctcaccgtccaggt-3′-TAMRA | (Seq ID NO:136) | 23 | 5449 |
| Reverse | 5′-actgtctctgtcttggggttct-3′ | (Seq ID NO:137) | 22 | 5486 |

TABLE GB

Probe Name Ag986

| | | | |
|---|---|---|---|
| Primers | Sequences | Length | Start Position |

| | | | | |
|---|---|---|---|---|
| Forward | 5′-ttttggctcctaccagttttg-3′ | (Seq ID NO:138) | 21 | 9305 |
| Probe | TET-5′-ccccttttcctgatgatatttctcaggg-3′-TAMRA | (Seq ID NO:139) 28 | 9326 | |
| Reverse | 5′-ttgatgtgcagtagaggatgaa-3′ | (Seq ID NO:140) | 22 | 9360 |

TABLE GC
General_screening_panel_v1.4

| | Rel. Exp. (%) | Rel. Exp. (%) | | Rel. Exp. (%) | Rel. Exp. (%) |
|---|---|---|---|---|---|
| | Ag3944, Run | Ag986, Run | | Ag3944, Run | Ag986, Run |
| Tissue Name | 219275295 | 219922603 | Tissue Name | 219275295 | 219922603 |
| Adipose | 1.7 | 4.9 | Renal ca. TK-10 | 0.0 | 3.2 |
| Melanoma* | 0.0 | 0.0 | Bladder | 6.3 | 3.0 |
| Hs688(A).T | | | | | |
| Melanoma* | 0.0 | 0.0 | Gastric ca. (liver | 1.3 | 17.8 |
| Hs688(B).T | | | met.) NCI-N87 | | |
| Melanoma* | 0.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. SW480 | 0.0 | 0.0 |
| SK-MEL-5 | | | | | |
| Squamous cell | 0.0 | 0.0 | Colon ca.* | 0.0 | 0.0 |
| carcinoma | | | (SW480 met) | | |
| SCC-4 | | | SW620 | | |
| Testis Pool | 7.5 | 3.6 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 17.6 | 13.9 | Colon ca. CaCo-2 | 0.0 | 5.8 |
| Placenta | 0.0 | 0.0 | Colon cancer | 3.0 | 0.0 |
| | | | tissue | | |
| Uterus Pool | 13.5 | 0.0 | Colon ca. | 0.0 | 0.0 |
| | | | SW1116 | | |
| Ovarian ca. | 24.3 | 27.9 | Colon ca. Colo- | 0.0 | 0.0 |
| OVCAR-3 | | | 205 | | |
| Ovarian ca. | 0.0 | 7.3 | Colon ca. SW-48 | 0.0 | 0.0 |
| SK-OV-3 | | | | | |
| Ovarian ca. | 0.0 | 0.0 | Colon Pool | 52.1 | 53.6 |
| OVCAR-4 | | | | | |
| Ovarian ca. | 0.0 | 4.2 | Small Intestine | 23.8 | 30.1 |
| OVCAR-5 | | | Pool | | |
| Ovarian ca. | 0.8 | 0.0 | Stomach Pool | 10.9 | 9.0 |
| IGROV-1 | | | | | |
| Ovarian ca. | 0.0 | 0.0 | Bone Marrow | 36.6 | 49.7 |
| OVCAR-8 | | | Pool | | |
| Ovary | 3.7 | 0.0 | Fetal Heart | 6.2 | 8.1 |
| Breast ca. | 0.0 | 0.0 | Heart Pool | 23.7 | 9.5 |
| MCF-7 | | | | | |
| Breast ca. | 0.0 | 0.0 | Lymph Node | 54.7 | 99.3 |
| MDA-MB-231 | | | Pool | | |
| Breast ca. BT | 0.0 | 0.0 | Fetal Skeletal | 35.4 | 36.3 |
| 549 | | | Muscle | | |
| Breast ca. | 1.5 | 4.9 | Skeletal Muscle | 60.3 | 25.3 |
| T47D | | | Pool | | |
| Breast ca. | 0.0 | 0.0 | Spleen Pool | 0.0 | 0.0 |
| MDA-N | | | | | |
| Breast Pool | 30.1 | 24.3 | Thymus Pool | 15.1 | 21.5 |
| Trachea | 5.9 | 0.0 | CNS cancer | 0.0 | 0.0 |
| | | | (glio/astro) U87- | | |
| | | | MG | | |
| Lung | 11.3 | 26.4 | CNS cancer | 0.0 | 0.0 |
| | | | (glio/astro) | | |
| | | | U-118-MG | | |
| Fetal Lung | 25.0 | 21.6 | CNS cancer | 11.7 | 22.7 |
| | | | (neuro; met) | | |
| | | | SK-N-AS | | |
| Lung ca. NCI- | 0.0 | 0.0 | CNS cancer | 0.0 | 0.0 |
| N417 | | | (astro) SF-539 | | |
| Lung ca. LX-1 | 0.0 | 0.0 | CNS cancer | 0.0 | 0.0 |
| | | | (astro) SNB-75 | | |
| Lung ca. NCI- | 1.2 | 0.0 | CNS cancer | 0.0 | 0.0 |
| H146 | | | (glio) SNB-19 | | |
| Lung ca. | 0.0 | 29.7 | CNS cancer | 0.0 | 3.4 |
| SHP-77 | | | (glio) SF-295 | | |
| Lung ca. | 0.0 | 0.0 | Brain | 0.0 | 0.0 |
| A549 | | | (Amygdala) Pool | | |
| Lung ca. NCI- | 0.0 | 0.0 | Brain | 1.1 | 0.0 |
| H526 | | | (cerebellum) | | |
| Lung ca. NCI- | 0.0 | 0.0 | Brain (fetal) | 0.0 | 8.8 |
| H23 | | | | | |
| Lung ca. NCI- | 0.0 | 0.0 | Brain | 1.2 | 0.0 |
| H460 | | | (Hippocampus) | | |
| | | | Pool | | |
| Lung ca. | 0.0 | 0.0 | Cerebral Cortex | 0.8 | 0.0 |
| HOP-62 | | | Pool | | |
| Lung ca. NCI- | 0.0 | 0.0 | Brain (Substantia | 0.0 | 0.0 |
| H522 | | | nigra) Pool | | |
| Liver | 0.0 | 0.0 | Brain (Thalamus) | 0.0 | 0.0 |
| | | | Pool | | |
| Fetal Liver | 1.3 | 5.9 | Brain (whole) | 1.1 | 7.3 |
| Liver ca. | 0.0 | 0.0 | Spinal Cord Pool | 4.0 | 0.0 |
| HepG2 | | | | | |
| Kidney Pool | 100.0 | 100.0 | Adrenal Gland | 3.0 | 0.0 |
| Fetal Kidney | 0.0 | 0.0 | Pituitary gland | 1.6 | 0.0 |
| | | | Pool | | |
| Renal ca. 786- | 0.0 | 0.0 | Salivary Gland | 1.8 | 0.0 |
| 0 | | | | | |
| Renal ca. | 0.0 | 0.0 | Thyroid (female) | 1.2 | 0.0 |
| A498 | | | | | |
| Renal ca. | 0.0 | 0.0 | Pancreatic ca. | 2.1 | 2.8 |
| ACHN | | | CAPAN2 | | |
| Renal ca. UO-31 | 0.0 | 0.0 | Pancreas Pool | 58.2 | 44.1 |

TABLE GD
Panel 2.1

| | Rel. Exp. | | Rel. Exp. |
|---|---|---|---|
| | (%) Ag3944, | | (%) Ag3944, |
| | Run | | Run |
| Tissue Name | 170686075 | Tissue Name | 170686075 |
| Normal Colon | 35.6 | Kidney Cancer | 0.0 |
| | | 9010320 | |
| Colon cancer | 0.0 | Kidney margin | 28.9 |
| (OD06064) | | 9010321 | |
| Colon cancer margin | 0.0 | Kidney Cancer | 0.0 |
| (OD06064) | | 8120607 | |
| Colon cancer | 0.0 | Kidney margin | 12.9 |
| (OD06159) | | 8120608 | |
| Colon cancer margin | 13.1 | Normal Uterus | 100.0 |
| (OD06159) | | | |
| Colon cancer | 5.1 | Uterus Cancer | 53.6 |
| (OD06298-08) | | | |
| Colon cancer margin | 28.7 | Normal Thyroid | 1.1 |
| (OD06298-018) | | | |
| Colon Cancer Gr.2 | 8.5 | Thyroid Cancer | 0.0 |
| ascend colon | | | |
| (ODO3921) | | | |
| Colon Cancer margin | 35.6 | Thyroid Cancer | 4.6 |
| (ODO3921) | | A302152 | |
| Colon cancer | 0.0 | Thyroid margin | 12.1 |
| metastasis | | A302153 | |
| (OD06104) | | | |
| Lung margin | 17.2 | Normal Breast | 0.0 |
| (OD06104) | | | |
| Colon mets to lung | 7.0 | Breast Cancer | 0.0 |
| (OD04451-01) | | | |
| Lung margin | 0.0 | Breast Cancer | 0.0 |
| (OD04451-02) | | | |
| Normal Prostate | 6.7 | Breast Cancer | 1.3 |
| | | (OD04590-01) | |
| Prostate Cancer | 10.8 | Breast Cancer Mets | 18.4 |
| (OD04410) | | (OD04590-03) | |
| Prostate margin | 21.2 | Breast Cancer | 0.0 |
| (OD04410) | | Metastasis | |
| Normal Lung | 22.4 | Breast Cancer | 0.0 |
| Invasive poor diff. | 0.0 | Breast Cancer | 1.1 |
| lung adeno 1 | | 9100266 | |
| (ODO4945-01) | | | |
| Lung margin | 14.7 | Breast margin | 1.3 |
| (ODO4945-03) | | 9100265 | |
| Lung Malignant | 1.3 | Breast Cancer | 0.0 |
| Cancer (OD03126) | | A209073 | |
| Lung margin | 17.2 | Breast margin | 4.9 |
| (OD03126) | | A2090734 | |
| Lung Cancer | 0.0 | Normal Liver | 2.1 |
| (OD05014A) | | | |
| Lung margin | 1.2 | Liver Cancer 1026 | 3.0 |
| (OD05014B) | | | |
| Lung Cancer | 0.0 | Liver Cancer 1025 | 5.1 |
| (OD04237-01) | | | |
| Lung margin | 2.5 | Liver Cancer | 6.9 |
| (OD04237-02) | | 6004-T | |
| Ocular Mel Met to | 0.0 | Liver Tissue | 0.0 |
| Liver (ODO4310) | | 6004-N | |
| Liver margin | 0.0 | Liver Cancer | 62.0 |
| (ODO4310) | | 6005-T | |
| Melanoma Mets to | 0.0 | Liver Tissue | 0.0 |
| Lung (OD04321) | | 6005-N | |
| Lung margin | 0.0 | Liver Cancer | 22.5 |
| (OD04321) | | | |
| Normal Kidney | 17.2 | Normal Bladder | 10.2 |
| Kidney Ca, Nuclear | 9.8 | Bladder Cancer | 3.5 |
| grade 2 (OD04338) | | | |
| Kidney margin | 5.2 | Bladder Cancer | 5.2 |
| (OD04338) | | | |
| Kidney Ca Nuclear | 0.0 | Normal Ovary | 27.5 |
| grade ½ (OD04339) | | | |
| Kidney margin | 17.7 | Ovarian Cancer | 4.9 |
| (OD04339) | | | |
| Kidney Ca, Clear | 2.3 | Ovarian cancer | 0.0 |
| cell type (OD04340) | | (OD06145) | |
| Kidney margin | 2.2 | Ovarian cancer | 10.2 |
| (OD04340) | | margin (OD06145) | |
| Kidney Ca, Nuclear | 0.0 | Normal Stomach | 27.4 |
| grade 3 (OD04348) | | | |
| Kidney margin | 0.0 | Gastric Cancer | 5.3 |
| (OD04348) | | 9060397 | |
| Kidney Cancer | 4.4 | Stomach margin | 1.0 |
| (OD04450-01) | | 9060396 | |
| Kidney margin | 2.6 | Gastric Cancer | 10.8 |
| (OD04450-03) | | 9060395 | |
| Kidney Cancer | 0.0 | Stomach margin | 9.9 |
| 8120613 | | 9060394 | |
| Kidney margin | 21.0 | Gastric Cancer | 4.5 |
| 8120614 | | 064005 | |

TABLE GE
Panel 3D

| | Rel. Exp. (%) | | Rel. Exp. (%) |
|---|---|---|---|
| | Ag986, Run | | Ag986, Run |
| Tissue Name | 170189539 | Tissue Name | 170189539 |
| Daoy-Medulloblastoma | 0.0 | Ca Ski-Cervical epidermoid | 0.0 |
| | | carcinoma (metastasis) | |
| TE671-Medulloblastoma | 4.4 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 0.0 | JM1-pre-B-cell lymphoma | 0.0 |
| Cerebellum | 0.0 | Jurkat-T cell leukemia | 2.7 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 54.3 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 2.9 |
| lung cancer | | carcinoma | |
| NCI-H82-Small cell lung | 0.0 | SW 839-Clear cell renal | 0.0 |
| cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.0 |
| carcinoid | | carcinoma (liver metastasis) | |
| NCI-UMC-11-Lung | 100.0 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.6 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE GF
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3944, | Ag986, | | Ag3944, | Ag986, |
| | Run | Run | | Run | Run |
| Tissue Name | 170684833 | 170285024 | Tissue Name | 170684833 | 170285024 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 1.6 | HUVEC IL-11 | 0.0 | 0.0 |
| Secondary Tr1 rest | 0.0 | 0.0 | Lung | 0.0 | 8.0 |
| | | | Microvascular EC | | |
| | | | none | | |
| Primary Th1 act | 0.0 | 0.0 | Lung | 0.0 | 0.0 |
| | | | Microvascular EC | | |
| | | | TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 0.0 | 4.5 | Coronery artery | 0.0 | 0.0 |
| lymphocyte act | | | SMC rest | | |
| CD45RO CD4 | 0.0 | 0.0 | Coronery artery | 0.0 | 1.8 |
| lymphocyte act | | | SMC TNFalpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.0 | 1.6 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 22.2 | 0.0 |
| LAK cells IL-2 +IL- | 0.0 | 0.0 | NCI-H292 none | 0.0 | 0.0 |
| 12 | | | | | |
| LAK cells IL- | 0.0 | 0.0 | NCI-H292 IL-4 | 0.0 | 0.0 |
| 2 + IFN gamma | | | | | |
| LAK cells IL-2 + | 0.0 | 0.0 | NCI-H292 IL-9 | 0.0 | 0.0 |
| IL-18 | | | | | |
| LAK cells | 0.0 | 0.0 | NCI-H292 IL-13 | 7.9 | 0.0 |
| PMA/ionomycin | | | | | |
| NK Cells IL-2 rest | 0.0 | 0.0 | NCI-H292 IFN | 0.0 | 0.0 |
| | | | gamma | | |
| Two Way MLR 3 | 0.0 | 0.0 | HPAEC none | 0.0 | 0.0 |
| day | | | | | |
| Two Way MLR 5 | 0.0 | 0.0 | HPAEC TNF | 0.0 | 0.0 |
| day | | | alpha + IL-1 beta | | |
| Two Way MLR 7 | 0.0 | 0.0 | Lung fibroblast | 0.0 | 0.0 |
| day | | | none | | |
| PBMC rest | 0.0 | 0.0 | Lung fibroblast | 0.0 | 0.0 |
| | | | TNF alpha + IL-1 | | |
| | | | beta | | |
| PBMC PWM | 0.0 | 0.0 | Lung fibroblast | 0.0 | 0.0 |
| | | | IL-4 | | |
| PBMC PHA-L | 0.0 | 0.0 | Lung fibroblast | 0.0 | 0.0 |
| | | | IL-9 | | |
| Ramos (B cell) | 0.0 | 0.0 | Lung fibroblast | 0.0 | 7.4 |
| none | | | IL-13 | | |
| Ramos (B cell) | 0.0 | 0.0 | Lung fibroblast | 0.0 | 0.0 |
| ionomycin | | | IFN gamma | | |
| B lymphocytes | 0.0 | 0.0 | Dermal fibroblast | 0.0 | 0.0 |
| PWM | | | CCD1070 rest | | |
| B lymphocytes | 0.0 | 0.0 | Dermal fibroblast | 0.0 | 0.0 |
| CD40L and IL-4 | | | CCD1070 TNF | | |
| | | | alpha | | |
| EOL-1 dbcAMP | 0.0 | 0.0 | Dermal fibroblast | 0.0 | 0.0 |
| | | | CCD1070 IL-1 | | |
| | | | beta | | |
| EOL-1 dbcAMP | 0.0 | 0.0 | Dermal fibroblast | 0.0 | 0.0 |
| PMA/ionomycin | | | IFN gamma | | |
| Dendritic cells | 0.0 | 0.0 | Dermal fibroblast | 0.0 | 0.0 |
| none | | | IL-4 | | |
| Dendritic cells LPS | 0.0 | 0.0 | Dermal | 0.0 | 0.0 |
| | | | Fibroblasts rest | | |
| Dendritic cells | 0.0 | 0.0 | Neutrophils | 0.0 | 0.0 |
| anti-CD40 | | | TNFa + LPS | | |
| Monocytes | 0.0 | 0.0 | Neutrophils rest | 0.0 | 0.0 |
| rest | | | | | |
| Monocytes LPS | 0.0 | 0.0 | Colon | 100.0 | 4.6 |
| Macrophages rest | 0.0 | 0.0 | Lung | 61.6 | 5.1 |
| Macrophages LPS | 0.0 | 0.0 | Thymus | 44.1 | 32.3 |
| HUVEC none | 0.0 | 0.0 | Kidney | 20.2 | 100.0 |
| HUVEC starved | 0.0 | 0.0 | | | |

CNS_neurodegeneration_v1.0 Summary: Ag986/Ag3944 Expression of the CG94702-01 gene is low/undetectable in all samples on this panel (CTs>35). (Data not shown.)

General_screening_panel_v1.4 Summary: Ag986/Ag3944 Two experiments with two different probe and primer sets produce results that are in excellent agreement, with highest expression of the CG94702-01 gene in the kidney (CTs=32-33). Significant expression appears to be generally associated with normal tisuse and is also seen in samples derived from pancreas, stomach, small intestine, fetal and adult skeletal muscle and colon. Thus, expression of this gene could be used to differentiate between kidney and fetal kidney (CTs=40) and between these samples and the other samples on this panel.

Panel 2.1 Summary: Ag3944 Highest expression of the CG947-02 gene is seen in the uterus (CT=32.8). Overall, expression appears to be higher in normal tissues when compared to expression in cancer as seen in the previous panel. Thus, expression of this gene could be used as a marker of uterine tissue.

Panel 3D Summary: Ag986 Expression of the CG94702-01 gene is restricted to two samples derived from lung cancer cell lines (CTs=32-33). Thus, expression of this gene could be used to differentiate between these samples and other samples on this panel and as a marker to detect the presence of lung cancer. Furthermore, therapeutic modulation of the expression or function of this gene may be effective in the treatment of lung cancer.

Panel 4.1D Summary: Ag986/Ag3944 Two experiments with two different probe and primer sets produce results that are in very good agreement, with expression of the CG94702-01 gene restricted to normal tissue samples derived from colon and kidney (CTs=33.5-34.5). This preferential expression in normal tissue is consistent with expression seen in previous panels and suggests that expression of this gene could be used to differentiate between these samples and other samples on this panel and as a marker for these tissues. Furthermore, expression of this gene is decreased in colon samples from patients with IBD colitis and Crohn's disease relative to normal colon. Therefore, therapeutic modulation of the activity of the protein encoded by this gene may be useful in the treatment of inflammatory bowel disease. Furthermore, therapies designed with the protein encoded by this gene may modulate kidney function and be important in the treatment of inflammatory or autoiminune diseases that affect the kidney, including lupus and glomerulonephritis.

H. NOV10a (COR_CG94661-01): Selectin Like

Expression of gene COR CG94661-01 was assessed using the primer-probe set Ag3954, described in Table HA. Results of the RTQ-PCR runs are shown in Tables HB, HC and HD.

TABLE HA

Probe Name Ag3954

| | | | |
|---|---|---|---|
| Primers | Sequences | Length | Start Position |

| | | | | |
|---|---|---|---|---|
| Forward | 5′-accagccagtcagctacaact-3′ | (Seq ID NO:141) | 21 | 927 |
| Probe | TET-5′-acatcaactccacatgcgcccag-3′-TAMRA | (Seq ID NO:142) | 23 | 950 |
| Reverse | 5′-ctggaagctgattccactcat-3′ | (Seq ID NO:143) | 21 | 983 |

TABLE HB
CNS_neurodegeneration_v1.0

| | Rel. Exp. (%) Ag3954, | | Rel. Exp. (%) Ag3954, |
|---|---|---|---|
| Tissue Name | Run 212347079 | Tissue Name | Run 212347079 |
| AD 1 Hippo | 60.3 | Control (Path) 3 | 7.8 |
| | | Temporal Ctx | |
| AD 2 Hippo | 76.3 | Control (Path) 4 | 12.5 |
| | | Temporal Ctx | |
| AD 3 Hippo | 10.4 | AD 1 Occipital Ctx | 15.1 |
| AD 4 Hippo | 18.2 | AD 2 Occipital Ctx | 0.0 |
| | | (Missing) | |
| AD 5 Hippo | 53.2 | AD 3 Occipital Ctx | 11.4 |
| AD 6 Hippo | 94.6 | AD 4 Occipital Ctx | 5.8 |
| Control 2 Hippo | 25.2 | AD 5 Occipital Ctx | 33.7 |
| Control 4 Hippo | 49.0 | AD 6 Occipital Ctx | 0.0 |
| Control (Path) 3 | 21.9 | Control 1 Occipital | 21.0 |
| Hippo | | Ctx | |
| AD 1 Temporal Ctx | 25.2 | Control 2 Occipital | 45.4 |
| | | Ctx | |
| AD 2 Temporal Ctx | 17.6 | Control 3 Occipital | 8.0 |
| | | Ctx | |
| AD 3 Temporal Ctx | 4.6 | Control 4 Occipital | 3.9 |
| | | Ctx | |
| AD 4 Temporal Ctx | 12.2 | Control (Path) 1 | 26.4 |
| | | Occipital Ctx | |
| AD 5 Inf Temporal | 59.9 | Control (Path) 2 | 3.5 |
| Ctx | | Occipital Ctx | |
| AD 5 Sup Temporal | 100.0 | Control (Path) 3 | 10.9 |
| Ctx | | Occipital Ctx | |
| AD 6 Inf Temporal | 43.8 | Control (Path) 4 | 11.3 |
| Ctx | | Occipital Ctx | |
| AD 6 Sup Temporal | 24.7 | Control 1 Parietal | 15.3 |
| Ctx | | Ctx | |
| Control 1 Temporal | 5.3 | Control 2 Parietal | 75.8 |
| Ctx | | Ctx | |
| Control 2 Temporal | 21.0 | Control 3 Parietal | 11.4 |
| Ctx | | Ctx | |
| Control 3 Temporal | 4.5 | Control (Path) 1 | 82.9 |
| Ctx | | Parietal Ctx | |
| Control 3 Temporal | 5.8 | Control (Path) 2 | 6.3 |
| Ctx | | Parietal Ctx | |
| Control (Path) 1 | 18.3 | Control (Path) 3 | 5.4 |
| Temporal Ctx | | Parietal Ctx | |
| Control (Path) 2 | 28.7 | Control (Path) 4 | 47.0 |
| Temporal Ctx | | Parietal Ctx | |

TABLE HC
General_screening_panel_v1.4

| | Rel. Exp. (%) Ag3954, | | Rel. Exp. (%) Ag3954, |
|---|---|---|---|
| Tissue Name | Run 219479041 | Tissue Name | Run 219479041 |
| Adipose | 4.8 | Renal ca. TK-10 | 7.3 |
| Melanoma* | 0.6 | Bladder | 6.0 |
| Hs688(A).T | | | |
| Melanoma* | 0.7 | Gastric ca. (liver met.) | 14.2 |
| Hs688(B).T | | NCI-N87 | |
| Melanoma* M14 | 0.0 | Gastric ca. KATO III | 0.0 |
| Melanoma* | 0.0 | Colon ca. SW-948 | 0.2 |
| LOXIMVI | | | |
| Melanoma* SK- | 3.2 | Colon ca. SW480 | 9.8 |
| MEL-5 | | | |
| Squamous cell | 0.3 | Colon ca.* (SW480 | 3.5 |
| carcinoma SCC-4 | | met) SW620 | |
| Testis Pool | 35.8 | Colon ca. HT29 | 1.8 |
| Prostate ca.* (bone | 17.2 | Colon ca. HCT-116 | 20.6 |
| met) PC-3 | | | |
| Prostate Pool | 1.5 | Colon ca. CaCo-2 | 15.3 |
| Placenta | 2.0 | Colon cancer tissue | 4.6 |
| Uterus Pool | 3.7 | Colon ca. SW1116 | 4.1 |
| Ovarian ca. | 14.8 | Colon ca. Colo-205 | 0.0 |
| OVCAR-3 | | | |
| Ovarian ca. SK-OV-3 | 1.6 | Colon ca. SW-48 | 0.2 |
| Ovarian ca. | 2.0 | Colon Pool | 19.3 |
| OVCAR-4 | | | |
| Ovarian ca. | 27.0 | Small Intestine Pool | 17.2 |
| OVCAR-5 | | | |
| Ovarian ca. IGROV-1 | 1.4 | Stomach Pool | 5.6 |
| Ovarian ca. | 11.0 | Bone Marrow Pool | 4.1 |
| OVCAR-8 | | | |
| Ovary | 19.3 | Fetal Heart | 0.1 |
| Breast ca. MCF-7 | 9.2 | Heart Pool | 4.0 |
| Breast ca. MDA- | 32.5 | Lymph Node Pool | 15.6 |
| MB-231 | | | |
| Breast ca. BT 549 | 0.8 | Fetal Skeletal Muscle | 1.3 |
| Breast ca. T47D | 100.0 | Skeletal Muscle Pool | 0.4 |
| Breast ca. MDA-N | 0.0 | Spleen Pool | 10.5 |
| Breast Pool | 16.7 | Thymus Pool | 11.9 |
| Trachea | 4.9 | CNS cancer (glio/astro) | 0.1 |
| | | U87-MG | |
| Lung | 14.9 | CNS cancer (glio/astro) | 0.0 |
| | | U-118-MG | |
| Fetal Lung | 5.6 | CNS cancer | 1.4 |
| | | (neuro; met) SK-N-AS | |
| Lung ca. NCI-N417 | 0.0 | CNS cancer (astro) SF- | 0.0 |
| | | 539 | |
| Lung ca. LX-1 | 6.4 | CNS cancer (astro) | 0.0 |
| | | SNB-75 | |
| Lung ca. NCI-H146 | 2.1 | CNS cancer (glio) | 1.1 |
| | | SNB-19 | |
| Lung ca. SHP-77 | 0.3 | CNS cancer (glio) SF- | 0.2 |
| | | 295 | |
| Lung ca. A549 | 0.8 | Brain (Amygdala) Pool | 4.3 |
| Lung ca. NCI-H526 | 0.0 | Brain (cerebellum) | 2.2 |
| Lung ca. NCI-H23 | 3.9 | Brain (fetal) | 0.3 |
| Lung ca. NCI-H460 | 0.5 | Brain (Hippocampus) | 2.6 |
| | | Pool | |
| Lung ca. HOP-62 | 20.9 | Cerebral Cortex Pool | 3.9 |
| Lung ca. NCI-H522 | 0.0 | Brain (Substantia nigra) | 3.5 |
| | | Pool | |
| Liver | 1.6 | Brain (Thalamus) Pool | 4.4 |
| Fetal Liver | 5.6 | Brain (whole) | 1.9 |
| Liver ca. HepG2 | 5.8 | Spinal Cord Pool | 4.4 |
| Kidney Pool | 22.5 | Adrenal Gland | 0.5 |
| Fetal Kidney | 3.9 | Pituitary gland Pool | 3.2 |
| Renal ca. 786-0 | 0.0 | Salivary Gland | 3.8 |
| Renal ca. A498 | 1.0 | Thyroid (female) | 6.8 |
| Renal ca. ACHN | 0.0 | Pancreatic ca. CAPAN2 | 0.4 |
| Renal ca. UO-31 | 1.5 | Pancreas Pool | 17.4 |

TABLE HD
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3954, | Ag3954, | | Ag3954, | Ag3954, |
| | Run | Run | | Run | Run |
| Tissue Name | 170737188 | 171619752 | Tissue Name | 170737188 | 171619752 |
| Secondary Th1 act | 21.9 | 26.1 | HUVEC IL-1beta | 0.0 | 0.4 |
| Secondary Th2 act | 30.6 | 27.9 | HUVEC IFN | 0.2 | 0.0 |
| | | | gamma | | |
| Secondary Tr1 act | 23.7 | 31.6 | HUVEC TNF | 0.0 | 0.0 |
| | | | alpha + IFN | | |
| | | | gamma | | |
| Secondary Th1 rest | 45.4 | 48.6 | HUVEC TNF | 0.0 | 0.0 |
| | | | alpha + IL4 | | |
| Secondary Th2 rest | 24.0 | 36.9 | HUVEC IL-11 | 0.0 | 0.0 |
| Secondary Tr1 rest | 59.5 | 62.4 | Lung | 0.0 | 0.0 |
| | | | Microvascular EC | | |
| | | | none | | |
| Primary Th1 act | 20.0 | 24.3 | Lung | 0.0 | 0.0 |
| | | | Microvascular EC | | |
| | | | TNFalpha + IL- | | |
| | | | 1beta | | |
| Primary Th2 act | 35.6 | 25.3 | Microvascular | 0.0 | 0.0 |
| | | | Dermal EC none | | |
| Primary Tr1 act | 28.9 | 35.1 | Microsvasular | 0.0 | 0.0 |
| | | | Dermal EC | | |
| | | | TNFalpha + IL- | | |
| | | | 1beta | | |
| Primary Th1 rest | 69.3 | 55.5 | Bronchial | 0.3 | 0.0 |
| | | | epithelium | | |
| | | | TNFalpha + | | |
| | | | IL1beta | | |
| Primary Th2 rest | 28.5 | 29.9 | Small airway | 0.2 | 0.0 |
| | | | epithelium none | | |
| Primary Tr1 rest | 44.1 | 56.6 | Small airway | 0.4 | 0.0 |
| | | | epithelium | | |
| | | | TNFalpha + IL- | | |
| | | | 1beta | | |
| CD45RA CD4 | 16.2 | 14.5 | Coronery artery | 0.2 | 0.0 |
| lymphocyte act | | | SMC rest | | |
| CD45RO CD4 | 55.1 | 57.0 | Coronery artery | 0.0 | 0.0 |
| lymphocyte act | | | SMC TNFalpha + | | |
| | | | IL-1beta | | |
| CD8 lymphocyte | 22.4 | 30.8 | Astrocytes rest | 0.2 | 0.4 |
| act | | | | | |
| Secondary CD8 | 40.9 | 42.3 | Astrocytes | 0.5 | 0.0 |
| lymphocyte rest | | | TNFalpha + IL- | | |
| | | | 1beta | | |
| Secondary CD8 | 7.3 | 18.2 | KU-812 | 3.7 | 8.4 |
| lymphocyte act | | | (Basophil) rest | | |
| CD4 lymphocyte | 38.4 | 51.8 | KU-812 | 6.5 | 12.3 |
| none | | | (Basophil) | | |
| | | | PMA/ionomycin | | |
| 2ry | 74.2 | 74.7 | CCD1106 | 0.0 | 0.2 |
| Th1/Th2/Tr1_anti- | | | (Keratinocytes) | | |
| CD95 CH11 | | | none | | |
| LAK cells rest | 19.5 | 21.5 | CCD1106 | 0.7 | 0.5 |
| | | | (Keratinocytes) | | |
| | | | TNFalpha + IL- | | |
| | | | 1beta | | |
| LAK cells IL-2 | 37.9 | 38.7 | Liver cirrhosis | 1.8 | 2.6 |
| LAK cells IL-2 + IL- | 13.4 | 22.8 | NCI-H292 none | 1.8 | 0.8 |
| 12 | | | | | |
| LAK cells IL- | 10.7 | 13.5 | NCI-H292 IL-4 | 1.1 | 1.5 |
| 2 + IFN gamma | | | | | |
| LAK cells IL-2 + | 18.0 | 25.3 | NCI-H292 IL-9 | 0.3 | 0.3 |
| IL-18 | | | | | |
| LAK cells | 6.8 | 5.1 | NCI-H292 IL-13 | 0.7 | 1.3 |
| PMA/ionomycin | | | | | |
| NK Cells IL-2 rest | 13.5 | 16.6 | NCI-H292 IFN | 1.1 | 0.8 |
| | | | gamma | | |
| Two Way MLR 3 | 26.6 | 33.9 | HPAEC none | 0.0 | 0.0 |
| day | | | | | |
| Two Way MLR 5 | 17.8 | 24.5 | HPAEC TNF | 0.0 | 0.0 |
| day | | | alpha + IL-1 beta | | |
| Two Way MLR 7 | 24.0 | 28.5 | Lung fibroblast | 0.8 | 1.4 |
| day | | | none | | |
| PBMC rest | 17.4 | 16.6 | Lung fibroblast | 0.0 | 0.7 |
| | | | TNF alpha + IL-1 | | |
| | | | beta | | |
| PBMC PWM | 17.9 | 28.1 | Lung fibroblast | 0.8 | 0.3 |
| | | | IL-4 | | |
| PBMC PHA-L | 41.2 | 70.2 | Lung fibroblast | 1.8 | 1.2 |
| | | | IL-9 | | |
| Ramos (B cell) | 78.5 | 61.1 | Lung fibroblast | 1.1 | 1.0 |
| none | | | IL-13 | | |
| Ramos (B cell) | 100.0 | 100.0 | Lung fibroblast | 1.0 | 0.9 |
| ionomycin | | | IFN gamma | | |
| B lymphocytes | 20.6 | 27.9 | Dermal fibroblast | 2.8 | 4.5 |
| PWM | | | CCD1070 rest | | |
| B lymphocytes | 42.6 | 53.6 | Dermal fibroblast | 14.1 | 14.9 |
| CD40L and IL-4 | | | CCD1070 TNF | | |
| | | | alpha | | |
| EOL-1 dbcAMP | 0.0 | 0.0 | Dermal fibroblast | 0.3 | 0.0 |
| | | | CCD1070 IL-1 | | |
| | | | beta | | |
| EOL-1 dbcAMP | 0.0 | 0.0 | Dermal fibroblast | 0.0 | 0.2 |
| PMA/ionomycin | | | IFN gamma | | |
| Dendritic cells | 4.5 | 5.0 | Dermal fibroblast | 0.0 | 0.0 |
| none | | | IL-4 | | |
| Dendritic cells LPS | 1.1 | 3.2 | Dermal | 0.2 | 0.5 |
| | | | Fibroblasts rest | | |
| Dendritic cells anti- | 1.4 | 3.3 | Neutrophils | 0.7 | 0.8 |
| CD40 | | | TNFa + LPS | | |
| Monocytes rest | 7.8 | 10.7 | Neutrophils rest | 3.2 | 4.1 |
| Monocytes LPS | 4.8 | 5.5 | Colon | 2.8 | 6.5 |
| Macrophages rest | 7.4 | 7.9 | Lung | 0.7 | 7.0 |
| Macrophages LPS | 5.4 | 6.7 | Thymus | 28.7 | 40.3 |
| HUVEC none | 0.0 | 0.0 | Kidney | 45.1 | 68.8 |
| HUVEC starved | 0.0 | 0.0 | | | |

CNS_neurodegeneration_v1.0 Summary: Ag3954 This panel confirms the expression of the CG94661-101 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3954 Highest expression of the CG94661-01 gene is seen in a breast cancer cell line (CT=27.6). Significant expression is also seen in a cluster of ovarian, breast and colon cancer cell lines. Thus, expression of this gene could be used to differentiate these samples from other samples on this panel and as a marker for the presence of these cancers. Furthermore, therapeutic modulation of the expression or function of this gene may be effective in the treatment of these cancers.

In addition, this gene is expressed at higher levels in the adult kidney (Ct-29.7) and heart (CT=32) when compared to expression in fetal kidney (CT=32.2) and heart (CT=37), respectively. Thus, expression of this gene could be used to differentiate between these two sources of tissues.

Among tissues with metabolic function, this gene is expressed in thyroid, pancreas, fetal skeletal, heart, adult and fetal liver, pituitary and adipose. This widespread expression suggests that this gene product may play a role in normal metabolic and neuroendocrine function and that disregulated expression of this gene may contribute to metabolic diseases (such as obesity and diabetes) or neuroendocrine disorders.

This gene is also expressed at moderate levels in all regions of the central nervous system examined, including amygdala, hippocampus, substantia nigra, thalamus, cerebellum, cerebral cortex, and spinal cord. This gene contains a region homologous to a sushi domain, which is a common motif of protein-protein interactions. These domains are found in receptors with important neuronal function, such as IL-15R and GABARs. Therefore, this gene may have utility as a small molecule or antibody target to modulate CNS processes involved in CNS disorders.

References:

Wei Xq, Orchardson M, Gracie J A, Leung B P, Gao Bm, Guan H, Niedbala W, Paterson G K, McInnes I B, Liew F Y. (2001) The Sushi domain of soluble IL-15 receptor alpha is essential for binding IL-15 and inhibiting inflammatory and allogenic responses in vitro and in vivo. J Immunol Jul. 1, 2001;167(1):277-82

IL-15 is a pleiotropic cytokine that plays important roles in both innate and adaptive immunity. It is associated with a range of immunopathology, including rheumatoid arthritis and allograft rejection. IL-15 functions through the trimeric IL-15R complex, which consists of a high affinity binding alpha-chain and the common IL-2R beta- and gamma-chains. Characterization of IL-15/IL-15R interactions may facilitate the development of improved IL-15 antagonists for therapeutic interventions. We previously constructed soluble murine IL-15Ralpha (sIL-15Ralpha) by deleting the cytoplasmic and transmembrane domains. To localize the functional domain of IL-15Ralpha, we have now constructed various truncated versions of sIL-15Ralpha. The shortest region retaining IL-15 binding activity is a 65-aa sequence spanning the Sushi domain of IL-15Ralpha. Sushi domains, common motifs in protein-protein interactions, contain four cysteines forming two disulfide bonds in a 1-3 and 2-4 pattern. Amino acid substitution of the first or fourth cysteine in sIL-15Ralpha completely abolished its IL-15 binding activity. This also abrogated the ability of sIL-15Ralpha to neutralize IL-15-induced proinflammatory cytokine production and anti-apoptotic response in vitro. Furthermore, the mutant sIL-15Ralpha lost its ability to inhibit carrageenan-induced local inflammation and allogenic cell-induced T cell proliferation and cytokine production in vivo. Thus, the Sushi domain is critical for the functional activity of sIL-15Ralpha.

PMID: 11418660

Panel 4.1D Summary: Ag3954 Two experiments with the same probe and primer set produce results that are in excellent agreement, with highest expression of the CG94661-01 gene in the ionomycin treated B cell line Ramos (CTs=27-29). Overall, this transcript is expressed in hematopietic cells, preferentially on B and T cells. The protein encoded by this transcript includes a sushi domain which is important in protein:protein interactions (see reference in panel 1.4). This protein appears to be similar to selectin and complement activation proteins and thus may function as a receptor or adhesion molecule. Therefore, therapeutic modulation of the protein encoded by this transcript could be important in the treament of inflammation including asthma, emphysema, arthritis, psoriasis, and inflammatory bowel disease.

I. NOV11 (CG94325-01): Novel Nuclear Protein

Expression of gene CG94325-01 was assessed using the primer-probe set Ag3913, described in Table 1A. Results of the RTQ-PCR runs are shown in Table 1B.

TABLE IA

Probe Name Ag3913

| | | | |
|---|---|---|---|
| Primers | Sequences | Length | Start Position |

| | | | | |
|---|---|---|---|---|
| Forward | 5′-ctgaaggcctttggaatttatc-3′ | (Seq ID NO:144) | 22 | 6093 |
| Probe | TET-5′-ccaagatgactaaagtcttcactcacca-3′-TAMRA | (Seq ID NO:145) | 28 | 6139 |
| Reverse | 5′-cttgccatacagagccacttt-3′ | (Seq ID NO:146) | 21 | 6171 |

TABLE IB
General_screening_panel_v1.4

| | Rel. Exp. (%) Ag3913, | | Rel. Exp. (%) Ag3913, |
|---|---|---|---|
| Tissue Name | Run 219174431 | Tissue Name | Run 219174431 |
| Adipose | 1.4 | Renal ca. TK-10 | 17.7 |
| Melanoma* | 4.3 | Bladder | 4.5 |
| Hs688(A).T | | | |
| Melanoma* | 2.9 | Gastric ca. (liver met.) | 8.4 |
| Hs688(B).T | | NCI-N87 | |
| Melanoma* M14 | 35.8 | Gastric ca. KATO III | 100.0 |
| Melanoma* | 22.4 | Colon ca. SW-948 | 9.2 |
| LOXIMVI | | | |
| Melanoma* SK- | 29.9 | Colon ca. SW480 | 67.4 |
| MEL-5 | | | |
| Squamous cell | 27.2 | Colon ca.* (SW480 | 37.1 |
| carcinoma SCC-4 | | met) SW620 | |
| Testis Pool | 40.3 | Colon ca. HT29 | 23.8 |
| Prostate ca.* (bone | 14.6 | Colon ca. HCT-116 | 69.7 |
| met) PC-3 | | | |
| Prostate Pool | 0.2 | Colon ca. CaCo-2 | 31.2 |
| Placenta | 0.2 | Colon cancer tissue | 9.0 |
| Uterus Pool | 0.2 | Colon ca. SW1116 | 8.1 |
| Ovarian ca. | 35.1 | Colon ca. Colo-205 | 9.7 |
| OVCAR-3 | | | |
| Ovarian ca. SK-OV-3 | 96.6 | Colon ca, SW-48 | 5.6 |
| Ovarian ca. | 11.9 | Colon Pool | 0.8 |
| OVCAR-4 | | | |
| Ovarian ca. | 22.7 | Small Intestine Pool | 0.5 |
| OVCAR-5 | | | |
| Ovarian ca. IGROV-1 | 5.3 | Stomach Pool | 1.3 |
| Ovarian ca. | 3.4 | Bone Marrow Pool | 0.6 |
| OVCAR-8 | | | |
| Ovary | 0.7 | Fetal Heart | 7.5 |
| Breast ca. MCF-7 | 21.3 | Heart Pool | 0.2 |
| Breast ca. MDA- | 77.9 | Lymph Node Pool | 1.5 |
| MB-231 | | | |
| Breast ca. BT 549 | 63.7 | Fetal Skeletal Muscle | 2.6 |
| Breast ca. T47D | 27.2 | Skeletal Muscle Pool | 0.0 |
| Breast ca. MDA-N | 20.7 | Spleen Pool | 5.2 |
| Breast Pool | 1.7 | Thymus Pool | 14.0 |
| Trachea | 0.8 | CNS cancer (glio/astro) | 14.4 |
| | | U87-MG | |
| Lung | 0.0 | CNS cancer (glio/astro) | 69.7 |
| | | U-118-MG | |
| Fetal Lung | 9.9 | CNS cancer | 38.4 |
| | | (neuro; met) SK-N-AS | |
| Lung ca. NCI-N417 | 8.9 | CNS cancer (astro) SF- | 34.4 |
| | | 539 | |
| Lung ca. LX-1 | 28.5 | CNS cancer (astro) | 52.1 |
| | | SNB-75 | |
| Lung ca. NCI-H146 | 10.4 | CNS cancer (glio) SNB-19 | 3.8 |
| Lung ca. SHP-77 | 55.1 | CNS cancer (glio) SF- | 2.7 |
| | | 295 | |
| Lung ca. A549 | 23.0 | Brain (Amygdala) Pool | 0.1 |
| Lung ca. NCI-H526 | 11.6 | Brain (cerebellum) | 0.1 |
| Lung ca. NCI-H23 | 37.1 | Brain (fetal) | 4.4 |
| Lung ca. NCI-H460 | 0.6 | Brain (Hippocampus) | 0.0 |
| | | Pool | |
| Lung ca. HOP-62 | 3.4 | Cerebral Cortex Pool | 0.7 |
| Lung ca. NCI-H522 | 20.7 | Brain (Substantia nigra) | 0.1 |
| | | Pool | |
| Liver | 0.0 | Brain (Thalamus) Pool | 0.2 |
| Fetal Liver | 46.7 | Brain (whole) | 0.3 |
| Liver ca. HepG2 | 10.7 | Spinal Cord Pool | 0.1 |
| Kidney Pool | 0.4 | Adrenal Gland | 0.1 |
| Fetal Kidney | 13.6 | Pituitary gland Pool | 0.1 |
| Renal ca. 786-0 | 32.8 | Salivary Gland | 0.0 |
| Renal ca. A498 | 5.4 | Thyroid (female) | 0.1 |
| Renal ca. ACHN | 3.1 | Pancreatic ca. CAPAN2 | 22.2 |
| Renal ca. UO-31 | 5.2 | Pancreas Pool | 1.5 |

General_screening_panel_v1.4 Summary: Ag3913 Highest expression of the CG94325-01 gene is seen in a gastric cancer cell line (CT=27.9). In addition, significant levels of expression are seen in a cluster of samples derived from ovarian, breast, colon, melanoma and lung cancer cell lines. Thus, expression of this gene could be used to differentiate between these samples and other samples on this panel and as a marker to detect the presence of these cancers. In addition, this gene is expressed at much higher levels in all fetal tissues on this panel (CTs=29-31) when compared to expression in the adult counterpart (CTs=35-40). Thus, expression of this gene may be used to differentiate between the fetal and adult sources of brain, liver, lung, skeletal muscle, kidney and heart. Furthermore, this predominant expression in fetal tissues and cancer cell lines further reinforces the suggestion that this gene product may be involved in cellular growth and proliferation. Therefore, therapeutic modulation of the expression or function of this gene may be effective in the treatment of ovarian, breast, colon, melanoma and lung cancers.

Among tissues with metabolic function, this gene is expressed at low levels in adipose and pancreas. This expression among these tissues suggests that this gene product may play a role in normal neuroendocrine and metabolic and that disregulated expression of this gene may contribute to obesity and diabetes.

J. NOV12 (CG94282-01): Novel Notch Domain Containing Protein

Expression of gene CG94282-01 was assessed using the primer-probe set Ag3910, described in Table JA.

TABLE JA

Probe Name Ag3910

| | | | |
|---|---|---|---|
| Primers | Sequences | Length | Start Position |

| | | | | |
|---|---|---|---|---|
| Forward | 5′-acattttcaatgttgcaaaacc-3′ | (Seq ID NO:147) | 22 | 863 |
| Probe | TET-5′-cccttctaccaatgtctcagttgttg-3′-TAMRA | (Seq ID NO:148) | 26 | 891 |
| Reverse | 5′-tgagcaatgtcccatcctta-3′ | (Seq ID NO:149) | 20 | 930 |

CNS_neurodegeneration_v1.0 Summary: Ag3910 Expression of the CG94282-01 gene is low/undetectable in all samples on this panel (CTs>35). (Data not shown.) The amp plot indicates that there is a high probability of a probe failure.

General_screening_panel_v1.4 Summary: Ag3910 Expression of the CG94282-01 gene is low/undetectable in all samples on this panel (CTs>35). (Data not shown.) The amp plot indicates that there is a high probability of a probe failure.

Panel 2.1 Summary: Ag3910 Expression of the CG94282-01 gene is low/undetectable in all samples on this panel (CTs>35). (Data not shown.) The amp plot indicates that there is a high probability of a probe failure.

Panel 4.1D Summary: Ag3910 Expression of the CG94282-01 gene is low/undetectable in all samples on this panel (CTs>35). (Data not shown.) The amp plot indicates that there is a high probability of a probe failure.

K. NOV13 (CG94399-01): BHLH Factor MATH6

Expression of gene CG94399-01 was assessed using the primer-probe set Ag3919, described in Table KA. Results of the RTQ-PCR runs are shown in Tables KB, KC and KD.

TABLE KA

Probe Name Ag3919

| | | | |
|---|---|---|---|
| Primers | Sequences | Length | Start Position |

| | | | | |
|---|---|---|---|---|
| Forward | 5′-gtgacttttgctcctcctgtt-3′ | (Seq ID NO:150) | 21 | 1432 |
| Probe | TET-5′-cccatctcaagccaaagatgagtcag-3′-TAMRA | (Seq ID NO:151) | 26 | 1461 |
| Reverse | 5′-ttccatgagttcctagcagaac-3′ | (Seq ID NO:152) | 22 | 1489 |

TABLE KB
CNS_neurodegeneration_v1.0

| | Rel. Exp. (%) Ag3919, | | Rel. Exp. (%) Ag3919, |
|---|---|---|---|
| Tissue Name | Run 212248495 | Tissue Name | Run 212248495 |
| AD 1 Hippo | 26.1 | Control (Path) 3 | 33.2 |
| | | Temporal Ctx | |
| AD 2 Hippo | 59.0 | Control (Path) 4 | 15.3 |
| | | Temporal Ctx | |
| AD 3 Hippo | 42.9 | AD 1 Occipital Ctx | 29.3 |
| AD 4 Hippo | 21.8 | AD 2 Occipital Ctx | 0.0 |
| | | (Missing) | |
| AD 5 Hippo | 84.1 | AD 3 Occipital Ctx | 38.4 |
| AD 6 Hippo | 99.3 | AD 4 Occipital Ctx | 36.1 |
| Control 2 Hippo | 39.2 | AD 5 Occipital Ctx | 36.9 |
| Control 4 Hippo | 44.4 | AD 6 Occipital Ctx | 40.1 |
| Control (Path) 3 | 32.3 | Control 1 Occipital | 16.8 |
| Hippo | | Ctx | |
| AD 1 Temporal Ctx | 38.7 | Control 2 Occipital | 45.7 |
| | | Ctx | |
| AD 2 Temporal Ctx | 45.7 | Control 3 Occipital | 26.6 |
| | | Ctx | |
| AD 3 Temporal Ctx | 47.3 | Control 4 Occipital | 17.4 |
| | | Ctx | |
| AD 4 Temporal Ctx | 73.7 | Control (Path) 1 | 25.9 |
| | | Occipital Ctx | |
| AD 5 Inf Temporal | 100.0 | Control (Path) 2 | 14.2 |
| Ctx | | Occipital Ctx | |
| AD 5 Sup Temporal | 58.6 | Control (Path) 3 | 42.3 |
| Ctx | | Occipital Ctx | |
| AD 6 Inf Temporal | 81.2 | Control (Path) 4 | 11.4 |
| Ctx | | Occipital Ctx | |
| AD 6 Sup Temporal | 83.5 | Control 1 Parietal | 12.9 |
| Ctx | | Ctx | |
| Control 1 Temporal | 13.4 | Control 2 Parietal | 53.6 |
| Ctx | | Ctx | |
| Control 2 Temporal | 34.2 | Control 3 Parietal | 20.2 |
| Ctx | | Ctx | |
| Control 3 Temporal | 28.3 | Control (Path) 1 | 22.2 |
| Ctx | | Parietal Ctx | |
| Control 3 Temporal | 20.0 | Control (Path) 2 | 17.1 |
| Ctx | | Parietal Ctx | |
| Control (Path) 1 | 26.6 | Control (Path) 3 | 45.7 |
| Temporal Ctx | | Parietal Ctx | |
| Control (Path) 2 | 20.0 | Control (Path) 4 | 24.8 |
| Temporal Ctx | | Parietal Ctx | |

TABLE KC
General_screening_panel_v1.4

| | Rel. Exp. (%) | | Rel. Exp. (%) |
|---|---|---|---|
| | Ag3919, | | Ag3919, |
| Tissue Name | Run 219824477 | Tissue Name | Run 219824477 |
| Adipose | 6.4 | Renal ca. TK-10 | 3.6 |
| Melanoma* | 26.8 | Bladder | 2.5 |
| Hs688(A).T | | | |
| Melanoma* | 66.0 | Gastric ca. (liver met.) | 1.3 |
| Hs688(B).T | | NCI-N87 | |
| Melanoma* M14 | 7.1 | Gastric ca. KATO III | 0.4 |
| Melanoma* | 0.2 | Colon ca. SW-948 | 0.4 |
| LOXIMVI | | | |
| Melanoma* SK- | 7.5 | Colon ca. SW480 | 0.3 |
| MEL-5 | | | |
| Squamous cell | 0.1 | Colon ca.* (SW480 | 1.4 |
| carcinoma SCC-4 | | met) SW620 | |
| Testis Pool | 3.6 | Colon ca. HT29 | 0.6 |
| Prostate ca.* (bone | 0.0 | Colon ca. HCT-116 | 0.4 |
| met) PC-3 | | | |
| Prostate Pool | 3.0 | Colon ca. CaCo-2 | 0.1 |
| Placenta | 2.3 | Colon cancer tissue | 5.0 |
| Uterus Pool | 6.4 | Colon ca. SW1116 | 0.1 |
| Ovarian ca. | 5.5 | Colon ca. Colo-205 | 0.1 |
| OVCAR-3 | | | |
| Ovarian ca. SK-OV-3 | 0.5 | Colon ca. SW-48 | 0.9 |
| Ovarian ca. | 1.0 | Colon Pool | 24.5 |
| OVCAR-4 | | | |
| Ovarian ca. | 26.2 | Small Intestine Pool | 14.9 |
| OVCAR-5 | | | |
| Ovarian ca. IGROV-1 | 0.4 | Stomach Pool | 1.6 |
| Ovarian ca. | 0.2 | Bone Marrow Pool | 10.7 |
| OVCAR-8 | | | |
| Ovary | 16.3 | Fetal Heart | 7.1 |
| Breast ca. MCF-7 | 0.1 | Heart Pool | 12.9 |
| Breast ca. MDA- | 0.9 | Lymph Node Pool | 19.1 |
| MB-231 | | | |
| Breast ca. BT 549 | 33.4 | Fetal Skeletal Muscle | 4.8 |
| Breast ca. T47D | 47.6 | Skeletal Muscle Pool | 18.8 |
| Breast ca. MDA-N | 0.0 | Spleen Pool | 5.8 |
| Breast Pool | 16.0 | Thymus Pool | 4.8 |
| Trachea | 11.3 | CNS cancer (glio/astro) | 4.0 |
| | | U87-MG | |
| Lung | 1.7 | CNS cancer (glio/astro) | 84.7 |
| | | U-118-MG | |
| Fetal Lung | 29.3 | CNS cancer | 100.0 |
| | | (neuro; met) SK-N-AS | |
| Lung ca. NCI-N417 | 33.4 | CNS cancer (astro) SF- | 3.1 |
| | | 539 | |
| Lung ca. LX-1 | 1.7 | CNS cancer (astro) | 49.3 |
| | | SNB-75 | |
| Lung ca. NCI-H146 | 0.1 | CNS cancer (glio) | 0.4 |
| | | SNB-19 | |
| Lung ca. SHP-77 | 14.0 | CNS cancer (glio) SF- | 20.2 |
| | | 295 | |
| Lung ca. A549 | 6.3 | Brain (Amygdala) Pool | 2.5 |
| Lung ca. NCI-H526 | 0.0 | Brain (cerebellum) | 4.5 |
| Lung ca. NCI-H23 | 0.3 | Brain (fetal) | 3.5 |
| Lung ca. NCI-H460 | 0.0 | Brain (Hippocampus) | 3.1 |
| | | Pool | |
| Lung ca. HOP-62 | 0.3 | Cerebral Cortex Pool | 2.5 |
| Lung ca. NCI-H522 | 0.2 | Brain (Substantia nigra) | 3.6 |
| | | Pool | |
| Liver | 12.1 | Brain (Thalamus) Pool | 2.5 |
| Fetal Liver | 0.8 | Brain (whole) | 2.7 |
| Liver ca. HepG2 | 0.0 | Spinal Cord Pool | 5.9 |
| Kidney Pool | 45.7 | Adrenal Gland | 4.0 |
| Fetal Kidney | 5.4 | Pituitary gland Pool | 0.4 |
| Renal ca. 786-0 | 20.2 | Salivary Gland | 1.7 |
| Renal ca. A498 | 0.6 | Thyroid (female) | 40.1 |
| Renal ca. ACHN | 0.0 | Pancreatic ca. CAPAN2 | 0.0 |
| Renal ca. UO-31 | 0.8 | Pancreas Pool | 20.0 |

TABLE KD
Panel 4.1D

| | Rel. Exp. | | Rel. Exp. |
|---|---|---|---|
| | (%) Ag3919, | | (%) Ag3919, |
| | Run | | Run |
| Tissue Name | 170188400 | Tissue Name | 170188400 |
| Secondary Th1 | 1.4 | HUVEC IL-1beta | 7.1 |
| act | | | |
| Secondary Th2 | 3.1 | HUVEC IFN gamma | 9.0 |
| act | | | |
| Secondary Tr1 | 1.8 | HUVEC TNF alpha + | 4.6 |
| act | | IFN gamma | |
| Secondary Th1 | 0.5 | HUVEC TNF alpha + | 4.0 |
| rest | | IL4 | |
| Secondary Th2 | 0.5 | HUVEC IL-11 | 5.3 |
| rest | | | |
| Secondary Tr1 | 0.0 | Lung Microvascular | 32.8 |
| rest | | EC none | |
| Primary Th1 act | 0.0 | Lung Microvascular | 38.2 |
| | | EC TNFalpha + | |
| | | IL-1beta | |
| Primary Th2 act | 1.4 | Microvascular | 20.3 |
| | | Dermal EC none | |
| Primary Tr1 act | 0.0 | Microsvasular | 15.2 |
| | | Dermal EC | |
| | | TNFalpha + IL- | |
| | | 1beta | |
| Primary Th1 rest | 0.0 | Bronchial | 0.7 |
| | | epithelium | |
| | | TNFalpha + | |
| | | IL-1beta | |
| Primary Th2 rest | 0.0 | Small airway | 0.0 |
| | | epithelium none | |
| Primary Tr1 rest | 0.3 | Small airway | 0.0 |
| | | epithelium | |
| | | TNFalpha + | |
| | | IL-1beta | |
| CD45RA CD4 | 28.7 | Coronery artery SMC | 15.2 |
| lymphocyte act | | rest | |
| CD45RO CD4 | 0.0 | Coronery artery SMC | 19.8 |
| lymphocyte act | | TNFalpha + | |
| | | IL-1beta | |
| CD8 lymphocyte | 0.0 | Astrocytes rest | 2.6 |
| act | | | |
| Secondary CD8 | 0.0 | Astrocytes | 3.2 |
| lymphocyte rest | | TNFalpha + IL- | |
| | | 1beta | |
| Secondary CD8 | 0.5 | KU-812 (Basophil) | 0.0 |
| lymphocyte act | | rest | |
| CD4 lymphocyte | 0.0 | KU-812 (Basophil) | 0.0 |
| none | | PMA/ionomycin | |
| 2ry Th1/Th2/Tr1_{—} | 1.6 | CCD1106 | 0.0 |
| anti-CD95 CH11 | | (Keratinocytes) | |
| | | none | |
| LAK cells rest | 0.0 | CCD1106 | 0.0 |
| | | (Keratinocytes) | |
| | | TNFalpha + | |
| | | IL-1beta | |
| LAK cells IL-2 | 0.0 | Liver cirrhosis | 6.3 |
| LAK cells IL-2 + | 0.8 | NCI-H292 none | 82.9 |
| IL-12 | | | |
| LAK cells IL-2 + | 0.0 | NCI-H292 IL-4 | 99.3 |
| IFN gamma | | | |
| LAK cells IL-2 + | 0.0 | NCI-H292 IL-9 | 90.8 |
| IL-18 | | | |
| LAK cells | 0.0 | NCI-H292 IL-13 | 81.2 |
| PMA/ionomycin | | | |
| NK Cells IL-2 | 0.3 | NCI-H292 IFN gamma | 37.1 |
| rest | | | |
| Two Way MLR 3 | 0.3 | HPAEC none | 10.2 |
| day | | | |
| Two Way MLR 5 | 0.0 | HPAEC TNF alpha + | 41.2 |
| day | | IL-1 beta | |
| Two Way MLR 7 | 0.0 | Lung fibroblast | 11.4 |
| day | | none | |
| PBMC rest | 1.4 | Lung fibroblast | 6.9 |
| | | TNF alpha + IL-1 | |
| | | beta | |
| PBMC PWM | 0.0 | Lung fibroblast | 26.1 |
| | | IL-4 | |
| PBMC PHA-L | 0.2 | Lung fibroblast | 12.4 |
| | | IL-9 | |
| Ramos (B cell) | 0.0 | Lung fibroblast | 17.4 |
| none | | IL-13 | |
| Ramos (B cell) | 0.0 | Lung fibroblast IFN | 20.6 |
| ionomycin | | gamma | |
| B lymphocytes | 0.0 | Dermal fibroblast | 100.0 |
| PWM | | CCD1070 rest | |
| B lymphocytes | 0.4 | Dermal fibroblast | 37.9 |
| CD40L and IL-4 | | CCD1070 TNF alpha | |
| EOL-1 dbcAMP | 0.5 | Dermal fibroblast | 33.0 |
| | | CCD1070 IL-1 beta | |
| EOL-1 dbcAMP | 0.0 | Dermal fibroblast | 15.4 |
| PMA/ionomycin | | IFN gamma | |
| Dendritic cells | 0.0 | Dermal fibroblast | 21.8 |
| none | | IL-4 | |
| Dendritic cells | 0.0 | Dermal Fibroblasts | 13.6 |
| LPS | | rest | |
| Dendritic cells | 0.5 | Neutrophils TNFa + | 7.9 |
| anti-CD40 | | LPS | |
| Monocytes rest | 6.4 | Neutrophils rest | 1.3 |
| Monocytes LPS | 3.1 | Colon | 4.0 |
| Macrophages | 0.0 | Lung | 13.6 |
| rest | | | |
| Macrophages LPS | 0.0 | Thymus | 1.7 |
| HUVEC none | 6.2 | Kidney | 5.7 |
| HUVEC starved | 11.1 | | |

CNS_neurodegeneration_v1.0 Summary: Ag3919 This panel does not show differential expression of the CG94399-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3919 Highest expression of the CG94399-01 gene is seen in a brain cancer cell line (CT=28.4). In addition, significant levels of expression are seen in a cluster of samples derived from ovarian, melanoma and lung cancer cell lines. Thus, expression of this gene could be used to differentiate between these samples and other samples on this panel and as a marker to detect the presence of these cancers. Furthermore, therapeutic modulation of the expression or function of this gene may be effective in the treatment of ovarian, melanoma, brain and lung cancers.

Among tissues with metabolic function, this gene is expressed at moderate to low levels in pituitary, adipose, adrenal gland, pancreas, thyroid, liver, and adult and fetal skeletal muscle, and heart. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at low levels in the CNS, including the hippocam pus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, this gene is expressed at much higher levels in fetal lung (CT=30) when compared to expression in the adult lung (CT=34), and at much higher levels in adult liver (CT=31) when compared to expression in fetal liver (CT=35). Thus, expression of this gene may be used to differentiate between the fetal and adult source of these tissue.

Panel 4.1D Summary: Ag3919 Ag3919 Highest expression of the CG94399-01 gene is seen in resting dermal fibroblasts (CT=29.2). Moderate levels of expression are also seen in treated and untreated lung fibroblasts, dermal fibroblasts, lung microvascular endothelium and the mucoepidermoid cell line, NCI-H292. The expression of this gene in cells derived from or within the lung and skin suggests that this gene may be involved in normal conditions as well as pathological and inflammatory lung disorders that include chronic obstructive pulmonary disease, asthma, allergy, psoriasis and emphysema.

L. NOV15 (CG95387-02): LRR Protein

Expression of gene CG95387-02 was assessed using the primer-probe set Ag4112, described in Table LA. Results of the RTQ-PCR runs are shown in Tables LB and LC.

TABLE LA

Probe Name Ag4112

| | | | |
|---|---|---|---|
| Primers | Sequences | Length | Start Position |

| | | | | |
|---|---|---|---|---|
| Forward | 5′-gctaggattacagccctcattc-3′ | (Seq ID NO:153) | 22 | 44 |
| Probe | TET-5′-tcttttgctcctcaggtgacacagga-3′-TAMRA | (Seq ID NO:154) | 26 | 66 |
| Reverse | 5′-taagttctcctggagctcatga-3′ | (Seq ID NO:155) | 22 | 113 |

TABLE LB
General_screening_panel_v1.4

| | Rel. Exp. (%) Ag4112, | | Rel. Exp. (%) Ag4112, |
|---|---|---|---|
| Tissue Name | Run 219542688 | Tissue Name | Run 219542688 |
| Adipose | 0.8 | Renal ca. TK-10 | 21.8 |
| Melanoma* | 6.3 | Bladder | 10.8 |
| Hs688(A).T | | | |
| Melanoma* | 2.6 | Gastric ca. (liver met.) | 37.1 |
| Hs688(B).T | | NCI-N87 | |
| Melanoma* M14 | 4.4 | Gastric ca. KATO III | 38.2 |
| Melanoma* | 2.0 | Colon ca. SW-948 | 10.7 |
| LOXIMVI | | | |
| Melanoma* SK- | 2.9 | Colon ca. SW480 | 26.6 |
| MEL-5 | | | |
| Squamous cell | 7.9 | Colon ca.* (SW480 | 10.2 |
| carcinoma SCC-4 | | met) SW620 | |
| Testis Pool | 0.9 | Colon ca. HT29 | 20.0 |
| Prostate ca.* (bone | 27.7 | Colon ca. HCT-116 | 35.6 |
| met) PC-3 | | | |
| Prostate Pool | 0.8 | Colon ca. CaCo-2 | 16.5 |
| Placenta | 6.7 | Colon cancer tissue | 8.2 |
| Uterus Pool | 0.0 | Colon ca. SW1116 | 15.9 |
| Ovarian ca. | 14.5 | Colon ca. Colo-205 | 3.7 |
| OVCAR-3 | | | |
| Ovarian ca. SK-OV-3 | 16.6 | Colon ca. SW-48 | 6.0 |
| Ovarian ca. | 12.3 | Colon Pool | 1.7 |
| OVCAR-4 | | | |
| Ovarian ca. | 34.2 | Small Intestine Pool | 2.5 |
| OVCAR-5 | | | |
| Ovarian ca. IGROV-1 | 31.9 | Stomach Pool | 0.0 |
| Ovarian ca. | 14.4 | Bone Marrow Pool | 3.4 |
| OVCAR-8 | | | |
| Ovary | 2.5 | Fetal Heart | 0.0 |
| Breast ca. MCF-7 | 25.5 | Heart Pool | 0.0 |
| Breast ca. MDA- | 21.9 | Lymph Node Pool | 3.7 |
| MB-231 | | | |
| Breast ca. BT 549 | 33.2 | Fetal Skeletal Muscle | 2.6 |
| Breast ca. T47D | 100.0 | Skeletal Muscle Pool | 0.0 |
| Breast ca. MDA-N | 15.2 | Spleen Pool | 1.0 |
| Breast Pool | 2.5 | Thymus Pool | 7.0 |
| Trachea | 4.0 | CNS cancer (glio/astro) | 66.9 |
| | | U87-MG | |
| Lung | 0.0 | CNS cancer (glio/astro) | 21.5 |
| | | U-118-MG | |
| Fetal Lung | 3.8 | CNS cancer | 4.0 |
| | | (neuro; met) SK-N-AS | |
| Lung ca. NCI-N417 | 1.0 | CNS cancer (astro) SF- | 7.8 |
| | | 539 | |
| Lung ca. LX-1 | 13.8 | CNS cancer (astro) | 57.0 |
| | | SNB-75 | |
| Lung ca. NCI-H146 | 4.8 | CNS cancer (glio) | 15.8 |
| | | SNB-19 | |
| Lung ca. SHP-77 | 13.5 | CNS cancer (glio) SF- | 16.8 |
| | | 295 | |
| Lung ca. A549 | 17.8 | Brain (Amygdala) Pool | 1.2 |
| Lung ca. NCI-H526 | 7.9 | Brain (cerebellum) | 2.5 |
| Lung ca. NCI-H23 | 33.2 | Brain (fetal) | 2.6 |
| Lung ca. NCI-H460 | 16.5 | Brain (Hippocampus) | 2.4 |
| | | Pool | |
| Lung ca. HOP-62 | 12.9 | Cerebral Cortex Pool | 0.0 |
| Lung ca. NCI-H522 | 7.3 | Brain (Substantia nigra) | 1.1 |
| | | Pool | |
| Liver | 0.0 | Brain (Thalamus) Pool | 3.3 |
| Fetal Liver | 3.7 | Brain (whole) | 1.3 |
| Liver ca. HepG2 | 10.7 | Spinal Cord Pool | 0.0 |
| Kidney Pool | 2.2 | Adrenal Gland | 8.5 |
| Fetal Kidney | 9.2 | Pituitary gland Pool | 1.3 |
| Renal ca. 786-0 | 12.6 | Salivary Gland | 8.2 |
| Renal ca. A498 | 13.2 | Thyroid (female) | 1.1 |
| Renal ca. ACHN | 15.7 | Pancreatic ca. CAPAN2 | 18.9 |
| Renal ca. UO-31 | 14.1 | Pancreas Pool | 11.5 |

TABLE LC
Panel 4.1D

| | Rel. Exp. (%) | | Rel. Exp. (%) |
|---|---|---|---|
| | Ag4112, Run | | Ag4112, Run |
| Tissue Name | 172775182 | Tissue Name | 172775182 |
| Secondary Th1 act | 0.0 | HUVEC IL-1beta | 0.0 |
| Secondary Th2 act | 0.0 | HUVEC IFN gamma | 6.5 |
| Secondary Tr1 act | 2.1 | HUVEC TNF alpha + IFN | 1.3 |
| | | gamma | |
| Secondary Th1 rest | 0.0 | HUVEC TNF alpha + IL4 | 2.2 |
| Secondary Th2 rest | 0.0 | HUVEC IL-11 | 2.0 |
| Secondary Tr1 rest | 1.8 | Lung Microvascular EC | 1.8 |
| | | none | |
| Primary Th1 act | 2.0 | Lung Microvascular EC | 0.0 |
| | | TNFalpha + IL-1beta | |
| Primary Th2 act | 2.1 | Microvascular Dermal EC | 0.0 |
| | | none | |
| Primary Tr1 act | 4.4 | Microsvasular Dermal EC | 1.4 |
| | | TNFalpha + IL-1beta | |
| Primary Th1 rest | 1.7 | Bronchial epithelium | 0.0 |
| | | TNFalpha + IL-1beta | |
| Primary Th2 rest | 0.0 | Small airway epithelium | 0.0 |
| | | none | |
| Primary Tr1 rest | 0.0 | Small airway epithelium | 7.6 |
| | | TNFalpha + IL-1beta | |
| CD45RA CD4 | 0.0 | Coronery artery SMC rest | 7.6 |
| lymphocyte act | | | |
| CD45RO CD4 | 4.0 | Coronery artery SMC | 0.0 |
| lymphocyte act | | TNFalpha + IL-1beta | |
| CD8 lymphocyte act | 0.0 | Astrocytes rest | 8.9 |
| Secondary CD8 | 2.3 | Astrocytes TNFalpha + | 2.3 |
| lymphocyte rest | | IL-1beta | |
| Secondary CD8 | 6.2 | KU-812 (Basophil) rest | 4.1 |
| lymphocyte act | | | |
| CD4 lymphocyte none | 0.0 | KU-812 (Basophil) | 4.4 |
| | | PMA/ionomycin | |
| 2ry Th1/Th2/Tr1_{—} | 3.1 | CCD1106 (Keratinocytes) | 2.1 |
| anti-CD95 CH11 | | none | |
| LAK cells rest | 1.8 | CCD1106 (Keratinocytes) | 4.1 |
| | | TNFalpha + IL-1beta | |
| LAK cells IL-2 | 4.4 | Liver cirrhosis | 0.0 |
| LAK cells IL-2 + | 4.3 | NCI-H292 none | 23.2 |
| IL-12 | | | |
| LAK cells IL-2 + | 0.0 | NCI-H292 IL-4 | 18.6 |
| IFN gamma | | | |
| LAK cells IL-2 + | 2.1 | NCI-H292 IL-9 | 34.9 |
| IL-18 | | | |
| LAK cells | 0.0 | NCI-H292 IL-13 | 17.2 |
| PMA/ionomycin | | | |
| NK Cells IL-2 rest | 2.0 | NCI-H292 IFN gamma | 22.4 |
| Two Way MLR 3 day | 4.5 | HPAEC none | 2.1 |
| Two Way MLR 5 day | 0.0 | HPAEC TNF alpha + IL-1 | 6.8 |
| | | beta | |
| Two Way MLR 7 day | 0.0 | Lung fibroblast none | 4.5 |
| PBMC rest | 0.0 | Lung fibroblast TNF | 4.2 |
| | | alpha + IL-1 beta | |
| PBMC PWM | 0.0 | Lung fibroblast IL-4 | 2.0 |
| PBMC PHA-L | 2.9 | Lung fibroblast IL-9 | 1.7 |
| Ramos (B cell) none | 2.5 | Lung fibroblast IL-13 | 2.3 |
| Ramos (B cell) | 5.8 | Lung fibroblast IFN gamma | 1.7 |
| ionomycin | | | |
| B lymphocytes PWM | 0.0 | Dermal fibroblast | 4.2 |
| | | CCD1070 rest | |
| B lymphocytes CD40L and | 0.0 | Dermal fibroblast | 10.2 |
| IL-4 | | CCD1070 TNF alpha | |
| EOL-1 dbcAMP | 2.4 | Dermal fibroblast | 7.7 |
| | | CCD1070 IL-1 beta | |
| EOL-1 dbcAMP | 0.0 | Dermal fibroblast IFN | 2.4 |
| PMA/ionomycin | | gamma | |
| Dendritic cells none | 0.0 | Dermal fibroblast IL-4 | 4.1 |
| Dendritic cells LPS | 2.7 | Dermal Fibroblasts rest | 7.9 |
| Dendritic cells anti- | 5.6 | Neutrophils TNFa + LPS | 0.0 |
| CD40 | | | |
| Monocytes rest | 0.0 | Neutrophils rest | 0.0 |
| Monocytes LPS | 0.0 | Colon | 0.0 |
| Macrophages rest | 0.0 | Lung | 2.4 |
| Macrophages LPS | 0.0 | Thymus | 15.0 |
| HUVEC none | 2.0 | Kidney | 100.0 |
| HUVEC starved | 2.1 | | |

CNS_neurodegeneration_v1.0 Summary: Ag4112 Expression of the CG95387-01 gene is low/undetectable in all samples on this panel (CTs>35). (Data not shown.)

General_screening_panel_v1.4 Summary: Ag4112 Expression of the CG95387-01 gene is highest in the T47D breast cancer cell line (CT=31.7), an estrogen receptor positive cell line. In addition, low but significant levels of expression are seen in a cluster of samples derived from prostate, ovarian, gastric, brain, colon, and lung cancer cell lines. Thus, expression of this gene could be used to differentiate between these samples and other samples on this panel and as a marker to detect the presence of these cancers. Overall, expression of this gene appears to be associated with the cancer cell lines when compared to expression in the normal samples. This gene encodes a protein with homology to a leucine rich repeat protein. This motif is believed to participate in protein-protein interactions. A novel member of the leucine rich repeat family has been shown to be upregulated in esrogen positive breast cancers. Therefore based on the published literature and the expression of this gene, therapeutic modulation of the expression or function of this gene may be effective in the treatment of breast, prostate, ovarian, gastric, brain, colon, and lung cancers.

References:

Charpentier A H, Bednarek A K, Daniel R L, Hawkins K A, Laflin K J, Gaddis S, MacLeod M C, Aldaz C M. Effects of estrogen on global gene expression: identification of novel targets of estrogen action. Cancer Res Nov. 1, 2000;60(21):5977-83

The important role played by the sex hormone estrogen in disease and physiological processes has been well documented. However, the mechanisms by which this hormone elicits many of its normal as well as pathological effects are unclear. To identify both known and unknown genes that are regulated by or associated with estrogen action, we performed serial analysis of gene expression on estrogen-responsive breast cancer cells after exposure to this hormone. We examined approximately 190,000 mRNA transcripts and monitored the expression behavior of 12,550 genes. Expression levels for the vast majority of those transcripts were observed to remain constant upon 17beta estradiol (E2) treatment. Only approximately 0.4% of the genes showed an increase in expression of > or =3-fold by 3 h post-E2 treatment. We cloned five novel genes (E2IG1-5), which were observed up-regulated by the hormonal treatment. Of these the most highly induced transcript, E2IG1, appears to be a novel member of the family of small heat shock proteins. The E2IG4 gene is a new member of the large family of leucine-rich repeat-containing proteins. On the basis of architectural and domain homology, this gene appears to be a good candidate for secretion in the extracellular environment and, therefore, may play a role in breast tissue remodeling and/or epithelium-stroma interactions. Several interesting genes with a potential role in the regulation of cell cycle progression were also identified to increase in expression, including Pescadillo and chaperonin CCT2. Two putative paracrine/autocrine factors of potential importance in the regulation of the growth of breast cancer cells were identified to be highly up-regulated by E2: stanniocalcin 2, a calcium/phosphate homeostatic hormone; and inhibin-beta B, a TGF-beta-like factor. Interestingly, we also determined that E2IGI and stanniocalcin 2 were exclusively overexpressed in estrogen-receptor-positive breast cancer lines, and thus they have the potential to serve as breast cancer biomarkers. This data provides a comprehensive view of the changes induced by E2 on the transcriptional program of human E2-responsive cells, and it also identifies novel and previously unsuspected gene targets whose expression is affected by this hormone.

PMID: 11085516

Panel 4.1D Summary: Ag4112 Expression of the CG95387-01 gene is restricted to the kidney (CT=33.7). The putative protein encoded by this gene could allow cells within the kidney to respond to specific microenvironmental signals. Therefore, therapies designed with the protein encoded for by this gene could modulate kidney function and be important in the treatment of inflammatory or autoimmune diseases that affect the kidney, including lupus and glomerulonephritis.

### Example 3

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8): 1249-1265, 2000).

Variants are reported individually but any combination of all or a select subset of variants are also included as contemplated NOVX embodiments of the invention.

NOV1 SNP Data:

In the following positions, one or more consensus positions (Cons. Pos.) of the nucleotide sequence have been identified as SNPs. NOV1 has one SNP variant (Variant 13377181), whose variant positions for its nucleotide and amino acid sequences is numbered according to SEQ ID NOS:1 and 2, respectively. The nucleotide sequence of the NOV1 variant differs as shown in Table 18.

TABLE 18
cSNP and Coding Variants for NOV1

| | Nucleotides | | | Amino Acids | | |
|---|---|---|---|---|---|---|
| Variant | Position | Initial | Modified | Position | Initial | Modified |
| 13377181 | 2496 | G | A | 828 | Gly | Arg |

NOV5 SNP Data:

In the following positions, one or more consensus positions (Cons. Pos.) of the nucleotide sequence have been identified as SNPs. NOV5 has single SNP variant (Variant 13377186), whose variant positions for its nucleotide and amino acid sequences is numbered according to SEQ ID NOS:9 and 10, respectively. The nucleotide sequence of the NOV5 variant differs as shown in Table 19.

TABLE 19
cSNP and Coding Variants for NOV5

| | Nucleotides | | | Amino Acids | | |
|---|---|---|---|---|---|---|
| Variant | Position | Initial | Modified | Position | Initial | Modified |
| 13377186 | 609 | G | A | 180 | Val | Val |

NOV8 SNP Data:

In the following positions, one or more consensus positions (Cons. Pos.) of the nucleotide sequence have been identified as SNPs. NOV8 has two SNP variants (Variant 1377197 and Variant 13377196), whose variant positions for its nucleotide and amino acid sequences is numbered according to SEQ ID NOS:15 and 16, respectively. The nucleotide sequence of the NOV8 variant differs as shown in Table 20.

TABLE 20
cSNP and Coding Variants for NOV8

| | Nucleotides | | | Amino Acids | | |
|---|---|---|---|---|---|---|
| Variant | Position | Initial | Modified | Position | Initial | Modified |
| 13377197 | 3048 | C | T | 1011 | Tyr | Tyr |
| 13377196 | 3799 | C | G | 0 | | |

NOV9 SNP Data:

In the following positions, one or more consensus positions (Cons. Pos.) of the nucleotide sequence have been identified as SNPs. NOV9 has a single SNP variant (Variant 13377850), whose variant positions for its nucleotide and amino acid sequences is numbered according to SEQ ID NOS:17 and 18, respectively. The nucleotide sequence of the NOV9 variants differs as shown in Table 21.

TABLE 21
cSNP and Coding Variants for NOV9

| | Nucleotides | | | Amino Acids | | |
|---|---|---|---|---|---|---|
| Variant | Position | Initial | Modified | Position | Initial | Modified |
| 13377850 | 11676 | G | A | 3892 | Ala | Ala |

NOV10b SNP Data:

In the following positions, one or more consensus positions (Cons. Pos.) of the nucleotide sequence have been identified as SNPs. NOV10b has seven SNP variants, whose variants positions for its nucleotide and amino acid sequences is numbered according to SEQ ID NOS:21 and 22, respectively. The nucleotide sequence of the NOV10b variant differs as shown in Table 22.

TABLE 22
cSNP and Coding Variants for NOV10b

| NT Position of | | |
|---|---|---|
| cSNP | Wild Type NT | Variant NT |
| 125 | C | T |
| 126 | G | C |
| 220 | T | C |
| 242 | C | T |
| 426 | C | T |
| 439 | A | G |
| 451 | T | C |

NOV11 SNP Data:

In the following positions, one or more consensus positions (Cons. Pos.) of the nucleotide sequence have been identified as SNPs. NOV11 has two SNP variants (Variant 13377199 and Variant 13377200), whose variant positions for its nucleotide and amino acid sequences is numbered according to SEQ ID NOS:23 and 24, respectively. The nucleotide sequence of the NOV11 variant differs as shown in Table 23.

TABLE 23
cSNP and Coding Variants for NOV11

| | Nucleotides | | | Amino Acids | | |
|---|---|---|---|---|---|---|
| Variant | Position | Initial | Modified | Position | Initial | Modified |
| 13377199 | 8495 | C | T | 0 | | |
| 13377200 | 8588 | C | A | 0 | | |

NOV12 SNP Data:

In the following positions, one or more consensus positions (Cons. Pos.) of the nucleotide sequence have been identified as SNPs. NOV12 has one SNP variant (Variant 13377201), whose variant positions for its nucleotide and amino acid sequences is numbered according to SEQ ID NOS:25 and 26, respectively. The nucleotide sequence of the NOV12 variant differs as shown in Table 24.

TABLE 24
cSNP and Coding Variants for NOV12

| | Nucleotides | | | Amino Acids | | |
|---|---|---|---|---|---|---|
| Variant | Position | Initial | Modified | Position | Initial | Modified |
| 13377201 | 7898 | T | C | 0 | | |

NOV13 SNP Data:

In the following positions, one or more consensus positions (Cons. Pos.) of the nucleotide sequence have been identified as SNPs. NOV13 has two SNP variants (Variant 13377852 and Variant 13377851), whose variant positions for its nucleotide and amino acid sequences is numbered according to SEQ ID NOS:27 and 28, respectively. The nucleotide sequence of the NOV13 variant differs as shown in Table 25.

TABLE 25
cSNP and Coding Variants for NOV13

| | Nucleotides | | | Amino Acids | | |
|---|---|---|---|---|---|---|
| Variant | Position | Initial | Modified | Position | Initial | Modified |
| 13377852 | 1396 | G | A | 0 | | |
| 13377851 | 1833 | G | A | 0 | | |

NOV15 SNP Data:

In the following positions, one or more consensus positions (Cons. Pos.) of the nucleotide sequence have been identified as SNPs. NOV15 has seven SNP variants, whose variant positions for its nucleotide and amino acid sequences is numbered according to SEQ ID NOS:31 and 32, respectively. The nucleotide sequence of the NOV15 variant differs as shown in Table 26.

TABLE 26
cSNP and Coding Variants for NOV15

| | Nucleotides | | | Amino Acids | | |
|---|---|---|---|---|---|---|
| Variant | Position | Initial | Modified | Position | Initial | Modified |
| 13377204 | 993 | A | G | 222 | Thr | Ala |
| 13377205 | 1114 | G | T | 262 | Ser | Ile |
| 3377206 | 1151 | C | G | 274 | Thr | Thr |
| 13377207 | 1262 | G | A | 311 | Glu | Glu |
| 13377208 | 2061 | T | A | 578 | Ser | Thr |
| 13377209 | 2112 | C | T | 595 | Leu | Leu |
| 13377210 | 2530 | G | C | 0 | | |

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, and 34; and
(d) a variant of an amino acid sequence selected from the group consisting of SEQ ID NOS:2, 4, 6, 8, 10, 12, 14, 16, 18, 20, 22, 24, 26, 28, 30, 32, and 34 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 26, 28, 30, 32, and 34.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 31, and 33.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, and 34;
(d) a variant of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, and 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, and 33.

9. The nucleic acid molecule of claim 5, wherein said nucleic acid molecule comprises a nucleotide sequence selected from the group consisting of
(a) a nucleotide sequence selected from the group consisting of SEQ ID NOS:1, 3, 5, 7, 9, 11, 13, 15, 17, 19, 21, 23, 25, 27, 29, 31, and 33;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31, and 33, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7,9, 11, 13, 15, 17, 19, 21, 23, 25, 27, 29, 31, and 33, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat) or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, and 34, or a biologically active fragment thereof.

41. A method of treating a pathological state in a mammal, the method comprising administering to the mammal the antibody of claim 15 in an amount sufficient to alleviate the pathological state.
